(19) United States
(12) Patent Application Publication
(10) Pub. No.: US 2003/0229079 A1
(43) Pub. Date: Dec. 11, 2003

(54) 1-(Aromatic- or heteroaromatic-substituted)-3-(heteroaromatic substituted)-1,3-propanediones and uses thereof

(76) Inventors: Linda S. Payne, Lansdale, PA (US); Lekhanh O. Tran, Norristown, PA (US); Linghang H. Zhuang, Chalfont, PA (US); Steven D. Young, Lansdale, PA (US); Melissa S. Egbertson, Ambler, PA (US); John S. Wai, Harleysville, PA (US); Mark W. Embrey, North Wales, PA (US); Thorsten E. Fisher, Hatfield, PA (US); James P. Guare, Quakertown, PA (US); H. Marie Langford, Lansdale, PA (US); Jeffrey Y. Melamed, Warminster, PA (US); David L. Clark, Albany, CA (US)

Correspondence Address: MERCK AND CO INC, P O BOX 2000, RAHWAY, NJ, 070650907

(21) Appl. No.: 10/260,029

(22) Filed: Jun. 6, 2003

Related U.S. Application Data
(63) Continuation of application No. 09/598,847, filed on Jun. 21, 2000, now abandoned.
(60) Provisional application No. 60/141,035, filed on Jun. 25, 1999.

(51) Int. Cl. A61K 31/542; A61K 31/538; A61K 31/522; A61K 31/498; A61K 31/506; A61K 31/497; A61K 31/4439

(52) U.S. Cl. 514/222.2; 514/230.5; 514/256; 514/255.05; 514/263.22; 514/341; 544/47; 544/105; 544/277; 544/331; 544/405; 544/350; 546/268.1; 514/249

(57) ABSTRACT

Certain 1-(aromatic- or heteroaromatic-substituted-3-(heteroaromatic substituted)-1,3-propanediones are described as inhibitors of HIV integrase and inhibitors of HIV replication. These compounds are useful in the prevention or treatment of infection by HIV and the treatment of AIDS, either as compounds, pharmaceutically acceptable salts, pharmaceutical composition ingredients, whether or not in combination with other antivirals, immunomodulators, antibiotics or vaccines. Methods of treating AIDS and methods of preventing or treating infection by HIV are also described.

This application claims the benefit of U.S. Provisional Application No. 60/141,035, filed Jun. 25, 1999, the disclosure of which is hereby incorporated by reference in its entirety.

### FIELD OF THE INVENTION

The present invention is directed to 1-(aromatic- or heteroaromatic-substituted)-3-(heteroaromatic substituted)-1,3-propanediones or tautomers thereof, their pharmaceutically acceptable salts, their synthesis, and their use as inhibitors of the HIV integrase enzyme. The compounds of the present invention are useful for preventing or treating infection by HIV and for treating AIDS.

References are made throughout this application to various publications in order to more fully describe the state of the art to which this invention pertains. The disclosures of these references are hereby incorporated by reference in their entireties.

### BACKGROUND OF THE INVENTION

A retrovirus designated human immunodeficiency virus (HIV) is the etiological agent of the complex disease that includes progressive destruction of the immune system (acquired immune deficiency syndrome; AIDS) and degeneration of the central, and peripheral, nervous system. This virus was previously known as LAV, HTLV-III, or ARV. A common feature of retrovirus replication is the insertion by virally-encoded integrase of proviral, DNA into the host cell genome, a required step in HIV replication in human T-lymphoid and monocytoid cells. Integration is believed to be mediated by integrase in three steps: assembly of a stable nucleoprotein complex with viral, DNA sequences; cleavage of two nucleotides from the 3′ termini of the linear proviral, DNA; covalent joining of the recessed 3′ OH termini of the proviral, DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277(1985)]. Amino acid sequence homology provides evidence that the pol sequence encodes reverse transcriptase, integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral, compounds which act as inhibitors of HIV replication are effective agents in the treatment of AIDS and similar diseases, e.g., azidothymidine or AZT. Applicants demonstrate that the compounds of this invention are inhibitors of HIV integrase and inhibitors of HIV replication. The applicants additionally demonstrate that inhibition of integrase in vitro and HIV replication in cells is a direct result of inhibiting the strand transfer reaction catalyzed by the recombinant integrase in vitro in HIV infected cells. The particular advantage of the present invention is highly specific inhibition of HIV integrase and HIV replication. The compounds of the present invention inhibit integrases of closely related lentiviruses such as HIV 2 and SIV, but not integrases from more distantly related retroviruses, for example RSV. These compounds do not inhibit binding or catalysis of other nucleic acid binding proteins, including enzymatic reactions such as those catalyzed by HIV reverse transcriptase, HIV Rnase H, Influenza transcriptase, Hepatitis C polymerase, Yeast DNA polymerase, DNase I, Eco RI endonuclease, or mammalian polymerase II.

Zhao et al. (J. Med Chem., vol. 40, pp. 937-941 and 1186-1194 (1997)) describe hydrazide and arylamide HIV integrase inhibitors. LaFeminia et al. (Antimicrobial Agents & Chemotherapy, vol. 39, no. 2, pp. 320-324, February 1995) describe bis-catechols useful for inhibiting HIV integrase. Lin et al. (J. Med. Chem., vol. 42, pp. 1401-1414 (1999)) describe chicoric acid analogues as HIV-1 integrase inhibitors.

U.S. Pat. No. 4,937,370 discloses certain 1,3-diaryl-1,3-propanediones and their use as sunscreen agents.

At the National Institutes of Health AIDS Structural, Biology Meeting held Jun. 9-11, 1999 at the Lister Hill Auditorium, David R. Davies disclosed 1-(5-chloroindol-3-yl)-3-(5-tetrazolyl)-1,3-propanedione as an integrase inhibitor. 1-(5-Chloroindol-3-yl)-3-(5-tetrazolyl)-1,3-propanedione is also disclosed in Goldgur et al., Proc. Nat'l Acad. Sci. U.S.A. 1999, vol. 96, pp. 13040-13043. A related document is WO 99/50245 (Fujishita et al.) which discloses the prparation of indole derivatives with antiviral, activity.

### SUMMARY OF THE INVENTION

The present invention provides a novel group of 1,3-propanedione derivatives which are potent inhibitors of HIV integrase. These compounds are useful in the inhibition of HIV integrase, the prevention of infection by HIV, the treatment of infection by HIV and in the treatment of AIDS and/or ARC, either as compounds, pharmaceutically acceptable salts or hydrates (when appropriate), pharmaceutical composition ingredients, whether or not in comnbination with other antivirals, anti-infectives, immunomodulators, antibiotics or vaccines. More particularly, the present invention includes a compound of Formula (I):

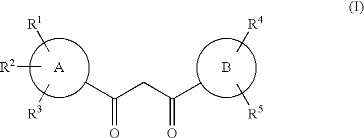

or a tautomer thereof; wherein

A is (i) a benzene ring; (ii) an 8- to 10-membered fused bicyclic carbocycle, wherein the ring of the carbocycle attached to the central, dione moiety is a benzene ring, and the other ring of the carbocycle is saturated or unsaturated; (iii) an 8- to 10-membered fused bicyclic heterocycle containing carbon atoms and from 1 to 3 heteroatoms selected from nitrogen, oxygen and sulfur, wherein the ring of the heterocycle attached to the central, dione moiety is a benzene ring, and the other ring of the heterocycle is a saturated or unsaturated heteroatom-containing ring; or (iv) a 5- or 6-membered heteroaromatic ring containing from 1 to 3 heteroatoms selected from nitrogen, oxygen and sulfur; and wherein A is attached to the central, dione moiety via a carbon atom;

R¹, R² and R³ are substituents attached to nitrogen or carbon in A;

R¹ is hydrogen, halo, nitro, C₁-C₆ alkyl, C₁-C₆ alkoxy, fluorinated C₁-C₆ alkyl, fluorinated C₁-C₆ alkoxy, C₂-C₈ alkoxyalkyl, fluorinated C₂-C₈ alkoxyalkyl, N(R^{a})(R^{b}), (CH₂)₁₋₃N(R^{a})(R^{b}), (CH₂)₀₋₃R^{c}; or O(CH₂)₀₋₃R^{c};

R² is hydrogen, halo, nitro, C₁-C₆ alkyl, C₁-C₆ alkoxy, fluorinated C₁-C₆ alkyl, fluorinated C₁-C₆ alkoxy, C₂-C₈ alkoxyalkyl, fluorinated C₂-C₈ alkoxyalkyl, N(R^{a})(R^{b}), (CH₂)₁₋₃N(R^{a})(R^{b}), (CH₂)₀₋₃R^{c}, O(CH₂)₀₋₃R^{c}, (CH₂)₀₋₃R^{d}, O(CH₂)₀₋₃R^{d}, C(═O)CH₂C(═O)R^{e} or R^{f}.

R³ is hydrogen, halo, nitro, oxo, C₁-C₆ alkyl, C₃-C₇ cycloalkyl, C₃-C₇ cycloalkyloxy, C₁-C₆ alkoxy, fluorinated C₁-C₆ alkyl, fluorinated C₁-C₆ alkoxy, C₂-C₈ alkoxyalkyl, fluorinated C₂-C₈ alkoxyalkyl, N(R^{a})(R^{b}), (CH₂)₁₋₄N(R^{a})(R^{b}), C(═O)N(R^{a})(R^{b}), (CH₂)₁₋₄C(═O)N(R^{a})(R^{b})N(R^{a})C(═O)R^{b}, (CH₂)₁₋₄N(R^{a})C(═O)R^{b}, SO₂R^{a}, (CH₂)₁₋₄SO₂R^{a}, SO₂N(R^{a})(R^{b}), (CH₂)₁₋₄SO₂N(R^{a})(R^{b}), (CH₂)₁₋₄N(R^{a})SO₂R^{b}, (CH₂)₀₋₃R^{c}, or (CH₂)₀₋₃R^{g};

B is (i) a 5- or 6-membered heteroaromatic ring containing from 1 to 4 nitrogen atoms, 0 to 2 sulfur atoms, and at least 1 carbon atom, or (ii) an 8- to 10-membered fused bicyclic heterocycle containing from 1 to 4 nitrogen atoms, 0 to 2 sulfur atoms, and carbon atoms, wherein the ring of the heterocycle attached to the central, dione moiety is a 5- or 6-membered heteroaromatic ring containing at least one nitrogen or sulfur atom and the other ring of the heterocycle is a saturated or unsaturated ring; wherein B is attached to the central, dione moiety via a carbon atom and at least one nitrogen or sulfur atom in B is adjacent to the point of attachment;

R⁴ and R⁵ are substituents attached to nitrogen or carbon in B, and are each independently selected from hydrogen, halo, hydroxy, (CH₂)₁₋₄OH, C₁-C₆ alkyl, C₁-C₆ alkoxy, fluorinated C₁-C₆ alkyl, fluorinated C₁-C₆ alkoxy, C₂-C₈ alkoxyalkyl, fluorinated C₂-C₈ alkoxyalkyl, N(R^{a})(R^{b}), (CH₂)₁₋₄N(R^{a})(R^{b}), C(═O)N(R^{a})(R^{b}), (CH₂)₁₋₄C(═O)N(R^{a})(R^{b}), N(R^{a})C(═O)R^{b}, (CH₂)₁₋₄N(R^{a})C(═O)R^{b}, SO₂R^{a}, (CH₂)₁₋₄SO₂R^{a}, SO₂N(R^{a})(R^{b}), (CH₂)₁₋₄SO₂N(R^{a})(R^{b}), (CH₂)₁₋₄N(R^{a})SO₂R^{b}, and (CH₂)₀₋₃R^{h};

R^{a} and R^{b} are each independently hydrogen, C₁-C₆ alkyl, or fluorinated C₁-C₆ alkyl;

R^{c} is (i) phenyl or substituted phenyl, wherein each substituent on the substituted phenyl is independently halo, cyano, hydroxy, (CH₂)₁₋₄OH, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a}) (R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, and fluorinated C₂-C₈ alkoxyalkl; or (ii) an 8- to 10-membered fused bicyclic carbocycle in which one ring is a benzene ring and the other ring is a saturated or unsaturated ring, wherein the fused carbocycle is unsubstituted or substituted with one or more substituents selected from halo, cyano, hydroxy, (CH₂)₁₋₄OH, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a}) (R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄S₂N(R^{a})(R^{b}), (CH₂)₁₋₄N(R^{a})SO₂R^{b}, C₂-C₈ alkoxyalkyl, and fluorinated C₂-C₈ alkoxyalkyl;

R^{d} is (i) a 5- or 6-membered monocyclic he terocycle which is saturated or unsaturated and which contains at least one carbon atom and from 1 to 4 heteroatoms selected from nitrogen, oxygen, and sulfur; (ii) an 8- to 10-membered fused bicyclic heterocycle in which either ring is saturated or unsaturated and which contains carbon atoms and from 1 to 4 nitrogen atoms; wherein the heterocycle of (i) or (ii) is unubstituted or substituted with one or more substituents selected from halo, cyano, hydroxy, (CH₂)₁₋₄OH, oxo, thio, N(R^{a})(R^{b}),C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, fluorinated C₂-C₈ alkoxyalkyl, phenyl and benzyl; or (iii) a 5- or 6-membered monocyclic heterocycle which is saturated or unsaturated and which contains carbon atoms and from 1 to 3 nitrogen atoms, the heterocycle being substituted with spiro-C₁-C₂ alkylenedioxy or with one of pyrrolidinyl, piperidinyl, piperazinyl, and morpholinyl, unsubstituted or substituted with one or more substituents selected from halo, cyano, hydroxy, (CH₂)₁₋₄OH, oxo, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, fluorinated C₂-C₈ alkoxyalkyl, phenyl and benzyl;

R^{e} is a 5- or 6-membered heteroaromatic ring containing from 1 to 4 nitrogen atoms, 0 to 2 sulfur atoms, and at least 1 carbon atom; wherein the point of attachment of the ring is a carbon atom and at least one nitrogen or sulfur atom in the ring is adjacent to the point of attachment; wherein the ring is unsubstitutedl or substituted with one or more substituents selected from halo, cyano, hydroxy, (CH₂)₁₋₄OH, oxo, N(R^{a})(R^{b}) C₁-C₆ alkyl, fluorinated C₁-C₆ alkyl, C₁-C₆ alkoxy, fluorinated C₁-C₆ alkoxy (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, fluorinated C₂-C₈ alkoxyalkyl, phenyl and benzyl;

R^{f} is X—NH(CH₂)₁₋₃Y, wherein X is a 5- or 6-membered monocyclic heterocycle which is saturated or unsaturated and which contains carbon atoms a nd from 1 to 3 nitrogen atoms and which is unsubstituted or substituted with one or more substituents selected from halo, cyano hydroxy, (CH₂)₁₋₄OH, oxo, N(R^{a})(R^{b}),C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, and fluorinated C₂-C₈ alkoxykyl ; Y is pyrrolidinyl, peridinyl, piperazinyl, or morpholinyl, which is unsubstituted or substituted with one or more substituents selected from halo, cyano, hydroxy, (CH₂)₁₋₄OH, oxo, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, and fluorinated C₂-C₈ alkoxyalkyl;

R^{g} is a 5- or 6-membered monocyclic heterocycle which is saturated or unsaturated and which contains one or more carbon atoms and from 1 to 4 nitrogen atoms, the heterocycle being unsubstituted or substituted with one or more substituents selected from halo, cyano, hydroxy, (CH₂)₁₋₄OH, oxo, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH ₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, fluorinated C₂-C₈ alkoxyalkyl, phenyl and benzyl;

R^{h} is (i) C₃-C₆ cycloalkyl; (ii) phenyl; (iii) substituted phenyl, wherein each substituent on the substituted phenyl is ind ependently halo, cyano, hydroxy, (CH₂)₁₋₄OH, C₁-C₆ alkyl, fluorinated C₁-C₆ akyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, N(R^{a})(R^{b}), (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, or fluorinated C₂-C₈ alkoxyalkyl; or (iv) a 5- or 6-membered monocyclic heterocycle which is saturated or unsaturated and which contains at least one carbon atom and from 1 to 4 heteroatoms selected from nitrogen, oiygen, and sulfur; wherein the heterocycle is unsubstituted or substituted with one or more substituents selected from halo, cyano, hydroxy, (CH₂)₁₋₄OH, oxo, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O) R^{b}, (CH₂)₀₋₄SO₂N(R^{a})(R^{b}), (CH₂)₁₋₄N(R^{a})SO₂R^{b}, C₂-C₈ alkoxyalkyl, fluorinated C₂-C₈ alkoxyalkyll phenyl and benzyl;

or a pharmaceutically acceptable salt thereof.

Many known active inhibitors of viral, integrase (e.g., bis-catechols and chicoric acid analogs) are very polar compounds which will not efficiently penetrate cell phospholipid layers. In contrast, the compounds of the presen t invention, in addition to being potent HIV integrase inhibitors, are sufficiently nonpolar to freely permeate cells where they exhibit potent antiviral, activity. More particularly, the compounds of the present invention typically have a value of log P (defined below). greater than 0.

The present invention also includes pharmaceutical compositions containing a compound of the present invention and methods of preparing such pharmaceutical compositions. The present invention further includes methods of treating AIDS, methods of preventing infection by HIV, and methods of treating infection by HIV.

These and other embodiments, aspects and features of the present invention are either further described in or will be apparent from the ensuing description, examples, and appended claims.

### DETAILED DESCRIPTION OF THE INVENTION

The present invention includes the 1,3-propanedione derivatives of Formula (I) above. These compounds, their tautomers, and pharmaceutically acceptable salts thereof are HIV integrase inhibitors.

In a first embodiment of the invention is a compound of Formula (I), or a tautomer thereof, wherein

R⁴ and R⁵ are substituents attached to nitrogen or carbon in B, and are each independently selected from hydrogen, halo, hydroxy, C₁-C₆ alkyl, C₁-C₆ alkoxy, fluorinated C₁-C₆ alkyl, fluorinated C₁-C₆ alkoxy, C₂-C₈ alkoxyalkyl, fluorinated C₂-C₈ alkoxyalkyl, N(R^{a})(R^{b}), (CH₂)₁₋₄N(R^{a})(R^{b}), C(═O)N(R^{a})(R^{b}), (CH₂)₁₋₄C(═O)N(R^{a})(R^{b}), N(R^{a})C(═O)R^{b}, (CH₂)₁₋₄N(R^{a})C(═O)R^{b}, SO₂R^{a}, (CH₂)₁₋₄SO₂R^{a}, SO₂N(R^{a})(R^{b}), (CH₂)₁₋₄SO₂N(R^{a})(R^{b}), (CH₂)₁₋₄N(R^{a})SO₂R^{b}, and (CH₂)₀₋₃R^{h};

R^{a} and R^{b} are each independently hydrogen, C₁-C₄ alkyl, or fluorinated C₁-C₄ alkyl;

R^{c} is (i) phenyl or substituted phenyl, wherein each substituent on the substituted phenyl is independently halo, cyano, hydroxy, C₁-C₆ alkyl, fluorinated C₁-C₆ alkyl, C₁-C₆ alkoxy, fluorinated C₁-C₆ alkoxy, N(R^{a})(R^{b}), (CH₂)₁₋₄N(R^{a})(R^{b}), (CH₂)₀₋₄CO₂R^{a}, (CH₂)₀₋₄C(═O)N(R^{a})(R^{b}), (CH₂)₀₋₄SO₂R^{a}, C₂-C₈ alkoxyalkyl, or fluorinated C₂-C₈ alkoxyalkyl; or (ii) an 8- to 10-membered fused bicyclic carbocycle in which one ring is a benzene ring and the other ring is a saturated or unsaturated ring, wherein the fused carbocycle is unsubstituted or substituted with one or more substituents selected from halo, cyano, hydroxy, C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, C₂-C₈ alkoxyalkyl, and fluorinated C₂-C₈ alkoxyalkyl; R^{d} is (i) a 5- or 6-membered monocyclic heterocycle which is saturated or unsaturated and which contains at least one carbon atom and from 1 to 4 heteroatoms selected from nitrogen, oxygen, and sulfur; (ii) an 8- to 10-membered fused bicyclic heterocycle in which either ring is saturated or unsaturated and which contains carbon atoms and from 1 to 4 nitrogen atoms; wherein the heterocycle of (i) or (ii) is unsubstituted or substituted with one or more substituents selected from halo, cyano, hydroxy, oxo, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₁₋₄SO₂R^{a}, C₂-C₈ alkoxyalkyl, and fluorinated C₂-C₈ alkoxyalkyl; or (iii) a 5- or 6-membered monocyclic heterocycle which is saturated or unsaturated and which contains carbon atoms and from 1 to 3 nitrogen atoms, the heterocycle being substituted with spiro-C₁-C₂ alkylenedioxy, or with one of pyrrolidinyl, piperidinyl, piperazinyl, and morpholinyl, unsubstituted or substituted with one or more substituents selected from halo, cyano, hydroxy, N(R^{a})(R^{b}), C₁-C₆ alkyl, fluorinated C₁-C₆ alkyl, C₁-C₆ alkoxy, and fluorinated C₁-C₆ alkoxy;

R^{e} is a 5- or 6-membered heteroaromatic ring containing from 1 to 4 nitrogen atoms, 0 to 2 sulfur atoms, and at least 1 carbon atom; wherein the point of attachment of the ring is a carbon atom and at least one nitrogen or sulfur atom in the ring is adjacent to the point of attachment; wherein the ring is unsubstituted or substituted with one or more substituents selected from halo, cyano, hydroxy, oxo, C₁-C₆ alkyl fluorinated C₁-C₆ alkyl, C₁-C₆ alkoxy, fluorinated C₁-C₆ alkoxy, (CH₂)₀₋₄CO₂R^{a}, N(R^{a})(R^{b}), (CH₂)₀₋₄C(═O)N(R^{a})(R^{b}), (CH₂)₀₋₄SO₂R^{a}, C₂-C₈ alkoxyalkyl, and fluorinated C₂-C₈ alkoxyalkyl;

R^{f} is X—NH(CH₂)₁₋₃Y, wherein X is a 5- or 6-membered monocyclic heterocycle which is saturated or unsaturated and which contains carbon atoms and from 1 to 3 nitrogen atoms and which is unsubstituted or substituted with one or more substituents selected from halo, cyano, hydroxy, N(R^{a})(R^{b}), C₁-C₆ alkyl, fluorinated C₁-C₆ alkyl, C₁-C₆ alkoxy, and fluorinated C₁-C₆ alkoxy; Y is pyrrolidinyl, piperidinyl, piperazinyl, or morpholinyl, which is unsubstituted or substituted with one or more substituents selected from halo, cyano, hydroxy, N(R^{a})(R^{b}), C₁-C₆ alkyl, fluorinated C₁-C₆ alkyl, C₁-C₆ alkoxy, and fluorinated C₁-C₆ alkoxy;

R^{g} is a 5- or 6-membered nionocyclic heterocycle which is saturated or unsaturated and which containSOne or more carbon atoms and from 1 to,4 nitrogen atoms, the heterocycle being unsubstituted or substituted with one or more substituents selected from halo, cyano, hydroxy, N(R^{a})(R^{b}), C₁-C₆ alkyl, fluorinated C₁-C₆ alkyl, C₁-C₆ alkoxy, and fluorinated C₁-C₆ alkoxy; and

R^{h} is C₃-C₆ cycloalkyl, phenyl or substituted phenyl, wherein each substituent on the substituted phenyl is independently halo, cyano, hydroxy, C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, C₂-C₈ alkoxyalkyl, or fluorinated C₂-C₈ alkoxyalkyl;

and all other variables are as originally defined;

or a pharmaceutically acceptable salt thereof.

An aspect of the invention is a compound of Formula (I), or a tautomer thereof, wherein all of the variables are as originally defined, with the proviso that. when A is (iii) an 8- to 10-membered fused bicyclic heterocycle, then A is other than indole. Simnilarly, in another aspect, the invention is a compound of Formula (I), or a tautomer thereof, wherein all of the variables are as defined in the first embodiment, with the proviso that when A is (iii) an 8- to 10-membered fused bicyclic heterocycle, A is other than indole. In still other aspects, the invention is a compound of Formula (I), wherein all of the variables are as originally defined or as defined in the first embodiment, provided that when A is (iii) an 8- to 10-membered fused bicyclic heterocycle and B is (ii) an 8- to 60-membered fused bicyclic heterocycle, then each of A and B is other than indole.

A second embodimen t of the invention is a compound of Formula (I), or a tautomer thereof, wherein

A 1 s a benzene ring; and

all other variables are as originally defined;

or a phaimaceutically acceptable salt thereof.

A third embodiment of the invention is a compound of Formula (I), or a tautomer thereof, wherein

A is a benzene ring; and

all other variables are as defined in the first embodiment;

or a pharmaceutically acceptable salt thereof.

An aspect of each. of the second and third embodiments is that when B is (ii) an 8- to 10-membered fused bicyclic heterocycle, then B is other than indole.

Fourth, fifth, and sixth embodiments of the invention are each a compound of Formula (I), or a tautomer thereof, wherein

B is a 5- or 6-membered heteroaromatic ring containing from 1 to 4 nitrogen atoms, 0 to 2 sulfur atoms, and at least 1 carbon atom; and

all other variables are respectively as originally defined, as defined in the first embodiment, as defined in the second embodiment, and as defined in the third embodiment;

or a pharmaceutically acceptable salt thereof.

A first class of the present invention is a compound of Formula (II):

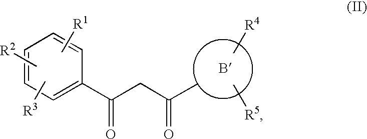

or a tautomer thereof, wherein

R¹ is hydrogen, fluoro, chioro,, brorno, methyl, ethyl, propyl, isopropyl, OCH₃, OCH₂CH₃, OCH₂CH₂CH₃, OCH(CH₃)₂, CF₃, CH₂CF₃, OCF₃ OCH₂CF₃, (CH₂)₁₋₃O(CH₂)₀₋₁CH₃, (CH₂)₁₋₃O(CH₂)₀₋₁CF₃, N(R^{a})(R^{b}), CH₂N(R^{a})(R^{b}), (CH₂)₀₋₂R^{c}, or O(CH₂)₀₋₂R^{c};

R² is hydrogen, fluoro, chloro, bromo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, N(R^{a})(R^{b}), CH₂N(R^{a})(R^{b}), (CH₂)₀₋₂R^{c}, O(CH₂)₀₋₂R^{c}, (CH₂)₀₋₂R^{d}, O(CH₂)₀₋₂R^{d}, C(═O)CH₂C(═O)R^{e}, or R^{f};

R³ is hydrogen, fluoro, chloro, bromo, oxo, methyl, ethyl, propyl, isopropyl, C₃-C₆ cycloalkyl, C₃-C₆ cycloalkyloxy, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a}) (R^{b}), (CH₂)₁₋₂SO₂N(R^{a})(R^{b}), (CH₂)₁₋₂N(R^{a})SO₂R^{b}, (CH₂)₀₋₂R^{c}, or (CH₂)₀₋₂R^{g};

B′ is a 5- or 6-membered heteroaromatic ring containing from 1 to 4 nitrogen atoms, 0 or 1 sulfur atoms, and one or more carbon atoms, wherein B′ is attached to the central dione moiety via a carbon atom and at least one nitrogen atom in B′ is adjacent to the point of attachment;

R⁴ and R⁵ are substituents attached to any nitrogen or carbon in B′ except for the ring carbon attached to the central, dione moiety, and are each independently selected from hydrogen, fluoro, chloro, bromo, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₂OH, (CH₂)₁₋₂O—C₁-C₄ alkyl, (CH₂)₁₋₃O(CH₂)₀₋₁CF₃, N(R^{a})(R^{b}), (CH₂)₁₋₂N(R^{a})(R^{b}), C(═O)N(R^{a})(R^{b}), (CH₂)₁₋₂C(═O)N(R)(R^{b}), N(R^{a})C(═O)R^{b}, (CH₂)₁₋₂N(R^{a})C(═O) R^{b}, SO₂R^{a}, (CH₂)₁₋₂SO₂R^{a} SO₂N(R^{a})(R^{b}), CH₂)₁₋₂SO₂N(R^{a})(R^{b}), (CH₂)₁₋₂N(R^{a})SO₂R^{b}, and (CH₂)₀₋₂R^{h};

R^{a} and R^{b} are each independently hydrogen, C₁-C₄ alkyl, or fluorinated C₁-C₄ alkyl;

R^{c} is (i) phenyl or substituted phenyl, wherein each substituent on the substituted phenyl is independently fluoro, chloro, bromo, hydroxy, (CH₂)₁₋₂OH, methyl, ethyl, propyl, isopropyl, OCH₃, OCH₂CH₃, OCR₂CR₂CR₃, OCH(CR₃)₂, CF₃, CH₂CF₃, OCF₃, OCH₂CF₃, (CH₂)₁₋₃O(CH₂)_{0-1CH}₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}) C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R^{b}; or (ii) an 8- to 10-membered fused bicyclic carbocycle in which one ring is a benzene ring and the other ring is a saturated or unsaturated ring, wherein the fused carbocycle is unsubstituted or substituted with one or more substituents selected from fluoro, chloro, bromo, hydroxy, (CH₂)₁₋₂OH, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R^{b};

R^{d} is (i) a 5- or 6-membered monocyclic heterocycle which is saturated or unsaturated and which contains at least one carbon atom and from 1 to 4 heteroatoms selected from nitrogen, oxygen, and sulfur, wherein each ring sulfur is. in a form selected from S, SO and SO₂; (ii) an 8- to 10-membered fused bicyclic heterocycle in which either ring is saturated or unsaturated and which contains carbon atoms and from 1 to 4 nitrogen atoms; wherein the heterocycle of (i) or (ii) is unsubstituted or substituted with one or more substituents selected from fluoro, chloro, bromo, hydroxy, (CH₂)₁₋₂OH, oxo, thio, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, N(R^{a})(R^{b}), (CH₂)₁₋₂N(R^{a})(R^{b}), C(═O)N(R^{a})(R^{b}), (CR₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl; or (iii) a 5- or 6-membered monocyclic heterocycle which is saturated or unsaturated and which contains carbon atoms and from 1 to 3 nitrogen atoms, the heterocycle being substituted with spiro-C₁-C₂ alkylenedioxy, or with one of pyrrolidinyl, piperidinyl, piperazinyl, or morpholinyl, which is unsubstituted or substituted with one or more substituents selected from. fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a)(R}^{b}), (CH₂)₁₋₂C(═O)N(R^{a})(R^{b}), N(R^{a})C(═O)R^{b}, (CH₂)₁₋₂N(R^{a})C(O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl;

R^{e} is a 5- or 6-membered heteroaromatic ring containing from 1 to 4 nitrogen atoms, 0 to 2 sulfur atoms, and at least 1 carbon atom; wherein the point of attachment of the ring is a carbon atom and at least one nitrogen or sulfur atom in the ring is adjacent to the point of attachment; wherein the ring is unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH_{3, (CH}₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl;

R^{f} is X—NH(CH₂)₁₋₂Y, wherein X is a 5- or 6-membered monocyclic heterocycle : which is saturated or unsaturated and which contains carbon atoms and from 1 to 3 nitrogen atoms and which is unsubstituted or substituted with one or more. substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{a}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R^{b}; Y is pyrrolidinyl, piperidinyl, piperazinyl, or morpholinyl, which is unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₂N(R^{a})(R^{b}) C(═O)N(R^{a})(R^{a}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R^{b};

R^{g} is a 5- or 6-membered monocyclic heterocycle which is saturated or unsaturated and which contains one or more carbon atoms and from 1 to 4 nitrogen atoms, the heterocycle being unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl; and

R^{h} is (i) C₃-C₆ cycloalkyl; (ii) phenyl; (iii) substituted phenyl, wherein each substituent on the substituted phenyl is independently fluoro, chloro, bromo, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, (CH₂)₁₋₂OH, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₄SO₂R^{a}, SO₂N(R^{a})(R^{b}), (CH₂)₁₋₂SO₂N(R^{a})(R^{b}), (CH₂)₁₋₂N(R^{a})SO₂R^{b}, or (iv) a 5- or 6-membered monocyclic heterocycle which is saturated or unsaturated and which contains at least one carbon atom and from 1 to 4 heteroatoms selected from nitrogen, oxygen, and sulfur; wherein the heterocycle is unsubstituted or substituted with one or more substituents selected from fluoro, chloro, bromo, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, (CH₂)_{1 2}OH, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (C₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl;

or a pharmaceutically acceptable salt thereof.

A first sub-class of the present invention is a compound of Formula (II), or a tautomer thereof, wherein

B′ is pyridyl, pyrazinyl, pyrimidinyl, imidazolyl, triazolyl, tetrazolyl, or thiazolyl;

R^{c} is (i) phenyl or substituted phenyl or (ii) an unsubstituted or substituted fused bicyclic carbocycle selected from

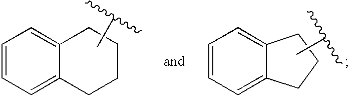

R^{d} is (i) an unsubstituted or substituted 5- or 6-membered monocyclic heterocycle selected from pyrazolyl, imidazolyl, pyrrolyl, pyrrolidinyl, pyridyl, piperidinyl,: pyrazinyl, piperazinyl, pyridazinyl, pyrimidinyl, 1,2,4-triazolyl, 1,2,3-triazolyl, tetrazolyl, morpholinyl, tetrahydrothienyl, tetrahydrodioxothienyl, thiadiazinanyl, dioxothiadiazinanyl, thiazinanyl, dioxothiazinanyl, thiazolidinyl, dioxothiazolidinyl, is othiazolidinyl, is odioxothiazolidinyl, thiazolyl, and is othiazolyl; (ii) an unsubstituted or substituted fused bicyclic heterocycle selected from

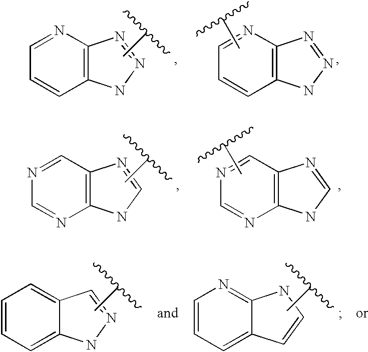

(iii) a monocyclic heterocycle selected from pyridyl, piperidinyl, pyraz,nyl, r piperazinyl, and pyriniidinyl, the hetero cycle being substituted with spiro-C₁-C₂ alkylenedioxy or with one of unsubstituted or substituted piperidinyl, unsubstituted or substituted piperazinyl, or unsubstituted or substituted morpholinyl;

R^{e} is an unsubstituted or substituted heteroaromatic ring selected from pyridyl, pyrazinyl, and pyrimidinyl;

R^{f} is X—NH(CH₂)₁₋₂y, wherein X is selected from unsubstituted or substituted pyridyl, unsubstituted or substituted pyrazinyl, and unsubstituted or substituted pyrimidinyl; and Y is unsubstituted or substituted pyrrolidinyl, unsubstituted or substituted piperidinyl, unsubstituted or substituted piperazinyl. or unsubstituted or ′ substituted morpholinyl;

R^{g} is an unsubstituted or substituted monocyclic heterocycle selected from pyridyl, pyrroly, 1,2,3-triazolyl, 1,2,4-triazolyl, pyrazolyl, imidazolyl, tetrazolyl, pipe,dinyl, and piperazinyl; and

R^{h} is C₃-C₆ cycloalkyl, phenyl, substituted phenyl, or an unsubstituted or substituted monocyclic heter ocycle selected from pyridyl, pyrrolyl, 1,2,3-triazolyl, 1,2,4-triazolyl, pyrazolyl, imidazolyl, tetrazolyl, piperodinyl, piperazinyl, and tetrahydrofuranyl;

and all other variables are as defined in the first class;

or a pharmaceutically acceptable salt thereof.

An aspect of the invention is a compound of Formula (II), or a tautomer thereof, wherein B′ is pyridyl; and

all other variables are as defined in the first sub-class;

or a pharmaceutically acceptable salt thereof.

A second class of the present invention is a compound of Formula (II), or a tautomner thereof, wherein

R¹ is hydrogen, fluoro, chioro, bromo, methyl, ethyl, propyl, isopropyl, OCH₃, OCH₂CH₃, OCH₂CH₂CU₃, OCH(CH₃)₂, CF₃, CH₂CF₃, OCF₃, OCH₂CF₃, (CH₂)₁₋₃OCH₃, (CH₂)₁₋₃OCF₃, N(R^{a}) (R^{b}), CH₂N(R^{a})(R^{b}),(CH₂)₀₋₂R^{c}, or O(CH₂)₀₋₂R^{c};

R² is hydrogen, fluoro, chloro, bromo, methyl, ethyl, propyl, isopropyl, OCH₃, OCH₂CH₃, OCH₂CH₂CU₃, OCH(CH₃)₂, CF₃, CH₂CF₃, OCF₃, OCH₂CF₃, (CH₂)₁₋₃OCH₃, (CH₂)₁₋₃OCF₃, N(R^{a})(R^{b}) CH₂N(R^{a})(R^{b}), (CH₂)₀₋₂R^{c}, O(CH₂)₀₋₂R^{c}, (CH₂)₀₋₂R^{d}, O(CH₂)₀₋₂R^{d}, C(═O)CH₂C(═O)R^{e}, or R^{f};

R³ is hydrogen, fluoro, chloro, bromo, oxo, methyl, ethyl, propyl, isopropyl, C₃-C₆ cycloalkyl, C₃-C₆ cyctoalkyloxy, OCH₃, OCH₂CH₃, OCH₂CH₂CH₃, OCH(CH₃)₂, CF₃, CH₂CF₃, OCF₃, OCH₂CF₃, (CH₂)₁₋₃OCH₃, (CH₂)₁₋₃OCF₃, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₄N(R^{a})C(═O)R^{b}, SO₂R^{a}, (CH₂)₁₋₄SO₂R^{a}, SO₂N(R^{a}) (R^{b}), (CH₂)₁₋₄SO₂N(R^{a})(R^{b}), (CH₂)₁₋₄N(R^{a})SO₂R^{b}, (CH₂)₀₋₂R^{c}, or (CH₂)₀₋₂R^{g};

B′ is a 5- or 6-membered heteroaromatic ring containing from 1 to 4 nitrogen atoms, 0 or 1 sulfur atoms, and one or more carbon atoms, wherein B′ is attached to the central dione moiety via a carbon atom and at least one nitrogen atom in B′ is adjacent to the point of attachment;

R⁴ and R⁵ are substituents attached to any nitrogen or carbon in B′ except for the ring carbon attached to the central, dione moiety, and are each independently selected from hydrogen, fluoro, chioro, bromo, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, N(R^{a})(R^{b}), (CH₂)₁₋₂N(R^{a})(R^{b}), C(═O)N(R^{a}) (R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₄SO₂R^{a}, SO₂N(R^{a})(R^{b}), (CH₂)₁₋₂SO₂N(R^{a})(R^{b}), (CH₂)₁₋₂N(R^{a})SO₂R^{b}, and (CH₂)₀₋₂R^{h}

R^{a} and R^{b} are each independently hydrogen, methyl, ethyl, CF₃, CH₂CF₃, OCF₃, or OCH₂CF₃;

R^{c} is (i) phenyl or substituted phenyl, wherein each substituent on the substituted phenyl is independently fluoro, chloro, bromo, cyano, hydroxy, methyl; ethyl, propyl, isopropyl, OCH₃, OCH₂CH₃, OCH₂CH₂CH₃, OCH(CH₃)₂, CF₃, CH₂CF₃, OCF_{3, OCH}₂CF₃, N(R^{a})(R^{b}), (CH₂)₁₋₂N(R^{a})(R^{b}), (CH₂)₀₋₂CO₂R^{a}, (CH₂)₀₋₂C(═O)N(R^{a})(R^{b}), (CH₂)₀₋₂SO₂R^{a}, (CH₂)₁₋₃OCH₃, or (CH₂)₁₋₃OCF₃; or (ii) an 8- to 10-membered fused bicyclic carbocycle in which one nng is a benzene ring and the other ring is a saturated or unsaturated ring, wherein the fused carbocycle is unsubstituted or substituted with one or more substituents selected from fluoro, chloro, bromo, cyano, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₀₋₂CO₂R^{a}, (CH₂)₀₋₂C(═O)N(R^{a})(R^{b})(CH₂)₀₋₂SO₂R^{a}, (CH₂)₁₋₃OCH₃, and (CH₂)₁₋₃OCF₃;

R^{d} is (i) a 5- or 6-membered monocyclic heterocycle which is saturated or unsaturated and which contains at least one carbon atom and from 1 to 4 heteroatoms selected from nitrogen, oxygen, and sulfur; (ii) an 8- to 10-membered fused bicyclic heterocycle in which either ring is saturated or unsaturated and which contains carbon atoms and from 1 to 4 nitrogen atoms; wherein the heterocycle of (i) or (ii) is unsubstituted or substituted with one or more substituents selected from fluoro, chloro, bromo, cyano, hydroxy, oxo, N(R^{a})(R^{b}), methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₀₋₂CO₂R^{a}, (CH₂)₀₋₂C(═O)N(R^{a})(R^{b}), (CH₂)₀₋₂SO₂R^{a}, (CH₂)₁₋₃OCH₃, and (CH₂)₁₋₃OCF₃; or (iii) a 5- or 6-membered monocyclic heterocycle which is saturated or unsaturated and which contains carbon atoms and from 1 to 3 nitrogen atoms, the heterocycle being substituted with spiro-C₁-C₂ alkylenedioxy, or with one of pyrrolidinyl, piperidinyl, piperazinyl, or morpholinyl, which is unsubstituted or substituted with one or more substituents selected from fluoro, chloro, bromo, cyano, hydroxy, N(R^{a})(R^{b}), methyl, ethyl, propyl, isopropyl, OCH₃, OCH₂CH₃, OCH₂CH₂CH₃, OCH(CH₃)₂, CF₃ CH₂CF₃, OCF₃, and OCH₂CF₃;

R^{e} is a 5- or 6-membered heteroaromatic ring containing from 1 to 4 nitrogen atoms, 0 to 2 sulfur atoms, and at least 1 carbon atom; wherein the point of attachment of the ring is a carbon atom and at least one nitrogen or sulfur atom in the ring is adjacent to the point of attachment; wherein the ring is unsubstituted or substituted with one or more substituents selected from fluoro, chloro, bromo, cyano, hydroxy, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₀₋₂CO₂R^{a}, N(R^{a})(R^{b}), (CH₂)₀₋₂C(═O)N(R^{a})(R^{b})(CH₂)₀₋₄SO₂R^{a}, (CH₂)₁₋₃OCH₃, and (CH₂)₁₋₃OCF₃;

R^{f} is X—NH(CH₂)₁₋₂Y, wherein X is a 5- or 6-membered monocyclic heterocycle which is saturated or unsaturated and which contains carbon atoms and from 1 to 3 nitrogen atoms and which is unsubstituted or substituted with one or more substituents selected from fluoro, chloro, bromo, cyano, hydroxy, N(R^{a})(R^{b}), methyl, ethyl, propyl, isopropyl, OCH₃, OCH₂CH₃, OCH₂CH₂CH₃, OCH(CH₃)₂, CF₃, CH₂CF₃, OCF₃, and OCH₂CF₃; Y is pyrrolidinyl, piperidinyl, piperazinyi, or morpholinyl, which is unsubstituted or substituted with one or more substituents selected from fluoro, chloro, bromo, cyano, hydroxy, N(R^{a})(R^{b}), methyl, ethyl, propyl, isopropyl, OCH₃, OCH₂CH₃, OCH₂CH₂CH₃, OCH(CH₃)₂, CF₃, CH₂CF₃, OCF₃, and OCH₂CF₃;

R^{g} is a 5- or 6-membered monocyclic heterocycle which is saturated or unsaturated and which containSOne or more carbon atoms and from 1 to 4 nitrogen atoms, the heterocycle being unsubstituted or substituted with one or more substituents selected from fluoro, chloro, bromo, cyano, hydroxy, N(R^{a})(R^{b}), methyl, ethyl, propyl, isopropyl, OCH₃, OCH₂CH₃, OCH₂CH₂CH₃, OCH(CH₃)₂, CF₃, CH₂CF₃, OCF₃, and OCH₂CF₃; and

R^{h} is C₃-C₆ cycloalkyl, phenyl or substituted phenyl, wherein each substituent on the substituted phenyl is independently fluoro, chloro, bromo, cyano, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₀₋₂CO₂R^{a}, (CH₂)₀₋₂C(═O)N(R^{a})(R^{b}), (CH₂)₀₋₂SO₂R^{a}, (CH₂)₁₋₃OCH₃, or (CH₂)₁₋₃OCF₃;

or a pharmaceutically acceptable salt thereof.

A second sub-class is a compound of Formula (II), or a tautomer thereof, wherein

B′ is pynrdyl, pyrazinyl, pyrimidinyl, imidazolyl, triazolyl, tetrazolyl, or thiazolyl;

R^{a} and R^{b} are each independently hydrogen or methyl;

R^{c} is (i) phenyl or substituted phenyl, wherein each substituent on the substituted phenyl is independently fluoro, chloro, cyano, methyl, ethyl, propyl, isopropyl, OCH₃, CF₃, OCF₃, or CH₂OCH₃; or (ii) a fused bicyclic carbocycle selected from

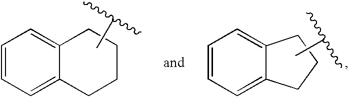

wherein the fused carbocycle is unsubstituted or substituted with one or more substituents selected from fluoro, chloro, cyano, methyl, ethyl, propyl, isopropyl, OCH₃, CF₃, OCF₃ and CH₂OCH₃;

R^{d} is (i) a 5- or 6-membered monocyclic heterocycle selected from pyrazolyl, imidazolyl, pyrrolyl, pyrrolidinyl, pyridyl, piperidinyl, pyrazinyl, piperazinyl, pyridazinyl, pyrimidinyl, 1,2,4-triazolyl, 1,2,3-triazolyl, tetrazolyl, and morpholinyl; (ii) a fused bicyclic heterocycle selected from

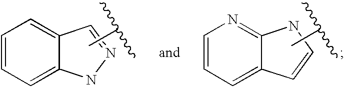

wherein the heterocycle of (i) or (ii) is unsubstituted or substituted with one or more substituents selected from fluoro, chloro, cyano, hydroxy, oxo, N(R^{a})(R^{b}), methyl, ethyl, propyl, isopropyl, OCH₃, CF₃, OCF₃, and CH₂OCH₃; or (iii) a monocyclic heterocycle selected from pyridyl, piperidinyl, pyrazinyl, piperazinyl, and pyrimidinyl, the heterocycle being substituted with spiro-C₁-C₂ alkylenedioxy, or with one of piperidinyl, piperazinyl, or morpholinyl, which is unsubstituted or substituted with one or more substituents selected from fluoro, chloro, N(R^{a})(R^{b}), methyl, ethyl, propyl, isopropyl, OCH₃, CF₃, and OCF₃;

R^{e} is a heteroaromatic ring selected from pyridyl, pyrazinyl, and pyrimidinyl; wherein the ring is unsubstituted or substituted with one or more substituents selected from fluoro, chloro, methyl, ethyl, propyl, isopropyl, OCH₃, CF₃, OCF₃ and CH₂OCH₃;

R^{f} is X—NH(CH₂)₁₋₂Y, wherein X is selected from pyridyl, pyrazinyl, and pyrimidinyl, which is unubstituted or substituted with one or more substituents selected from fluoro, chloro, N(R^{a})(R^{b}), methyl, ethyl, propyl, isopropyl, OCH₃, CF₃, and OCF₃; and Y is pyrrolidinyl, piperidinyl, piperazinyl; or morpholinyl, which is unsubstituted or substituted with one or more substituents selected from flutoro, chlro, N(R^{a})(R^{b}), methyl, ethyl, propyl, isopropyl, OCH₃, CF₃, and OCF₃;

R^{g} is a monocyclic heterocycle selected from pyridyl, pyrrolyl, 1,2,3-triazolyl, 1,2,4-triazolyl, pyrazolyl, and imidazolyl, the heterocycle being unsubstituted or substituted with one or more substituents selected from fluro, chloro, N(R^{a})(R^{b}), methyl, ethyl, propyl, isopropyl, OCH₃, CF₃, and OCF₃;

R^{h} is C₃-C₆ cycloalkyl, phenyl or substituted phenyl, wherein each substituent on the substituted phenyl is independently fluoro, chloro, methyl, ethyl, propyl, isopropyl, OCH₃, CF₃, and OCF₃; and

all other variables are as defined in the second class;

or a pharmaceutically acceptable salt thereof.

An aspect of the invention is a compound of Formula (II), or a tautomer thereof, wherein B′ is pyridyl; and

all other variables are as defined in the second subclass;

or a pharmaceutically acceptable salt thereof.

Exemplary compounds of the invention include compounds selected from the group consisting of

1-(3-Benzyl-5-pyrazin-2-ylmethylphenyl)-3-(4-methylpyridin-2-yl)-propane-1,3-dione;

1-(3-Benzyl-5-pyradin-2-ylmethylphenyl)-3-(4-methylpyridin-2-yl)-propane-1,3-dione;

3-[3-(2-Chlorobenzyl)-5-pyridin-2-ylmethylphenyl]-1-(4-methylpyridin-2-yl)-propane-1,3-dione;

3-[3-(3-Chlorobenzyl)-5-pyridin-2-ylmethylphenyl]-1-(4-methylpyridin-2-yl)-propane-1,3-dione;

1-[3-Benzyl-5-(2-oxo-2H-pyridin-1-ylmethyl)phenyl]-3-(4-methylpyridin-2-yl)-propane-1,3-dione;

1-[3-Benzyl-5-(2-oxo-piperidin-1-ylmethyl)phenyl]-3-(4-methylpyridin-2-yl)-propane-1,3-dione;

1-[3-Benzyl-5-(4-methylpiperazin-1-ylmethyl)phenyl]-3-(4-methylpyridin-2-yl)-propane,1,3-dione;

1-(3-Benzylphenyl)-3-(4-methylpyridin-2-yl)-propane-1,3-dione;

1-[3-(2,6-difluoro-benzyl)-phenyl]-3-(4-methoxy-pyrdin-2-yl)-propane-1,3-dione;

1-(3-Benzyl-phenyl)-3-(4-methoxy-pyridin-2-yl)-propane-1,3dione;

1-[3-(2,6-Difluoro-benzyl)-phenyl]-3-(4-methyl-pyridin-2-yl)-propane-1,3-dione;

1-{3-Benzyl-5-[6(2-morpholin-4-yl-ethylamino)-pyrazin-2-yl]-phenyl}-3-(4-methyl-pyridin-2-yl)-propane-1,3-dione;

1-[3-Benzyl-5-(6-methoxypyridin-2-yl)-phenyl]-3-(4-methyl-pyridin-2-yl)-propane-1,3-dione;)

1-[3-Benzyl-5-(6-morpholin-4-yl-pyrazin-2-yl)-phenyl]-3-(4-methyl-pyridin-2-yl)-propane-1,3-dione;

1-[3-Benzyl-5-(4-methyl-3,4,5,6-tetrahydro-2H-[1,2′]bipyrazinyl-6′-yl)-phenyl]-3-(4-methyl-pyridin-2-yl)-propane-1,3-dione;

1-[5-(4-Fluoro-benzyl)-2,3-dimethoxy-phenyl]-3-(4-methyl-pyridin-2-yl)-propane-1,3-dione;

1-[2,3-Dimethoxy-5-(2-methyl-benzyl)-phenyl]-3-(4-methyl-pyridin-2-yl)-propane-1,3-dione;

1-(5-Benzyl-2-methoxy-3-morpholin-4-ylmethylphenyl)-3-(4-methyl-pyridin-2-yl)propane-1,3-dione;

1-(5-Benzyl-2-isopropoxy-3-pyrrolidin-1-ylmethyl-phenyl)-3-(4-methyl-pyridin-2-yl)propane-1,3-dione;

1-(5-Benzyl-2-isopropoxy-3-[1,2,4]triazol-1-ylmethylphenyl)-3-(4-methyl-pyridin-2-yl)propane 1,3-dione;

1-[5-Benzyl-2-(pyridin-2-yloxy)phenyl]-3-(4-methylpyridin-2-yl)propane-1,3-dione;

1-[3-Benzyl-5-(4-methylpiperazin-1-yl)phenyl]-3-(4-methylpyridin-2-yl)-propane-1,3-dione;

1-(3-Benzyl-5-[1,2,4]triazol-1-ylmethylphenyl)-3-(4-methylpyridin-2-yl)-propane-1,3-dione;

1-(3-Benzyl-5-imidazol-1-ylphenyl)-3-(4-methylpyridin-2-yl)-propane-1,3-dione;

1-(3-Benzyl-5-[1,2,3]triazol-1-ylmethylphenyl)-3-(4-methylpyridin-2-yl)-propane-1,3-dione;

1-(3-Benzyl-5-[1,2,3]triazol-1-ylmethylphenyl)-3-(6-methylpyridin-2-yl)-propane-1,3-dione;

1-(3-Benzyl-5-tetrazol-1-ylmethylphenyl)-3-(4-methylpyridin-2-yl)-propane-1,3-dione;

1-(3-Benzyl-5-[1,2,3]triazol-1-ylmethylphenyl)-3-(4-methylpyridin-2-yl)-propane-1,3-dione;

1-(3-Benzyl-5-tetrazol-2-ylmethylphenyl)-3-(4-methylpyridin-2-yl)-propane-1,3-dione;

1-(3-Benzyl-5-[2,3]pyridin-1-ylmethylphenyl)-3-(4-methylpyridin-2-yl)-propane-1,3-dione;

1-(3-Benzylphenyl)-3-(3-isopropoxypyridin-2-yl)-propane-1,3-dione;

1-(3-Benzylphenyl)-3-(3-propoxypyridin-2-yl)-propane-1,3-dione;

1-(3,5-Bis-pyrazol-1-ylmethyl-phenyl)-3-(4-methyl-pyridin-2-yl)-propane-1,3-dione;

1-(4-Methyl-pyridin-2-yl)-3-(3-pyrazol-1-ylmethyl-phenyl)-propane-1,3-dione;

1-(4-Methyl-pyridin-2-yl)-3-(3-pyrrol-1-ylmethyl-phenyl)-propane-1,3-dione;

1-(4-Methyl-pyridin-2-yl)-3-(3-tetrazol-2-ylmethyl-phenyl)-propane-1,3-dione;

1-(4-Methyl-pyridin-2-yl)-3-(3-[1,2,3]triazol-2-ylmethyl-phenyl)-propane-1,3-dione;

1-[3-(3-Methyl-pyrazol-1-ylmethyl)-phenyl]-3-(4-methyl-pyridin-2-yl)-propane-1,3-dione;

1-[3-(5-Methyl-pyrazol-1-ylmethyl)-phenyl]-3-(4methyl-pyridin-2-yl)-propane-1,3-dione;

1-(4-Methyl-pyridin-2-yl)-3-(3-[1,2,3]triazol-2-ylmethyl-5-[1,2,3]triazol-1-ylmethyl phenyl)-propane-1,3-dione;

1-(3,5-Bis-pyrrol-1-ylmethyl-phenyl)-3-(4-methyl-pyridin-2-yl)propane-1,3-dione;

1-(3-Indazol-1-ylmethyl-phenyl)-3-(4-methyl-pyridin-2-yl)-propane-1,3-dione;

1-(4-methyl-pyridin-2-yl)-3-(3-pyrimidin-2-ylmethyl-phenyl)-propane-1,3-dione;

1-(3-Benzylphenyl)-3-(5-dimethylaminopyridin-2-yl)-propane-1,3-dione;

1-(3-benzyl-5-pyrazin-2-yl-phenyl)-3-(4-methyl-pyridin-2-yl)-propane-1,3-dione;

1-(3-benzyl-5-pyrimidin-2-yl-phenyl)-3-(4-methyl-pyridin-2-yl)-propane-1,3-dione;

tautomers thereof;

and pharmaceutically acceptable salts thereof.

Exemplary compounds of the invention also include compounds selected from the group consisting, of

1-(3,5-bis-pyridin-2-ylmethylphenyl)-3-(4-methylpyridin-2-yl)propane-1,3-dione;

1-[3,5-bis-(2-methyl-2H-pyrazol-3-ylmethyl)phenyl]-3-(4-methylpyridin-2-yl)propane-1,3-dione;

1-(3-Pyridin-2-ylmethyl)phenyl-3-(4-methylpyridin-2-yl)propane-1,3-dione;

1-[3-(1,1-Dioxoisothiazolin-2-ylmethyl)-5-(pyridin-2-ylmethyl)phenyl]-3-(4-methylpyridin-2-yl)propane-1,3-dione;

1-[5-(2,6-Difluorobenzyl)-2,3-dimethoxyphenyl]-3-(4-methylpyridin-2-yl)propane-1,3-dione;

1-(5-benzyl-2-fluorophenyl)-3-(4-methylpyridin-2-yl)propane-1,3-dione;

1-(2-Methoxy-5-[1,2,3]triazol-2-ylmethylphenyl)-3-(4-methylpyridin-2-yl)propane-1,3-dione;

1-(3-benzyl-5-indazol-1-ylmethyl)phenyl-3-(4-methylpyridin-2-yl)propane-1,3-dione;

1-(3-benzyl-5-pyrazol-1-ylmethyl)phenyl-3-(4-methylpyridin-2-yl)propane-1,3-dione;

1-(3-benzyl-5-[1,2,3]triazolo[4,5,b]pyridin-1-ylmethyl)phenyl-3-(4-methylpyridin-2-yl)-propane-1,3-dione;

1-[3-benzyl-5-(3-methyl-2,6-dioxo-3,6-dihydro-2H-pyrimidin-1-ylmethyl)phenyl]-3-(4-methylpyridin-2-yl)propane-1,3-dione;

1-[3-benzyl-5-(2-oxo-1,2-dihydro-2H-pyrimidin-1-ylmethyl)phenyl]-3-(4-methylpyridin-2-yl)propane- 1,3-dione;

1-(3-benzyl-5-purin-9-ylmethyl)phenyl-3-(4-methylpyridin-2-yl)propane-1,3-dione;

1-[3-benzyl-5-(1,1-dioxoisothiazolidin-2-ylmethyl)phenyl]-3-(4-methylpyridin-2-yl)-propane-1,3-dione;

1-[3-benzyl-5-(1,1-dioxothiazinan-2-ylmethyl)phenyl]-3-(4-methylpyridin-2-yl)-propane-1,3-dione;

1-[3-benzyl-5-(1,1-dioxo-[1,2,6]-thiadiazinan-2-ylmethyl)phenyl]-3-(4-methylpyridin-2-yl)propane-1,3-dione;

1-[3-benzyl-5-(2-oxo-2H-pyrimidin-1-ylmethyl)phenyl]-3-(pyridin-2-yl)propane-1,3-dione;

1-[3-benzyl-5-(1,1-dioxotetrahydrothiophen-2-ylmethyl)phenyl]-3-(4-methylpyridin-2-yl)propane-1,3-dione;

1-[3-benzyl-5-(1,1-dioxotetrahydrothiophen-2-ylmethyl)-2-isopropoxyphenyl]-3-(4-methylpyridin-2-yl)propane-1,3-dione;

1-[3-benzyl-5-(1,3-dimethyl-2,3,6,1-tertrahydro-2,6-dioxopurin-9-ylmethyl)phenyl]-3-(4-methylpyridin-2-yl)propane-1,3-dione;

1-[3-benzyl-5-(6-dimethylaminopurin-7-ylmethyl)phenyl]-3-(4-methylpyridin-2-yl)-propane-1,3-dione;

1-[3-benzyl-5-(4-methyl-5-thioxo-3-trifluoromethyl-4,5-dihydro-[1,24]-triazol-1-ylmethyl)phenyl]-3-(4-methylpyridin-2-yl)propane-1,3-dione;

1-[3-benzyl-5-(3,7-dimethyl-3,7-dihydro-2,6-dioxopurin-1-ylmethyl)phenyl]-3-(4-methylpyridin-2-yl)propane-1,3-dione;

1-[3-benzyl-5-(2-oxo-2H-pyridin-1-ylmethyl)phenyl]-3-(4-methylpyridin-2-yl)propane-1,3-dione;

1-[3-benzyl-5-([1,2,3]triazolo[4,5-b]pyridinyl-2-ylmethyl)phenyl]-3-(4-methylpyridin-2-yl)propane-1,3-dione;

1-(3,5-bis-pyrazol-1-ylmethylphenyl)-3-pyridin-2-yl-propane-1,3-dione;

1-(4-Methylpyridin-2-yl)-3-(3-pyrazol-1-ylmethyl-5-[1,2,4]triazol-1-ylmethylphenyl-propane-1,3-dione;

1-[3,5-bis(3,5-dimethylpyrazol-1-ylmethyl)phenyl]-3-(4-methylpyridin-2-yl)-propane-1,3-dione;

1-(3-benzylphenyl)-3-(5-bromopyridin-2-yl)propane-1,3-dione;

1-(3-benzylphenyl)-3-(5-methoxypyridin-2-yl)propane-1,3-dione;

1-(3-benzylphenyl)-3-(4-imidazol-1-ylmethylpyridin-2-yl)propane-1,3-dione;

1-(3-benzylphenyl)-3-(4(1,2,4-triazol-1-yl)methyl)pyridin-2-yl)propane-1,3-dione;

1-(3-benzylphenyl)-3-(4-(pyrazol-1-ylmethylpyridin-2-yl)propane-1,3-dione;

1-(3-benzylphenyl)-3-(4-(1,2,3,4-tetrazol-2-yl)methyl)pyridin-2-yl)propane-1,3-dione;

1-(3-benzyl-2-([1,2,3]-triazol-1-ylmethyl)phenyl)-3-(4-imidazol-1-ylmethylpyridin-2-yl)propane-1,3-dione;

1-(3-benzylphenyl)-3-(4-methoxymethylpyridin-2-yl)propane-1,3-dione;

1-(3-benzylphenyl)-3-(4-hydroxymethylpyridin-2-yl)propane-1,3-dione;

1-(3-benzylphenyl)-3-[4-(tetrahydrofuran-2-yl)-pyridin-2-yl] propane-1,3-dione;

1-(3,5-bis-pyrazol-1-ylmethyl-phenyl)-3-(4-[1,2,4]triazol-1-ylmethyl-pyridin-2-yl)-propane-1,3-dione;

1-(3,5-bis-pyridin-2-ylmethylphenyl)-3-(4-imidazol-1-ylmethyl-pyridin-2-yl)propane-1,3-dione;

tautomers thereof;

and pharmaceutically acceptable salts thereof.

Additional aspects of the present invention include a compound of Formula (II), or a tautomer thereof, wherein B′ is pyrazinyl, pyrimidinyl, imidazolyl, triazolyl, tetrazolyl, or thiazolyl; and

all other variables are as defined in the first sub-class or as in the second sub-class;

or a pharmaceutically acceptable salt thereof.

Exemplary compounds of the invention also iclude compounds. selected from the group consisting of

1-(3-Benzylphenyl)-3-(1-H-imidazol-2-yl)-propane-1,3-dione;

1-(3-benzylphenyl)-3-(1benzyl-1H-imidazol-2-yl)-propane1,3dione;

1-(3-Benzylphenyl)-3-(imidazole-4-yl)propane-1,3-dione;

1-(3-Benzylphenyl)-3-pyrazin-2-ylpropane-1,3-dione;

1-(3-Benzylphenyl)-3-(2-methylthiazol-4-yl)-propane-1,3-dione;

1-[3-Benzyl-5-(5-methylpyrazin-2-ylmethyl)phenyl]-3-(5-methylpyrazin-2-yl)-propane-1,3-dione;

1-(3-benzylphenyl)-3-(4H-[1,2,4]triazol-3-yl)propane-1,3-dione;

tautomers thereof;

and pharmaceutically acceptable salts thereof.

Exemplary compounds of the invention also include compounds selected from the group consisting of

1-[3-(1,1-Dioxoisothiazolin-2-ylmethyl)-5-(pyridin-2-ylmethyl)phenyl]-3-(1-methyl-1H-imidazol-4-yl)propane-1,3-dione;

1-(3-benzylphenyl)-3-(1-N-methyl-imidazole-4-yl)propane-1,3-dione;

1-(3-benzylphenyl)-3-[1-N-(pyridin-4-yl)methylimidazole-4-yl]propane-1,3-dione;

1-(3-benzylphenyl)-3-[1-N-(pyridin-2-yl)methylimidazole-4-yl]propane-1,3-dione;

1-(3-benzylphenyl)-3-[1-N-(pyridin-3-yl)methylimidazole-4-yl]propane-1,3-dione;

1-(3-benzylphenyl)-3-{1-N-[(1-N-tert-butylcarbamyl)-piperidine-4-yl]methylimidazole-4-yl}propane-1,3-dione;

1-(3-benzylphenyl)3-[1-N-(piperidine-4-yl)methylimidazole-4-y]propane-1,3-dione;

1-(3-benzylphenyl)-3-{1-N-[(1-N-methanesulfonyl)piperidine-4-yl]methylimidazole-4-yl}propane-1,3-dione;

1-(3-benzylphenyl)-3-{1-N-[2-(1-N-tert-butylcarbamylpiperiazin-4-yl)ethyl]imidazole-4-yl}propane-1,3-dione;

1-(3-benzylphenyl)-3-{1-N-[2-(piperiazin-1-yl)ethyl]imidazole-4-yl}propane-1,3-dione;

1-(3-benzylphenyl)-3-{1-N-[2-(1-N-methanesulfonyl-piperazin-4-yl)ethyl]-imidazole-4-yl}propane-1,3-dione;

1-(3-benzylphenyl)-3-{1-N-[2-(1-N-benzylpiperiazin-4-yl)ethyl]imidazole-4-yl}propane-1,3-dione;

1-[3-benzyl-5-(6-oxo-6H-pyrimidin-1-ylmethyl)phenyl]-3-(1-methylimidazole-4-yl)propane-1,3-dione;

1-[3-benzyl-5-(1,1-dioxoisothiazolidin-2-ylmethyl)phenyl]-3-(1-methylimidazole-4-yl)propane-1,3-dione;

1-(3-benzylphenyl)-3-pyrimidin-2-yl-propane-1,3-dione;

1-(3-benzylphenyl)-3-(4-methylpyrimidin-2-yl)propane-1,3-dione;

1-(3,5-bis-pyrazol-1-ylmethylphenyl)-3-pyrimidin-2-yl-propane-1,3-dione;

1-(3,5-bis-pyrazol-1-ylmethylphenyl)-3-(4-methylpyrimidin-2-yl)propane-1,3-dione;

1-(3,5-bis-pyrazol-1-ylmethyl-phenyl)-3-(1H-imidazol-2-yl)propane-1,3-dione;

1-(3,5-bis-pyrazol-1-ylmethyl-phenyl)-3-(1-methyl-1H-imidazol-4-yl)propane-1,3-dione;

1-{3-benzyl-5-[(1,1-dioxido-1,2-thiazinan-2-yl)methyl]phenyl}-3-(1,3-thiazol-2-yl)-1,3-propanedione;

1-{3-benzyl-5-[(1,1-dioxido-2-isothiazolidinyl)methyl]phenyl}-3-(1,3-thiazol-2-yl)-1,3-propanedione;

1-{3-benzyl-5-[(6-oxo-1(6H)-pyrimidinyl)methyl]phenyl}-3-(1,3-thiazol-2-yl)-1,3-propanedione;

tautomers thereof;

and pharmaceutically acceptable salts thereof.

Additional embodiments of the invention include a compound of Formula (I), or a tautomer thereof, wherein

B is an 8- to 10-membered fused bicyclic heterocycle containing from 1 to 4 nitrogen atoms, 0 to 2 sulfur atoms, and carbon atoms, wherein the ring of the heterocycle attached to the central, dione moiety is a 5- or 6-membered heteroaromatic ring containing at least one nitrogen or sulfur atom and the other ring of the heterocycle is a saturated or unsaturated ring;

and all other variables are as originally defined or as defined in any one of the first, second, and third embodiments;

or a pharmaceutically acceptable salt thereof.

An aspect of each of the immediately preceding embodiments is a compound of Formula (I) in which B is other than indole. Another aspect of each of these embodiments is a compound of Formula (I) in which when A is (ii) fused bicyclic heterocycle, then each of A and B is other than indole.

An exemplary compound of the invention includes.

1-(3-benzylphenyl)-3-(5,6,7,8-tetrahydro-[1,8]naphthyridin-2-yl)-propane1,3-dione;

a tautomer thereof;

or a pharmaceutically acceptable salt thereof.

Still other embodiments of the invention include a compound of Formula (I), or a tautomer thereof, wherein

A is an 8- to 10-membered fused bicyclic heterocycle containing carbon atoms and from 1 to 3 heteroatoms selected from nitrogen, oxygen and sulfur, wherein the ring of the heterocycle attached to the central, dione moiety is a benzene ring, and the other ring of the heterocycle is a saturated or unsaturated heteroatom-containing ring;

and all other variables are respectively as originally defined above and as defined in the first embodiment;

or a pharmaceutically acceptable salt thereof.

An aspect of each of the immediately preceding embodiments is a compound of Formula (I) in which A is other than indole. Another aspect of each of these embodiments is a compound of Formula (I) in which each of A and B is other than indole.

An exemplary compound of the invention includes

1-(6-benzyl-3-oxo-3,4-dihydro-2H-benzo[1,4]oxazin-8-yl)-3-(4-methylpyridin-2-yl)propane-1,3-dione;

a tautomer thereof;

or a pharmaceutically acceptable salt thereof.

Still further embodiments of the invention include a compound of Formula (I), or a tautomer thereof, wherein

A is a 5- or 6-membered heteroaromatic ring containing 0, 1 or 2 nitrogen atoms and 0 or 1 sulfur atoms; and embodiment;

or a pharmaceutically acceptable salt thereof.

Additional embodiments of the present invention include a compound of Formula (I) or a tautomer thereof, wherein

A is a 5- or 6-membered heteroaromatic ring containing 0, 1 or 2 nitrogen atoms and 0 or 1 sulfur atoms;

B is (i) a 5- or 6-membered heteroaromatic ring containing from 1 to 4 nitrogen atoms, 0 or 1 sulfur atoms, and at least 1 carbon atom, or (ii) an 8- to 10-membered fused bicyclic heterocycle containing from 1 to 3 nitrogen atoms and carbon atoms, wherein the ring of the heterocycle attached to the central dione moiety is a 5- or 6-membered heteroaromatic ring containing at least one nitrogen atom and the other ring of the heterocycle is a saturated or unsaturated ring; wherein B is attached to the central dione moiety via a carbon atom and at least one nitrogen or sulfur atom in B is adjacent to the point of attachment; and

all other variables are respectively as originally defined and as defined in the first embodiment;

or a pharmaceutically acceptable salt thereof.

An aspect of each of the immediately preceding embodiments is a compound of Formula (I) in which when B is (ii) a fused bicyclic heterocycle, B is other than indole.

A third class of the present invention is a compound of Formula (I), or a tautomer thereof, wherein

A is pyrrolyl, thienyl, or pyridyl; and

B is (i) a heteroaromatic ring selected from pyridyl, pyrazinyl, pyrimidinyl, imidazolyl, triazolyl, tetrazolyl, and thiazolyl,or (ii) a fused bicyclic heterocycle selected from

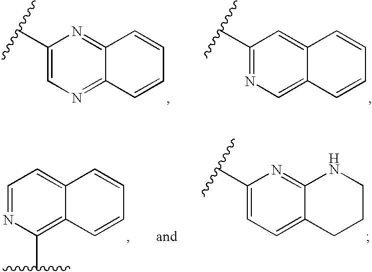

and all other variables are as originally defined;

or a pharmaceutically acceptable salt thereof.

A third sub-class of the present invention is a compound of Formula (I), or a tautomer thereof, wherein

R¹ is hydrogen, fluoro, chloro, bromo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, N(R^{a}) (R^{b}), CH₂N(R^{a})(R^{b}), (CH₂)₀₋₂R^{c}, or O(CH₂)₀₋₂R^{c};

R² is hydrogen, fluoro, chloro, bromo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, N(R^{a})(R^{b}), CH₂N(R^{a})(R^{b}), (CH₂)₀₋₂R^{c}, O(CH₂)₀₋₂R^{c}, O(CH₂)₀₋₂R^{d}, O(CH₂)₀₋₂R^{d},C(═O)CH₂C(═O)R^{e}, or R^{f};

R³ is hydrogen, fluoro, chloro, bromo, oxo, methyl, ethyl, propyl, isopropyl, C₃-C₆ cycloalkyl, C₃-C₆ cycloalkyloxy, OCH₃, OCH₂CH₃, OCH₂CH₂CH₃, OCH(CH₃)₂, CF₃, CH₂OF₃, OCF₃, OCH₂CF₃, (CH₂)₁₋₃O(CH₂)₀₋₁CH₃, (CH₂)₁₋₃O(CH₂)₀₋₁CF₃ N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, (CH₂)₀₋₂R^{c}, or (CH₂)₀₋₂R^{g};

R⁴ and R⁵ are substituents attached to any nitrogen or carbon in B′ except for the ring carbon attached to the central, dione moiety, and are each independently selected from hydrogen, fluoro, chloro, bromo, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₂OH, (CH₂)₁₋₂O—C₁-C₄ alkyl, (CH₂)₁₋₃O(CH₂)₀₋₁CF₃, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, and (CH₂)₀₋₂R^{h};

R^{a} and R^{b} are each independently hydrogen, C₁-C₄ alkyl, or fluorinated C₁-C₄ alkyl;

R^{c} is (i) phenyl or substituted phenyl, wherein each substituent on the substituted phenyl is independently fluoro, chloro, bromo, hydroxy, (CH₂)₁₋₂OH, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R_{b}, or (ii) an 8- to 10-membered fused bicyclic carbocycle in which one ring is a benzene ring and the other ring is a saturated or unsaturated ring, wherein the fused carbocycle is unsubstittuted or substituted with one or more substituents selected from fluoro, chioro, bromo hydroxy, (CH₂)₁₋₂OH, methyl, ethyl ,propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (C₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R_{b};

R^{d}, a 5- or 6-membered monocyclic heterocycle which is saturated or unsaturated and which contains at least one carbon atom and from 1 to 4 heteroatoms selected from nitrogen, oxygen, and sulfur, wherein each ring sulfur is in a form selected from S, SO and SO₂; (ii) an 8- to 10-membered fused bicyclic heterocycle in which either ring is saturated or unsaturated and which contains carbon atoms and from 1 to 4 nitrogen atoms; wherein the heterocyle of (i) or (ii) is unsubstituted or substituted with one or more substituents selected from fluoro, chloro, bromo, hydroxy, (CH₂)₁₋₂OH, oxo, thio, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl; or (iii) a 5- or 6-membered monocyclic heterocycle which is saturated or unsaturated and which contains carbon atoms and from 1 to 3 nitrogen atoms, the heterocycle being substituted with spiro-C₁-C₂ alkylenedioxy, or with one of pyrrolidinyl, piperidinyl, piperazinyl, or morpholinyl, which is unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N (R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl;

R^{e} is a 5- or 6-membered heteroaromatic ring containing from 1 to 4 nitrogen atoms, 0 to 2 sulfur atoms, and at least 1 carbon atom; wherein the point of attachment of the ring is a carbon atom and at least one nitrogen or sulfur atom in the ring is adjacent to the point of attachment; wherein the ring is unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₂N(R^{a})SO₂R^{b}, phenyl, and benzyl;

R^{f} is X—NH(CH₂)₁₋₂Y, wherein X is a 5- or 6-membered monocyclic heterocycle which is saturated or unsaturated and which contains carbon atoms and from 1 to 3 nitrogen atoms and which is unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R_{b}; Y is pyrrolidinyl, piperidinyl, piperazinyl, or morpholinyl, which is unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R_{b};

R^{g} is a 5- or 6-membered monocyclic heterocycle which is saturated or unsaturated and which contains one or more carbon atoms and from 1 to 4 nitrogen atoms, the heterocycle being unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl; and

R^{h} is (i) C₃-C₆ cycloalkyl; (ii) phenyl; (iii) substituted phenyl, wherein each substituent on the substituted phenyl is independently fluoro, chloro, bromo, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, (CH₂)₁₋₂OH, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₄SO₂R^{a}, SO₂N(R^{a})(R^{b}), (CH₂)₁₋₂SO₂N(R^{a})(R^{b}), (CH₂)₁₋₂N(R^{a})SO₂R^{b}, or (iv) a 5- or 6-membered monocyclic heterocycle which is saturated or unsaturated and which contains at least one carbon atom and from 1 to 4 heteroatoms selected from nitrogen, oxygen, and sulfur; wherein the heterocycle is unsubstituted or substituted with one or more substituents selected from fluoro, chloro, bromo, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, (CH₂)₁₋₂OH, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl;

and A and B are as defined in the third class;

or a pharmaceutically acceptable salt thereof.

A fourth sub-class of the present invention is a compound of Formula (I), or a tautomer thereof, wherein

R^{c} is (i) phenyl or substituted phenyl or (ii) an unsubstituted or substituted fused bicyclic carbocycle selected from

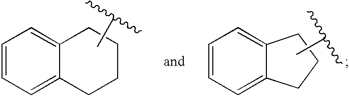

R^{d} is (i) an unsubstituted or substituted 5- or 6-membered monocyclic heterocycle selected from pyrazolyl, imidazolyl, pyrrolyl, pyrrolidinyl, pyridyl, piperidinyl, pyrazinyl, piperazinyl, pyridazinyl, pyrimidinyl, 1,2,4-triazolyl, 1,2,3-triazolyl, tetrazolyl, morpholinyl, tetrahydrothienyl, tetrahydrodioxothienyl, thiadiazinanyl t dioxothiadiazinanyl, thiazinanyl, dioxothiazinanyl, thiazolidinyl, dioxothiazolidinyl, isothiazolidinyl, isodioxothiazolidinyl, thiazolyl, and isothiazolyl; (ii) an unsubstituted or substituted fused bicyclic heterocycle selected from

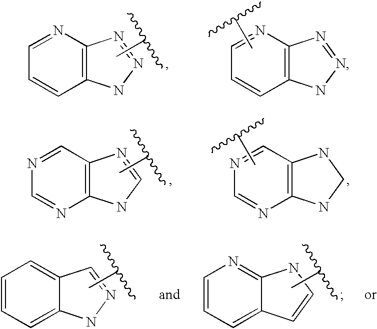

(iii) a monocyclic heterocycle selected from pyridyl, piperidinyl, pyrazinyl, piperazinyl, and pyrimidinyl, the heterocycle being substituted with spiro-C₁-C₂ alkylenedioxy or with one of unsubstituted or substituted piperidinyl, unsubstituted or substituted piperazinyl, or unsubstituted or substituted morpholinyl;

R^{e} is an unsubstituted or substituted heteroaromatic ring selected from pyridyl, pyrazinyl, and pyrimidinyl;

R^{f} is X—NH(CH₂)₁₋₂Y, wherein X is selected from unsubstituted or substituted pyridyl, unsubstituted or substituted pyrazinyl, and unsubstituted or substituted pyrimidinyl; and Y is unsubstituted or substituted pyrrolidinyl, unsubstituted or substituted piperidinyl, unsubstituted or substituted piperazinyl, or unsubstituted or substituted morpholinyl;

R^{g} is an unsubstituted or substituted monocyclic heterocycle selected from pyridyl, pyrrolyl, 1,2,3-triazolyl, 1,2,4-triazolyl, pyrazolyl, imidazolyl, tetrazolyl, piperidinyl, and piperazinyl; and

R^{h} is C₃-C₆ cycloalkyl, phenyl, substituted phenyl, or an unsubstituted or substituted monocyclic heterocycle selected from pyridyl, pyrrolyl, 1,2,3-triazolyl, 1,2,4-triazolyl, pyrazolyl, irmidazolyl, tetrazolyl, piperidinyl, piperazinyl, and tetrahydrofuranyl;

and all other variables are as defined in the third sub-class;

or a pharmaceutically acceptable salt thereof.

A fourth class of the present invention is a compound of Formula (I), or a tautomer thereof, wherein

A is pyrrolyl, thienyl, or pyridyl;

R¹ is hydrogen, fluoro, chloro, bromo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃OCH₃, (CH₂)₁₋₃OCF₃, N(R^{a})(R^{b}), CH₂N(R^{a})(R^{b}), (CH₂)₀₋₂R^{c}, or O(CH₂)₀₋₂R^{c};

R² is hydrogen, fluoro, chloro, bromo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃OCH₃, (CH₂)₁₋₃OCF₃, N(R^{a})(R^{b}), CH₂N(R^{a})(R^{b}), (CH₂)₀₋₂R^{c}, O(CH₂)₀₋₂R^{c}, (CH₂)₀₋₂R^{d}, or O(CH₂)₀₋₂R^{d};

R³ is hydrogen, fluoro, chloro, bromo, oxo, methyl, ethyl, propyl, isopropyl, C₃-C₆ cycloalkyl, C₃-C₆ cycloalkyloxy, OCH₃, OCH₂CH₃, OCH₂CH₂CH₃, OCH(CH₃)₂, CF₃, CH₂CF₃, OCF₃, OCH₂CF₃, (CH₂)₁₋₃OCF₃, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₄N(R^{a})C(═O)R^{b}, SO₂R^{a}, (CH₂)₁₋₄SO₂R^{a}, SO₂N(R^{a})(R^{b}), (CH₂)₁₋₄SO₂N(R^{a})(R^{b}), (CH₂)₁₋₄N(R^{a})SO₂R^{b} (CH₂)₀₋₂R^{c}, or (CH₂)₀₋₂R^{g};

B is (i) a heteroaromatic ring selected from pyridyl, pyrazinyl, pyrimidinyl, imidazolyl,triazolyl, tetrazoly, and thiazolyl, or (ii) a fused bicyclic heterocycle selected from

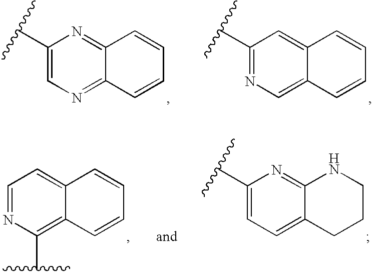

R⁴ and R⁵ are substituents attached to any nitrogen or carbon in B except for the ring carbon attached to the central dione moiety, and are each independently selected from hydrogen, fluoro, chloro, bromo, hydroxy, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₄SO₂R^{a}, SO₂N(R^{a})(R^{b}), (CH₂)₁₋₂SO₂N(R^{a})(R^{b}), (CH₂)₁₋₂N(R^{a})SO₂R^{b}, and (CH₂)₀₋₂R^{h};

R^{a} and R^{b} are each independently, hydrogen, methyl, ethyl, CF₃ CH₂CF₃, OCF₃, or OCH₂CF₃;

R^{c} is (i) phenyl or substituted phenyl, wherein each substituent on the substituted phenyl is independently fluoro, chloro, bromo, cyano, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, N(R^{a})(R^{b}), (CH₂)₁₋₂N(R^{a})(R^{b}), (CH₂)₀₋₂CO₂R^{a}, (CH₂)₀₋₂C(═O)N(R^{a})(R^{b}), (CH₂)₀₋₂SO₂R^{a}, (CH₂)₁₋₃OCH₃, or (CH₂)₁₋₃OCF₃; or (ii) an 8- to 10-membered fused bicyclic carbocycle in which one ring is a benzene ring and the other ring is a saturated or unsaturated ring, wherein the fused carbocycle

is unsubstituted or substituted with one or more substituents selected from fluoro, choro, bromo, cyano, hydroxy, methyl, ethyl, propyl, isoprpyl, OCH₃, OCH₂CH₃, OCH₂CH₂CH₃, OCH(CH₃)₂, CF₃, CH₂CF₃, OCF₃, OCH₂CF₃, (CH₂)₀₋₂CO₂R^{a}, (CH₂)₀₋₂C(═O)N(R^{a})(R^{b}), (CH₂)₀₋₂SO₂R^{a}, (CH₂)₁₋₃OCH₃, and (CH₂)₁₋₃OCF₃,

R^{d} is (i)a 5- or 6-membered monocyclic which is saturated or unsaturated and which contains at least one carbon atom and from 1 to 4 heteroatoms selected from nitrogen, oxygen, and sulfur; (ii) an 8- to 10-membered fused bicyclic heterocycle in which either ring is saturated or unsaturated and which contains carbon atoms and from 1 to 4 nitrogen atom; wherein the heterocycle of (i) or (ii) is unsubstituted or substituted with one or more substituents selected from fluoro, chloro, bromo, cyano, hydroxy, oxo, N(R^{a})(R^{b}), methyl, ethyl, propyl, isopropyl, OCH₃, OCH₂CU₃, OCH₂CH₂CH₃, OCH(CH₃)₂, CF₃, CH₂CF₃, OCF₃, OCH₂CF₃, (CH₂)₀₋₂CO₂R^{a}, (CH₂)₀₋₂C(═O)N(R^{a})(R^{b}), (CH₂)₁₋₂SO₂R^{a}, (CH₂)₁₋₃OCH₃, and (CH₂)₁₋₃OCF₃; or (iii) a 5- or 6-membered monocyclic heterocycle which is saturated or unsaturated and which contains carbon atoms and from 1 to 3 nitrogen atoms, the heterocycle being substituted with spiro-C₁-C₂ alkylenedioxy, or with one of pyrrolidinyl, piperidinyl, piperazinyl, or morpholinyl which is unsubstituted or, substituted with one or more substituents selected from fluoro, chloro, bromo, cyano, hydroxy, N(R^{a})(R^{b}), methyl, ethyl, propyl, isopropyl, OCH₃, OCH₂CH₃, OCH₂CH₂CH₃, OCH(CH₃)₂, CF₃, CH₂CF₃, OCF₃, and OCH₂CF₃;

R^{g} is a 5- or 6-membered monocyclic heterocycle which is saturated or unsaturated and which contains one or more carbon atoms and from 1 to 4 nitrogen atoms, the heterocycle being unsubstituted or substituted with one or more substituents selected from fluoro, chloro, bromo, cyano, hydroxy, N(R^{a})(R^{b}) methyl, ethyl, propyl, isopropyl, OCH₃, OCH₂CH₃, OCH₂CH₂CH₃, OCH(CH₃)₂, CF₃, CH₂CF₃, OCF₃, and OCH₂CF₃; and

R^{h} is C₃-C₆ cycloalkyl, phenyl or substituted phenyl, wherein each substituent on the substituted phenyl is independently fluoro, chloro, bromo, cyano, hydroxy, methyl, ethyl, propyl, isopropyl, OCH₃, OCH₂CH₃, OCH₂CH₂CH₃, OCH(CH₂CF₃, CH₂CF₃, OCF₃, OCH₂CF₃, (CH₂)₀₋₂CO₂R^{a}, (CH₂)₀₋₂C(═O)N(R^{a})(R^{b}), (CH₂)₀₋₂SO₂R^{a}, (CH₂)₁₋₃OCH₃, or (CH₂)₁₋₃OCF₃;

or a pharmaceutically acceptable salt thereof.

A fifth sub-class of the present invention is a compound of Formula (T), or a tautomer thereof, wherein

R^{a} and R^{b} are each independently hydrogen or methyl;

R^{c} is (i) phenyl or substituted phenyl, wherein each substituent on the substituted phenyl is independently fluoro, chloro, cyano, methyl, ethyl, propyl, isopropyl, OCH₃, CF₃, OCF₃, or C₂CH₃; or(ii) a fused bicyclic carbocycle selected from

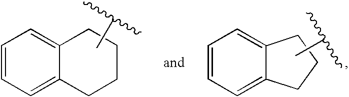

wherein the fused carbocycle is unsubstituted or substituted with one or more substituents selected from fluoro, chloro, cyano, methyl, ethyl, propyl, isopropyl, OCH₃, CF₃, OCF₃ and CH₂OCH₃;

R^{d} is (i) a 5- or 6-membered monocyclic heterocycle selected from pyrazolyl, imidazolyl, pyrrolyl, pyrrolidinyl, pyridyl, piperidinyl, pyrazinyl, piperazinyl, pyridazinyl, pyrimidinyl, 1,2,4-triazolyl, 1,2,3-triazolyl, tetrazolyl, and morpholinyl; (ii) a fused bicyclic heterocycle selected from

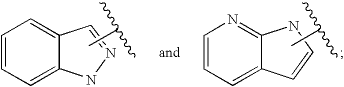

wherein the heterocycle of (i) or (ii) is unsubstituted or substituted with one or more substituents selected from fluoro, chloro, cyano, hydroxy, oxo, N(R^{a})(R^{b}), methyl, ethyl, propyl, isopropyl, OCH₃, CF₃, OCF₃, and CH₂OCH₃; or (iii) a monocyclic heterocycle selected.from pyridyl, piperidinyl, pyrazinyl, piperazinyl, and pyrimidinyl, the heterocycle being substituted with spiro-C₁-C₂ alkylenedioxy, or with one of piperidinyl, piperazinyl, or morpholinyl, which is unsubstituted or substituted with one or more substituents selected from fluoro, chloro, N(R^{a})(R^{b}), methyl, ethyl, propyl, isopropyl, OCH₃, CF₃, and OCF₃;

R^{g} is a monocyclic heterocycle selected from pyridyl, pyrrolyl, 1,2,3-triazolyl, 1,2,4-triazolyl, pyrazolyl, and imidazolyl, the heterocycle being unsubstituted or substituted with one or more substituents selected from fluoro, chloro, N(R^{a})(R^{b}), methyl, ethyl, propyl, isopropyl, OCH₃, CF₃, and OCF₃; and

R^{h} is C₃-C₆ cycloalkyl, phenyl or substituted phenyl, wherein each substituent on the substituted phenyl is independently fluoro, chloro, methyl, ethyl, propyl, isopropyl, OCH₃, CF₃, and OCF₃; and

all other variables are as defined above in the fourth class;

or a pharmaceutically acceptable salt thereof.

Exemplary compounds of the invention include compounds selected from the group consisting of

1-[1-(4-fluorobenzyl)-1H-pyrrol-2-yl]-3-pyrimidin-4-yl-propan-1,3-dione;

1-[1-(4-fluorobenzyl)-1H-pyrrol-2-yl]-3-thiazol-2-yl-propan-1,3-dione;

1-[1-(4-Fluorobenzyl)-1H-pyrrol-2-yl]-3-(4-methylpyridin-2-yl)propan-1,3-dione;

1-(1-Benzyl-1H-imidazol-2-yl)-3-[1-(4-fluorobenzyl)-1H-pyrrol-2-yl)propan-1,3-dione;

1-[1-(4-Fluorobenzyl)-1H-pyrrol-3-yl]-3-pyridin-2-ylpropan-1,3-dione;

1-(1-(4-Fluorobenzyl)-1H-imidazol-2-yl)-3-[1-(4-fluorobenzyl)-1H-pyrrol-2-yl)propan-1,3-dione;

1-[1-(4-Fluorobenzyl)-1H-pyrrol-2-yl]-3-(4H-[1,2,4]triazol-3-yl-propan-1,3-dione;

1-[1-(4-Fluorobenzyl)-4-(2-oxo-2H-pyridin-1-yl)-1H-pyrrol-2-yl]-3-pyridin-2-yl-propan-1,3-dione;

1-(1H-Imidazol-2-yl)-3-(5-phenethylthiophen-2-yl)propane-1,3-dione;

1-(5-Benzyl-thiophen-2-yl)-3-pyridin-2-yl-propane-1,3-dione;

1-(5-Benzyl-thiophen-2-yl)-3-(4-methyl-pyridin-2-yl)propane-1,3-dione;

1-[5-(3-Chlorobenzyl)thiophen-2-yl]-3-pyridin-2-yl-propane-1,3-dione;

1-[5-(4-Fluorobenzyloxy)thiophen-2-yl]-3-(4-methylpyridin-2-yl)propane-1,3-dione;

1-[5-(4-Fluorobenzyloxy)thiophen-2-yl]-3-pyridin-2-yl-propane-1,3-dione;

tautomers thereof;

and pharmaceutically acceptable salts thereof.

A preferred embodiment of the invention is a compound selected from the group consisting of

1-(3,5-bis-pyrazol-1-ylmethylphenyl)-3-(4-methylpyridin-2-yl)propane-1,3-dione;

1-(3-benzyl-5-tetrazol-2-ylmethylphenyl)-3-(4-methylpyridin-2-yl)propane-1,3-dione;

1-(3-benzyl-5-[1,2,4]triazol-1-ylmethylphenyl)-3-(4-methylpyridin-2-yl)-propane-1,3-dione;

1-(3-benzyl-5-tetrazol-1-ylmethylphenyl)-3-(4-methylpyridin-2-yl)propane-1,3-dione;

1-(3-benzyl-5-[1,2,3]triazol-2-ylmethylphenyl)-3-(4-methylpyridin-2-yl)-propane-1,3-dione;

1-[3-benzyl-5-(4-methylpiperazin-1ylmethyl)phenyl]-3-(4-methylpyridin-2-yl)propane-1,3-dione;

1-(3-benzyl-5-[1,2,3]triazol-1-ylmethylphenyl)-3-(4-methylpyridin-2-yl)propane-1,3-dione;

1-(3-benzyl-5-pyrazin-2-ylmethylphenyl)-3-(4-methylpyridin-2-yl)propane-1,3-dione; 1-[3-benzyl-5-(2-oxopiperidin-1-ylmethyl)phenyl]-3-(4-methylpyridin-2-yl)propane-1,3-dione;

1-(3-Benzyl-5-[1,2,4]triazol-1-ylmethylphenyl)-3-(4-methylpyridin-2-yl)-propane-1,3-dione;

1-(3,5-bis-pyridin-2-ylmethylphenyl)-3-(4-methylpyridin-2-yl)propane-1,3-dione; 1-[3-benzyl-5-(3-methyl-2,6-dioxo-3,6-dihydro-2H-pyrimidin-1-ylmethyl)phenyl]-3-(4-methylpyridin-2-yl)propane-1,3-dione; 1-[3-benzyl-5-(2-oxo-1,2-dihydro-2H-pyrimidin-1-ylmethyl)phenyl]-3-(4-methylpyridin-2-yl)propane-1,3-dione;

1-[3-benzyl-5-(1,1-dioxoisothiazolidin-2-ylmethyl)phenyl]-3-(4-methylpyridin-2-yl)-propane-1,3-dione;

1-[3-benzyl-5-(1,1-dioxothiazinan-2-ylmethyl)phenyl]-3-(4-methylpyridin-2-yl)-propane-1,3-dione;

1-[3-benzyl-5-(1,1-dioxo-[1 ,2,6]-thiadiazinan-2-ylmethyl)phenyl]-3-(4-methylpyridin-2-yl)propane-1,3-dione;

1-(3-benzylphenyl)-3-(1H-imidazol-2-yl)propane-1,3-dione;

1-(3-benzylphenyl)-3-(1H-imidazol-4-yl)propane-1,3-dione;

1-[3-benzyl-5-(1,1-dioxoisothiazolidin-2-ylmethyl)phenyl]-3-(1-methylimidazole-4-yl)propane-1,3-dione;

1-{3-benzyl-5-[(6-oxo-1(6H)-pyrimidinyl)methyl]phenyl}-3-(1,3-thiazol-2-yl)-1,3-propanedione;

tautomers thereof;

and pharmaceutically acceptable salts thereof.

Other embodiments of the present invention include the following:

(a) A pharmaceutical composition comprising a compound of Formula (I) and a pharmnaceutically acceptable carrier.

(b) The pharmaceutical composition of (a), further comprising at least one antiviral, selected from the group consisting of HIV protease inhibitors, non-nucleoside HIV reverse transcriptase inhibitors, and nucleoside HIV reverse transcriptase inhibitors.

(c) A method of inihibiting HIV integrase in a subject in need thereof which comprises administering to the subject a therapeutically effective amount of a compound of Formula (I).

(d) A method of preventing or treating infection by HIV in a subject in need thereof which comprises administering to the subject a therapeutically effective amount of a compound of Formula (I).

(e) The method of (d), wherein the compound of Formula (I) is administered in combination with a therapeutically effective amount of at least one antiviral, selected from the group consisting of HIV protease inhibitors, non-nucleoside HIV reverse transcriptase inhibitors, and nucleoside HIV reverse transcriptase inhibitors.

(f) A method of treating AIDS in a subject in need thereof which comprises administering to the subject a therapeutically effective amount of a compound of Formula (I).

(g) Thee method of (f), wherein the compound is administered in combination with a therapeutically effective amount of at least one antiviral, selected from the group consisting of HIV protease inhibitors non-nucleoside HIV reverse transcriptase inhibitors, and nucleoside HIV reverse transcriptase inhibitors

h) A method of inhibiting HIV integrase in a subject in need thereof which comprises administering to the subject a therapeutically effective amount of the composition of (a) or (b).

(i) A method of preventing or treating infection by HIV in a subject in need thereof which comprises administering to the subject a therapeutically effective amount of the composition of (a) or (b).

(j) A method of preventing or treating HIV infection in a subject in need thereof which comprises administering to the subject a therapeutically effective amount of the composition of (a) or (b).

(k) A method of treating AIDS in a subject in need thereof which comprises administering to the subject a therapeutically effective amount of the composition of (a) or (b).

(l) A method of treating AIDS in a subject in need thereof which comprises administering to the subject a therapeutically effective amount of the composition of (a) or (b).

Additional embodiments of the invention include the pharmaceutical compositions and methods set forth in (a)-(l) above, wherein the compound employed therein is a compound of one of the embodiments, classes, sub-classes, or aspects of compounds described above.

As used herein, the term “C₁-C₆ alkyl” means linear or branched chain alkyl groups having from 1 to 6 carbon atoms and includes all of the hexyl alkyl and pentyl alkyl isomers as well as n-, iso-, sec- and t-butyl, n- and isopropyl, ethyl and methyl. “C₁-C₄ alkyl” means n-, is o-, sec- and t-butyl, n- and isopropyl, ethyl and methyl.

The term “C₁-C₆ alkoxy” means an —O-alkyl group wherein alkyl is C₁ to C₆ alkyl. “C₁-C₄ alkoxy” has an analogous meaning; i.e., it is an alkoxy group selected from methoxy, ethoxy, n-propoxy, isopropoxy, n-butoxy, is obutoxy, tert-butoxy, and sec-butoxy.

The term “C₂-C₈ alkoxyalkyl” means a linear or branched C₁-C₆ alkyl group as defined above having as a substituent a C₁-C₆ alkoxy group as defined above, wherein the alkoxyalkyl group has a total of from 2 to 8 carbon atoms. Representative examples of suitable alkoxyalkyl groups include, but are not limited to, the C₁-C₆ alkoxy-substituted methyl groups (methoxymethyl, ethoxymethyl, n-propoxymethyl, isopropoxymethyl, and the butyloxymethyl, pentyloxymethyl, and hexyloxymethyl isomers), and the C₁-C₆ alkoxy-substituted ethyl groups. Other suitable alkoxyalkyl groups include the series (CH₂)₁₋₆OCH₃, (CH₂)₁₋₄OCH₃, (CH₂)₁₋₃OCH₃, (CH₂)₁₋₆OCH₂CH₃, (CH₂)₁₋₄OCH₂CH₃ and (CH₂)₁₋₃OCH₂CH₃.

The term “C₃-C₇ cycloalkyl” means a cyclic ring of an alkane having three to seven total carbon atoms (i.e., cyclopropyl, cyclobutyl, cyclopentyl, cyclohexyl, or cycloheptyl). The term “C₃-C₆ cycloalkyl” refers to a cyclic ring selected from cyclopropyl, cyclobutyl, cyclopentyl, and cyclohexyl. “C₃-C₅ cycloalkyl” has an analogous meaning.

The term “C₃-C₇ cycloalkyloxy” mean a group —OR* wherein R* is C₃-C₇ cycloalkyl as defined above. Each of the terms “C₃-C₆ cycloalkyloxy” and “C₃-C₅ cycloalkyloxy” has an analogous meaning.

The term “halogen” (which may alternatively be referred to as “halo”) refers to fluorine, chlorine, bromine and iodine (alternatively, fluoro, chloro, bromo, and iodo).

The term “thio” (also referred to herein as “thioxo”) means divalent sulfur; i.e., ═S.

The term “fluorinated C₁-C₆ alkyl” (which may alternatively be referred to as “C₁-C₆ fluoroalkyl”) means a C₁ to C₆ linear or branched alkyl group as defined above with one or more fluorine substituents. The term “fluorinated C₁-C₄ alkyl” has an analogous meaning. Representative examples of suitable fluoroalkyls include the series (CH₂)₀₋₄CF₃ (i.e., trifluoromethyl, 2,2,2-trifluoroethyl, 3,3,3-trifluoro-n-propyl, etc.), 1-fluoroethyl, 2-fluoroethyl, 2,2-difluoroethyl, 3,3,3-trifluoroisopropyl, 1,1,1,3,3,3-hexafluoroisopropyl, and perfluorohexyl.

The term “fluorinated C₁-C₆ alkoxy” (which may alternatively be referred to as “C₁-C₆ fluoroalkoxy”) means a C₁-C₆ alkoxy group as defined above wherein the alkyl moiety has one or more fluorine substituents. The term “fluorinated C₁-C₄ alkoxy” has an analogous meaning. Representative examples include the series O(CH₂)₀₋₄CF₃ (i.e., trifluoromethoxy, 2,2,2-trifluoroethoxy, 3,3,3-trifluoro-n-propoxy, etc.), 1,1,1,3,3,3-hexafluoroisopropoxy, and so forth.

The term “fluorinated C₂-C₈ alkoxyalkyl” means C₂-C₈ alkoxyalkyl as defined above, wherein either or both the alkoxy moiety and the alkyl moiety has one or more fluorine substituents. Representative examples of suitable fluorinated alkoxyalkyl groups include, but are not limited to, the C₁-C₆ fluoroalkoxy-substituted methyl groups (e.g., fluoromethoxymethyl, 2-fluoroethoxymethyl, and 3-fluoro-n-propoxymethyl), C₁-C₆ difluoroalkoxymethyl groups (e g., difluoromethoxymethyl and 2,2-difluoroethoxymethyl, C₁-C₆ trifluoroalkoxy-substituted methyl groups (e.g., trifluoromethoxymethyl and 2,2,2-trifluoroethoxymethyl), C₁-C₆ alkoxy-substituted fluoromethyl groups (e.g., methoxy- or ethoxy-fluoromethyl), and C₁-C₆ alkoxy-substituted difluoromethyl groups (e.g., methoxy- or ethoxy-difluoromethyl). Other suitable fluorinated alkoxyalkyl groups include the series (CH₂)₁₋₆OCF₃, (CH₂)₁₋₄OCF₃, (CH₂)₁₋₃OCF₃, (CH₂)₁₋₆OCH₂CF₃, (CH₂)₁₋₄OCH₂CF₃, (CH₂)₁₋₃OCH₂CF₃.

The term “carbocycle” (which may alternatively be referred to herein as “carbocyclic”) as used herein broadly refers to a C₃ to C₈ monocyclic, saturated or unsaturated ring or a C₇ to C₁₀ bicyclic ring system in which each ring is saturated or unsaturated. The carbocycle may be attached at any carbon atom which results in a stable compound. The fused bicyclic carbocycles are a subset of the carbocycles; i.e., the term “fused bicyclic carbocycle” generally refers to a C₇ to C₁₀ bicyclic ring system in which each ring is saturated or unsaturated and two adjacent carbon atoms are shared by each of the rings in the ring system. A subset of the fused bicyclic carbocycles are the fused bicyclic carbocycles in which one ring is a benzene ring and the other ring is saturated or unsaturated, with attachment via any carbon atom that results in a stable compound. Representative examples of this subset include the following:

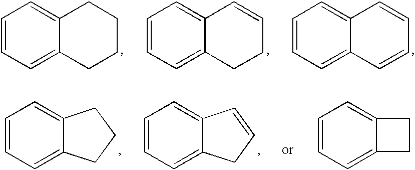

The term “heterocycle” (which may alternatively be referred to as “heterocyclic”) broadly refers to a 5- to 7-membered monocyclic ring or 7- to 10-membered bicyclic ring system any ring of which is saturated or unsaturated, and which consists of carbon atoms and one or more heteroatoms selected from N, O and S, and wherein the nitrogen and sulfur heteroatoms may optionally be oxidized, and the nitrogen heteroatom may optionally be quaterized. The heterocyclic ring may be attached at any heteroatom or carbon atom, provided that attachment results in the creation of a stable structure. Representative examples of heterocyclics include piperidinyl, piperazinyl, azepinyl, pyrrolyl, pyrrolidinyl, pyrazolyl, pyrazolidinyl, imidazolyl, imidazolidinyl, imidazolinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinoxazolinyl, isothiazolidinyl, quinolinyl, isoquinolinyl, benzimidazolyl, thiadazolyl, benzopyranyl, benzothiazolyl, benzoazolyl, furyl, tetrahydrofuryl, tetrahydropuranyl, thienyl (also referred to as thiophenyl), benzothiophenyl, and oxadiazolyl. Representative examples of heterocyclics also include tetrahydrothienyl, tetrahydrodioxothienyl, thiadiazinanyl, dioxothiadiazinanyl, thiazinanyl, dioxothiazinanyl, dioxothiazolidinyl, and isodioxothiazolidinyl.

Fused ring heterocycles form a subset of the heterocycles as defined above; i.e., the term “fused bicyclic heterocycle” refers to a heteroatom-containing bicyclic ring system as defined in the preceding paragraph in which two adjacent atoms are shared by both rings. A subset of the fused bicyclic heterocycles is the fused bicyclic heterocycle containing carbon atoms and one or more heteroatoms selected from nitrogen, oxygen and sulfur, wherein one ring is a benzene ring and the other is a saturated or unsaturated heteroatom-containing ring. Representative examples of this subset include, but are not limited to, the following:

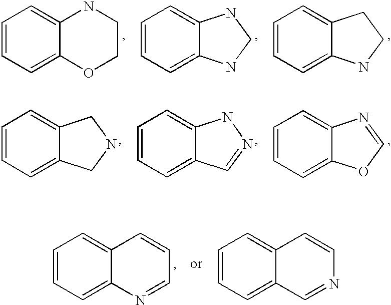

Heteroaromatics form another subset of the heterocycles as defined above; i.e., the term “heteroaromatic” generally refers to a heterocycle as defined * above in which the ring system (whether mono- or bicyclic) is an aromatic ring system. The term “heteroaromatic ring” refers to a monocyclic hetero cycle as defined above which is an aromatic heterocycle. Representative examples of heteroaromatics include pyridyl, pyrrolyl, pyrazinyl, pyrimidinyl, thienyl (alternatively referred to as thiophenyl), thiazolyl, furanyl, imidazolyl, pyrazolyl, triazolyl, tetrazolyl, oxazolyl, oxadiazolyl, thiazolyl, and thiadiazolyl.

It is important to note that the foregoing defininitions of carbocycle, fused bicyclic carbocycle, heterocycle, fused bicyclic heterocycle, heteroaromatic, and heteroaromatic ring are offered as general guidance on the scope of these terms as used herein. Additional restrictions are typically imposed on these terms in the definition of the variables A, B, and R¹ to R⁵in Formula (I). For example, the carbon number range of a carbocycle may be narrowed, or the type and/or number of heteroatoms in a heterocycle may be more limited, or the point of attachment of the heterocycle may be restricted (e.g., limited to a ring carbon atom), or certain heterocycles may be altogether excluded (e.g., provisos excluding indole from the definition of A and/or B).

The compounds of the invention typically have a value of log P greater than 0. P is the partition coefficient of a molecule in a defined charged state in a biphasic octanol/aqueous system; i.e.,

$$P = \frac{{Conc}^{′} n of the molecule in octanol}{{Conc}^{′} n of the molecule in pH = 7.4 phospate buffer}$$

The coefficient is measured at ambient temperature. Log P is the base 10 logarithm of P. Log P is a well-established measure of hydrophobicity and is often indicative of the cell membrane permeability of a compound. Compounds with log P values less than zero (and especially less than −0.5) often have low or no cell permeability, whereas compounds with a log P greater than zero typically can permeate cells freely where they can exhibit potent antiviral activity.

The present invention includes pharmaceutical compositions useful for inhibiting HIV integrase, comprising an effective amount of a compound of this invention, and a pharmaceutically acceptable carrier. Pharmaceutical compositions useful for treating infection by HIV, or for treating AIDS or ARC, are also encompassed by the present invention, as well as a method of inhibiting HIV integrase, and a method of treating infection by HIV, or of treating AIDS or ARC. Additionally, the present invention is directed to a pharmaceutical composition comprising a therapeutically effective amount of a compound of the present invention in combination with a therapeutically effective amount of an AIDS treatment agent selected from:

(1) an AIDS antiviral agent,

(2) an anti-infective agent, and

(3) an immunomodulator.

The present invention also includes the use of a compound of the present invention as described above in the preparation of a medicament for (a) inhibiting HIV integrase, (b) preventing or treating infection by HIV, or (c) treating AIDS or ARC.

The present invention further includes the use of any of the HIV integrase inhibiting compounds of the present invention as described above in combination with one or more AIDS treatment agents selected from an AIDS antiviral agent, an anti-infective agent, and an immunomodulator for the manufacture of a medicament for (a) inhibiting HIV integrase, (b) preventing or treating infection by HIV, or (c) treating AIDS or ARC, said medicament comprising an effective amount of the HIV integrase inhibitor compound and an effective amount of the one or more treatment agents.

The compounds of the present invention may have asymmetric centers and may occur, except when specifically noted, as mixtures of stereoisomers or as individual diastereomers, or enantiomers, with all isomeric forms being included in the present invention.

As is recognized by one of ordinary skill in the art, the 1,3-propanedione compounds of the present invention can exist as tautomers, and thus by using the phrase “or a tautomer thereof” in describing a compound of structural formula (I), it is understood that the following tautomeric forms (IA) and (IB) are included in the present invention:

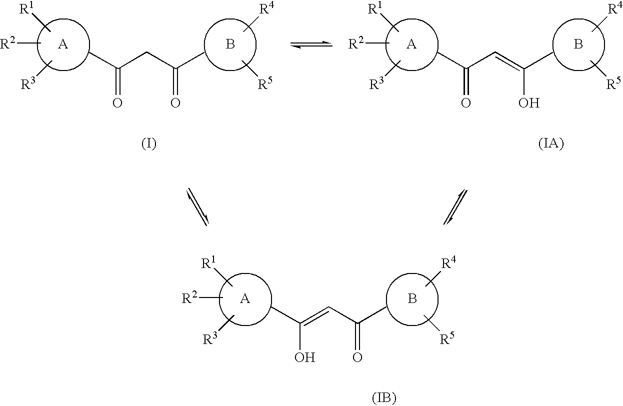

By naming or referring to compound (I) or a tautomer thereof, it is understood for the purposes of the present application that the tautomers (IA) and (IB) are included. Similarly, by referring to compound (IA), it is understood for the purposes of the present application that the tautomers (I) and (IB) are included. The same holds true for references to tautomer (IB).

When any variable (e.g., R^{a}, R^{b}, R^{c}, etc.) occurs more that one time in any constituent or in formula I, its definition on each occurrence is independent of its definition at every other occurrence. Also, combinations of substituents and/or variables are permissible only if such combinations result in stable compounds.

The term “substituted” (e.g., as in “substituted phenyl”) includes mono- and poly-substitution by a named substituent to the extent such single and multiple substitution is chemically allowed.

The compounds of the present inventions are useful in the inhibition of HIV integrase, the prevention or treatment of infection by human immunodeficiency virus (HIV) and the treatment of consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by e.g., blood transfusion, exchange of body fluids, bites, accidental needle stick, or exposure to patient blood during surgery.

The compounds of this invention are useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV integrase, e.g., by competitive inhibition. Thus the compounds of this invention are commercial products to be sold for these purposes.

The present invention also provides for the use of a compound of structural formula (I) to make a pharmaceutical composition useful for inhibiting HIV integrase and in the treatment of AIDS or ARC.

The compounds of the present invention may be administered in the form of pharmaceutically acceptable salts. The term “pharmaceutically acceptable salt” is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. Depending on the particular functionality of the compound of the present invention, pharmaceutically acceptable salts of the compounds of this invention include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide. These salts may be prepared by standard procedures, e.g. by reacting a free acid with a suitable organic or inorganic base. Where a basic group is present, such as amino, an acidic salt, i.e. hydrochloride, hydrobromide, acetate, pamoate, and the like, can be used as the dosage form.

Also, in the case of an acid (—COOH) or alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

For these purposes, the compounds of the present invention may be administered orally, parenterally (including subcutaneous injections, intravenous, intramuscular, intrasternal injection or infusion techniques), by inhalation spray, or rectally, in dosage unit formulations containing conventional non-toxic pharmaceutically-acceptable carriers, adjuvants and vehicles.

The term “administration” and variants thereof (e.g., “administering” a, compound) in reference to a compound of the invention each mean providing the compound or a prodrug of the compound to the individual in need of treatment. When a compound of the invention or prodrug thereof is provided in combination with one or more other active agents (e.g., AIDS antivirals), “administration” and its variants are each understood to include concurrent and sequential provision of the compound or prodrug thereof and other agents.

Thus, in accordance with the present invention there is further provided a method of treating and a pharmaceutical composition for treating HIV infection and AIDS. The treatment involves administering to a subject in need of such treatment a pharmaceutical composition comprising a pharmaceutical carrier and a therapeutically-effective amount of a compound of the present invention.

As used herein, the term “composition” is intended to encompass a product comprising the specified ingredients in the specified amounts, as well as any product which results, directly or indirectly, from combination of the specified ingredients in the specified amounts.

By “pharmaceutically acceptable” it is meant the carrier, diluent or excipient must be compatible with the other ingredients of the formulation and not deleterious to the recipient thereof.

The term “subject,” (alternatively referred to herein as “patient”) as used herein refers to an animal, preferably a mammal, most preferably a human, who has been the object of treatment, observation or experiment.

The term “therapeutically effective amount” as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated.

These pharmaceutical compositions may be in the form of orally-administrable suspensions or tablets or capsules, nasal sprays, sterile injectible preparations, for example, as sterile injectible aqueous or oleagenous suspensions or suppositories.

When administered orally as a suspension, these compositions are prepared according to techniques well-known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The injectible solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

The compounds of this invention can be administered orally to humans in a dosage range of 0.1 to 1000 mg/kg body weight in divided doses. One preferred dosage range is 0.1 to 200 mg/kg body weight orally in divided doses. Another preferred dosage range is 0.5 to 100 mg/kg body weight orally in divided doses. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The present invention is also directed to combinations of the HIV integrase inhibitor compounds with one or more agents useful in the treatment of AIDS. For example, the compounds of this invention may be effectively administered, whether at periods of pre-exposure and/or post-exposure, in combination with effective amounts of the AIDS antivirals, imunomodulators, antiinfectives, or vaccines, such as those in the following table.

ANTIVIRALS

| Drug Name | Manufacturer | Indication |
|---|---|---|
| 097 | Hoechst/Bayer | HIV infection, AIDS, ARC |
| | | (non-nucleoside |
| | | reverse transcriptase |
| | | inhibitor) |
| 141 W94 | Glaxo Wellcome | HIV infection, AIDS, |
| | | ARC |
| | | (protease inhibitor) |
| 1592U89 | Glaxo Wellcome | HIV infection, AIDS, |
| | | ARC |
| Acemannan | Carrington Labs | ARC |
| | (Irving, TX) | |
| Acyclovir | Burroughs Wellcome | HIV infection, AIDS, |
| | | ARC, in |
| | | combination with |
| | | AZT |
| AD-439 | Tanox Biosystems | HIV infection, AIDS, |
| | | ARC |
| AD-519 | Tanox Biosystems | HIV infection, AIDS, |
| | | ARC |
| Adefovir dipivoxil | Gilead Sciences | HIV infection |
| AL-721 | Ethigen | ARC, PGL |
| | (Los Angeles, CA) | HIV positive, AIDS |
| Alpha Interferon | Glaxo Wellcome | Kaposi's sarcoma, HIV in |
| | | combination |
| | | w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| neutralizes pH | Concepts | |
| labile alpha aberrant Interferon | (Rockville, MD) | |
| AR177 | Aronex Pharm | HIV infection, AIDS, |
| | | ARC |
| beta-fluoro-ddA | Nat'l Cancer Institute | AIDS-associated diseases |
| (−)6-Chloro-4(S)- | Merck | HIV infection, AIDS, |
| cyclopropylethynyl- | | ARC |
| 4(S)-trifluoro-methyl- | | (non-nucleoside |
| 1,4-dihydro-2H-3,1- | | reverse transcriptase |
| benzoxazin-2-one | | inhibitor) |
| CI-1012 | Warner-Lambert | HIV-1 infection |
| Cidofovir | Gilead Science | CMV retinitis, herpes, |
| | | papillomavirus |
| Curdlan sulfate | AJI Pharma USA | HIV infection |
| Cytomegalovirus immune | MedImmune | CMV retinitis |
| globin | | |
| Cytovene | Syntex | sight threatening CMV |
| Ganciclovir | | peripheral CMV |
| | | retinitis |
| Delaviridine | Pharmacia-Upjohn | HIV infection, AIDS, |
| | | ARC |
| | | (protease inhibitor) |
| Dextran Sulfate | Ueno Fine Chem. | AIDS, ARC, HIV |
| | Ind. Ltd. (Osaka, Japan) | positive asymptomatic |
| ddC | Hoffman-La Roche | HIV infection, AIDS, |
| Dideoxycytidine | | ARC |
| ddI | Bristol-Myers Squibb | HIV infection, AIDS, |
| Dideoxyinosine | | ARC; combination with |
| | | AZT/d4T |
| DMP-450 | AVID | HIV infection, AIDS, |
| | (Camden, NJ) | ARC |
| | | (protease inhibitor) |
| EL10 | Elan Corp, PLC | HIV infection |
| | (Gainesville, GA) | |
| Efavirenz | DuPont (SUSTIVA ®), | HIV infection, AIDS, |
| (DMP 266) | Merck (STOCRIN ®) | ARC |
| (−)6-Chloro-4(S)- | | (non-nucleoside RT |
| cyclopropylethynyl- | | inhibitor) |
| 4(S)-trifluoro-methyl- | | |
| 1,4-dihydro-2H-3,1- | | |
| benzoxazin-2-one, | | |
| Famciclovir | Smith Kline | herpes zoster, herpes |
| | | simplex |
| FTC | Emory University | HIV infection, AIDS, |
| | | ARC |
| | | (reverse transcriptase |
| | | inhibitor) |
| GS 840 | Gilead | HIV infection, AIDS, |
| | | ARC |
| | | (reverse transcriptase |
| | | inhibitor) |
| GW 141 | Glaxo Welcome | HIV infection, AIDS, |
| | | ARC |
| | | (protease inhibitor) |
| GW 1592 | Glaxo Welcome | HIV infection, AIDS, |
| | | ARC |
| | | (reverse transcriptase |
| | | inhibitor) |
| HBY097 | Hoechst Marion Roussel | HIV infection, AIDS, |
| | | ARC |
| | | (non-nucleoside reverse |
| | | transcriptase inhibitor) |
| Hypericin | VIMRx Pharm. | HIV infection, AIDS, |
| | | ARC |
| Recombinant Human | Triton Biosciences | AIDS, Kaposi's |
| Interferon Beta | (Almeda, CA) | sarcoma, ARC |
| Interferon alfa-n3 | Interferon Sciences | ARC, AIDS |
| Indinavir | Merck | HIV infection, AIDS, |
| | | ARC, asymptomatic HIV |
| | | positive, also in |
| | | combination with |
| | | AZT/ddI/ddC |
| Compound A | Merck | HIV infection, AIDS, |
| | | ARC, asymptomatic |
| | | HIV positive |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, ADS, |
| | | ARC (reverse |
| | | transcriptase |
| | | inhibitor); also with |
| | | AZT |
| Lobucavir | Bristol-Myers Squibb | CMV infection |
| Nelfinavir | Agouron | HIV infection, AIDS, |
| | Pharmaceuticals | ARC |
| | | (protease inhibitor) |
| Nevirapine | Boeheringer Ingleheim | HIV infection, AIDS, |
| | | ARC |
| | | (protease inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc | infection, other CMV |
| | | infections |
| PNU-140690 | Pharmacia Upjohn | HIV infection, AIDS, |
| | | ARC |
| | | (protease inhibitor) |
| Probucol | Vyrex | HIV infection, AIDS |
| RBC-CD4 | Sheffield Med. Tech | HIV infection, AIDS, |
| | (Houston TX) | ARC |
| Ritonavir | Abbott | HIV infection, AIDS, |
| | | ARC |
| | | (protease inhibitor) |
| Saquinavir | Hoffmann-LaRoche | HIV infection, AIDS, |
| | | ARC |
| | | (protease inhibitor) |
| Stavudine; d4T | Bristol-Myers Squibb | HIV infection, AIDS, |
| Didehydrodeoxy- | | ARC |
| thymidine | | |
| T-20 | Trimeris | HIV infection, AIDS, |
| | | ARC |
| Valaciclovir | Glaxo Wellcome | genital HSV & CMV |
| | | infections |
| Virazole | Viratek/ICN | asymptomatic HIV |
| Ribavirin | (Costa Mesa, CA) | positive, LAS, ARC |
| Amprenivir | Vertex | HIV infection, AIDS, |
| VX-478 | | ARC |
| Zalcitabine | Hoffmann-La Roche | HIV infection, AIDS, |
| | | ARC, with AZT |
| Zidovudine; AZT | Glaxo Wellcome | HIV infection, AIDS, |
| | | ARC, Kaposi's sarcoma, in |
| | | combination with other |
| | | therapies |
| ABT-378 | Abbott | HIV infection, AIDS, |
| | | ARC (protease inhibitor) |
| JE2147/AG1776 | Agouron | HIV infection, AIDS, |
| | | ARC (protease inhibitor) |
| T-20 | Trimeris | HIV infection, AIDS, |
| T-1249 | | ARC (fusion inhibitor) |
| BMS 232632 | Bristol-Myers-Squibb | HIV infection, AIDS, |
| | | ARC (protease inhibitor) |

IMMUNO-MODULATORS

| Drug Name | Manufacturer | Indication |
|---|---|---|
| AS-101 | Wyeth-Ayerst | AIDS |
| Bropirimine | Pharmacia Upjohn | advanced AIDS |
| Acemannan | Carrington Labs, Inc. | AIDS, ARC |
| | (Irving, TX) | |
| CL246,738 | American Cyanamid | AIDS, Kaposi's |
| | Lederle Labs | sarcoma |
| EL10 | Elan Corp, PLC | HIV infection |
| | (Gainesville, GA) | |
| Gamma Interferon | Genentech | ARC, in combination |
| | | w/TNF (tumor necrosis factor) |
| Granulocyte | Genetics Institute | AIDS |
| Macrophage Colony | Sandoz | |
| Stimulating | | |
| Factor | | |
| Granulocyte | Hoeschst-Roussel | AIDS |
| Macrophage Colony | Immunex | |
| Stimulating | | |
| Factor | | |
| Granulocyte | Schering-Plough | AIDS, combination |
| Macrophage Colony | | w/AZT |
| Stimulating Factor | | |
| HIV Core Particle | Rorer | seropositive HIV |
| Immunostimulant | | |
| IL-2 | Cetus | AIDS, in combination |
| Interleukin-2 | | w/AZT |
| IL-2 | Hoffman-La Roche | AIDS, ARC, HIV, in |
| Interleukin-2 | Immunex | combination w/AZT |
| IL-2 | Chiron | AIDS, increase in CD4 |
| Interleukin-2 | | cell counts |
| (aldeslukin) | | |
| Immune Globulin | Cutter Biological | pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating Factor | | w/AZT |
| Remune | Immune Response Corp. | immunotherapeutic |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma |
| Alfa 2a | | AIDS, ARC, in |
| | | combination w/AZT |
| SK&F106528 | Smith Kline | HIV infection |
| Soluble T4 | | |
| Thymopentin | Immunobiology Research | HIV infection |
| | Institute | |
| Tumor Necrosis | Genentech | ARC, in combination |
| Factor; TNF | | w/gamma Interferon |
| etanercept | Immunex Corp (Enbrel ®) | rheumatoid arthritis |
| infliximab | Centocor (Remicade ®) | rheumatoid arthritis and |
| | | Crohn's disease |

| | | |
|---|---|---|
| Drug Name | Manufacturer | Indication |

ANTI-INFECTIVES

| | | |
|---|---|---|
| Clindamycin with | Pharmacia Upjohn | PCP |
| Primaquine | | |
| Fluconazole | Pfizer | cryptococcal |
| | | meningitis, candidiasis |
| Pastille | Squibb Corp. | prevention of |
| Nystatin Pastille | | oral candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | antibacterial |
| Trimethoprim/sulfa | | antibacterial |
| Piritrexim | Burroughs Wellcome | PCP treatment |
| Pentamidine | Fisons Corporation | PCP prophylaxis |
| isethionate for | | |
| inhalation | | |
| Spiramycin | Rhone-Poulenc | cryptosporidial |
| | | diarrhea |
| Intraconazole- | Janssen Pharm. | histoplasmosis; |
| R51211 | | cryptococcal |
| | | meningitis |
| Trimetrexate | Warner-Lambert | PCP |

OTHER

| | | |
|---|---|---|
| Daunorubicin | NeXstar, Sequus | Karposi's sarcoma |
| Recombinant Human | Ortho Pharm. Corp. | severe anemia |
| Erythropoietin | | assoc. with AZT |
| | | therapy |
| Recombinant Human | Serono | AIDS-related wasting, |
| Growth Hormone | | cachexia |
| Leukotriene B4 Receptor | — | HIV infection |
| Antagonist | | |
| Megestrol Acetate | Bristol-Myers Squibb | treatment of |
| | | anorexia assoc. |
| | | w/AIDS |
| Soluble CD4 Protein and | — | HIV infection |
| Derivatives | | |
| Testosterone | Alza, Smith Kline | AIDS-related wasting |
| Total Enteral | Norwich Eaton | diarrhea and |
| Nutrition | Pharmaceuticals | malabsorption |
| | | related to AIDS |

It will be understood that the scope of combinations of the compounds of this invention with AIDS antivirals, immunomodulators, anti-infectives or, vaccine is not limited to the list in the above Table, but includes in principle any combination with any pharmaceutical composition useful for the treatment of AIDS.

Preferred combinations are simultaneous or sequential treatments of a compound of the present invention and an inhibitor of HIV protease and/or a non-nucleoside inhibitor of HIV reverse transcriptase. An optional fourth component in the combination is a nucleoside inhibitor of HIV reverse transcriptase, such as AZT, 3TC, ddC or ddI. A preferred inhibitor of HIV protease is the sulfate salt of indinavir, which is N-(2(R)-hydroxy-1(S)-indanyl)-2(R)-phenylmethyl-4-( S)-hydroxy-5-(1-(4-(3-pyridyl-methyl)-2(S)-N′-(t-butylcarboxamido)-piperazinyl))-pentaneamide ethanolate, and is synthesized according to U.S. Pat. No. 5,413,999. Indinavir is generally administered at a dosage of 800 mg three times a day. Other preferred protease inhibitors are nelfinavir and ritonavir. Another preferred inhibitor of HIV protease is saquinavir which is administered in a dosage of 600 or 1200 mg tid. Still another preferred protease inhibitor is Compound A, which is N-(2(R)-hydroxy-1(S)-indanyl)-2(R)-phenylmethyl-4(S)-hydroxy-5-(1-(4-(2-benzo[b]furanylmethyl)-2(S)-N′-(t-butylcarboxamido)piperazinyl))pentaneamide, preferably administered as the sulfate salt. Compound A can be prepared as described in U.S. Pat. No. 5,646,148. Preferred non-nucleoside inhibitors of HIV reverse transcriptase include efavirenz. The preparation of ddC, ddI and AZT are also described in EPO 0,484,071. These combinations may have unexpected effects on limiting the spread and degree of infection of HIV. Preferred combinations include a compound of the present invention with the following (1) indinavir with efavirenz, and, optionally, AZT and/or 3TC and/or ddI and/or ddC; (2) indinavir, and any of AZT and/or ddI and/or ddC and/or 3TC, in particular, indinavir and AZT and 3TC; (3) stavudine and 3TC and/or zidovudine; (4) zidovudine-and lamivudine and 141W94 and 1592U89; (5) zidovudine and lamivudine.

Another preferred combination is a compound of the present invention with indinavir and Compound A and optionally with one or more of efavirenz, AZT, 3TC, ddI and ddC. In one embodiment of this combination, the weight ratio of indinavir to Compound A is from about 1:1 to about 1:2, wherein the amount of indinavir employed is in the range of from about 200 to about 1000 mg. Indinavir and Compound A can be administered concurrently or sequentially in either order from, one to three times per day.

In such combinations the compound of the present invention and other active agents may be administered together or separately. In addition, the administration of one agent may be prior to, concurrent to, or subsequent to the administration of other agent(s).

It will be understood that the scope of combinations of the compounds of this invention with AIDS antivirals, immunomodulators, anti-infectives or vaccines is not limited to the list in the above Table, but includes in principle any combination with any pharmaceutical composition useful for the treatment of AIDS.

Abbreviations used in the instant specification, particularly the Schemes and Examples, are as follows:

Ac=acetyl

Et=ethyl

EtOAc=ethyl acetate

Bu=butyl

n-BuLi=n-butyl lithium

Calc'd=calculated

DMF=N,N-dimethylformamide

DMSO=dimethylsulfoxide

DPPP=1,3-bis(diphenylphosphino)propane

ES MS=electrospray mass spectrometry

Et₃N=triethylamine

EtOH=ethanol

FAB MS=fast atom bombardment mass spectrometry

UPLC=high performance liquid chromatography

LDA=lithium diisopropylamide

Me methyl

MEOH=methanol

m.p.=melting point

NaOMe=sodium methoxide

NMR=nuclear magnetic resonance

Ph=phenyl

rt and RT=room temperature

TFA=trifluoroacetic acid

THF=tetrahydrofuran

p-TsOH=p-toluenesulfonic acid

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

Compounds of the present invention can be prepared via the methods set forth in Schemes 1-3 as follows:

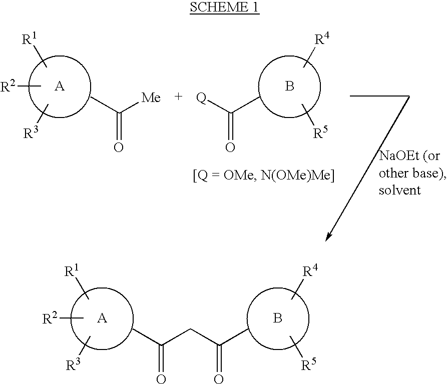

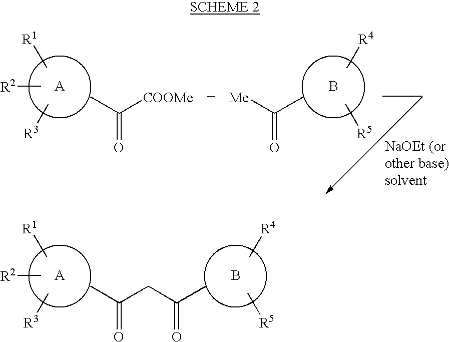

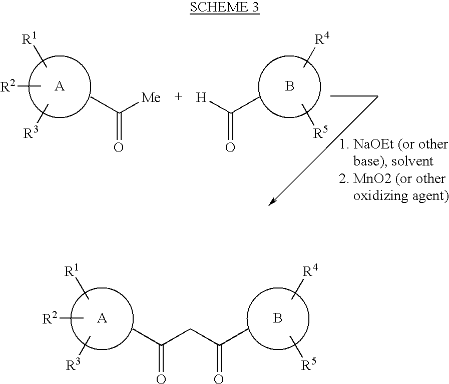

The following examples serve only to illustrate the invention and its practice. The examples are not to be construed as limitations on the scope or spirit of the invention.

### EXAMPLE 1

1-[1-(4-Fluorobenzyl)-1H-pyrrol-2-yl]-3-pyrimidin-4-yl-propan-1,3-dione (1C)

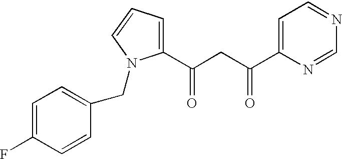

1-[1-(4-Fluorobenzyl)-1H -pyrrol-2-yl]ethanone (1B)

A solution of 2-acetyl pyrrole (1A) (1.09 g, 0.01 mole) in 20 mL of DMF was treated with sodium hydride (0.48 g 60 % dispersion in oil, 0.012 mole) followed by 4-fluorobenzyl) bromide (1.73 g 0.012 mole) and stirred overnight at room temperature. The solution was poured into 300 mL saturated NaHCO₃ and extracted with EtOAc three times, the combined organic layers were washed with NaHCO₃ and dried over MgSO₄, filtered and evaporated to give a clear yellow oil that was taken on to the next step without further purification.

Rf=0.58 (20% EtOAc/Hexanes)

¹H NMR (400 MHz, CDCl₃) δ7.1 (m, 2H), 7.0 (m, 3H), 6.9 (m, 1H), 6.2 (m, 1H), 5.52 (s, 2H), 2.4 (s, 3H).

1-[1-(4-Fluorobenzyl)-1H-pyrrol-2-yl]-3-pyrimidin-4-yl-propan-1,3-dione (1C)

To an oven dried 50 mL three necked round bottomed flask with a stirring bar, septum, argon inlet and thermometer was added THF (5 mL) and diisopropylamine (1.5 mmol, 0.21 mL). This solution was cooled to −78C. and n-butyllithium (1.5 mmol, 0.6 mL of a 1.5 M solution in hexane) was added. To this well stirred solution was added a solution of 1-[1-(4-fluorobenzyl)-1H-pyrrol-2-yl]ethanone (1.0 mmol, 0.217 g) in THF (5 mL), maintaining the temperature <−65C. This solution was aged 40 min then a solution of 6-carbomethoxypyrimidine (1.24 mmol, 0.172 g) in THF (5 mL) was added dropwise. The cooling bath was removed and the mixture was warmed to ambient temperature overnight. The mixture was diluted with EtOAc and washed with saturated aqueous NaHCO₃ solution and brine. Drying (MgSO₄), filtration and removal of the solvent in vacuo gave a solid. This material was chromatographed on silica gel using 25% EtOAc in hexanes as eluant to give 1-[1-(4-fluorobenzyl)-1H-pyrrol-2-yl]-3-pyrimidin-4-yl-propan-1,3-dione (1C) as a crystalline solid.

m.p.: 128-129° C.

¹H NMR (400 MHz, CDCl₃) δ9.26(s, 1H), 8.90(d, j=5 Hz, 1H), 7.90(d, j=5 Hz, 1H), 7.30 (s, 1H), 7.11(m, 2H), 7.00(m, 3H), 6.30(m, 1H), 5.64 (s, 2H).

| | | | |
|---|---|---|---|
| Anal. Calc'd for: C₁₈H₁₄FN₃O₂ | C, 66.87; | H, 4.36; | N, 13.00. |
| Found: | C, 66.53; | H, 4.32; | N, 12.99. |

1-[1-(4-Fluorobenzyl)-1H-pyrrol-2-yl]-3-thiazol-2-yl-propan-1,3-dione (1D)

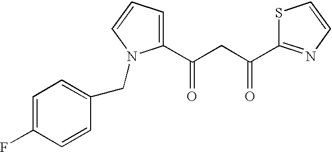

In a manner similar to that for 1C, 1-[1-(4-fluorobenzyl)-1H-pyrrol-2-yl]-3-thiazol-2-yl-propan-1,3-dione (1D) was prepared.

| | | | | |
|---|---|---|---|---|
| Anal. Calc'd for: | C, 62.18; | H, 3.99; | N, 8.53; | S, 9.76. |
| C₁₇H₁₃FN₂O₂S | | | | |
| Found: | C, 62.27; | H, 3.96; | N, 8.57; | S, 9.84. |

1-[1-(4-Fluorobenzyl)-1H-pyrrol-2-yl]-3-(4-methylpyridin-2-yl)propan-1,3-dione (1E)

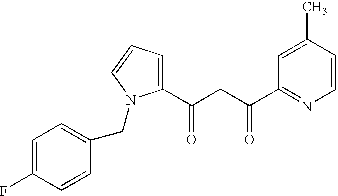

In a manner similar to that for 1C, 1-[1-(4-fluorobenzyl)-1H-pyrrol-2-yl]-3-(4-methylpyridin-2-yl)propan-1,3-dione (1E) was prepared.

| | | | |
|---|---|---|---|
| Anal. Calc'd for: C₂₀H₁₇FN₂O₂ | C, 71.42; | H, 5.09; | N, 8.33. |
| Found: | C, 71.11; | H, 5.16; | N, 8.26. |

1-[1-Benzyl-1H-imidazol-2-yl)-3-[1-(4-fluorobenzyl)-1H-pyrrol-2-yl)propan-1,3-dione (1F)

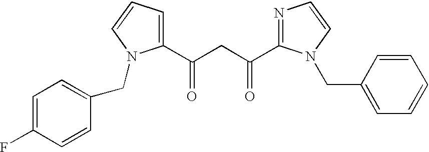

In a manner similar to that for 1C, 1-(1-benzyl-1H-imidazol-2-yl)-3-[1-(4-fluorobenzyl)-1H-pyrrol-2-yl)propan-1,3-dione (1F) was prepared.

| | | | |
|---|---|---|---|
| Anal. Calc'd for: C₂₄H₂₀FN₃O₂ | C, 71.81; | H, 5.02; | N, 10.47. |
| Found: | C, 71.62; | H, 5.13; | N, 10.33. |

1-[1-(4-Fluorobenzyl)-1H-pyrrol-3-yl]-3-pyridin-2-ylpropan-1,3-dione (1G)

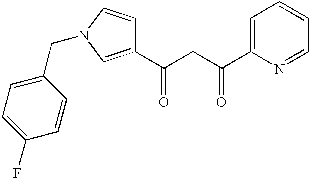

In a manner similar to that for 1C, 1-[1-(4-fluorobenzyl)-1H-pyrrol-3-yl]-3-pyridin-2-ylpropan-1,3-dione (1G) was prepared.

| | | | |
|---|---|---|---|
| Anal. Calc'd for: C₁₉H₁₅FN₂O₂ | C, 70.80; | H, 4.69; | N, 8.69. |
| Found: | C, 70.70; | H, 4.66; | N, 8.62. |

1-(1-(4-Fluorobenzyl)-1H-imidazol-2-yl)-3-[1-(4-fluorobenzyl)-1H-pyrrol-2-yl)propan-1,3-dione (1H)

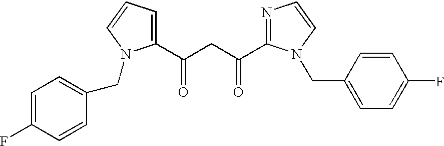

In a manner similar to that for 1C, 1-(1-(4-fluorobenzyl)-1H-imidazol-2-yl)-3-[1-(4-fluorobenzyl)-1H-pyrrol-2-yl)propan-1,3-dione (1H) hydrochloride salt was prepared.

| | | | |
|---|---|---|---|
| Anal. Calc'd for: C₂₄H₁₉F₂N₃O₂.HCl | C, 63.33; | H, 4.42; | N, 9.22. |
| Found: | C, 63.13; | H, 4.24; | N, 9.03. |

1-[1-(4-Fluorobenzyl)-1H-pyrrol-2-yl]-3-(4H-[1,2,4]triazol-3-yl-propan-1,3-dione (1I)

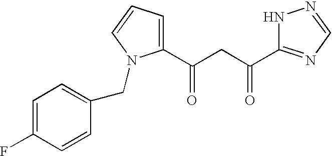

In a manner similar to that for 1C, 1-[1-(4-fluorobenzyl)-1H-pyrrol-2-yl]-3-(4H-[1,2,4]triazol-3-yl-propan-1,3-dione (1I) was prepared.

Anal. Calc'd for: C₁₆H₁₃FNO₂.0.35TFA C, 56.65; H, 3.86; N, 15.83.

Found: C, 56.71l H, 3.82; N, 15.71.

### EXAMPLE 2

1-[1-(4-Fluorobenzyl)-4-(2-oxo-2H-pyridin-1-yl)-1H-pyrrol-2-yl]-3-pyridin-2-yl-propan-1,3-dione (2C)

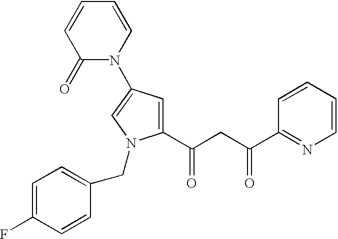

1-[1-(4-Fluorobenzyl)-1H-(4-iodopyrrol)-2-yl]ethanone (2A)

To a 200 mL round bottomed flask with a stirring bar and an argon inlet was added of 1-[1-(4-fluorobenzyl)-1H-pyrrol-2-yl]ethanone (1.00 g, 4.60 mmol) and dry acetone (55 mL). This solution was cooled to −78° C. and N-iodosuccinimide (1.24 g 5.52 mmol) was added in one portion. The cooling bath was allowed to expire and the mixture was stirred at ambient temperature 72 h. The acetone was removed in vacuo and the residue was dissolved in EtOAc. This solution was washed with water (2×), 15% aqueous NaHSO₃ solution and brine; Drying (MgSO₄), filtration and removal of the solvent in vacuo gave a brown oil. This material was chromatographed on silica gel using 10% EtOAc in hexanes as eluant to give 1-[1-(4-fluorobenzyl)-1H-(4-iodopyrrol)-2-yl]ethanone as white crystals.

¹H NMR (400 MHz, CDCl₃) δ6.95-7.12 (m, 6H), 5.45 (s, 2H), 2.39 (s, 3H).

1-[1-(4-Fluorobenzyl)-1H-(4-(2-oxo-2H-pyridin-1-yl)pyrrol)-2-yl]ethanone (2B)

To a 100 mL round bottomed flask with a heavy duty stirring bar and an argon inlet was added a mixture of 1-[1-(4-fluorobenzyl)-1H-(4-iodopyrrol)-2-yl]ethanone (1.03 g, 3.00 mmol), 2-hydroxypyridine (2.85 g, 30 mmol), copper powder (0.189 g, 3.00 mmol) and powdered potassium carbonate (0.829 g, 6.00 mmol). This well stirred mixture was heated in a 200° C. oil bath for 4 h. The cooled mixture was diluted with EtOAc and filtered. The filtrate was transferred to a separatory funnel and washed with saturated aqueous sodium potassium tartrate solution, water and brine. Drying (MgSO₄), filtration and removal of the solvent in vacuo gave an oil. This material was chromatographed on silica gel using 90% EtOAc in hexanes as eluate to give 0.225 g of 1-[1-(4-fluorobenzyl)-1H-(4-(2-oxo-2H-pyridin-1-yl)pyrrol)-2-yl]ethanone as white crystals.

¹H NMR (400 MHz, CDCl₃) δ7.46(m, 2H), 7.34(m, 1H), 7.20 (m, 2H), 7.19 (d, j=5 Hz, 1H), 6.99(t, j=8.5 Hz, 2H), 6.62(dd, j=1.9 Hz, 1H), 6.25 (dt, j=1.6 Hz, 1H0, 5.55(s, 2H), 2.44(s, 3H).

1-[1-(4-Fluorobenzyl)-4-(2-oxo-2H-pyridin-1-yl)-1H-pyrrol-2-yl]-3-pyridin-2-yl-propan-1,3-dione (2C)

To a 50 mL round bottomed flask with a stirring bar, reflux condenser and an argon inlet was added 1-[1-(4-fluorobenzyl)-1H-(4-(2-oxo-2H-pyridin-1-yl)pyrrol)-2-yl]ethanone (0.225 g, 0.73 mmol), dry THF (5 mL), ethyl picolinate (0.195 mL, 1.45 mmol), and sodium hydride (0.067 g of a 60% w/w oil suspension, 1.00 mmol). This well stirred mixture was heated at 50° C. for 1 h. The reaction was quenched with aqueous NH₄Cl and the mixture was extracted with EtOAc. The EtOAc extract was washed with brine, dried (MgSO₄), filtered and concentrated in vacuo. This material was chromatographed on silica gel using EtOAc as eluant. The product was then crystallized from diethyl ether.

m.p.: 157-158° C.

¹H NMR (400 MHz, CDCl₃) δ8.65(br d, j=4 Hz, 1H), 8.02(d, j=8 Hz, 1H), 7.81 (m, 1H), 7.65(d, j=2 Hz, 1H), 7.54(dd, j=2.7 Hz, 1H), 7.30-7.40(m, 2H), 7.16-7.29(m,5H), 7.00(t, j=10 Hz, 2H), 6.64(br d, j=9 Hz, 1H), 6.25(dt, j=1.9 Hz, 1H),5.68(s, 2H), 5.54(s, 1H), 4.66(s, 1H).

| | | | |
|---|---|---|---|
| Anal. Calc'd for C₂₄H₁₈FN₃O_{3:} | C, 68.74; | H, 4.43; | N, 10.02 |
| Found: | C, 68.73; | H, 4.34; | N, 10.02. |

### EXAMPLE 3

1-(3-Benzyl-5-pyrazin-2-ylmethylphenyl)-3-(4-methylpyridin-2-yl)-propane-1,3-dione (3I)

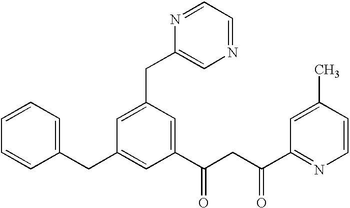

1-Benzyl-3,5-dibromobenzene (3B)

To a cold (−78 C.) solution of 1,3,5-tribromobenzene (30 g) in diethyl ether (500 mL), a solution of n-BuLi in hexanes (2.5 M, 38.1 mL) was added. The resultant mixture was stirred at −78 C. for 1 h and was treated with benzaldehyde (10.2 mL). The reaction mixture was allowed to warm up slowly to 0 C. and was stirred at that temperature for 1.5 hr. The product mixture was diluted with ethyl acetate and partitioned with aq. HCl (1M, 95 mL). The organic extract was washed with brine, dried over magnesium sulfate, filtered, and concentrated under vacuum. The resultant (3,5-dibromophenyl)phenylmethanol was taken on to the next step without further purification.

To a cold (0 C.) solution of (3,5-dibromophenyl)phenylmethanol (32.5 g) and triethylsilane (27.7 g) in dichloromethane (500 mL), boron trifluoride diethyl etherate (30 mL) was added dropwise over a period of 45 min. The resultant mixture was stirred at 0 C. for 1 hr, and at room temperature overnight. The product mixture was diluted with dichloromethane, and neutralized with saturated aq. sodium bicarbonate. The organic extract was washed with brine, dried over magnesium sulfate, filtered, and concentrated under vacuum. The residue was subjected to column chromatography on silica gel eluted with hexane. Collection and concentration of appropriate fractions provided the title dibromide.

¹H NMR (400 MHz, CDCl₃) δ7.50 (br s, 1H), 7.40−7.10 (m, 7H), 3.91 (s,2H).

(3-Benzyl-5-bromophenyl) pyrazin-2-yl ketone (3C)

To a cold (−78 C) solution of 1-benzyl-3,5-dibromobenzene (1.5 g) in diethyl ether (20 mL), a solution of n-BuLi in hexanes (2.5 M, 2 mL) was added. The resultant mixture was stirred at −78 C for 1 h and was treated with a solution of N-methoxy-N-methylpyrazinecarboxyamide (0.84 g) in diethyl ether (5 mL). The reaction mixture was allowed to warm up slowly to room temperature and was stirred at that temperature overnight. The product mixture was diluted with ethyl acetate and partitioned with aq. HCl. The organic extract was washed with brine, dried over magnesium sulfate, filtered, and concentrated under vacuum. The residue was subjected to column chromatography on silica gel eluting with 20% ethyl acetate in hexane. Collection and concentration of appropriate fractions provided the title pyrazine.

¹H NMR (400 MHz, CDCl₃) δ9.24 (br s, 1H), 8.79 (br s, 1H), 8.66 (br s, 1H), 8.07 (br s, 1 H), 7.86 (br s, 1H), 7.57 (br s, 1H), 7.34−7.18 (m, 5H), 4.03 (s, 2H).

3-Benzyl-5-pyrazin-2-ylmethyl-1-bromobenzene (3D)

A mixture of (3-benzyl-5-bromophenyl) pyrazin-2-yl ketone (0.97 g) and anhydrous hydrazine (2 mL) in ethylene glycol (6 mL) was heated at 110 C. for 4 hr. Excess hydrazine was removed under reduced pressure. The residue ethylene glycol solution was treated with powdered solid KOH (0.4 g) and heated at 160 C. under an atmosphere of argon for 4 h. The product mixture was partitioned between benzene and water. The organic extract was washed with brine, dried over magnesium sulfate, filtered, and concentrated under vacuum. The residue was subjected to column chromatography on silica gel eluting with 20-30% ethyl acetate in hexane gradient. Collection and concentration of appropriate fractions provided the title bromide.

¹H NMR (400 MHz, CDCl₃) δ8.51 (br s, 1H), 8.43 (br s, 1H), 7.31−7.14 (m, 8 H), 7.03 (br s, 1H), 4.09 (s, 2H), 3.91 (s, 2H).

3-Benzyl-5-pyrazin-2-ylmethylacetophenone (3E)

To a mixture of 3-benzyl-5-pyrazin-2-ylmethyl-1-bromobenzene (0.77 g), thallium acetate (0.66 g), 1,3-bis(diphenylphosphino)propane (0.263 g) and triethylamine (1.27 mL) in DMF (5 mL) in a pressure tube, purged with argon for a period of 10 minutes, palladium acetate (128 mg) and n-butyl vinyl ether (1.5 mL) was added. The reaction tube was sealed and stirred at 100 C. overnight. The reaction mixture was filtered through a bed of Celite, and the filtrate concentrated under vacuum. The residue was dissolved in TMF (5 mL) and treated with aq. HCl (3M, 4 mL). The resultant mixture was stirred at rt for 3 hr., diluted with ethyl acetate, basified with aq. sodium bicarbonate. The organic extract was dried over magnesium sulfate, filtered, and concentrated under vacuum. The residue was subjected to column chromatography on silica gel eluting with 50% ethyl acetate in hexane. Collection and concentration of appropriate fractions provided the title ketone.

¹H NMR (400 MHz, CDCl₃) δ8.50 (br s, 1H), 8.46 (br s, 1H), 8.43 (br s, 1H), 7.70(br s, 1H), 7.66 (br s, 1H), 7.31−7.15 (m, 6 H), 4.18 (s, 2H), 4.01 (s, 2H), 2.54 (s, 3H).

2-Cyano-4-methylpyridine (3G)

To a cold (0 C.) solution of 4-methyl-pyridine-1-oxide (3F) (10 g; commercially available) and trimethylsilyl cyanide (14.7 mL) in CH₂Cl₂ (150 mL) under an atmosphere of argon, N,N-dimethylcarbamoyl chloride (10.6 mL) was added dropwise with the temperature of the reaction maintained below 3 C. After the addition was complete, the resultant mixture was allowed to warm to room temperature and was stirred at room temperature overnight. The product mixture was treated with a 10% solution of K₂CO₃ (300 mL) and was stirred for 15 minutes. The aqueous layer was separated and extracted three times with CH₂Cl₂. The organic extracts were combined, washed with brine and concentrated under vacuum. The residue was subjected to column chromatography on silica gel eluted with 30% EtOAc in hexanes. Collection and concentration of appropriate fractions provided 2-cyano-4-methylpyridine (3G) as a white solid.

¹H NMR (400 MHz, CDCl₃) δ8.58 (d, 1H), 7.5 (s, 1H), 7.32 (m, 1H), 7.25 (s, 1H), 2.4 (s, 3H).

Methyl 4-methylpyridine-2-carboxylate (3H).

A cold (0 C.) solution of 2-cyano-4-methylpyridine (3G) (7.2 g, 0.061 mol) and water (1.11 mL, 0.061 mol) in MeOH (150 mL) was saturated with HCl gas. The resultant mixture was refluxed under an atmosphere of argon for 3 hr. The reaction mixture was concentrated under vacuum, and the residue partitioned between saturated aqueous NaHCO₃ and CHCl₃. The organic extracts were combined, dried over magnesium sulfate, filtered, and concentrated under vacuum. The residue was subjected to column chromatography on silica gel eluted with 5% Et₂O/CHCl₃. Collection and concentration of appropriate fractions gave methyl 4-methylpyridine-2-carboxylate (3H) as a clear oil.

¹H NMR (400 MHz, CDCl₃) δ8.6 (d, 1H), 8.0(s, 15H), 7.3(m, 1H), 4.0 (s, 3H), 2.43(s, 3H).

1-(3-Benzyl-5-pyrazin-2-ylmethylphenyl)-3-(4-methylpyridin-2-yl)-propane-1,3-dione (3I)

To a cold (−78 C.) solution of 3-benzyl-5-(5-methylpyrazin-2-yl)methylacetophenone (0.3 g) in THF (10 mL), a solution of n-BuLi in hexanes (2.5 M, 0.48 mL) was added. The resultant mixture was stirred at −78 C. for 45 minutes and was treated with a solution of methyl 4-methylpyridine-2-carboxylate (3H) (0.22 g) in THF (4 mL). The reaction mixture was allowed to warm up slowly to room temp in 2 hours. The product mixture was diluted with ethyl acetate and partitioned with aq. HCl. The organic extract was washed with brine, dried over magnesium sulfate, filtered, and concentrated under vacuum. The residue was subjected to column chromatography on silica gel eluting with ethyl acetate. Collection and concentration of appropriate fractions provided the title dione.

¹H NMR (400 MHz, CDCl₃) δ8.56 (br s, 1H), 8.51 (br s, 1H), 8.47 (br s, 1H), 8.43 (br s, 1H), 7.99 (br s, 1H), 7.83 (br s, 1H), 7.81 (br s, 1H), 7.49 (br s, 1H), 7.31−7.16 (m, 7 H), 4.20 (s, 2H), 4.03 (s, 2H), 2.45 (s, 3H).

1-(3-Benzyl-5-pyridin-2-ylmethylphenyl)-3-(4-methylpyridin-2-yl)-propane-1,3-dione (3J)

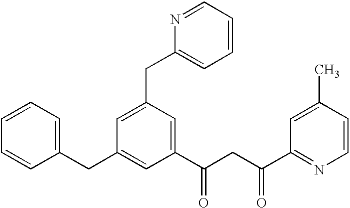

In a manner similar to that for 3I, 1-(3-Benzyl-5-pyridin-2-ylmethylphenyl)-3-(4-methylpyridin-2-yl)-propane-1,3-dione (3J) was prepared.

| | | | |
|---|---|---|---|
| Anal. Calc'd for C₂₈H₂₄N₂O₂ | C, 79.98; | H, 5.75; | N, 6.66. |
| Found: | C, 79.78; | H, 5.85; | N, 6.38. |

3-[3-(2-Chlorobenzyl)-5-pyridin-2-ylmethylphenyl]-1-(4-methylpyridin-2-yl)-propane-1,3-dione (3K)

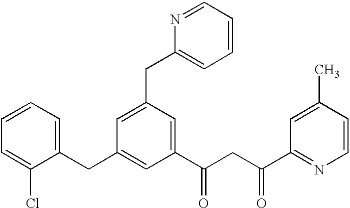

In a manner similar to that for 3I, 3-[3-(2-Chlorobenzyl)-5-pyridin-2-ylmethylphenyl]-1-(4-methylpyridin-2-yl)-propane-1,3-dione (3K) was prepared.

| | | | |
|---|---|---|---|
| Anal. Calc'd for C₂₈H₂₃ClN₂O₂ | C, 73.92; | H, 5.10; | N, 6.16. |
| Found: | C, 74.05; | H, 5.19; | N, 6.19. |

3-[3-(3-Chlorobenzyl)-5-pyridin-2-ylmethylphenyl]-1-(4-methylpyridin-2-yl)-propane-1,3-dione (3L)

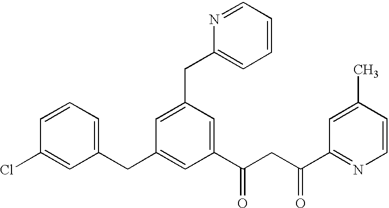

In a manner similar to that for 3I, 3-[3-(3-Chlorobenzyl)-5-pyridin-2-ylmethylphenyl]-1-(4-methylpyridin-2-yl)-propane-1,3-dione (3L) was prepared.

| | | | |
|---|---|---|---|
| Anal. Calc'd for | C, 62.81; | H, 4.23; | N, 4.87. |
| C₂₈H₂₃ClN₂O₂.1.05 TFA & 0.05 H₂O | | | |
| Found: | C, 63.17; | H, 4.56; | N, 4.47. |

1-(3,5-Bis-pyridin-2-ylmethylphenyl)-3-(4-methylylpyridin-1,3-dione (3M)

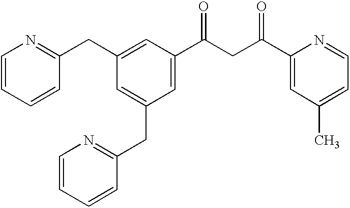

5-Bromoisophthalic acid

To a solution of dimethyl 5-bromoisophthalate (5.00 g, 18.3 mmol) in 150 mL of MeOH was added KOH (5.14 g, 91.5 mmol). The reaction was refluxed for 3 hours. The reaction was cooled and the solvent was removed in vacuo. The remaining residue was dissolved in 100 mL water and acidified with 35 mL of 3N HCl solution and extracted with EtOAc. The combined organic extracts were washed with brine, dried over Na₂SO₄, filtered, and concentrated to give 5-bromoisophthalic acid as a white solid.

¹H NMR (400 MHz, DMSO) δ13.44 (b, 2H), 8.41(m, 1H), 8.25(m, 2H).

5-Bromo-N,N′-dimethoxy-N,N′-dimethylisophthalamide

Oxalyl chloride (3.19 mL, 36.6 mmol) and DMF (one drop, catalyst) were added dropwise to a solution of 5-bromoisophthalic acid (4.48 g, 18.3 mmol) in anhydrous THF (150 mL). After stirring for 1 hour the solvent was removed in vacuo. The crude yellow oil was dissolved in CHCl₃(150 mL), N,O-dimethylhydroxylamine hydrochloride (1.96 g, 20.1 mmol) was added, and the reaction was chilled (0° C.) as the triethylamine (5.07 mL, 36.6 mmol) was added dropwise. The ice bath was removed and the reaction was allowed to stir at room temperature for 2 hours. The solvent was removed in vacuo and the residue was partitioned between saturated NH₄Cl solution and CHCl₃. The organic extracts were combined, washed with brine, dried over Na₂SO₄, filtered, and concentrated to give a brown oil. This material was chromatographed on silica gel eluting with 60% EtOAc in hexanes to give 5-bromo-N,N′-dimethoxy-N,N′-dimethylisophthalamide as a white solid.

¹H NMR (400 MHz, CDCl₃) δ7.94(s, 1H), 7.93(s, 2H), 3.57(s, 6H), 3.38 (s, 6H).

1-Bromo-3,5-bis(1-pyridin-2-yl-carbonyl)benzene

1,2-dibromoethane (2.34 mL, 27.2 mmol) and then 2-bromopyridine (2.59 mL, 27.2 mmol) were added slowly to Mg metal (1.32 g, 54.4 mmol) suspended in 75 mL of distilled THF in a dried 500 mL round bottom flask. After the Mg turnings were consumed, 5-bromo-N,N′-dimethoxy-N,N′-dimethylisophthalamide (1.50 g, 4.53 mmol) in 25 mL of distilled THF was added and the reaction was stirred at room temperature overnight. The reaction was quenched with saturated NH₄Cl solution and extracted with CH₂Cl₂. The combined organic extracts were washed with brine, dried over Na₂SO₄, filtered, and concentrated to give a brown oil. This material was chromatographed on silica gel eluting with 40% EtOAc in hexanes to give 1-bromo-3,5-bis(1-pyridin-2-yl-carbonyl)benzene as a yellow solid.

¹H NMR (400 MHz, CDCl₃) δ8.79(m, 1H), 8.74(m, 2H), 8.50(m, 2H), 8.12(m, 2H), 7.93(m, 2H), 7.53(m, 2H).

1-Bromo-3,5-bis(2-pyridinylmethyl)benzene

A mixture of 1-bromo-3,5-bis(1-pyridin-2-yl-carbonyl)benzene (0.65 g, 1.77 mmol) in 4.75 mL ethylene glycol and 2.75 mL hydrazine was heated (110° C.) for 6 hours in dried glassware under argon. Powdered KOH (275 mg, 4.91 mmol) was added to the now homogeneous reaction and the reaction was heated (160° C.) for 45 minutes, stirred at room temperature overnight. The reaction was quenched with water, the pH of the solution was adjusted to 7 using 1N HCl, and the solution was extracted with EtOAc. The combined organic extracts were dried over Na₂SO₄, filtered, and concentrated to give a yellow oil. This material was chromatographed on silica gel using 80% EtOAc in hexanes as the elutant to give 1-bromo-3,5-bis(2-pyridinylmethyl)benzene as a white solid.

¹H NMR (400 MHz, CDCl₃) δ8.5(m, 2H), 7.58(m, 2H), 7.25(m, 2H), 7.11(m, 4H), 7.08(s, 1H), 4.08(s, 4H).

1-(3,5-Bis-pyridin-2-ylmethylphenyl)ethanone

A mixture of 1-bromo-3,5-bis(2-pyridinylmethyl)benzene (430 mg, 1.27 mmol), triethylamine (353 TL, 2.54 mmol), n-butyl vinyl ether (819 μl, 6.34 mmol), thallium acetate (367 mg, 1.39 mmol), 1,3-bisdiphenylphosphinopropane (131 mg, 0.32 mmol), and palladium acetate (57 mg, 0.25 mmol) in DMF (3 mL) in a seal tube was purged with argon for 15 minutes. The tube was capped and the mixture was heated with stirring at 100° C. for 48 hours. After cooling to room temperature the mixture was filtered through Celite and concentrated in vacuo. The residue was treated with a mixture of THF (10 mL) and a HCl (3 mL, 1M). After 1 hour, the reaction was diluted with saturated aq NH₄Cl and EtOAc. The organic extracts were * washed with brine, dried over Na₂SO₄, filtered, and concentrated to give a brownish oil. This material was chromatographed on silica gel using 5% MeOH in EtOAc as the elutant to give 1-(3,5-bis-pyridin-2-ylmethylphenyl)ethanone as a yellow oil.

¹H NMR (400 MHz, CDCl₃) δ8.55(m, 2H), 7.71(m, 2H), 7.59(m, 2H), 7.41(s, 1H), 7.12(m, 4H), 4.18(s, 4H), 2.54(s,3H).

1-(3,5-Bis-pyridin-2-ylmethyl-phenyl)-3-(4-methyl-pyridin-2-yl)propane-1,3-dione (3M)

A solution of 1-(3,5-bis-pyridin-2-ylmethylphenyl)ethanone (140 mg, 0.46 mmol), 5-methyl 4-methylpydridine-2-carboxylate (154 mg, 1.02 mmol), and sodium methoxide (55 mg, 1.02 mmol) in THF (1 mL) was stirred at room temp for 1.5 hours. The reaction was quenched with water, the pH of the solution was adjusted to 4 using 1N HCl, and the solution was extracted with CHCl₃. The combined organic extracts were dried over Na₂SO₄, filtered, and concentrated to give a yellow solid. This material was purified by reverse phase HPLC. Collection and lyophilization of appropriate fractions provided 1-(3,5-bis-pyrazol-1-ylmethyl-phenyl)-3-(4-methyl-pyridin-2-yl)propane-1,3-dione (3M) as bis TFA salt.

¹H NMR (400 MHz, CDCl₃) δ8.87(m, 2H), 8.62(m, 1H), 8.13(m, 2H), 8.02(s, 1H), 7.95(m, 2H), 7.60(m, 4H), 7.53(m, 2H), 7.32(m, 1H), 4.48(s, 4H), 2.48 (s, 3H).

| | | | |
|---|---|---|---|
| Anal. Calc'd for: | C, 56.10; | H, 4.03; | N, 6.25. |
| C₂₇H₂₃N₃O₂.2TFA 0.10 Et₂O 0.85 H₂O | | | |
| Found: | C, 56.10; | H, 3.98; | N, 6.10. |

1-[3,5-Bis-(2-methyl-2H-pyrazol-3-ylmethyl)phenyl]-3-(4-methylpyridin-2-yl)propane-1,3-dione (3N)

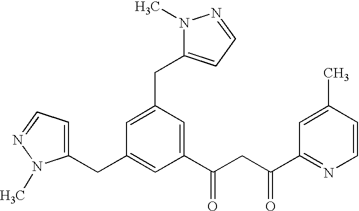

3,5-Bis-(2-methyl-2H-pyrazol-3-ylmethyl)-1-bromobenzene

To a cold (−78° C.) solution of N-methylpyrazole (0.31 g) in anhydrous THF (10 mL), a solution of n-BuLi in hexanes (2.5 M, 1.5 mL) was added. The resultant mixture was stirred at −78° C. for 3 h and was treated with a solution of 1-bromo-3,5bis-(N-methoxy-N-methyl)carboxyamidobenzene (0.50 g) in THF (5 mL) The reaction mixture was allowed to warm up slowly to room temperature and was stirred at that temperature overnight. The product mixture was diluted with ethyl acetate and partitioned with aq. HCl. The organic extract was washed with brine, dried over magnesium sulfate, filtered, and concentrated under vacuum. The residue was subjected to column chromatography on silica gel eluting with 50% ethyl acetate in hexane: Collection and concentration of appropriate fractions provided the title bispyrazole.

¹H NMR (400 MHz, CDCl₃) δ8.25 (s, 1H), 8.22 (br s, 2H), 7.55 (br s, 2H), 6.68 (br s, 2H) 4.24 (s, 6H).

In a manner similar to that for 3I, substituting 3C with 3,5-bis-(2-methyl-2H-pyrazol-3-ylmethyl)-1-bromobenzene, 1-[3,5-bs-(2-methyl-2H-pyrazol-3-ylmethyl)phenyl]-3-(4-methylpyridin-2-yl)-propane-1,3-dione (3N) was prepared.

| | | | |
|---|---|---|---|
| Anal. Calc'd for C₂₅H₂₅N₅O₂.1.5 TFA | C, 56.19; | H, 4.46; | N, 11.70. |
| Found: | C, 56.47; | H, 4.42; | N, 11.63. |

1-(3-Pyridin-2-ylmethyl)phenyl-3-(4-methylpyridin-2-yl)propane-1,3-dione (3O)

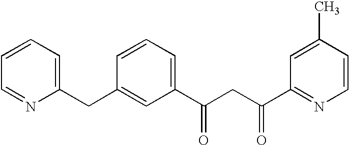

3-Bromophenyl pyridin-2-yl ketone

To a cold (−78° C.) solution of 1,3-dibromobenzene (0.75 mL) in diethyl ether (25 mL), a solution of n-BuLi in hexanes (2.5 M, 2.6 mL) was added. The resultant mixture was stirred at −78° C. for 1 h and was treated with a solution of N-methoxy-N-methylpyridine-2-carboxyamide(1.07 g) in ether (4 mL). The reaction mixture was allowed to warm up slowly to room temp. and was stirred at that temp. overnight. The product mixture was diluted with ether and partitioned with aq. HCl. The organic extract was washed with brine, dried over magnesium sulfate, filtered, and concentrated under vacuum. The residue was subjected to column chromatography on silica gel eluting with 20% ethyl acetate in hexane. Collection and concentration of appropriate fractions provided the title ketone.

¹H NMR (400 MHz, CDCl₃) δ8.73 (br d, 1H), 8.23 (br s, 1H), 8.07 (d, 1H), 8.02 (d, 1H), 7.92 (td, 1H), 7.72 (br d, 1H), 7.52 (m, 1H), 7.37 (t, 1H).

In a manner similar to that for 3I, substituting 3C with 3-bromophenyl pyridin-2-yl ketone, 1-(3-pyridin-2-ylmethyl)phenyl-3-(4-methylpyridin-2-yl)propane-1,3-dione (3O) was prepared.

¹H NMR (400 MHz, CDCl₃) δ8.89 (d, 1H), 8.68 (d, 1H), 8.14 (m, 1H), 8.05-8.06 (m, 3H), 7.64 (t, 1H), 7.48-7.57 (m, 4H) 7.36(d, 1H), 4.54 (s, 2H), 2.51 (s, 3H).

1-[3-(1,1-Dioxoisothiazolin-2-ylmethyl)-5-(pyridin-2-ylmethyl)phenyl]-3-(4-methylpyridin-2-yl)propane-1,3-dione (3P)

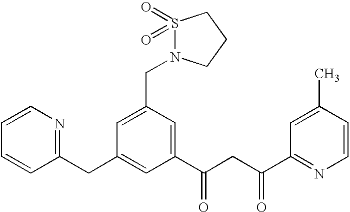

tert-Butyldimethylsilyl 3,5-dibromobenzyl ether

A mixture of 3,5-dibromobenzyl alcohol (9.77 g, 36.74 mmol), tert-butyldimethylchlorosilane (6.80 g, 45.1 mmol) and imidazole (6.08 g, 89.3 mmol) in DMF (100 mL) was stirred under argon over night. The reaction mixture was diluted with ether, washed with water three times, dried over magnesium sulfate, and concentrated under vacuum to provide the title compound.

¹H NMR (400 MHz, CDCl₃) δ7.58 (s, 1H), 7.39 (s, 2H), 4.62 (s, 2H), 0.95 (s, 9H), 0.05 (s, 6H).

(Pyridin-2-yl) 1-[3-bromo-5-(tert-butyldimethylsilyloxymethyl)phenyl] ketone

In a manner similar to that for the preparation of 3C substituting with tert-butyldimethylsilyl 3,5-dibromobenzyl ether, the title compound was prepared.

¹H NMR (400 MHz, CDCl₃) δ8.64 (d, 1H), 7.96-8.22 (m, 2H), 7.82-7.88 (m, 2H), 7.62 (s, 1H), 7.41-7.44 (m, 1H), 4.72 (s, 2H), 0.85 (s, 9H), 0.05 (s, 6 H).

3-bromo-5-(pyridin-2-yl-methyl)benzyl alcohol

In a manner similar to that for the preparation of 3D substituting with (pyridin-2-yl) 1-[3-bromo-5-(tert-butyldimethylsilyloxymethyl)phenyl] ketone, the title compound was prepared.

¹H NMR (400 MHz, CDCl₃) δ8.53 (d, 1H), 7.61 (m, 1H), 7.38 (s, 1H), 7.32 (s, 1H), 7.18 (s, 1H), 7.12-7.16 (m, 2H), 4.62 (s, 2H), 4.12 (s, 2H).

1-[3-Hydroxymethyl-5-(pyridin-2-yl-methyl)phenyl]ethanone

In a manner similar to that for the preparation of 3E substituting with 3-bromo-5-(pyridin-2-yl-methyl)benzyl alcohol, the title compound was prepared.

¹H NMR (400 MHz, CDCl₃) δ8.53 (d, 1H), 7.82 (s 1H) 7.78 (s, 1H), 7.61 (m, 1H), 7.49 (s, 1H), 7.12-7.16 (m, 2H), 4.72 (s, 2H), 4.20 (s, 2H), 2.60 (s, 3H).

1-[3-(1,1-Dioxo-isothiazolin-2-ylmethyl)-5-(pyridin-2-yl-methyl)phenyl]ethanone

A mixture of 1-[3-hydroxymethyl-5-(pyridin-2-yl-methyl)-phenyl]ethanone (750 mg, 3.11 mmol), methanesulfonyl chloride (0.36 mL, 4.65 mmol) and triethylamine (0.78 mL, 5.60 mmol) in dichloromethane (10 mL) was stirred under argon for 30 min. The solvent was removed and the residue was coevaporated with toluene, and dissolved in 10 mL DMF. The solution was added to a mixture prepared separately by addition of sodium hydride (60% dispersion in mineral oil, 785 mg, 19.6 mmol) to a solution of 3-chloropropanesulfonamide (985 mg, 6.25 mmol) in DMF (30 mL) at 0° C. and stirred at that temp for 2 h. The resultant mixture was stirred rt for another 3 h. and concentrated under vacuum. The residue was treated with saturated aqueous NH₄Cl, extracted with chloroform. The organic layer was washed with brine, dried over magnesium sulfate, concentrated. The resultant residue was purified by flash chromatography on silica gel eluting with hexanes/ethyl acetate to give title ketone

¹H NMR (400 MHz, CDCl₃) δ8.54-8.58 (m, 1H), 7.78-7.81 (m, 2H), 7.61 (m, 1H), 7.49 (s, 1H), 7.12-7.16 (m, 2H), 4.22 (s, 2H), 4.20 (s, 2H), 3.20 (t, 2H), 3.12 (t, 2H), 2.60 (s, 3H), 2.32 (m, 2H).

1-[3-(1,1-Dioxoisothiazolin-2-ylmethyl-5-(pyridin-2-ylmethyl)phenyl]-3-(4-methylpyridin-2-yl)propane-1,3-dione (3P)

In a manner similar to that for the preparation of 3M substituting with 1-[3-(1,1-dioxo-isothiazolin-2-ylmethyl)-5-(pyridin-2-yl-methyl)phenyl]ethanone, the title compound was prepared.

¹H NMR (400 MHz, CDCl₃) δ8.56−7.59 (m, 2H), 8.00 (s, 1H), 7.92 (s, 1H), 7.87 (s, 1H), 7.60 (t, 1H), 7.52 (s, 1H), 7.48 (br s, 1H), 7.25 (m, 1H), 7.12-7.16 (m, 2H), 4.22 (s, 2H), 4.20 (s, 2H), 3.22 (t, 2H), 3.12 (t, 2H), 2.46 (s, 3H), 2.32 (m, 2H).

1-[3-(1,1-Dioxoisothiazolin-2-ylmethyl)-5-(pyridin-2-ylmethyl)phenyl]-3-(1-methyl-1H-imidazol-4-yl)propane-1,3-dione (3Q)

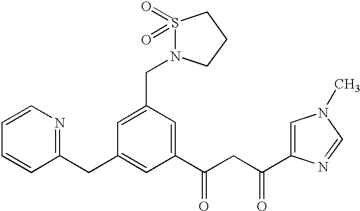

In a manner similar to that for the preparation of 3P, substituting with methyl 1-methylimidazol-4-carboxylate, the title compound (3Q) was prepared.

¹H NMR (400 MHz, CDCl₃) δ8.56 (d, 1H), 7.86 (s, 1H), 7.80 (s, 1H), 7.64 (s, 1H), 7.60 (t, 1H), 7.51 (s, 1H), 7.44 (s, 1H), 7.12-7.16 (m, 2H), 7.05 (s, 1H), 4.22 (s, 2H), 4.20 (s, 2H), 3.80 (s, 3H), 3.20 (t, 2H), 3.12 (t, 2H), 2.32 (m, 2H).

### EXAMPLE 4

1-(3-Benzylphenyl)-3-(1H-imidazol-2-yl)-propane-1,3-dione TFA salt (4E)

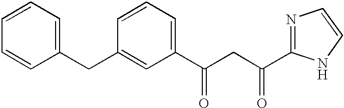

1-Trityl-1-H-imidazole-2-carboxaldehyde (4A)

A suspension of 2-imidazole carboxaldehyde (7.3 g, 0.076 mole, Aldrich) in 76 mL of degassed DMF was treated with triethylamine (9.2 g, 12.7 mL, 0.091 mole) followed by trityl bromide (27 g, 0.084 mole) and stirred overnight at room temperature. The light purple suspension was poured into 800 mL H₂O and extracted with CH₂Cl₂ three times the combined organic layers were washed with NaHCO₃ and dried over Na₂SO₄, filtered and evaporated to give solids that were triturated with CH₂Cl₂/petroleum ether to give 1-Trityl-1-H-imidazole-2-carboxaldehyde (4A) as a light yellow solid.

¹H NMR (300 MHz, CDCl₃) δ9.2 (s, 1H), 7.3 (m, 9H overlaps CHCl₃), 7.22 (s, 1H), 7.1 (m, 6H), 7.0 (s, 1H).

1-(3-Benzylphenyl)ethanone (4B).

Step 1: (3-Bromophenyl)phenylmethanol

To an oven dried 500 ml 3-neck flask fitted with temperature probe, magnetic stir bar, and argon inlet was added a solution of 2.5M n-butyl lithium in hexanes (20.8 ml, 0.052 mole) chilled to −78° C. then diluted with diethyl ether (90 ml). To this was added dropwise by syringe over 30 minutes 1,3-dibromobenzene. (11.80 g, 6.043 ml, 0.05 mole; activated basic alumina pretreatment) keeping the internal temperature between −74° C. and −78° C. The reaction was aged at −78° C. for 2.5 h before adding neat benzaldehyde (5.52 g, 5.29 ml, 0.052 mole) over 15 minutes then allowing the reaction mixture to slowly warm to room temperature as the bath discharged overnight. The reaction was quenched with 20 mL H₂O then acidified with 5.4 ml conc. HCl and extracted with EtOAc three times. The combined organic layers were washed with NaHCO₃, brine and dried over NaSO₄, filtered and evaporated in vacuo to give a clear yellow oil (3-bromophenyl)phenylmethanol which crystallized to afford a white solid after washing with pet ether. Rf=0.14 (10% EtOAc/Hexanes). ¹H NMR (300 MHz, CDCl3) δ7.56 (s, 1H), 7.36-7.40 (m, 3H), 7.32-7.35 (m, 2H), 7.25-7.31 (m, 2H), 7.19 (m, 1H), 5.79 (s, 1H), 2.25 (s, 1H).

Step 2: (3-Benzyl)phenyl bromide

A solution of (3-bromophenyl)ethanone (4.10 g, 0.0156 mole) and triethylsilane (2.72 g, 3.71 ml, 0.0234 mole) in methylene chloride (40 ml) was chilled to 0° C. under argon with stirring followed by addition of neat boron trifluoride etherate (3.32 g, 2.96 ml, 23.4 mmol). The reaction stirred at room temprature overnight. The reaction mixture was poured into 160 ml saturated NaHCO₃ and extracted with EtOAc three times, the combined organic layers were washed with brine and dried over Na₂SO₄, filtered and evaporated to afford colorless oil. Chromatographic purification using 5% EtOAc/hexanes afforded pure (3-benzyl)phenyl bromide; Rf=0.44 (5% EtOAc/Hexanes) ¹H NMR (400 MHz, CDCl₃) δ7.27-7.33 (m, 4H), 7.09-7.23 (m, 5H),3.93 (s, 2H).

Step 3: 1-(3-Benzylphenyl)ethanone (4B)

To an oven-dried 100 ml 3-neck flask fitted with temperature probe, magnetic stir bar, and argon inlet was added 1.10 g of (3-benzyl)phenyl bromide in 26 ml THF and cooled to −78° C. Following dropwise addition of 1.6 M n-butyl lithium in hexanes (4.90 ml, 49 mmol) over 15 minutes, the reaction was stirred for 1 h at −78° C. before adding neat N-methoxy-N-methylacetamide (551 mg, 53.4 mmol) over 20 minutes. The reaction mixture warmed slowly to room temperature as the bath discharged overnight. The reaction was quenched with 60 ml 10% KHSO₄ and extracted with Et2O three times. The combined organic layers were washed with NaHCO₃, brine and dried over Na2SO₄, filtered and evaporated in vacuo to give a clear yellow oil. Chromatographic purification using EtOAc/hexanes afforded pure 4B. Rf=0.10 (5% EtOAc/hexanes); 0.40 (30 acetone, 70 hexane, 1.5 HOAc); ¹H NMR (400 MHz, CDCl₃) δ7.80 (m, 2H), 7.39 (m, 2H),7.29 (m, 2H), 7.19 (m, 3H), 4.05 (s, 2H), 2.6 (s, 3H).

1-(3-Benzylphenyl)-3-hydroxy-3-(1-trityl-1-H-imidazol-2-yl)propane-1-one (4C)

To an oven dried 100 mL three necked round bottomed flask with a stirring bar, septum, argon inlet and thermometer was added TMF (15 mL) and 4B (1-(3-benzyphenyl)ethanone (0.5 g, 0.0024 mole). The reaction was cooled to −78° C. and treated with lithiodiisopropylamine (0.0029 mole, Aldrich) for 30 minutes. To this well stirred solution was added a solution of 4A (0.88 g, 0.0026 mole,) in THF (10 mL)) dropwise over 12 minutes, maintaining the temperature <−65° C. This solution was aged 15 min then quenched at −78° C. with a solution of NH₄Cl. The mixture was diluted with EtOAc and the layers separated. The aqueous layer was extracted further with EtOAc and the organic layers were combined and washed with saturated aqueous NaHCO₃ solution and brine. Drying (Na₂SO₄), filtration and removal of the solvent in vacuo gave 1-(3-Benzylphenyl)-3-hydroxy-3-(1-trityl-1-H-imidazol-2-yl)propane-1-one (4C).

Rf=0.09 (50% EtOAc/Hexanes)

¹H NMR (400 MHz, CDCl₃) δ7.5(s, 1H), 7.28(m, 15H), 7.19(m, 15H), 7.02 (m, 1H), 6.9(s, 1H), 4.8(d, j=10 Hz,1H), 3.98 (s, 2H), 3.25(dd, j=10, 18 Hz, 1H), 3.1 (bs, 1H), 1.55(dd, j=10, 18 Hz, 1H).

1-(3-Benzylphenyl)-3-hydroxy-3-(1-H-imidazol-2-yl)propane-1-one (4D)

To an oven dried 100 mL three necked round bottomed flask with a stirring bar, septum, argon inlet and thermometer was added 4C (0.2g, 0.0018 mole) and trifluoroacetic acid (2 mL). To this well stirred solution was added EtSiH (0.078 mL, 0.0008 mole), and a white solid precipitated. After 1 hr the volatile components were removed in vacuo and residue was then quenched at with a saturated solution of NaHCO₃. The mixture was diluted with EtOAc and the layers separated. The aqueous layer was extracted further with EtOAc and the organic layers were combined and washed with brine. Drying (Na₂SO₄), filtration and removal of the solvent in vacuo to gave crude product that was chromatographed on silica with 30% isopropylacetate/chloroform saturated with ammonia to give pure 1-(3-Benzylphenyl)-3-hydroxy-3-(1-H-imidazol-2-yl)propane-1-one (4D).

¹H NMR (400 MHz, CDCl₃) δ9.5 (bs, 1H), 7.85(m, 2H), 7.4−7.1(m, 7H), 7.02 (s, 2H), 5.4(dd, j=3.8 Hz,1H), 4.01(s,2H), 3.82 (dd, j=3.18 Hz, 1H), 3.48(dd, j=8.18 Hz, 1H).

1-(3-Benzylphenyl)-3-(1-H-imidazol-2-yl)propane-1,3-dione (4E)

To an oven dried 100 mL three necked round bottomed flask with a stirring bar, septum, argon inlet and thermometer was added 4D(0.246 g, 0.00085 mole) and CHCl₃ (7 mL). To this well stirred solution was added MnO₂ (1.05 g, 0.012 mole). After 1 hr the reaction was filtered through celite and evaporated to dryness. The residue was purified by HPLC to give pure 1-(3-Benzylphenyl)-3-(1-H-imidazol-2-yl)propane-1,3-dione (4E) as the TFA salt.

| | | | |
|---|---|---|---|
| Anal. Calc'd for: C₁₉H₁₆N₂O₂.TFA | C, 60.28; | H, 4.10; | N, 6.70. |
| Found: | C, 60.27; | H, 4.17; | N, 6.65. |

¹H NMR (300 MHz, CDCl₃+d6DMSO) δ7.9 (s, 2H), 7.3(m, 11H), 4.05(s,2H).

1-(3-Benzyphenyl)-3-(1-benzyl-1H-imidazol-2-yl)propane-1,3-dione TFA salt (4F)

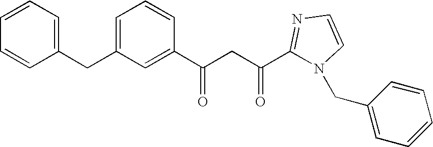

1-Benzyl-1-H-imidazole-2-carbaldehyde

A suspension of 1-H-imidazole-2-carbaldehyde (2.35 g, 0.025 mole) in, 120 mL of CH₃CN was treated with K₂CO₃ (4.0 g, 0.029 mole) followed by benzyl bromide (3.7 g, 0.022 mole) and stirred for three hr at 40° C. The solvent was removed in vacuo and the residue dissolved in EtOAc/CH₂Cl₂. The solution was washed with NaHCO₃, pH 7 buffer,and brine, filtered and evaporated to give solids that were triturated with CH₂Cl₂/petroleum ether to give 1-Benzyl-1-H-imidazole-2-carboxaldehyde as a light yellow solid.

¹H NMR (300 MHz, CDCl₃) δ9.8 (s, 1H), 7.4−7.3 (m, 4H), 7.2 (m, 2H), 7.15 (s, 1H), 5.5 (s, 2H).

1-(3-Benzylphenyl)-3-(1-benzyl-1H-imidazol-2-yl)propane-1,3-dione TFA salt (4F)

4F was prepared as described for 4E except that 1-Benzyl-1-H-imidazole-2-carboxaldehyde (see above) was used in the aldol condensation step, and the Et₃SiH/TFA step was omitted.

| | | | |
|---|---|---|---|
| Anal. Calc'd for: C₂₆H₂₂N₂O₂.TFA.H₂0 | C, 65.09; | H, 4.66; | N, 5.42. |
| Found: | C, 65.15; | H, 4.67; | N, 5.34. |

1-(3-Benzylphenyl)-3-(5,6,7,8-tetrahydro-[1,8]naphthyridin-2-yl)-propane-1,3-dione (4G)

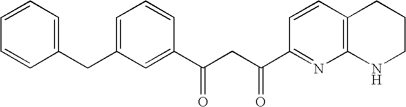

2-Dimethoxymethyl-[1,8]naphthyridine

A mixture containing 2-amino-3-formylpyridine (30 g, 0.245 mol) (prepared as described in J. Org. Chem., 1983, vol. 48, p. 3401), pyruvaldehyde dimethylacetal (87 g, 0.737 mol), and L-proline (7.0 g, 0.062 mol) in MeOH (300 mL) was refluxed under argon for 16 h. The cooled solution was filtered, evaporated and the residue dissolved in CH₂Cl₂ (500 mL) and washed with water and brine then dried and concentrated to a volume of ca. 100 mL. Hexane (300 mL) was added and the mixture was kept at 0° C. for 3 h, then filtered affording 2-dimethoxymethyl-[1,8]naphthyridine as an off-white crystalline solid.

¹H NMR (300 MHz, CDCl₃) δ9.14 (d, J=2.2 Hz, 1H); 8.26 (d, J=8.7 Hz, 1H); 8.21 (dd, J=8.7, 2.2 Hz, 1H); 7.8 (d, J=8.3 Hz, 1H); 7.5 (m, 1H); 5.48 (s, 1H); 3.53 (s, 6H).

2-Dimethoxymethyl-5,6,7,8-tetrahydro-[1,8]naphthyridine

A solution of 2-dimethoxymethyl-[1,8]naphthyridine (10 g, 0.049 mol) in ethanol (100 ml) was treated with 10% Pd on C (1.5 g) and the resulting mixture stirred under a H₂ filled balloon for 12.5 h. The catalyst was removed by filtration through celite and the solution concentrated to afford 2-dimethoxymethyl-5,6,7,8-tetrahydro-[1,8]naphthyridine as a yellow crystalline solid.

¹H NMR (300 MHz, CDCl₃) δ7.18 (d, J=7.12 Hz, 1H); 6.71 (d, J=7.12 Hz, 1H); 5.18 (s, 1H); 4.96 (br, s, 1H); 3.43 (s, 6H); 3.4 (m, 2H); 2.65 (m, 2H); 1.91 (m, 2H).

5,6,7,8-Tetrahydro-[1,8]naphthyridine-2-carboxaldehyde

2-Dimethoxymethyl-5,6,7,8-tetrahydro-[1,8]naphthyridine (10 g, 0.048 mol) was treated with trifluoroacetic acid (50 mL) and the resulting solution stirred under argon for 12.5 h. The TFA was removed at reduced pressure and the residue partitioned between sat. NaHCO₃ and CH₂Cl₂. The organic layer was dried, concentrated and passed through a 3 in. pad of silica gel (10% acetone/CH₂Cl₂) and concentrated to afford 5,6,7,8-tetrahydro-[1,8]naphthyridine-2-carboxaldehyde as a yellow crystalline solid.

¹H NMR (300 MHz, CDCl₃) δ9.80 (s, 1H); 7.31 (d, J=7.32 Hz, 1H); 7.16 (d, J=7.32 Hz, 1H); 5.31 (br, s, 1H); 3.48 (m, 2H); 2.81 (m, 2H); 1.94 (m, 2H).

1-(3-Benzylphenyl)-3-(5,6,7,8-tetrahydro-[1,8]naphthyridin-2-yl)-propane-1,3-dione (4G)

In a manner similar to that for 4E, 1-(3-benzylphenyl)-3-(5,6,7,8-tetrahydro-[1,8]naphthyridin-2-yl)-propane-1,3-dione (4G) was prepared, using 5,6,7,8-tetrahydro-[1,8]naphthyridine-2-carbaldehyde.

| | | | |
|---|---|---|---|
| Anal. Calc'd for: | C, 63.32; | H, 5.04; | N, 5.33. |
| C₂₀H₁₆N₂O₂ 1.05.TFA.0.4 Dioxane | | | |
| Found: | C, 63.49; | H, 4.64; | N, 4.96. |

### EXAMPLE 5

1-(3-Benzylphenyl)-3-(imidazole-4-yl)propane-1,3-dione (5D)

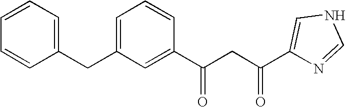

Methyl (1-N-triphenylmethyl)imidazole-4-carboxylate (5B)

To a suspension of methyl 4-imidazole carboxylate (0.5 g, 3.96 mmol) in DMF (4 mL) was added triethylamine (0.828 mL, 5.94 mmol) and triphenylmethyl bromide (1.54 g, 4.76 mmol) at 0° C. under argon. The reaction mixture was allowed to warm to ambient temperature and stirred for 5 h. The reaction was then quenched with water and the mixture was extracted with ethyl acetate. The organic extraxt was washed with brine, dried over magnesium sulfate, filtered and concentrated under vacuum. The resultant residue was purified by flash chromatograph on silica gel (hexane/ethyl acetate) to give 5B.

1H NMR (400 MHz, CDCl₃) δ7.58 (d, 1H), 7.46 (d, 1 H), 7.33-7.38 (m, 9 H), 7.10-7.14(m, 6 H), 3.87 (s, 3 H).

1-(3-Benzylphenyl)-3-(1-N-triphenylmethyl-imidazole-4-yl)propane-1,3-dione (5C)

To a flask charged with potassium t-butoxide (0.133 g, 1.19 mmol) and p-xylene (2 mL) was added 1-(3-benzylphenyl) methyl ketone (4B) (0.1 g, 0.476 mmol) in p-xylene (2 mL) under argon and the reaction mixture was stirred at ambient temperature for 3 h. 5B (0.21 g, 0.57 mmol) was then added. After all starting material was consumed, the reaction mixture was treated with saturated aqueous ammonium chloride solution and extracted with dichloromethane. The organic extract was dried over magnesium sulfate and concentrated under vacuum. The residue was redissolved in small amount of chloroform and filtered. The filtrate was concentrated and used for next reaction without further purification.

1-(3-Benzylphenyl)-3-(imidazole-4-yl)propane-1,3-dione (5D)

The crude 5C in dichloromethane (1 mL) was treated with trifluoroacetic acid (0.5 mL) and titrated with triethylsilane (about 0.2 mL) until the orange-red color disappeared. The reaction mixture was then concentrated under vacuum. The residue was purified by reverse-phase HPLC and then recrystallized with dichloromethane/hexane to give 5D as light yellow solid.

1H NMR (400 MHz, CD₃OD) δ8.74 (s, 1 H), 8.35 (s, 1 H), 7.91 (s, 1 H), 7.86-7.90 (m, 1 H), 7.42-7.46 (m, 2 H), 7.16-7.30 (m, 5 H), 7.01 (s, 1 H), 4.06 (s, 2 H).

1-(3-Benzylphenyl)-3-(1-N-methyl-imidazole-4-yl)propane-1,3-dione (5E)

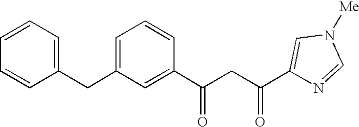

Methyl (1-N-methyl)imidazole-4-carboxylate

A mixture of 4-iodo-1-methyl-1H-imidazole (500 mg, 2.40 mmol), trans-dichlorobis(triphenylphosphine)palladium(II) (255 mg, 0.30 mmol) and triethylamine (3 mL) in methanol (30 mL) in a pressure vessel was purged with argon for 10 min. The vessel was then pressurized with carbon monoxide to 250 psi and heated at 100° C. for 2 days. After cooling to room temperature, the reaction mixture was filtered through Celite, and concentrated under vacuum. The resultant residue was purified by flash chromatograph on silica gel (5% MeOH in CHCl₃) to give title compound.

¹H NMR (400 MHz, CDCl₃) δ7.60 (s, 1H), 7.28 (s, 1H), 3.90 (s, 3H), 3.78 (s, 3H).

1-(3-Benzylphenyl)-3-(1-N-methylimidazole-4-yl)propane-1,3-dione (5E)

To a solution of 1-(3-benzylphenyl) methyl ketone (4B) (213 mg, 1.01 mmol) in dry THF (5 mL) under argon was added potassium bis(trimethylsilyl)amide (0.5 M solution in toluene, 2.0 mL, 1.0 mmol) at 0° C. and stirred for 30 min. Methyl (1-N-methyl)imidazole-4-carboxylate was then added and the reaction mixture was stirred at 0° C. for 10 min then ambient temperature for 15 min. The reaction was quenched with 1N HCl to pH 8. The reaction mixture was extracted with CHCl₃ three times. The combined organic phases were washed with brine, dried over sodium sulfate and concentrated under vacuum. The residue was triturated with ether/hexanes to give 5E.

¹H NMR (400 MHz, CDCl₃) δ7.86-7.90 (m, 2H), 7.63 (d, 1H), 7.50 (d, 1H), 7.25-7.40 (m, 4H), 7.18-7.22 (m, 3H), 7.07 (s, 1H), 4.04 (s, 2H), 3.77 (s, 3H).

1-(3-Benzylphenyl)-3-[1-N-(pyridin-4-yl)methylimidazole-4-yl]propane-1,3-dione (5F)

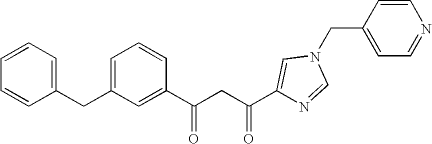

Methyl (1-N-(pyridin-4-yl)methyl)imidazole-4-carboxylate

To a mixture of methyl 4-imidazolecarboxylate (500 mg, 3.96 mmol) and 4-picolyl chloride hydrochloride (743 mg, 4.53 mmol) in DMF (10 mL) sodium hydride (210 mg, 8.75 mmol) was added. The reaction mixture was then heated at 60° C. for 3 h, and concentrated under vacuum. The residue was partitioned between CHCl₃ and brine. The organic phase was dried over sodium sulfate and concentrated under vacuum. The residue was dissolved in a mixture of ethyl acetate/ether and a white solid was obtained upon cooling to provide the title compound.

¹H NMR (400 MHz CDCl₃) δ8.64 (d, 2H), 7.61 (s, 1H), 7.58 (s, 1H), 7.03 (d, 2H), 5.18 (d, 2H), 3.90 (s, 3H).

1-(3-Benzylphenyl)-3-[1-N-(pyridin-4-yl)methylimidazole-4-yl]propane-1,3-dione (5F)

To a solution of 1-(3-benzylphenyl) methyl ketone (4B) (313 mg, 1.49 mmol) and methyl [1-N-(pyridin-4-yl)methyl]imidazole-4-carboxylate (127 mg, 0.58 mmol) in dry THF (7 mL) under argon was added sodium methoxide (86 mg, 1.59 mmol) at 0° C. and stirred at ambient temperature. After 2 h, the reaction mixture was neutralized to pH 7 with 1N HCl in ether. The mixture was extracted with CHCl₃ three times. The combined organic phases were dried over sodium sulfate and concentrated under vacuum. The residue was recrystallized with ether/hexanes to give 5F as a solid.

¹H NMR (400 MHz, CDCl₃) δ8.63 (d, 2H), 7.84-7.90 (m, 2H), 7.66 (s, 1H), 7.61 (s, 1H), 7.25-7.40 (m, 4H), 7.18-7.22 (m, 3H), 7.10 (s, 1H), 7.06 (d, 2H), 5.21 (s, 2H), 4.04 (s, 2H).

1-(3-Benzylphenyl)-3-[1-N-(pyridin-2-yl)methylimidazole-4-yl]propane-1,3-dione (5G)

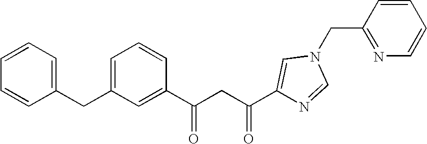

In a manner similar to that for 5F, 1-(3-benzylphenyl)-3-[1-N-(pyridin-2-yl)methylimidazole-4-yl]propane-1,3-dione (5G) was prepared.

¹H NMR (400 MHz, CDCl₃) δ8.62 (d, 1H), 7.83-7.90 (m, 3H), 7.67-7.75 (m, 3H), 7.25-7.40 (m, 4H), 7.18-7.22 (m, 3H), 7.08-7.10 (m, 2H), 5.28 (s, 2H), 4.04 (s, 2H).

1-(3-Benzylphenyl)-3-[1-N-(pyridin-3-yl)methylimidazole-4-yl]propane-1,3-dione (5H)

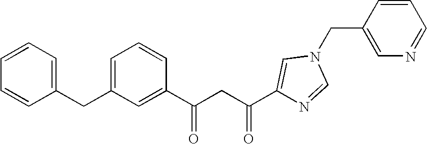

In a manner similar to that for 5F, 1-(3-benzylphenyl)-3-[1-N-(pyridin-3-yl)methylimidazole-4-yl]propane-1,3-dione (5H) was prepared.

¹H NMR (400 MHz, CDCl₃) δ8.57-8.66 (m, 2H), 7.83-7.90 (m, 2H), 7.60-7.65 (m, 2H), 7.47-7.51 (m, 1H), 7.25-7.40 (m, 5H), 7.18-7.22 (m, 3H), 7.08 (s, 1H), 5.20 (s, 2H), 4 04 (s, 2H).

1-(3-Benzylphenyl)-3-{1-N-[(1-N-tert-butylcarbamyl)-piperidine-4-yl]methylimidazole-4-yl}propane-1,3-dione (5I)

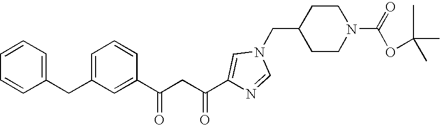

In a manner similar to that for 5F, 1-(3-benzylphenyl)-3-{1-N[(1-N-tert-butylcarbamyl)piperidine-4-yl]methylimidazole-4-yl}propane-1,3-dione (5I) was prepared.

¹H NMR (400 MHz, CDCl₃) δ7.8-7.95 (m, 2H), 7.63 (s, 1H), 7.51 (s, 1H), 7.25-7.42 (m, 5H), 7.18-7.22 (m, 2H), 7.08 (s, 1H), 4.10-4.20 (m, 2H), 4.04 (s, 2H), 3.93 (d, 2H), 2.63 (t, 2H), 1.90-2.00 (m, 1H), 1.50-1.70 (m 2H), 1.45 (s, 9H), 1.10-1.30 (m, 2H).

1-(3-Benzylphenyl)-3-[1-N-(piperidine-4-yl)methylimidazole-4-y]propane-1,3-dione (5J)

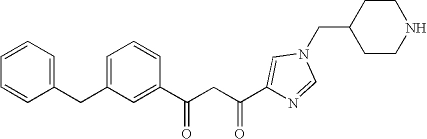

To a solution of 5I (750 mg, 1.5 mmol) in dichloromethane (20 mL) was added trifluoroacetic acid (4 mL) and the reaction mixture was stirred for 1 hr at ambient temperature. Trifluoroacetic acid and solvent were removed under reduced pressure. The residue was partitioned between CHCl₃ and brine. The organic phase was dried over sodium sulfate and concentrated under vacuum. The residue was recrystallized with methanol/ether to give 5J.

¹H NMR (400 MHz, CDCl₃) δ7.84-7.90 (m, 2H), 7.74 (s, 1H) 7.60 (s, 1H), 7.52 (s, 1H), 7.17-7.42 (m, 6H), 7.06 (s, 1H), 4.04 (s, 2H), 4.00-4.04 (m, 2H), 3.42 (d, 2H), 2.70-2.90 (m, 3H), 2.00-2.20 (m, 1H), 1.72-1.82 (m, 4H).

1-(3-Benzylphenyl)-3-{1-N-[(1-N-methanesulfonyl)piperidine-4-yl]methylimidazole-4-yl}propane-1,3-dione (5K)

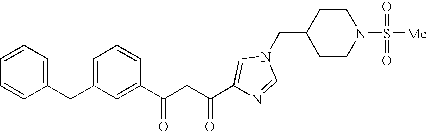

To a solution of 5J (50 mg, 0.13 mmol) in DMF (2 mL) was added diisopropylethylamine (0.035 mL, 0.20 mmol) and methanesulfonyl chloride (0.012 mL, 0.16 mmol) under argon at ambient temperature. After 40 min, the solvent were removed under reduced pressure. The residue was partitioned between CHCl₃ and brine. The organic phase was dried over sodium sulfate and concentrated under vacuum. The residue was recrystallized with dichloromethane/ether to give 5K as a solid.

¹H NMR (400 MHz, CDCl₃) δ7.84-7.90 (m, 2H), 7.64 (s, 1H), 7.51 (s, 1H), 7.17-7.42 (m, 7H), 7.08 (s, 1H)) 4.04 (s, 2H), 3.82-3.95 (m, 4H), 2.79 (s, 3H), 2.62 (t, 2H), 1.80-1.95 (m, 1H), 1.70-1.80 (m, 2H), 1.42-1.50 (m, 2H).

1-(3-Benzylphenyl)-3-{1-N-[2-(1-N-tert-butylcarbamylpiperiazin-4-yl)ethyl]imidazole-4-yl}propane-1,3-dione (5L)

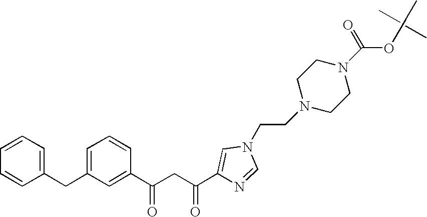

In a manner similar to that for 5I, 1-(3-benzylphenyl)-3-{1-N-[2-(1-N-tert-butylcarbamylpiperiazin-4-yl)ethyl]imidazole-4-yl}propane-1,3-dione (5L) was prepared.

¹H NMR (400 MHz, CDCl₃) δ7.84-7.90 (m, 2H), 7.71 (d, 1H), 7.61 (d, 1H), 7.25-7.40 (m, 5H), 7.18-7.22 (m, 2H), 7.08 (s, 1H), 4.08 (t, 2H), 4.04 (s, 2H), 3.40-3.44 (m, 4H), 2.73 (t, 2H), 2.40-2.48 (m, 4H), 1.45 (s, 9H).

1-(3-Benzylphenyl)-3-{1-N-[2-(piperiazin-1-yl)ethyl]imidazole-4-yl}propane-1,3-dione (5M)

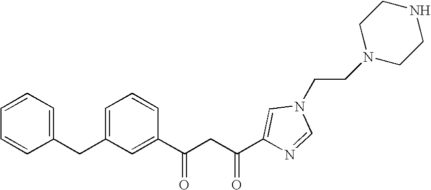

In a manner similar to that for 5J, 1-(3-benzylphenyl)-3-{1-N-[2-(piperiazin-1-yl)ethyl]imidazole-4-yl}propane-1,3-dione (5M) was prepared.

¹H NMR (400 CDCl₃) δ7.84-7.90 (m, 2H), 7.69 (s, 1H), 7.58 (s, 1H), 7.25-7.40 (m, 5H) 7.18-7.22 (m, 2H), 7.08 (s, 1H), 4.08 (t, 2H), 4.04 (s, 2H), 3.16-3.22 (m, 4H), 2.80 (t, 2H), 2.74-2.77 (m, 4H).

1-(3-Benzylphenyl)-3-{1-N-[2-(1-N-methanesulfonyl-piperazin-4-yl)ethyl]-imidazole-4-yl}propane-1,3-dione (5N)

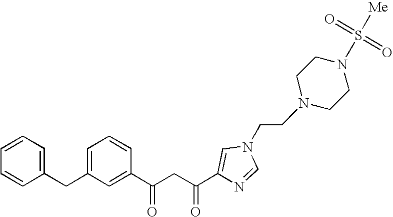

In a manner similar to that for 5K, 1-(3-benzylphenyl)-3-{1-N-[2-(1-N-methanesulfonylpiperiazin-4-yl)ethyl]imidazole-4-yl}propane-1,3-dione (5N) was prepared.

¹H NMR (400 MHz, CDCl₃) δ7.83-7.90 (m, 2H), 7.72 (s, 1H), 7.58 (s, 1H), 7.25-7.40 (m, 5H), 7.18-7.22 (m, 2H), 7.08 (s, 1H), 4.08 (t, 2H), 4.04 (s, 2H), 3.20-3.26 (m, 4H), 2.76-2.82 (m, 5H), 2.58-2.64 (m, 4H).

1-(3-Benzylphenyl)-3-{1-N-[2-(1-N-benzylpiperiazin-4-yl)ethyl]imidazole-4-yl}propane-1,3-dione (5O)

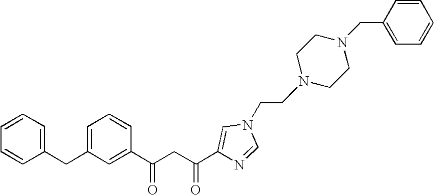

In a manner similar to that for 5F, 1-(3-benzylphenyl)-3-{1-N-[2-(1-N-benzylpiperiazin-4-yl)ethyl]imidazole-4-yl}propane-1,3-dione (5O) was prepared.

¹H NMR (400 MHz, CDCl₃) δ8.14 (s, 1H), 7.96 (s, 1H), 7.78-7.86 (m, 2H), 7.44-7.52 (m, 8H), 7.20-7.36 (m, 4H), 7.04 (s, 1H), 4.15-4.39 (m, 4H), 4.04 (s, 2H), 2.80-3.60 (m, 8H), 2.30-2.45 (m, 2H).

1-[3-Benzyl-5(6-oxo-6-pyrimidin-1-ylmethyl)phenyl[-3-(1-methylimidazole-4-yl)propane-1,3-dione (5P)

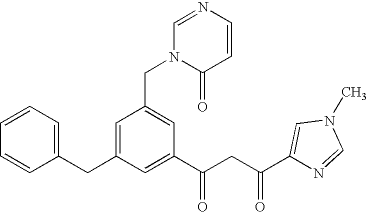

In a manner similar to that for 16O and 5E, 1-[3-benzyl-5-(6-oxo-6H-pyrimidin-1-ylmethyl)phenyl]-3-(1-methylimidazole-4-yl)propane-1,3-dione (5P) was prepared.

| | | | |
|---|---|---|---|
| Anal. Calc'd for C₂₅H₂₂N₄O₃.2 HCl | C, 60.13; | H, 4.84; | N, 11.22. |
| Found: | C, 59.98; | H, 4.54; | N, 10.82. |

1-[3-Benzyl-5-(1,1-dioxoisothiazolidin-2-ylmethyl)phenyl]-3(1-methylimidazole-4-yl)propane-1,3-dione (5Q)

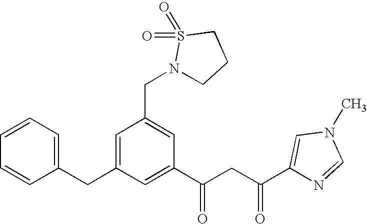

In a manner similar to that for 5P, 1-[3-benzyl-5(1,1-dioxoisothiazolidin-2ylmethyl)phenyl]-3-(1-methylimidazole-4-yl)propane-1,3-dione (5Q) was prepared.

| | | | |
|---|---|---|---|
| Anal. Calc'd for C₂₄H₂₅N₃O₄S | C, 63.84; | H, 5.58; | N, 9.31. |
| Found: | C, 63.54; | H, 5.36; | N, 8.99. |

1-(3-Benzylphenyl)-3-pyrimidin-2-yl-propane-1,3dione (5R)

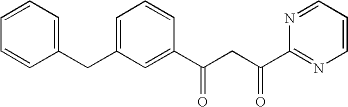

Pyrimidine-2-carbonitrile

To a solution of 2-bromopyrimidine (1.5 g, 9.43 mmol) in DMSO (30 mL) was,added 1,4-diazabicyclo(2.2.2)octane (0.22 g, 1.88 mmol) and sodium cyanide (0.69 g, 14.2 mmol) at room temperature under argon. As the reaction proceeded the sodium cyanide gradually dissolved into the solution. After one hour the reaction was quenched with water and was extracted five times with diethyl ether. The organic extract was washed with brine, dried over sodium sulfate, filtered and concentrated under vacuum. The resultant residue was purified by flash chromatography on, silica gel (hexane/ethyl acetate) to give title compound as a light yellow solid.

¹H NMR (300 MHz, CDCl₃) δ8.92−8.91 (d, j=4.9 Hz, 2 H), 7.61-7.65 (t, j=5.0 Hz, 1 H).

Methyl pyrimidine-2-carboxylate

To a solution of pyrimidine-2-carbonitrile (0.5 g, 4.76 mmol) and water (4.76 mmol) in methanol (15 mL) was bubbled HCl gas until the solution was saturated. Once saturation was completed the solution was refluxed for 2 hours. The reaction was cooled in dry ice to-induce crystallization of a white powder which was discarded. The resultant solvate was concentrated and the residue was partitioned between saturated aqueous sodium bicarbonate solution and ethyl acetate. The sodium bicarbonate layer was extracted two more times with ethyl acetate and the combined organic layers were dried over sodium sulfate, filtered and concentrated to afford title ester as a white solid powder.

¹H NMR (300 MHz, CDCl₃) δ8.96−8.94 (d, j=4.9 Hz, 2 H), 7.26-7.52 (t, j=4.9 Hz, 1 H,), 4.07 (s, 1H).

1-(3-Benzylphenyl)-3-pyrimidin-2-yl-propane-1,3-dione (5R)

To a solution of 4B (0.3 g, 1.43 mmol) and methyl pyrimidine-2-carboxylate (0.197 g, 1.43 mmol) in THF (7.0 ml) was added sodium methoxide (0.154 g, 2.85 mmol). After one hour of stirring at room temperature the yellow solution was poured into a saturated solution of ammonium chloride, extracted with ethyl acetate, dried over sodium sulfate, filtered and concentrated to give a yellow oil. The oil was dissolved in DMSO and purified using reverse phase HPLC on a C18 column to give a light colored solid, (5R) after lyophilization

¹H NMR (400 MHz, CDCl₃) δ8.97−8.96 (d, j=4.8 Hz, 2 H), 7.94−7.91 (m, 2 H), 7.61 (s, 1 H), 7.46−7.39 (m, 3 H), 7.32−7.19 (m, 5 H), 4.07 (s,2 H).

| | | | |
|---|---|---|---|
| Anal. Calc'd for C₂₀H₁₆N₂O₂. | C, 75.93; | H, 5.10; | N, 8.86. |
| Found: | C, 75.89; | H, 5.27; | N, 8.95. |

1-(3-Benzylphenyl)-3-(4-methylpyrimidin-2-yl)propane-1,3-dione (5S)

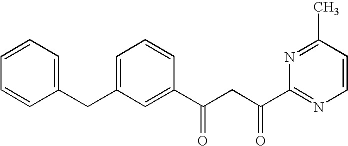

2-Chloro-4-methyl-pyrimidine

A mixture of 2,6-dichloropyrimidine (1.5 g, 10.07 mmol), trimethylaluminum (0.87 g, 12.08 mmol), tetrakis(triphenylphosphine)palladium(0) (0.81 g, 0.7 mmol) in THF (30 mL) was heated under reflux for several hours. The reultant mixture was quenched with addition of water. (Note: allowing reaction to go overnight was detrimental). The product mixture was extracted with ethyl acetate three times, dried over sodium sulfate, filtered and concentrated to give a dark yellow oil. The resultant oil was purified by flash chromatography on silica gel (hexane/ethyl acetate) to give title compound as a light yellow solid.

¹H NMR (400 MHz, DMSO) δ8.62−8.61 (d, j=5.1 Hz, 1 H), 7.47−7.46 (d, j=5.1 Hz, 1 H), 2.56 (s, 3H).

4-Methyl-pyrimidine-2-carbonitrile

In manner similar to the preparation of pyrimidine-2-carbonitrile in the synthesis of 5R.

1H NMR (400 MHz, CDCl₃) δ8.70−8.68 (d, j=5.3 Hz, 1 H), 7.40 (d, j=5.1 Hz, 1H,), 2.63 (s, 3H).

Methyl 4-methylpyrimidine-2-carboxylate

In a manner similar to the preparation of methyl pyrimidine-2-carboxylate, title ester was synthesized. In this case, following the workup, the crude product was chromatographed on silica gel eluting with ether/dichloromethane to afford the ester as a white solid.

¹H NMR (400 MHz, CDCl₃) ε8.78−8.76(dxd, j=2.6 Hz, 5.1 Hz, 1 H), 7.36−7.34 (dxd, 1.6 Hz, 5.0 Hz, 1 H), 4.07 (s, 3 H), 2.68 (s, 3 H).

1(3-Benzylphenyl)-3-(4-methylpyrimidin-2-yl)propane-1,3-dione (5S)

In a manner similar to the preparation of 5R, 5S was synthesized.

¹H NMR (400 MHz, CDCl₃) δ8.76−8.75 (d, j=5.1 Hz, 1 H), 7.94−7.91 (m, 2 H), 7.59 (s, 1 H), 7.44−7.38 (m, 2 H) 7.32−7.20 (m, 6 H), 4.08 (s, 2 H), 2.67 (s, 3 H).

### EXAMPLE 6

1-[3-Benzyl-5-(2-oxo-2H-pyridin-1-ylmethyl)phenyl]-3-(4-methylpyridin-2-yl)-propane-1,3-dione (6F)

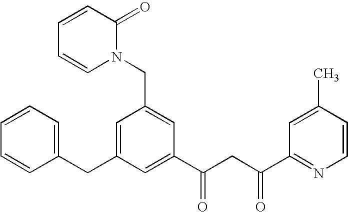

3Benzyl-5-bromobenzaldehyde (6A)

To a cold (−78° C.) solution of 1-benzyl-3,5-dibromobenzene (1.15 g) in THF (30 mL), a solution of n-BuLi in hexanes (2.5 M, 2 mL) was added. The resultant mixture was stirred at −78° C. for 1 h and was treated with anhydrous DMF (0.3 mL). The reaction mixture was allowed to warm up slowly to room temperature and was stirred at the temperature overnight. The product mixture was diluted with ethyl acetate and partitioned with aq. HCl. The organic extract was washed with brine, dried over magnesium sulfate, filtered, and concentrated under vacuum. The residue was subjected to column chromatography on silica gel eluting with 10% ethyl acetate in hexane. Collection and concentration of appropriate fractions provided the title benzaldehyde.

¹H NMR (400 MHz, CDCl₃) δ9.90 (s, 1H), 7.85 (s, 1H), 7.63 (s, 1H), 7.59 (s, 1H), 7.35−7.18 (m, 5H), 4.03 (s, 2H).

3-Benzyl-5-bromobenzyl alcohol (6B)

To a cold (0 C.) solution of 3-benzyl-5-bromobenzaldehyde (0.465 g) in methanol (5 mL), sodium borohydride (0.123 g) was added. The reaction mixture was stirred at room temperature for 3 hr. The product mixture was concentrated, and the residue partitioned between ethyl acetate and aq. HCl. The organic extract was washed with brine, dried over magnesium sulfate, filtered, and concentrated under vacuum to provide the title alcohol.

¹H NMR (400 MHz, CDCl₃) δ7.36(s, 1H), 7.31−7.15 (m, 6H),7.10 (s, 1H), 4.62 (br s, 2H), 3.94 (s, 2H).

3-Benzyl-5bromobenzyl bromide (6C)

To a cold (0 C.) solution of 3-benzyl-5-bromobenzyl alcohol (0.32 g) and carbon tetrabromide (0.57 g) in dichloromethane (6 mL), a solution of triphenylphosphine (0.45 g) in dichloromethane (4 mL) was added dropwise. The reaction mixture was stirred at room temperature for 2 hr. The product mixture was concentrated, and the residue was subjected to column chromatography on silica gel eluting with 15% ethyl acetate in hexane. Collection and concentration of appropriate fractions provided the title dibromide.

¹H NMR (400 MHz, CDCl₃) δ7.38 (s, 1H), 7.32−7.12 (m, 7H), 4.37 (s, 2H), 3.94 (s, 2H).

3-Benzyl-5-(2-oxo-2H-pyridin-1-ylmethyl)-1-bromobenzene (6D)

A mixture of 3-benzyl-5-bromobenzyl bromide (6C) (0.39 g) 2-hydroxypyridine (0.13 g) and cesium carbonate(0.443 g,) in DMF was heated to 110° C. for 2 hours. The product mixture was concentrated, and the residue partitioned between ethyl acetate and water. The organic extract was washed with brine, dried over magnesium sulfate, filtered, and concentrated under vacuum. The residue was subjected to column chromatography on silica gel eluting with 50% ethyl acetatel in hexane. Collection and concentration of appropriate fractions provided 6D.

¹NMR (400 MHz, CDCl₃) δ7.35−7.19 (m, 7H), 7.14 (d, J=7.5 Hz, 2H), 7.06 (s, 1H), 6.61 (d, J=8.6 Hz, 1H), 6.15 (t, J=6.7 Hz, 1H), 5.02 (s, 2H), 3.91 (s, 2H).

3-Benzyl-5-(2-oxo-2H-pyridin-1-ylmethyl)acetophenone (6E)

To a mixture of 3-benzyl-5-(2-oxo-2H-pyridin-1-ylmethyl)-1-bromobenzene (6D) (0.58 g), thallium acetate (0.47 g), 1,3-bis(diphenylphosphino)propane (0.18 g) and triethylamine (0.91 mL) in DMF (5 mL) in a pressure tube, purged with argon for a period of 10 minutes, palladium acetate (92 mg) and n-butyl vinyl ether (1.07 mL) was added. The reaction tube was sealed and stirred at 100° C. overnight. The reaction mixture was filtered through a bed of Celite, and the filtrate concentrated under vacuum. The residue was dissolved in THF (5 mL) and treated with aq. HCl (3M, 5 mL). The resultant mixture was stirred at rt for 3 hr., diluted with ethyl acetate, basified with aq. sodium bicarbonate. The organic extract was dried over magnesium sulfate, filtered, and concentrated under vacuum. The residue was subjected to column chromatography on silica gel eluting with 50% ethyl acetate in hexane. Collection and concentration of appropriate fractions provided the title ketone.

¹H NMR (400 MHz, CDCl₃) δ7.70 (br s, 2H), 7.35−7.21 (m, 6H), 7.15 (d, J=6.7 Hz, 2H), 6.6 (d, J=8.6 Hz, 1H), 6.15 (d, J=5.3 Hz, 1H), 5.14 (s, 2H), 4.00 (s, 2H), 2.35 (s, 3H).

1-[3-Benzyl-5-(2-oxo-2H-pyridin-1-ylmethyl)phenyl]-3-(4-methylpyridin-2-yl)-propane-1,3-dione (6F)

A solution of 3-benzyl-5-(2-oxo-2H-pyridin-1-ylmethyl)acetophenone (6E) (0.19 g) and methy 4-methylpyridine-2-carboxylate (3H) (0.28 g) in THF (1.2 mL) under an atmosphere of argon was treated with NaH (60 mg; 60% dispersion in mineral oil). The resultant mixture was stirred at room temperature for 15 minutes, and was quenched with saturated aq. ammonium chloride and partitioned with ethyl acetate. The organic extract was washed with brine, dried over magnesium sulfate, filtered, and concentrated under vacuum. The residue was subjected to column chromatography on silica gel eluting with 10% CH₃OH in CHCl₃. Appropriate fractions were collected and concentrated. The residue was triturated with ethyl acetate. Filtration and collection of the solid provided the title compound.

¹H NMR (400 MHz, CDCl₃) δ8.57 (d, J=4.88 Hz, 1 H), 7.98 (s, 1H), 7.82 (d, J=7.57 Hz, 2H), 7.49 (s, 1H), 7.35−7.14 (m, 9H), 6.62 (d, J=9.03 Hz, 1H), 6.15 (t, J=6.6 Hz, 1H), 5.17 (s, 2H), 4.03 (s, 2H), 2.45 (s, 3H). (t, J=6.6 Hz, 1H), 5.17 (s, 2H), 4.03 (s, 2H) 2.45 (s, 3H).

| | | | |
|---|---|---|---|
| Anal. Calc'd for C₂₈H₂₄N₂O₃. | C, 77.04; | H, 5.54; | N, 6.42. |
| Found: | C, 76.77; | H, 5.38; | N, 6.15. |

1-[3-Benzyl-5-(2-oxo-piperidin-1-ylmethyl)phenyl]-3-(4-methylpyridin-2-yl)-propane-1,3-dione (6G)

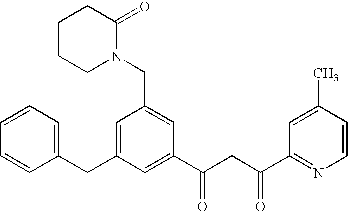

In a manner similar to that for 6F, 1-[3-Benzyl-5-(2-oxo-piperidin-1-ylmethyl)phenyl]-3-(4-methylpyridin-2-yl)-propane-1,3-dione (6G) was prepared.

| | | | |
|---|---|---|---|
| Anal. Calc'd for C₂₈H₂₈N₂O₃.0.15 hexane | C, 76.54; | H, 6.69; | N, 6.18. |
| Found: | C, 76.91; | H, 6.61; | N, 6.14. |

1-[3-Benzyl-5-(4-methylpiperazin-1-ylmethyl)phenyl]-3-(4-methylpyridin-2-yl)-propane-1,3-dione (6H)

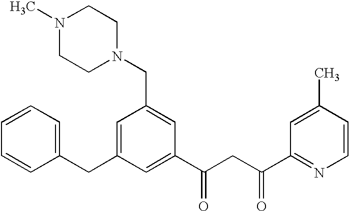

In a manner similar to that for 6F, 1-[3-Benzyl-5-(4-methylpiperazin-1-ylmethyl)phenyl]-3-(4-methylpyridin-2-yl)-propane-1,3-dione (1H) was prepared.

| | | | |
|---|---|---|---|
| Anal. Calc'd for | C, 56.64; | H, 4.92; | N, 6.15. |
| C₃₂H₃₃N₃O₂.2.10 TFA & 0.1 H₂O | | | |
| Found: | C, 56.84; | H, 5.11; | N, 5.76. |

ES MS M+1=442.

### EXAMPLE 7

1-(3-Benzylphenyl)-3-(4-methylpyridin-2-yl)-propane-1,3-dione (7B)

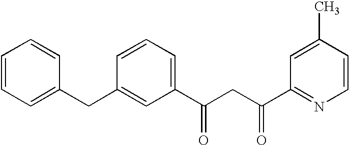

A mixture of 1-(3-benzylphenyl)ethanone (4B) (0.05 g; 0.24 mmol) and 4-methylpicolinic acid methyl ester (3H) (0.036 g, 0.24 mmol) was dissolved in THF (1 mL) in a flamed dried 5 mL round bottomed flask equipped with a magnetic stirring bar and nitrogen inlet. To this solution was added sodium ethoxide (0.033 g, 0.48 mmol), and the mixture allowed to stir for 1 hr after which 1 mL of an aqueous saturated ammonium chloride solution was added. After stirring for 5 min, the mixture was partitioned between EtOAc/H₂O and extracted. The combined organic extracts were washed with H₂O, brine, dried over anhydrous sodium sulfate, filtered, the solvent removed and the residue triturated with ether to afford 1-(3-benzylphenyl)-3-(4-methylpyridin-2-yl)-propane-1,3-dione (7B) as a yellow crystalline solid.

¹H NMR (400 MHz, CDCl₃) δ8.56(d, j=5 Hz, 1H), 7.99(s, 1H) 7.94(s, 1H), 7.91 (d, j=7.6 Hz, 1H), 7.54(s, 1H), 7.18-7.44(m, 8H), 4.07(s, 2H), 2.45 (s, 3H).

| | | | |
|---|---|---|---|
| Anal. Calc'd for: C₂₂H₁₉NO₂..2 EtOAc | C, 78.91; | H, 5.98; | N, 4.04. |
| Found: | C, 78.69; | H, 5.85; | N, 4.06. |

1-(3-Benzylphenyl)-3-pyrazin-2-ylpropane-1,3-dione (7C)

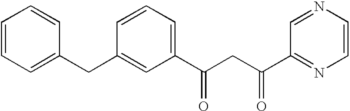

In a manner similar to that for 7B, 1-(3-benzylphenyl)-3-pyrazin-2-ylpropane-1,3dione (7C) was prepared.

| | | | |
|---|---|---|---|
| Anal. Calc'd for: C₂₀H₁₆N₂O₂ | C, 75.93; | H, 5.10; | N, 8.85. |
| Found: | C, 75.99; | H, 5.30; | N, 8.71. |

1-(3-Benzylphenyl)-3-(2-methylthiazol-4-yl)-propane-1,3-dione (7D)

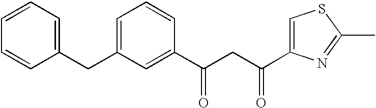

In a manner similar to that for 7B, 1-(3-benzylphenyl)-3-(2-methylthiazol-4-yl)-propane-1,3-dione (7D) was prepared.

FAB MS: m/z 336 (m+1)

### EXAMPLE 8

1-[3(2,6-difluoro-benzyl)-phenyl]-3-(4-methoxy-pyridin-2-yl)-propane-1,3-dione (8B)

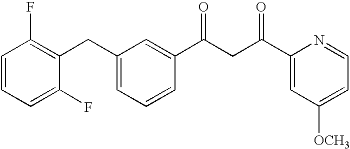

1-[3-(2,6-difluorobenzyl)-phenyl]-ethanone (8A)

A suspension of Dibromoethane (0.28 mL, 0.0032 mole) in 35 mL of THF under Argon was treated with activated zinc (10.5 g, 0.161 mole) and heated to reflux 2 times for 30 seconds each time. The suspension was then cooled in a salt water ice bath to −8° C. and 2,6-Difluorobenzyl bromide (16.8 g, 0.081 mole) in 20 mL of THF was added dropwise. After addition was completed the reaction was allowed to stir at 0° C., for 1 hour. In a separate flask was added Bis(dibenzylideneacetone)palladium (3.1 g, 0.00537 mole), Tri(2-furyl) phosphine (2.5 g, 0.0107 mole), and 3-iodoacetophenone (13 g, 0.0537 mole) to 40 mL of THF and cooled to 0° C. The zinc suspension was then cannulated into the 3-iodoacetophenone mixture and the ice bath was removed. After 18 hours the mixture was filtered through a pad of celite and washed several times with EtOAc and then the solvents were removed under reduced pressure. The residue was treated with 150 mL of a saturated aqueous solution of NH₄Cl and extracted with EtOAc three times, the combined organic layers were dried over NaSO₄, filtered and evaporated to give a brown oil. This crude product was flash chromatographed with 15% EtOAc/Hexanes to give 1-[3-(2,6-difluoro-benzyl)-phenyl]-ethanone (8A) as a yellow oil.

Rf=0.56 (15% EtOAc/Hexanes)

¹H NMR (400 MHz, CDCl₃) δ7.86 (s, 1H), 7.78 (d, j=7.6 Hz, 1H), 7.45 (d, j=8.3 Hz, 1H), 7.36 (t, j=7.6 Hz 1H), 7.19 (m, 1H), 6.89 (m, 2H), 4.07 (s, 2H), 2.57 (s, 3H)

1-[3-(2,6-difluoro-benzyl)-phenyl]-3-(4-methoxy-pyridin-2-yl)-propane-1,3-dione (8B)

To an oven dried 100 mL round bottomed flask equipped with a stirring bar, septum, argon inlet in THF (3 mL) was added 8A (0.35 g, 0.00142 mole), 4-methoxypyridine-2-carboxylic acid methyl ester (0.24 g, 0.0014 mole, prepared as described by R. J. Sundberg et.al. Organic Preparations and Procedures Int., 29(1), 117-122(1997)) and NaOMe (0.15 g, 0.0028 mole). After 45 minutes this solution was poured into saturated aqueous NH₄Cl and extracted with EtOAc three times. The combined organic layers were dried over NaSO₄, filtered and evaporated to give a brown syrup. Recrystallized with Et₂O, then hot petroleum ether to give solids which were-filtered and collected in a fritted funnel to give 1-[3-(2,6difluoro-benzyl)-phenyl]-3-(4-methoxy-pyridin-2-yl)-propane-1,3-dione (8B) as a light yellow powder.

¹H NMR (400 MHz, CDCl₃) δ8.56 (d, j=5.7 Hz, 1H), 8.04(s. 1H), 7.97(b, 1H), 7.74 (s, 1H), 7.70 (b, 1H), 7.41 (m, 2H), 7.19(m, 1H), 7.02 (b, 1H), 6.89 (m, 2H), 4.11 (s, 2H), 3.98 (s, 3H).

| | | | |
|---|---|---|---|
| Anal. Calc'd for: | C, 68.31; | H, 4.59; | N, 3.62. |
| C₂₂H₁₇F₂NO₃ + add'l 0.30 water | | | |
| Found: | C, 68.31; | H, 4.57; | N, 3.20. |

1-(3-Benzyl-phenyl)-3-(4-methoxy-pyridin-2-yl)-propane-1,3-dione (8C)

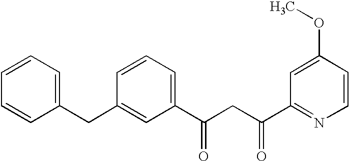

A suspension of 4B (0.2 g, 0.001 mole), NaH (0.08 g 60% dispersion, 0.002 mole) and 4-methoxypyridine-2-carboxylic acid methyl ester (0.167 g, 0.0011 mole, prepared as described by R. J. Sundberg et.al. Organic Preparations and Procedures Int., 29(1), 117-122(1997)) in 1.5 mL of distilled THF was heated to 40° C. for 10 minutes upon which an orange color was observed. The react ion was quenched with NH₄Cl, extracted with EtOAc and the organic layer was washed with saturated NaCl solution and dried with Na₂SO₄, filtered and concentrated. The residue was purified by trituration with CH₂Cl₂/Et₂O/pet.Ether to give 1-(3-benzyl-phenyl)-3-(4-methoxy-pyridin-2-yl)-propane-1,3-dione (8C) as a yellow solid.

¹H NMR (300 MHz, CDCl₃) δ8.52 (d, 1H), 7.92 (m, 2H), 7.70 (d, 1H), 7.54 (s, 1H), 7.4−7.2 (m, 7H), 6.95 (dd, 1H), 4.08 (s, 3), 3.94(s, 3).

| | | | |
|---|---|---|---|
| Anal. Calc'd for: C₂₂H₁₉NO₃ | C, 76.50; | H, 5.54; | N, 4.06. |
| Found: | C, 76.65; | H, 5.65; | N, 3.95. |

1-[3-(2,6-Difluoro-benzyl)-phenyl]-3-(4-methyl-pyridin-2-yl)-propane-1,3-dione (8D)

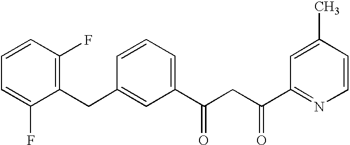

In a manner similar to that for 8B, using 8A and 3H, 1-[3(2,6-difluoro-benzyl)-phenyl]-3-(4-methyl-pyridin-2-yl)-propane-1,3-dione (8D) was prepared.

¹H NMR (400 MHz; CDCl₃) δ8.61 (d, j=5.0 Hz, 1H), 8.04(d, j=6.2 Hz, 2H), 7.97(d, j=7.0 Hz, 1H), 7.69 (s, 1H), 7.38(m, 3H), 7.19 (m, 1H), 6.89(m, 2H), 4.11 (s, 2H), 2.50 (s, 3H).

| | | | |
|---|---|---|---|
| Anal. Calc'd for: | C, 71.43; | H, 4.77; | N, 3.79. |
| C₂₂H₁₇F₂NO₂ + add'l 0.25 water | | | |
| Found: | C, 71.46; | H, 4.89; | N, 3.55. |

### EXAMPLE 9

1-{3-Benzyl-5-[6-(2-morpholin-4-yl-ethylamino)-pyrazin-2-yl]-phenyl}3-(4-methyl-pyridin-2-yl) (9F)

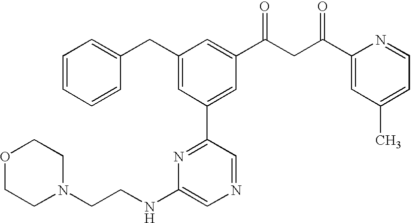

1-(3-Benzyl-5-bromo-phenyl)-ethanone (9A)

To an oven dried 500 mL three neck flask with a stirring bar, septum, argon inlet and, and thermometer was added 200 mL of Et₂O and (B) (6.9 g. 0.0212 mole). This was cooled to −78° C. in a dry ice acetone bath followed by the addition of nBuLi (12.53 mL, 0.0278 mole, 2.2M) dropwise with a syringe pump. Once addition was completed the pink solution was allowed to stir for 15 minutes at which time N-methoxy-N-methyl acetamide (2.18 mL, 0.0214 mole) was added dropwise. After addition was completed the ice bath was removed and the reaction was allowed to warm to ambient temperature. After I hour the reaction was poured into 100 mL of 1N HCl, extracted with EtOAc, dried over NaSO₄, filtered and removed solvent to give (9A) which was taken on.

Rf=0.48 (10% EtOAc/Hexanes)

¹H NMR (400 MHz, CDCl₃), δ7.92 (s, 1H), 7.72 (s, 1H), 7.52 (s, 1H), 7.40−7.13 (m, 5H), 4.01 (s 2H), 2.56 (s, 3H).

2-(3-Benzyl-5-bromo-phenyl)-2-methyl-[1,3]dioxolane (9B)

To a solution of 9A (5.25 g, 0.0182 mole) in 70 mL of toluene was added ethylene glycol (3.04 mL, 0.0545 mole) and p-TsOH (0.35 g, 0.00182 mole) under argon. This was heated to reflux, collecting water formed using a Dean Stark trap, and allowed to stir overnight. Next morning the reaction was poured into saturated NaCO₃ solution and extracted 2 times with EtOAc, dried over NaSO₄, filtered and the solvent removed to afford a crude oil. Flash chromatography using 10% EtOAc/Hexanes on silica afforded pure 2-(3-benzyl-5-bromo-phenyl)-2-methyl-[1,3]dioxolane (2B).

Rf=0.55 (10% EtOAc/Hexanes)

¹H NMR (300 MHz, CDCl₃) δ7.46 (s, 1H), 7.32−7.15 (m, 7H), 4.02 (m, 2H), 3.94 (s, 2H), 3.75 (m, 2H), 1.61 (s, 3H).

1-(3-Benzyl-5-boronic acid-phenyl)-ethanone (9C)

To an oven dried 250 mL three neck flask eqipped with a stirring bar, septum, argon inlet and, and thermometer was added 80 mL of THF and 9B (4.0 g. 0.012 mole). This was cooled to −78° C. in a dry ice acetone bath followed by the addition of nBuLi (12.0 mL, 0.0264 mole, 2.2M ) dropwise with a syringe pump. Once addition was completed the solution was allowed to stir for 60 minutes at which time B(OMe)₃ (4.04 mL, 0.036 mole) was added dropwise. After addition was completed the ice bath was removed and the reaction was allowed to warm to ambient temperature and stir overnight. Next morning the solution was poured into 75 mL of 3N HCl and allowed to stir for 2 hours. The solution was extracted with EtOAc, dried over NaSO₄, filtered and removed solvent to give (9C) as a white solid which was taken on without further purification.

1-[3-Benzyl-5-(6-bromo-pyrazin-2-yl)-phenyl]-ethanone (9D)

To a 100 mL flask under argon was combined 9C (2 g, 0.00787 mole,) dissolved in a minimum amount of EtOH, 2,6-dibromopyrazine (1.87 g, 0.0787 mole prepared in 2 steps starting from commercially available 2-aminopyrazine using the preparation from JACS volume 68, page 400, 1946.), Pd(Ph₃)₄ (0.39 g, 0.00034 mole), and Na₂CO₃ (1.67 g, 0.0157 mole) dissolved in 8 mL of water, in 30 mL of toluene. This mixture was refluxed for 4 hours and after cooling was poured into 50 mL of water and extracted with EtOAc, dried over NaSO₄, filtered and evaporated to give a brown oil. This was flashed with column chromatography using 25% EtOAc/Hexanes to give a pure white solid (9D).

Rf=0.34 (25% EtOAc/Hexanes)

¹H NMR, (400 MHz, CDCl₃) δ8.95 (s, 1H), 8.64 (s, 1H), 8.41 (s, 1H), 8.06 (s, 1H), 7.91 (s, 1H), 7.34−7.21 (m, 5H), 4.14 (s, 2H), 2.65 (s, 3H).

1-{3-Benzyl-5-[6-(2-morpholin-4-yl-ethylamino)-pyrazin-2-yl]-phenyl}-ethanone (9E)

To a solution of (9D) (0.15 g, 0.000408 mole) in 2 mL of dioxane under argon was added N-(2-aminoethyl)morpholine (0.54 mL, 0.00408 mole) and Et₃N (0.17 mL, 0.00122 mole). After refluxing overnight the solution was cooled and then poured into water and extracted with EtOAc, dried over NaSO₄, filtered and evaprorated. Purification using flash chromatography with 2.5% MeOH/CHCl₃ (saturated with NH₃) to afford pure yellow oil (9E).

Rf=0.33 (2.5% MeOH/Cl₃ saturated with NH₃)

¹H NMR (300 MHz, CDCl₃) δ8.40 (s, 1H), 8.27 (s, 1H) 8.00 (s, 1H), 7.88 (s 1H), 7.84 (s, 1H), 7.34−7.21 (m, 5H), 5.29 (b, 1H), 4.12 (s, 2H), 3.75. (m, 4H), 3.53 (m, 2H), 2.67 (m, 2H); 2.64 (s, 3H), 2.52 (m, 4H) .

1-{3-Benzyl-5-[6-(2-morpholin-4-yl-ethylamino)-pyrazin-2-yl]-phenyl}-3-(4-methyl-pyridin-2-yl)-propane-1,3-dione (9F)

In an oven dried flask equipped with a rubber septum and with bubbling argon was dissolved 9E (50 mg, 0.00012 mole) and 3H (37 mg, 0.00024 mole) in 0.5 mL of anhydrous THF. Upon addition of NaOMe (20 mg, 0.00036 mole) the reaction turned a brown color. After 1 hour the reaction was acidified with HOAc to pH 5, concentrated and the residue dissolved in DMSO and purified by HPLC. Solvent was removed from pure fractions to then the yellow residue was taken up in dioxane and this was lyophilized over the weekend to give a light yellow solid (9F).

¹H NMR (400 MHz, CDCl₃) δ8.76 (d, j=5.1 Hz, 1H), 8.49 (s, 1H), 8.31 (s, 1H), 8.11 (s, 1H), 8.05 (s, 1H), 7.97 (s, 1H), 7.87 (s, 1H), 7.59 (s; 1H), 7.46 (d, j=4.5 Hz, 1H), 7.33 (t, j=7.4 Hz, 2H), 7.24 (m, 3H), 4.16 (s, 2H), 4.00 (t, j=5.7 Hz, 2H), 3.96 (m, 4H), 3.63 (d, j=10.7 Hz, 2H), 3.37 (t, j=5.8 Hz, 2H), 2.96 (b, 2H), 2.56 (s, 3H).

| | | | |
|---|---|---|---|
| Anal. Calc'd for: C₃₂H₃₃N₅O₃ + | C, 52.47; | H, 4.57; | N, 8.27. |
| add'l 1.45 water and 2.50 TFA | | | |
| Found: | C, 52.49; | H, 4.03; | N, 8.66. |

1-[3-Benzyl-5-(6-methoxypyridin-2-yl)-phenyl]-3-(4-methyl-pyridin-2-yl)-propane-1,3-dione (9G)

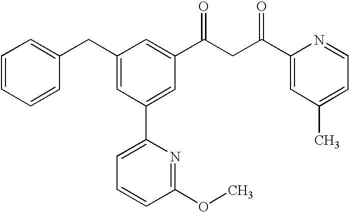

In a manner similar to that for 9F, using 9C and 2-bromo-6-methoxypyridine (prepared from 2,6-dibromopyridine as described in the Journal of Organic Chemistry 55 (1), pgs. 69-73, 1990), 1-{3-benzyl-5-[6-(2-morpholin-4-yl-ethylamino)-pyrazin-2-yl]-phenyl}-3-(4-methyl-pyridin-2-yl)-propane-1,3-dione (9G) was prepared.

| | | | |
|---|---|---|---|
| Anal. Calc'd for: C₂₈H₂₄N₂O₃ + | C, 76.25; | H, 5.60; | N, 6.35. |
| add'l 0.25 water | | | |
| Found: | C, 76.21; | H, 5.55; | N, 6.20. |

1-[3-Benzyl-5-(6-morpholin-4-yl-pyrazin-2-yl)-phenyl]-3-(4-methyl-pyridin-2-yl)-propane-1,3-dione(di-tfa-salt) (9H)

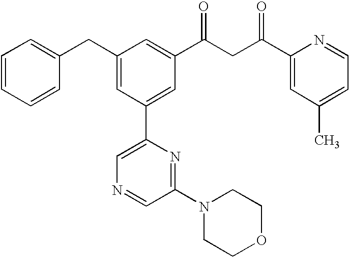

In a manner similar to that for 9F, 1-{3-benzyl-5-[6-(2-morpholin-4-yl-ethylamino)-pyrazin-2-yl]-phenyl}-3-(4-methyl-pyridin-2-yl)-propane-1,3-dione (9H) was prepared.

| | | | |
|---|---|---|---|
| Anal. Calc'd for: C₃₀H₂₈N₄O₃ + | C, 76.25; | H, 5.60; | N, 6.35 |
| add'l 0.55 dioxane and 2.45 TFA | | | |
| Found: | C, 7 6.21; | H, 5.55; | N, 6.20. |

1-[3-Benzyl-5-(4-methyl-3,4,5,6-tetrahydro-2H-[1,2′]bipyrazinyl-6′-yl)-phenyl]-3-(4-methyl-pyridin-2-yl)-propane-1,3-dione (di-TFA-salt) (9I)

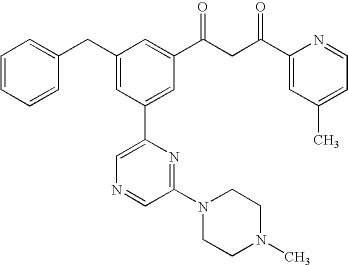

In a manner similar to that for 9F, 1-{3-benzyl-5-[6-(2-morpholin-4-yl-ethylamino)-pyrazin-2-yl]-phenyl}-3-(4-methyl-pyridin-2-yl)-propane-1,3-dione (9I) was prepared.

| | | | |
|---|---|---|---|
| Anal. Calc'd for: C₃₁H₃₁N₅O₂ + | C, 52.59; | H, 4.54; | N, 8.52 |
| add'l 1.75 water and 2.45 TFA | | | |
| Found: | C 52.58; | H, 4.29; | N, 8.20. |

### EXAMPLE 10

1-[3-Benzyl-5-(5-methylpyrazin-2-ylmethyl)phenyl]-3-(5-methylpyrazin-2-yl)-propane-1,3-dione (10D)

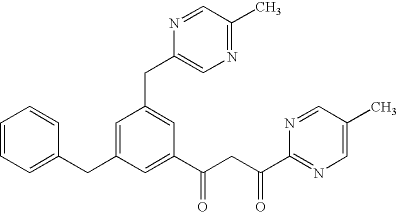

(3-benzyl-5-bromophenyl)(5-methylpyrazin-2-yl)ketone (10A)

To a cold (−78° C.) solution of 1-benzyl-3,5-dibromobenzene (3B) (1.5 g) in diethyl ether (30 mL), a solution of n-BuLi in hexanes (2.5 M, 2.2 mL) was added. The resultant mixture was stirred at −78° C. for 1 hour and was treated with a solution of N-methoxy-N-methyl-5-methylpyrazine-2-carboxyamide (1.0 g) in diethyl ether (3 mL). The reaction mixture was allowed to warm up slowly to room temperature and was stirred at that temperature overnight. The product mixture was diluted with ethyl acetate and partitioned with aq. HCl. The organic extract was washed with brine, dried over magnesium sulfate, filtered and concentrated under vacuum. The residue was subjected to column chromatography on silica gel eluting with 20-40% ethyl acetate in hexane gradient. Collection and concentration of appropriate fractions provided the title pyrazine.

¹H NMR (400 MHz, CDCl₃) δ9.15 (br s, 1H), 8.53 (br s, 1H), 8.06 (br s, 1 H), 7.86 (br s, 1H), 7.56 (br s, 1H), 7.34−7.18 (m, 5H), 4.40.(s, 2H), 2.70 (s, 3H).

3-Benzyl-5-(5-methylpyrazin-2-ylmethyl)-1-bromobenzene (10B)

A mixture of (3-benzyl-5-bromophenyl)(5-methylpyrazin-2-yl)ketone (0.73 g) and anhydrous hydrazine (3 mL) in ethylene glycol (7 mL) was heated at 110 C. for 5 hr. Excess hydrazine was removed under reduced pressure. The residue ethylene glycol solution was treated with powdered solid KOH (0.5 g) and heated at 140 C. under an atmosphere of argon for 4 h. The product mixture was partitioned between benzene and water. The organic extract was washed with brine, dried over magnesium sulfate, filtered, and concentrated under vacuum. The residue was subjected to column chromatography on silica gel eluting with 50% ethyl acetate in hexane. Collection and concentration of appropriate fractions provided the title bromide.

¹H NMR (400 MHz, CDCl₃) δ8.38 (br s, 1H), 8.33 (br s, 1H), 7.30−7.14 (m, 7H), 7.03 (br s, 1H) 4.04 (s, 2H), 3.91 (s, 2H), 2.54 (s, 3H).

3-Benzyl-5-(5-methylpyrazin-2-yl)methylacetophenone (10C)

To a mixture of 3-benzyl-5-(5-methylpyrazin-2-ylmethyl)-1-bromobenzene (0.45 g), thallium acetate (0.37 g), 1,3-bis(diphenylphosphino)propane (0.12 g) and triethylamine (0.71 mL) in DMF (5 mL) in a pressure tube, purged with argon for a period of 10 minutes, palladium acetate (57 mg) and n-butyl; vinyl ether (0.77 mL) was added. The reaction tube was sealed and stirred at 100 C. overnight. The reaction mixture was filtered through a bed of Celite, and the filtrate concentrated under vacuum. The residue was dissolved in THF (3 mL) and treated with aq. HCl (3M, 3 mL). The resultant mixture was stirred at rt for 3 hr., diluted with ethyl acetate, basified with aq. sodium bicarbonate. The organic extract was dried over magnesium sulfate, filtered, and concentrated under vacuum. The residue was subjected to column chromatography on silica gel eluting with a 30-50% ethyl acetate, in hexane gradient. Collection and concentration of appropriate fractions provided the title ketone.

¹H NMR (400 MHz, CDCl₃) δ8.38 (br s, 1H), 8.34 (br s, 1H), 7.69 (br s, 1H), 7.66 (br s, 1H), 7.31−7.15 (m, 6 H), 4.14 (s, 2H), 4.01 (s, 2H), 2.542 (s, 3H), 2.539 (s, 3H).

1-[3-Benzyl-5-(5-methylpyrazin-2-ylmethyl)phenyl]-3-(5-methylpyrazin-2-yl)-propane-1,3-dione (10D)

To a cold (−78° C.) solution of 3benzyl-5-(5-methylpyrazin-2-yl)methylacetophenone (0.336 g) in THF (10 mL), a solution of n-BuLi in hexanes (2.5 M, 1.5 mL) was added. The resultant mixture was stirred at −78° C. for 15 minutes and was treated with a solution of N-methoxy-N-methyl-5-methylpyrazine-2-carboxyamide (0.27 g) in THF (3 mL). The reaction mixture was allowed to warm up slowly to room temp in 2 hours. The product mixture was diluted with ethyl acetate and partitioned with aq. HCl. The organic extract was washed with brine, dried over magnesium sulfate, filtered, and concentrated under vacuum. The residue was subjected to column chromatography on silica gel eluting with 50-100% ethyl acetate in hexane gradient. Collection and concentration of appropriate fractions provided the title pyrazine.

¹H NMR (400 MHz, CDCl₃) δ9.21 (br s, 1H), 8.52 (br s, 1H), 8.38 (br s, 1H), 8.35 (br s, 1H), 7.79 (br s, 1H), 7.76 (br s, 1H), 7.39 (br s, 1H), 7.31−7.16 (m, 6 H), 4.16 (s, 2H), 4.03 (s, 2H), 2.67 (s, 3H), 2.53 (s, 3H).

### EXAMPLE 11

1-[5-(4-Fluoro-benzyl)-2,3-dimethoxy-phenyl]-3-(4-methyl-pyridin-2-yl)-propane-1,3-dione (11E)

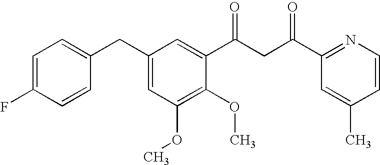

(3-Bromo-4,5-dimethoxy-phenyl)-(4-fluoro-phenyl)-methanol (11B)

To a solution of 5-bromoveratraldehyde (11A) (2.5 g, mmol) in 150 mL of distilled THF at 0° C. was added slowly 1M 4-fluorophenylmagnesium bromide in THF (25.5 mL, 25.5 mmol). The solution allowed to slowly warm to room temperature at which time the solvent was removed in vacuo and the remaining material was partitioned between 10% KHSO₄ and EtOAc. The combined organic layers were washed with brine, dried over Na₂SO₄, filtered and evaporated to give a yellow oil that was taken on to the next step without further purification.

Rf=0.11 (10% EtOAc/Hexanes)

¹H NM (400 MHz, CDCl₃) δ7.33 (m, 2H), 7.04 (m, 3H), 6.86 (m, 1H) 5.74 (s, 1H), 3.83(s, 3H), 3.81(s, 3H).

1-Bromo-2,3-dimethoxy-5-(4-fluorobenzylmethyl)-benzene (11C)

To crude (3-Bromo-4,5-dimethoxy-phenyl)-(4-fluoro-phenyl)-methanol (11B) (2.95 g, 8.65 mmol) dissolved in 30 ml anhydrous CH₂Cl₂ at 0° C. under argon was added Et₃SiH (3.54 mL, 22.2 mmol) and BF₃.Et₂O (2.27 mL, 22.2 mmol). The reaction was then left to stir overnight at room temperature after the ice bath dissipated. The solvent was removed in vacuo and the residue was partitioned between sat. NaHCO₃ solution and CH₂Cl₂. The combined organic extracts were washed with brine, dried over Na₂SO₄, filtered, and concentrated to give a clear oil. This oil was chromatographed on silica gel using 20% EtOAc in hexanes as the elutant to afford 1-Bromo-2,3-dimethoxy-5-(4-fluoro-benzylmethyl)-benzene (11C) as a clear oil.

Rf=0.47 (10% EtOAc/Hexanes).

¹H NMR (400 MHz, CDCl₃) δ7.12(m, 2H) 6.93-7.02 (m, 3H), 6.62 (m, 1H), 3.86 (s, 2H), 3.83(s, 3H), 3.81(s, 3H).

1-[5-(4-Fluorobenzyl)-2,3-dimethoxy-phenyl]-ethanone (11D)

A solution of 2.5 M n-BuLi in hexanes (3.79 mL, 9.48 mmol) was slowly added to 1-bromo-2,3-dimethoxy-5-(4-fluoro-benzylmethyl)-benzene (11C) (2.50 g, 7.69 mmol) dissolved in 45 mL distilled THF at −78° C. under argon. After aging 30 minutes, N-methoxy-N-methylacetamide (1.11 mL, 10.8 mmol) was added dropwise. The solution was slowly allowed to warm to room temperature as the dry ice bath dissipated. The reaction was quenched with sat. NH₄Cl solution and extracted with EtOAc. The combined organic extracts were washed with brine, dried over Na₂SO₄, filtered and concentrated to give a yellow oil. This oil was chromatographed on silica gel using 5% EtOAc in hexanes as the elutant to give 1-[5-(4-fluoro-benzyl)-2,3-dimethoxy-phenyl]-ethanone (11D) as a yellow oil.

Rf=0.25 (10% EtOAc/Hexanes)

¹H NMR (400 MHz, CDCl₃) δ7.12(m, 2H), 7.05 (m, 1H), 6.92 (m, 2H), 6.81 (m, 1H), 3.90 (s, 2H), 3.88(s, 3H), 3.83(s, 3H) 2.61(s, 3H).

1-[5-(4-Fluorobenzyl)-2,3-dimethoxy-phenyl]-3-(4-methyl-pyridin-2-yl)propane-1,3-dione (11E)

In a dried flask under argon was added NaOMe (45mg, 0.83 mmol) to 1-[5-(4-fluoro-benzyl)-2,3-dimethoxy-phenyl]-ethanone (11D) dissolved in 3 mL distilled THF. After 5 minutes methyl 4-methylpydridine-2-carboxylate (3H) (69 mg, 0.46 mmol) in THF was added and the reaction was stirred 1.5 hours. The reaction was quenched with water, the pH of the solution was adjusted to 4 using 1N HCl, and the solution was extracted with CHCl₃. The combined organic extracts were dried over Na₂SO₄, filtered, and concentrated to give a yellow oil. The oil was crystallized using Et₂O to give 1-[5-(4-fluoro-benzyl)-2,3-dimethoxy-phenyl]-3-(4-methyl-pyridin-2-yl)-propane-1,3-dione (11E) as a yellow solid.

¹H NMR (400 MHz, CDCl₃) δ8.54(m, 1H), 7.96(m, 1H), 7.58(m, 1H), 7.23(m, 2H), 7.15(m, 2H), 6.99(m, 2H), 6.80(m, 1H), 3.94(s, 2H), 3.89(s, 3H), 3.84(s, 3H), 2.44(s, 3H).

| | | | |
|---|---|---|---|
| Anal. Calc'd for: C₂₄H₂₂FNO₄ 0.25 H₂O | C, 69.97; | H, 5.51; | N, 3.40. |
| Found: | C, 70.01; | H, 5.34; | N, 3.19. |

1-[2,3-Dimethoxy-5-(2-methyl-benzyl)-phenyl]-3-(4-methyl-pyridin-2-yl)-propane-1,3-dione (11F)

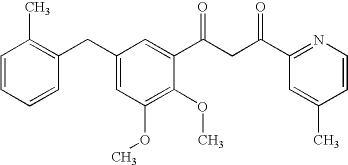

In a manner similar to that for 11E, 1-[2,3-dimethoxy-5-(2-methyl-benzyl)-phenyl]-3-(4-methyl-pyridin-2-yl)-propane-1,3-dione (11F) TFA salt was prepared.

1H NMR (400 MHz, CDCl₃) δ8.90(m, 1H), 8.04(m, 1H), 7.55(m, 1H), 7.42(s, 1H), 7.25(m, 1H), 7.18(m, 3H), 7.10(m, 1H), 6.83(m, 1H), 3.99(s, 2H), 3.90 (s, 3H), 3.826(s. 3H), 2.61(s, 3H).

1-[5-(2,6-Difluorobenzyl)-2,3-dimethoxyphenyl]-3-(4-methylpyridin-2-yl)propane-1,3-dione (11G)

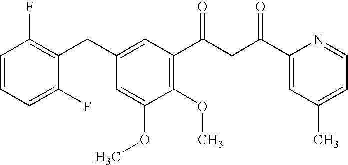

4-[1-(2,6-Difluorophenyl)-1-hydroxymethyl]-2-methoxy-phenol

To a cold solution (−78 C.) of 4-bromoguaiacol (Aldrich) (2.00 g, 9.9 mmol) in 200 mL of Et₂O in dried glassware under argon was added dropwise t-BuLi (26.1 mL, 44.3 mmol, 1.7 M). After 15 minutes, 2,6-difluorobenzaldehyde (1.17 mL, 10.8 mmol) was added dropwise. The reaction was allowed to stir overnight as the dry ice bath dissipated. The reaction was quenched with 10% KHSO₄ and extracted with EtOAc. The combined organic extracts were washed with brine, dried over Na₂SO₄, filtered, and concentrated to give 4-[1-(2,6-difluoro-phenyl)-1-hydroxy-methyl]-2-methoxy-phenol as a brownish oil.

4-(2,6-Difluorobenzyl)-2-methoxyphenol

To crude 4-[1-(2,6-difluorophenyl)-1-hydroxy-methyl]-2-methoxy-phenol (262 g, 9.9 mmol) dissolved in 30 mL anhydrous CH₂Cl₂ at 0° C. under argon was added Et₃SiH (3.93 mL, 24.6 mmol) and BF₃.Et₂O (3.12 mL, 24.6 mmol). The reaction was then left to stir overnight at room temperature after the ice bath dissipated. The solvent was removed in vacuo and the residue was partitioned between sat. NaHCO₃ solution and CH₂Cl₂. The combined organic extracts were washed with brine, dried over Na₂SO₄, filtered, and concentrated to give a brown oil. This oil was chromatographed on silica gel using 5% EtOAc in hexanes as the elutant. Collection and concentration of appropriate fractions provided 4-(2,6-difluoro-benzyl)-2-methoxy-phenol as a yellow oil.

Rf=0.33 (10% EtOAc/Hexanes)

¹H NMR (400 MHz, CDCl₃) δ7.15(m, 1H), 6.75-6.88 (m, 5H) 4.78 (s, 1H), 3.93 (s, 2H), 3.85 (s, 3H).

2-Bromo-4-(2,6-difluorobenzyl)-6-methoxyphenol

To a cooled solution (0 C.) of 4-(2,6-difluorobenzyl)-2-methoxyphenol (590 mg, 2.36 mmol) in 20 mL anhydrous MeOH under argon bromine (134 μL, 2.59 mmol) was added dropwise. The reaction was stirred 3 hours as the ice bath warmed to room temperatures The reaction was quenched with saturated NH₄Cl solution and extracted with EtOAc. The combined organic extracts were washed with brine, dried over Na₂SO₄, filtered, and concentrated to give a brown oil. This material was chromatographed on silica gel using 5% EtOAc in hexane as the eluant to give 2-bromo-4-(2,6-difluorobenzyl)-6-methoxyphenol as a yellow oil.

Rf=0.24 (10% EtOAc Hexanes)

¹H NMR (400 MHz, CDCl₃) δ7.18(m, 1H), 6.98(m, 1H), 6.88(m, 2H), 6.74(m, 1H), 5.76(s, 1H), 3.90(s, 2H), 3.87(s, 3H).

1-Bromo-5-(2,6-difluorobenzyl)-2,3-dimethoxybenzene

To a chilled solution (0 C.) of 2-bromo-4-(2,6-difluorobenzyl)-6-methoxy-phenol (505 mg, 1.53 mmol) in 10 mL dry DMF under argon was added Cs₂CO₃ (1.50 g, 4.60 mmol) and methyl iodide (119 μL, 1.92 mmol). The reaction was stirred overnight as the bath warmed to room temperature. The reaction was quenched with saturated NH₄Cl solution and extracted with EtOAc. The combined organic extracts were washed with brine, dried over Na₂SO₄, filtered, and concentrated to give a brown oil. This oil was chromatographed on silica gel using 10% EtOAc in hexane as the eluant to give 1-bromo-5-(2,6-difluorobenzyl)-2,3-dimethoxybenzene as a yellow oil.

Rf=0.71 (10% EtOAc/Hexanes)

¹H NMR (400 MHz, CDCl₃) δ7.19(m, 1H), 7.00(s, 1H), 6.89(m, 2H), 6.77(s, 1H), 3.92(s, 2H), 3.83(s, 3H), 3.81(s, 3H).

1-[5-(2,6-Difluoro-benzyl)-2,3-dimethoxy-phenyl]-ethanone

To a cold (−78 C.) solution of 1-bromo-5-(2,6-difluorobenzyl)-2,3-dimethoxybenzene (390 mg, 1.14 mmol) in 6 mL of Et₂O in dried glassware under argon was added n-BuLi (500 μL, 1.25 mmol, 2.5 M) dropwise. After 10 minutes N-methoxy-N-methylacetamide (139 μL, 1.36 mmol) was added and the reaction was stirred overnight as the ice bath dissipated. The reaction was quenched with saturated NH₄Cl solution and extracted with EtOAc. The combined organic extracts were washed with brine, dried over Na₂SO₄, filtered, and concentrated to give a brown oil. This oil was chromatographed on silica gel using 5% EtOAc in hexane as the eluant to give 1-[5-(2,6-difluorobenzyl)-2,3-dimethoxyphenyl]ethanone as a yellow solid.

Rf=0.21 (10% EtOAc/Hexanes)

¹H NMR (400 MHz, CDCl₃) δ7.18(m, 1H), 7.10(m, 1H), 6.96(m, 1H), 6.88(m, 2H), 3.97(s, 2H), 3.85(s, 6H), 2.59(s, 3H).

1-[5-(2,6-Difluorobenzyl)-2,3-dimethoxyphenyl]-3-(4-methylpyridin-2-yl)propane-1,3-dione (11G)

To a solution of 1-[5-(2,6-difluorobenzyl)-2,3-dimethoxyphenyl]ethanone (48 mg, 0.16 mmol) and 5-methyl 4-methylpydridine-2-carboxylate (3H) (52 mg, 0.34 mmol) in THF (2 mL) at room temp., sodium methoxide (19 mg, 0.34 mmol) was added The reaction was stirred for 1.5 hours, quenched with water. The pH of the solution was adjusted to 4 using 1N HCl, and the solution was extracted with CHCl₃. The combined organic extracts were dried over Na₂SO₄, filtered, and concentrated to give a yellow solid. This material was purified over reverse phase HPLC to give 1-[5-(2,6-difluoro-benzyl)-2,3-dimethoxyphenyl]-3-(4-methylpyridin-2-yl)propane-1,3-dione (11G) as a yellow solid after lyophilization and crystallization from Et₂O.

¹H NMR.(400 MHz, CDCl₃) δ8.54(m, 1H), 7.94(s, 1H), 7.55(s, 1H), 7.29(s, 1H), 7.14-7.23(m,2H), 6.95(s, 1H), 6.89(m, 2H), 4.00(s, 2H), 3.87 (s, 3H), 3.86 (s, 3H), 2.44(s, 3H).

Exact Mass: C₂₄H₂₁F₂NO₄

Theor. Mass 426.1511

Meas. Mass 426.1521

### EXAMPLE 12

1-(5-Benzyl-2-methoxy-3-morpholin-4-ylmethylphenyl)-3-(4-methyl-pyridin-2-yl)propane-1,3-dione (12H)

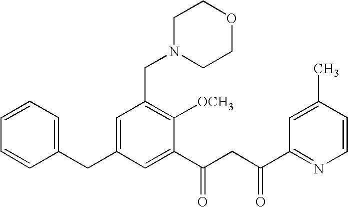

4-Benzyl-2,6-dibromophenol (12B)

To a solution of 4-hydroxydiphenylmethane (15.3 g, 83 mmol) in glacial acetic acid (200 mL) at room temperature, a solution of bromine (8.6 mL, 167 mmol) in acetic acid (20 mL) was added dropwise over a period of half an hour. The resultant mixture was stirred at room temperature for 3 hr, poured into ice water, and partitioned with toluene. The organic extract was washed successively with 10% aq. sodium hydrogensulfite and brine, dried over sodium sulfate, filtered, and concentrated in vacuo. The residue was subjected to column chromatography on silica gel eluting with 7% ethyl acetate in hexane. Collection and concentration of appropriate fractions provided 4-benzyl-2,6-dibromophenol as a yellow oil.

¹H NMR (400 MHz, CDCl₃) δ7.3 (m, 5H ), 7.2 (m, 2H), 5.7 (s, 1H), 3.9 (s, 2H).

4-Benzyl-2,6-dibromo-1-methoxybenzene (12C)

To a cold (0 C.) solution of 4-benzyl-2,6-dibromophenol (10 g, 29 mmol) in diethyl ether (100 mL), was added a solution of diazomethane in diethyl ether over a period of 20 minutes. The diazomethane solution was prepared by the addition of 1-methyl-3-nitro-1-nitrosoguanidine (8.5 g, 44 mmol) in several portions to a mixture of 40% aq. KOH (100 mL) and ether (50 mL) at 0 C. over a period of 15 min. The resultant solution was stirred at room temperature for two days, and concentrated under vacuum. The residue was subjected to column chromatography on silica gel eluting with 1-2% ethyl acetate in hexane gradient. Collection and concentration of appropriate fractions provided 4-benzyl-2,6-dibromo-1-methoxybenzene as a syrup.

¹H NMR (400 MHz, CDCl₃) δ7.3 (m, 5H), 7.2 (m, 2H), 3.9 (s, 2H), 3.85 (s, 3H).

5-Benzyl-3-bromo-2-methoxybenzaldehyde (12D)

To a cold (−78 C.) solution of 4-benzyl-2,6-dibromo-1-methoxybenzene (4.0 g, 11.3 mmol) in 60 mL dry diethyl ether, under an atmosphere of argon, was added n-butyllithium (4.5 mL of a 2.5 M solution in hexanes, 11.3 mmol) over 10 minutes. The solution was stirred at −78 C. for 1 hour and then treated with neat N,N-dimethylformamide (0.96 mL, 12.4 mmol). External cooling was removed and the reaction was stirred 2 hours after warming to room temperature. The reaction mixture was then diluted with water and extracted with ethyl acetate (3 times). The combined extracts were washed with brine, dried (MgSO₄), filtered and concentrated in vacuo to afford the title compound as an oil which was used without further purification.

¹H NMR (300 MHz, CDCl₃) δ10.3 (s, 1H),7.6 (m, 2H), 7.25 (m, 5H), 3.96 (s, 5H).

4-Benzyl-2-bromo-5-bromomethylbenzene (12E)

To a cold (0 C.) solution of 5-benzyl-3-bromo-2-methoxybenzaldehyde (3.4 g, 11.1 mmol) in 60 mL of methanol was added sodium borohydride (0.42 g, 11.1 mmol) in several portions. The reaction was stirred 30 minutes at ambient temperature and then treated with 1M aqueous HCl (20 mL) and stirred 10 minutes. The mixture was concentrated in vacuo to remove most of the methanol and the residue partitioned between ethyl acetate and water. The layers were separated and the aqueous layer further extracted with ethyl acetate (2 times). The combined organic extracts were washed with brine, dried (MgSO₄), filtered and concentrated in vacuo to a syrup 2.4 g (approximately 7.8 mmol) of this crude alcohol was then dissolved in dry THF (80 mL), under an atmosphere of argon, and treated with carbon tetrabromide (3.9 g, 11.8 mmol) followed by triphenylphosphine (3.1 g, 11.8 mmol). After 30 min Florisil was added to the mixture and the whole concentrated in vacuo to afford a solid which was loaded onto a silica gel column for chromatography. Eluting with a 1-2% ethyl acetate in hexane gradient afforded 4-benzyl-2-bromo-5-bromomethylbenzene (2E) as a syrup.

¹H NMR (300 MHz, CDCl₃) δ7.25 (m, 7H), 4.5 (s, 2H), 3.95 (s, 3H), 3.9 (s, 2H).

4-(5-Benzyl-3-bromo-2-methoxybenzyl)morpholine (12F)

To a solution of 4-benzyl-2-bromo-5-bromomethylbenzene (0.5 g, 1.4 mmol) in dry acetonitrile (7 mL) under an atmosphere of argon, was added morpholine (0.59 mL, 6.8 mmol) and the mixture heated at reflux for 20 hours and aged 2 days at ambient temperature. The mixture was concentrated in vacuo and the residue purified by column chromatography on silica gel with a mixture of 1:50:49 of methanol:chloroform:NH₃ saturated chloroform as eluent to afford 4-(5-benzyl-3-bromo-2-methoxybenzyl)morpholine as a gum.

¹H NMR (300 MHz, CDCl₃) δ7.25 (m, 7H), 3.9 (s, 2H),3.8 (s, 3H), 3.7 (m, 4H), 3.5 (s, 2H), 2.45 (m, 4H).

1-(5-Benzyl-2-methoxy-3-morpholin-4-ylmethylphenyl)ethanone (12G)

A thick walled glass pressure vessel was charged with 4-(5-benzyl-3-bromo-2-methoxybenzyl)morpholine (0.45 g, 1.2 mmol), thallium acetate (0.35 g, 1.3 mmol), 1,3-bis(diphenylphosphino)propane (124 mg, 0.3), and dry N,N-dimethylformamide (3 mL). The slurry was purged for 15 minutes with argon and then treated with palladium acetate (68 mg, 0.3 mmol), triethylamine (0.49 ml, 3.6 mmol), and n-butyl vinyl ether (0.78 mL, 6.0 mmol). The pressure vessel was sealed and heated in a 100 C. oil bath with magnetic stirring overnight. The dark reaction mixture was allowed to cool to ambient temperature and filtered through Celite. The filtrate was concentrated in vacuo and the residue diluted with THF (20 mL), treated with 1M aqueous HCl (3 mL), and stirred for 1 hour. The mixture was made basic with saturated aqueous NaHCO₃ and extracted with diethyl ether (3 times). The combined extracts were washed with brine, dried (MgSO₄), filtered, and concentrated in vacuo to a brown oil. Purification by column chromatography on silica gel with 1.5% methanol in chloroform afforded 1-(5-benzyl-2-methoxy-3-morpholin-4-ylmethylphenyl)ethanone as a syrup.

¹H NMR (400 MHz, CDCl₃) δ7.3 (m, 7H), 3.95.(s, 2H), 3.8 (s, 3H), 3.7 (m,4H), 3.5 (s, 2H), 2.6 (s, 3H), 2.4 (m, 4H).

1-(5-Benzyl-2-methoxy-3-morpholin-4-ylmethylphenyl)-3-(4-methyl-pyridin-2-yl)propane-1,3-dione (12H)

To a solution of 1-(5benzyl-2-methoxy-3-morpholin-4-ylmethylphenyl)-ethanone (89 mg, 0.17 mmol) in dry THF (2 mL) under an atmosphere of argon, was added 4-methylpyridine-2-carboxilic acid methyl ester (3H) (40 mg, 0.26 mmol) and sodium ethoxide (20 mg, 0.29 mmol) in single portions. The mixture was stirred 1 hour at ambient temperature then diluted with diethyl ether and washed with saturated aqueous NaHCO₃ ,brine, dried over MgSO₄, filtered and concentrated in vacuo to a gum. Purification by preparative HPLC on C18 reverse, stationary phase eluting with a water/acetonitrile/trifluoroacetic acid mobile phase afforded the title compound as a lyophilized solid.

¹H NMR (400 MHz, CDCl₃) δ8.6 (d, J=4.9 Hz, 1H), 8.0 (s, 1H), 7.7 (d, J=2.3 Hz, 1H), 7.45 (d, J=2.3 Hz, 1H), 7.44 (s, 1H), 7.35 (d, J=5.0 Hz, 1H), 7.3−7.2 (m, 5H), 4.3 (s, 2H), 3.9 (br m, 6H), 3.8 (s, 3H), 3.5 (br m, 2H), 2.9 (br m, 2H), 2.5 (s, 3H).

| | | | |
|---|---|---|---|
| Anal. Calc'd for: | C, 56.87; | H, 4.80; | N, 4.18 |
| C₂₈H₃₀N₂O₄ 1.85 CF₃CO₂H | | | |
| Found: | C, 56.90; | H, 4.72; | N, 3.88. |

1-(5-Benzyl-2-isopropoxy-3-pyrrolidin-1-ylmethyl-phenyl)-3-(4-methyl-pyridin-2-yl)propane-1,3-dione (12I)

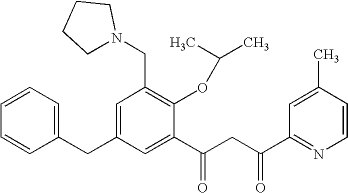

In a manner similar to that for 12H, 1-(5-Benzyl-2-isopropoxy-3-pyrrolidin-1-ylmethyl-phenyl)-3-(4-methyl-pyridin-2-yl)propane-1,3-dione(12I) was prepared.

ES MS found=471.3 m/z [M+1].

1-(5-Benzyl-2-isopropoxy-3-[1,2,4]triazol-1-ylmethylphenyl)-3-(4-methyl-pyridin-2-yl)propane-1,3-dione (12J)

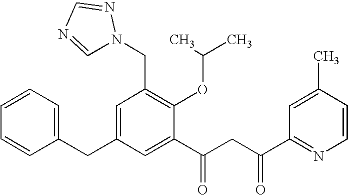

In a manner similar to that for 12H, 1-(5-Benzyl-2-isopropoxy-3-[1,2,4]triazol-1-ylmethylphenyl)-3-(4-methyl-pyridin-2-yl)propane-1,3-dione (12J) was prepared.

¹H NMR (400 MHz, CDCl₃) δ8.58 (d, J=4.9 Hz, 1H), 8.30 (s, 1H), 8.03 (s, 1H), 7.97 (s, 1H), 7.60 (d, J=2.3 Hz, 1H), 7.51 (s, 1H), 7.30−7.11 (m, 8H), 5.42 (s, 2H), 4.31 (brm, 1H), 3.95 (s, 2H), 2.46 (s, 3H), 1.24 (d, J=6.1 Hz, 6H).

### EXAMPLE 13

1-[5-Benzyl-2-(pyridin-2-yloxy)phenyl]-3-(4-methylpyridin-2-yl)propane-1,3-dione (13E)

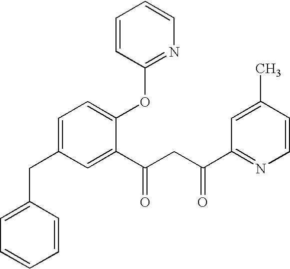

4-Benzyl-2-bromophenol (13B)

To a solution of 4-benzylphenol (10.0 g, 54.3 mmol) in 60 mL of CHCl₃ was added bromine (2.9 mL, 56.6 mmol) in 20 mL Of CHCl₃ over 2 hours. The reaction was stirred overnight at room temperature. Saturated NaHCO₃ was added and then extracted with CHCl₃ three times. The combined organic extracts were washed with brine, dried over MgSO₄, filtered, and concentrated in vacuo to provide the titled bromide. The product was taken on to the next step without further purification.

¹H NMR (400 MHz, CDCl₃) δ7.25 (m, 6H) 7.04 (m, 1H), 6.93 (m, 1H), 5.4 (s, 1H), 3.9 (s, 2H)

2-(4-Benzyl-2-bromophenoxy)pyridine (13C)

To an oven-dried flask under an argon atmosphere was added sodium hydride (300 mg, 12.5 mmol) and 20 mL of DMSO. To this was added 4-benzyl-2-bromophenol (3.0 g, 11.4 mmol) dropwise and stirred for 10 minutes. This was treated with 2-fluoropyridine (2.0 mL, 23.2 mmol) and heated overnight at 150 C. The reaction was treated with 1N HCl to obtain a pH of 7, and extracted with CHCl₃ three times. The combined organic layers were washed with water, brine, dried over Na₂SO₄, filtered and concentrated in vacuo. This residue was chromatographed on silica gel using CHCl₃ as eluant to afford 2-(4-benzyl-2-bromophenoxy)pyridine (13C)

¹H NMR (400 MHz, CDCl₃) δ8.15 (m, 1H), 7.7 (m, 1H), 7.45 (m, 1H), 7.3−7.1 (m, 7H), 6.95 (m, 2H), 4.0 (s,2H)

1-[5-Benzyl-2-(pyridin-2-yloxy)phenyl]ethanone (13D)

To an oven-dried flask under an argon atmosphere was added 2-(4-benzyl-2-bromophenoxy)pyridine (1.68 g, 4.93 mmol) and 40 mL of anhydrous diethyl ether. The solution was cooled to −78 C. and n-butyl lithium (2.1 mL of a 2.5M hexane solution, 5.3 mmol) was added dropwise. This was stirred 75 minutes at −85 C., followed by addition of N-methoxy-N-methylacetamide (0.60 mL, 5.9 mmol). The reaction was warmed to room temperature overnight and then treated with 1N HCl to obtain a pH of 7, and extracted with diethyl ether three times. The combined organic layers were washed with brine, dried over Na₂SO₄, filtered and concentrated in vacuo. This material was chromatographed on silica gel using CHCl₃-30% EtOAc/CHCl₃ as eluant to afford 1-[5-benzyl-2-(pyridin-2-yloxy)phenyl]ethanone (13D) as an orange gum.

¹H NMR (400 MHz, CDCl₃) δ8.2 (m, 1H), 7.7 (m, 2H), 7.25 (m, 6H), 7.0 (m, 3H), 4.0 (s, 2), 2.5 (s, 3)

1-[5-Benzyl-2-(pyridin-2-yloxy)phenyl]-3-(4-methylpyridin-2-yl)-propane-1,3-dione (13E)

To an oven-dried flask under an argon atmosphere was added 1-[5-benzyl-2-(pyridin-2-yloxy)phenyl]ethanone (300 mg, 0.99 mmol) and 15 mL of anhydrous THF. The solution was cooled to −78 C. and LDA (0.55 mL of a 2.0 M solution in heptane/THF/ethylbenzene, 1.1 mmol) was added dropwise. This was stirred at −78 C. for 50 minutes, followed by addition of methyl 4-methylpyridine-2-carboxylate (3H) (213 mg, 1.41 mmol). The reaction was warmed to room temperature overnight and then treated with 1N HCl to obtain a pH of 9, and extracted with ethyl acetate three times. The combined organic layers were washed with brine, dried over Na₂SO₄, filtered and concentrated in vacuo. It was purified by preparative HPLC on C18 reverse stationary phase eluted with a water/acetonitrile/TFA mobile phase. Concentration in vacuo, followed by azeotroping twice with benzene afforded 1-[5-benzyl-2-(pyridin-2-yloxy)phenyl]-3-(4-methylpyridin-2-yl)propane-1,3-dione (13E).

¹H NMR (400 MHz, CDCl₃) δ8.8 (d, J=5.3 Hz, 1H), 8.25 (m, 1H), 8.0 (s, 1H), 7.85 (d, J=1.65 Hz, 1H), 7.75 (m, 1H), 7.45 (d, J=5.1 Hz, 1H), 7.4−7.2 (m, 7H), 7.1 (m, 2H), 6.9 (d, J=8.42 Hz, 1H), 4.1 (s, 2H), 2.55 (s, 3H)

| | | | |
|---|---|---|---|
| Anal. Calc'd for: C₂₇H₂₂N₂O₃ 0.25 | C, 64.54; | H, 4.34; | N, 4.89. |
| benzene and 1.15 TFA | | | |
| Found: | C, 64.56; | H, 4.35; | N, 4.87. |

1-(5-Benzyl-2-fluorophenyl)-3-(4-methylpyridin-2-yl)propane-1,3-dione (13F)

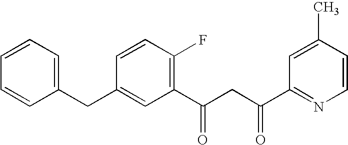

1-Benzyl-3-bromo-4-fluorobenzene

To a cold (0° C.) solution of 3-bromo-4-fluorobenzaldehyde (25.5 g) in THF (300 mL) under an atmosphere of argon, a solution of phenylmagnesium bromide in diethyl ether (3 M, 45 mL) was added. The resultant solution was stirred at room temp. for 2.5 h, and treated with aq. HCl. The resultant mixture was diluted with ethyl acetate, and neutralized with aq. HCl. The organic extract was dried over magnesium sulfate, filtered, and concentrated under vacuum to provide the intermediate alcohol. Without further purification, to a cold (0° C.) solution of the above crude alcohol (35 g) and triethylsilane (100 g) in dichloromethane (400 mL), boron trifluoride diethyl etherate (24 mL) was added dropwise over a period of 45 min. The resultant mixture was stirred at 0° C. for 1hr, diluted with dichloromethane, and neutralized with saturated aq. sodium bicarbonate. The organic extract was washed with brine, dried over magnesium sulfate, filtered, and concentrated under vacuum. The residue was subjected to column chromatography on silica gel eluted with hexane. Collection and concentration of appropriate fractions provided the title bromide.

In a manner similar to that for 3I, substituting 3D with the above bromide, 1-(5-benzyl-2-fluorophenyl)-3-(4-methylpyridin-2-yl)propane-1,3-dione (13F) was prepared.

| | | | |
|---|---|---|---|
| Anal. Calc'd for C₂₂H₁₈FNO₂. | C, 74.60; | H, 5.32; | N, 3.92. |
| Found: | C, 74.53; | H, 4.85; | N, 3.79. |

1-(2-Methoxy-5-[1,2,3]triazol-2-ylmethylphenyl)-3-(4-methylpyridin-2-yl)propane-1,3-dione (13G)

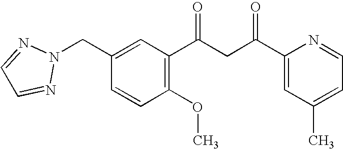

In a mannner similar to that described for 12C and 19B, 1-[5-(bromomethyl)-2-methoxyphenyl]ethanone was prepared from 1-(2-hydroxy-5-methylphenyl)ethanone (Aldrich) and coupled with 1,2,3 triazole to give a mixture of 1-(2-methoxy-5-[1,2,3]triazol-2-ylmethylphenyl)ethanone and 1-(2-methoxy-5-[1,2,3]triazol-1-ylmethylphenyl)ethanone. 1-(2-Methoxy-5-[1,2,3]triazol-2-ylmethyl-phenyl)-3-(4-methylpyridin-2-yl)propane-1,3-dione (13G) was consequently prepared in a similar manner to that described for 19E using the 1-(2-methoxy-5-[1,2,3]triazol-2-ylmethylphenyl)ethanone.

| Anal. Calc'd for: | C, 60.48; | H, 4.65; | N, 14.32. |
|---|---|---|---|
| C₁₉H₁₈N₄O₃ 0.35 C₂HF₃O₂ 0.05 H₂O | | | |
| Found: | C, 60.51; | H, 4.70; | N, 14.29. |
| Exact Mass: | C₁₉H₁₈N₄O₃ | | |
| Theor. Mass | 351.1452 | | |
| Meas. Mass | 351.1456 | | |

### EXAMPLE 14

1-[3-Benzyl-5-(4-methylpiperazin-1-yl)phenyl]-3-(4-methylpyridin-2-yl)-propane-1,3-dione (14E)

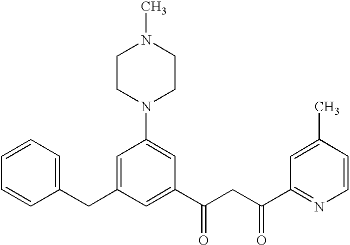

1-Azido-3-benzyl-5-bromobenzene (14A)

To a cold (−78 C.) solution of 1-benzyl-2,5-dibromobenzene (3B (4.0 g, 12.3 mmol) in 90 mL dry diethyl ether, under an atmosphere of argon, was added n-butyllithium (4.9 mL of a 2.5 M solution in hexanes, 12.3 mmol), over 10 minutes. The solution was stirred at −78 C. for 1 hour and then treated with a solution of tosyl azide (2.9 g, 14.7 mmol) in dry ether (25 mL), over 5 minutes. After warming to ambient temperature overnight, the reaction mixture was diluted with water and acidified to pH 5 with 5% aqueous KHSO₄. The mixture was extracted with ethyl acetate (3 times) and the combined extracts washed with brine, dried (MgSO₄), filtered and concentrated in vacuo to afford an oily solid. Purification by column chromatography on silica gel with hexane as the eluent afforded 1-azido-3-benzyl-5-bromobenzene as a light brown oil.

¹H NMR (400 MHz, CDCl₃) δ7.2 (m, 5H), 7.1 (m, 1H), 7.0 (m, 1H), 6.8 (m, 1H), 3.9 (s, 2H).

3-Benzyl-5-bromoaniline (14B)

A solution of 1-azido-3-benzyl-5-bromobenzene (2.4 g, 8.2 mmol) in 75 mL methanol was degassed under an atmosphere of argon, treated with 5% platinum an charcoal catalyst and hydrogenated in a Parr shaker at 40 p.s.i. for 2 hours. The mixture was degassed under argon, filtered through Celite and the filtrate concentrated in vacuo to a syrup. Purification by column chromatography on silica gel, with 15% ethyl acetate in hexane as the eluent, afforded 3-benzyl-5-bromoaniline as a light brown syrup.

¹H NMR (300 MHz, CDCl₃) δ7.25 (m, 5H), 6.74 (m, 1H), 6.68 (m, 1H), 6.4 (m, 1H), 3.8 (s, 2H), 3.7 (br s, 2H).

1-(3-Benzyl-5-bromophenyl)-4-methylpiperazine (14C)

A mixture of 3-benzyl-5-bromoaniline (1.0 g, 3.8 mmol) and Mechlorethamine hydrochloride (0.73 g, 3.8 mmol) in dry n-butanol (40 mL) was heated to reflux under an argon atmosphere for 2 days. Upon cooling to ambient temperature, a solid precipitated from solution. The solid was collected by filtration, washed with diethyl ether (2 times), and air dried. This material was then partitioned between CH₂Cl₂ and saturated aqueous NaHCO₃. The layers were separated and the aqueous layer extracted with CH₂Cl₂ (2 times). The combined CH₂Cl₂ extracts were dried (MgSO₄), filtered and concentrated in vacuo to afford 1-(3-benzyl-5-bromophenyl)-4-methylpiperazine as a colorless syrup without further purification.

¹H NMR (400 MHz, CDCl₃) δ7.25 (m, 5H) 6.9 ( m, 1H), 6.8 (m, 1H), 6.65 (m, 1H), 3.9 (s, 2H), 3.2 (m, 4H), 2.55 (m, 4H), 2.3 (s, 3H)

1-[3-Benzyl-5-(4-methylpiperazin-1-yl)phenyl]ethanone (14D)

A thick walled glass pressure vessel was charged with 1-(3-benzyl-5-bromo-phenyl)-4-methylpiperazine (0.51 g, 1.5 mmol), thallium acetate (0.43 g, 1.6 mmol), 1,3-bis(diphenylphosphino)propane (150 mg, 0.36), and dry N,N-dimethylformamide (3 mL). The slurry was purged for 15 minutes with argon and then treated with palladium acetate (80 mg, 0.36 mmol), triethylamine (0.61 ml, 4.5 mmol), and n-butyl vinyl ether (0.96 mL, 7.4 mmol). The pressure vessel was sealed and heated in a 100 C. oil bath with magnetic stirring overnight. The dark reaction mixture was allowed to cool to ambient temperature and filtered through Celite. The filtrate was concentrated in vacuo and the residue diluted with THF (10 mL), treated with 1M aqueous HCl (6 mL), and stirred for 2 hours. The mixture was made basic with saturated aqueous NaHCO₃ and extracted with ethyl acetate (3 times). The combined extracts were washed with brine, dried (MgSO₄), filtered, and concentrated in vacuo to a brown gum. Purification by column chromatography on silica gel with 3.5% methanol in chloroform as eluent afforded 1-[3-benzyl-5-(4-methylpiperazin-1-yl)phenyl]ethanone as a brown solid.

¹H NMR (400 MHz, CDCl₃) δ7.4 (m, 1H), 7.25 (m, 6H), 7.0 (m, 1H), 4.0 (s, 2H), 3.2 (m, 4H), 2.57 (m, 4H), 2.55 (s, 3H), 2.35 (s, 3H)

1-[3-Benzyl-5-(4-methylpiperazin-1-yl)phenyl]-3-(4-methylpyridin-2-yl)-propane-1,3-dione (14E)

To a solution 1-[3-benzyl-5-(4-methylpiperazin-1-yl)phenyl]ethanone (155 mg, 0.5 mmol) in dry THF (5 mL) under an atmosphere of argon, was added 4-methylpyridine-2-carboxilic acid methyl ester (3H) (113 mg, 0.75 mmol) and sodium, ethoxide (58 mg, 0.85 mmol) in single portions. The mixture was stirred 1 hour at ambient temperature, diluted with 1M aqueous HCl (0.8 mL), and concentrated in vacuo to a gum. Purification by preparative. HPLC on C18 reverse stationary phase eluting with a water/acetonitrile/trifluoroacetic acid mobile phase afforded the title compound as a lyophilized solid.

1H NMR (400 MHz, CDCl₃) δ8.7 (d, J=5.0 Hz, 1H), 8.1 (s, 1H), 7.6 (s, 1H), 7.5 (m, 2H), 7.4 (d, J=5.1 Hz, 1H), 7.35−7.2 (m, 5H), 6.9 (s, 1H), 4.0 (s, 2H), 3.7 (br m, 4H ), 3.4 (br m, 2H), 3.0 (br m, 2H), 2.9 (s, 3H), 2.5 (s, 3H).

| | | | |
|---|---|---|---|
| Anal. Calc'd for: | C, 59.46; | H, 5.06; | N, 6.89 |
| C₂₇H₂₉N₃O₂ 1.60 CF₃CO₂H | | | |
| Found: | C, 59.31; | H, 5.32; | N, 6.75. |

### EXAMPLE 15

1-(6-Benzyl-3-oxo-3,4-dihydro-2H-benzo[1,4]oxazin-8-yl)-3-(4-methylpyridin-2-yl)propane-1,3-dione (15E)

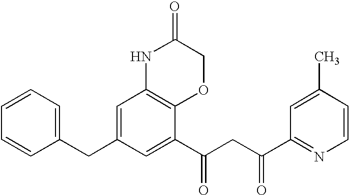

4-Benzyl-2-bromo-6-nitro-phenol (15A)

To a solution of 4-benzyl-2-bromophenol (13A) (25.8 g, 98 mmol) in glacial acetic acid (230 mL) was added a solution of concentrated nitric acid (6.2 mL, 98 mmol) in glacial acetic acid (35 mL) over a period of 1 hour. After stirring an additional 2 hours the reaction mixture was poured over ice, aged 15 minutes, and then the pH adjusted to ˜5. with concentrated NH₄OH. The mixture was extracted with ethyl acetate (3 times) and the combined extracts washed with brine, dried (MgSO₄), filtered, and concentrated in vacuo to a red oil. Purification by column chromatography on silica gel with 30% CH₂Cl₂ in hexane as the eluent afforded 4-benzyl-2-bromo-6-nitro-phenol as a yellow syrup.

¹H NMR (300 MHz, CDCl₃) δ11.0 (s, 1H), 7.9 (m, 1H), 7.7 ( m, 1H), 7.3 (m, 3H), 7.2 (m, 2H), 4.0.( s, 2H).

2-Amino-4-benzyl-6-bromophenol (15B)

A solution of 4-benzyl-2-bromo-6-nitrophenol (8.65 g, 28 mmol) in ethanol (100 mL) was degassed under an atmosphere of argon, treated with glacial acetic acid ( 8 mL) and 5% platinum on charcoal catalyst. The mixture was hydrogenated in a Parr shaker at 43 p.s.i. for 1 hour and then degassed under argon, filtered through Celite, and the filtrate concentrated in vacuo to a brown solid and used without further purification.

¹H NMR (400 MHz, CDCl₃) δ7.2 (m, 5H), 6.7 ( d, J=1.9 Hz, 1H), 6.5 (d, J=1.9 Hz, 1H), 5.8−4.2 (br s, 2H), 3.8 (s, 2H).

6-Benzyl-8-bromo-4H-benzo[1,4]oxazin-3-one (15C)

To a cold (0 C.) mixture of 2-amino-4-benzyl-6-bromophenol (4.0 g, 14.4 mmol), benzyl triethyl ammonium chloride (3.28 g, 14.4 mmol), and NaHCO₃ 4.84 g, 57.6 mmol) in CHCl₃, was added a solution of chloroacetyl chloride (1.38 mL, 17.3 mmol) in CHCl₃ (20 mL) over 30 minutes. The mixture was maintained at 0 C. for 1 hour and then heated in a 55 C. oil bath for 5 hours. After standing overnight at ambient temperature the mixture was concentrated in vacuo and the residue diluted with deionized water (100 mL). The resulting gummy solid was washed with water (4 times 100 mL), air dried, then washed with diethyl ether. Drying in vacuo afforded the title compound, an off white solid, which was used without further purification.

¹H NMR (400 Mz, CDCl₃) δ8.0 (br s, 1H), 7.25 (m, 5H), 7.05 (d, J=1.5 Hz, 1H), 6.5 (d, J=1.5 Hz, 1H), 4.7 (s, 2H), 3.9 (s, 2H).

8-Acetyl-6-benzyl-4H-benzo[1,4]oxazin-3-one (15D)

A thick walled glass pressure vessel was charged 6-benzyl-8-bromo-4H-benzo[1,4]oxazin-3-one (0.64 g, 2.0 mmol), thallium acetate (0.58 g, 2.2 mmol), 1,3-bis(diphenylphosphino)-propane (206 mg, 0.5 mmol), palladium acetate (112 mg, 0.5 mmol), and dry N,N-dimethylformamide (4 mL). The slurry was purged for 15 minutes with argon and then treated with triethylamine (0.82 ml,6.0 mmol), and n-butyl vinyl ether (1.0 mL, 7.7 mmol). The pressure vessel was sealed and heated in a 100 C. oil bath with magnetic stirring overnight. The dark reaction mixture was allowed to cool to ambient temperature and filtered through celite. The filtrate was concentrated in vacuo and the residue diluted with THF (20 mL), treated with 1M aqueous HCl (6 mL), and stirred for 1 hour. The mixture was diluted with water and extracted with ethyl acetate (3 times). The combined extracts were washed with brine, dried (MgSO₄), filtered, and concentrated in vacuo to a brown gum. Purification by column chromatography on silica gel with a 30%-40% ethyl acetate in hexane gradient afforded the title compound as a white solid.

¹H NMR (400 MHz, CDCl₃) δ7.8 (br s, 1H), 7.25 (m, 4H), 7.2 (m, 2H), 6.7 (d, j=2.0 Hz, 1H), 4.7 (s, 2H), 3.9 (s, 2H), 2.6 (s, 3H).

1-[6-Benzyl-3-oxo-3,4-dihydro-2H-benzo[1,4]oxazin-8-yl)-3-(4-methylpyridin-2-yl)propane-1,3-dione (15E)

To a solution of 8-acetyl-6-benzyl-4H-benzo[1,4]oxazin-3-one (150 mg, 0.5 mmol) in dry THF (5 mL) under an atmosphere of argon, was added 4-methylpyridine-2-carboxilic acid methyl ester (3H) (120 mg, 0.79 mmol) and sodium ethoxide (122 mg, 1.8 mmol) in single portions. The mixture was stirred 4 hours at ambient temperature, diluted with water and acidified to pH ˜2 with 1M aqueous HCl. The mixture was extracted with ethyl acetate (3 times), the combined extracts washed with brine, dried (MgSO₄), filtered, and concentrated in vacuo to a pale yellow solid. Recrystallization from CHCl₃/hexane afforded the title compound as a beige solid.

¹H NMR (400 MHz, CDCl₃) δ8.6 (d, J=4.8 Hz, 1H), 8.0 (s, 1H), 7.7 (s, 1H), 7.6 (s, 1H), 7.5 (d, J=1.6 Hz, 1H), 7.25 (m, 6H), 6.65 (d, J=1.7 Hz, 1H), 4.75 (s, 2H), 3.95 (s, 2H), 2.45(s, 3H).

| | | | |
|---|---|---|---|
| Anal. Calc'd for: C₂₄H₂₀N₂O₄ 0.35 H₂O | C, 70.87; | H, 5.13; | N, 6.89 |
| Found: | C, 70.79; | H, 5.13; | N, 6.51. |

### EXAMPLE 16

1-(3-Benzyl-5-[1,2,4]triazol-1-ylmethylphenyl)-3-(4-methylpyridin-2-yl)-propane-1,3-dione (16C)

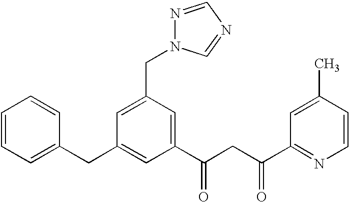

3-Benzyl-5-[1,2,4]triazol-1-ylmethyl-1-bromobenzene (16A)

A mixture of sodium hydride (132 mg, 60% dispersion in minera oil, washed with hexane) and 1,2,4triazole (228 mg) in DMF was stirred at room temperature for 10 min. The resultant mixture was treated with a solution of 3-benzyl-5-bromobenzyl bromide in DMF. The reaction mixture was stirred at room temperature over night. The product mixture was concentrated under vacuum and the residue partitioned between ethyl acetate and aqueous ammonium chloride. The organic extract was washed with brine, dried over magnesium sulfate, filtered, and concentrated under vacuum. The crude triazole product was used for next step without further purification.

¹H NMR (400 MHz, CDCl₃) δ8.07 (s, 1H), 8.01 (s, 1H), 7.31−7.21 (m, 5H), 7.12 (d, J=7.1 Hz; 2H), 7.0 (s, 1H), 5.26 (s, 2H), 3.92 (s, 2H).

3-Benzyl-5-[1,2,4]triazol-1-ylmethylacetophenone (16B)

To a mixture of 3-benzyl-5-[1,2,4]triazol-1-ylmethyl-1-bromobenzene (0.7 g), thallium acetate (0.62 g), 1,3-bis(diphenylphosphino)propane (0.22 g) and triethylamine (1.18 mL) in DMF (4 mL) in a pressure tube, purged with argon for a period of 10 minutes, palladium acetate (95 mg) and n-butyl vinyl ether (1.4 mL) was added. The reaction tube was sealed and stirred at 100° C. overnight. The reaction mixture was filtered through a bed of Celite, and the filtrate concentrated under vacuum. The residue was dissolved in THF (5 mL) and treated with aqueous HCl (3 M, 4 mL). The resultant mixture was stirred at rt for 3 hr., diluted with ethyl acetate, basified with aqueous sodium bicarbonate. The organic extract was dried over magnesium sulfate, filtered, and concentrated under vacuum. The residue was subjected to column chromatography on silica gel eluting with 50% ethyl acetate in hexane. Collection and concentration of appropriate fractions provided the title ketone.

¹H NMR (400 MHz, CDCl₃) δ8.38 (br s, 1H), 7.87 (br s, 1H), 7.80 (br s, 1H), 7.69 (br s, 1H), 7.36−7.2 (m, 4H), 7.16 (d, J=6.7 Hz, 2H), 5.38 (s, 2H), 4.0 (s, 2H), 2.58 (s, 3H).

1-(3-Benzyl-5-[1,2,4]triazol-1-ylmethylphenyl)-3-(4-methylpyridin-2-yl)-propane-1,3-dione(16C)

A solution of 3-benzyl-5-[1,2,4]triazol-1-ylmethylacetophenone (6E) (60 mg) and methyl 4-methylpyridine-2-carboxylate (3H) (62 mg) in THF (3 mL) under an atmosphere of argon was treated with sodium ethoxide (21 mg). The resultant mixture was stirred at room temperature for 4 hours, and was quenched with saturated aqueous ammonium chloride and partitioned with ethyl acetate. The organic extract was washed with brine, dried over magnesium sulfate, filtered, and concentrated under vacuum. The residue was triturated with 50% ethyl acetate in hexane. Filtration and collection of the solid provided the title compound.

¹H NMR (400 MHz, CDCl₃) δ8.56 (d, J=4.9 Hz, 1H), 8.07(s, 1H), 7.98 (d, J=6.05 Hz, 2H), 7.90 (s, 1H), 7.82 (s, 1H), 7.49 (s, 1H), 7.31−7.17 (m, 5H), 7.16 (d, J=6.96, 2H), 5.36 (s, 2H), 4.04 (s, 2H), 2.45 (s, 3H).

| | | | |
|---|---|---|---|
| Anal. Calc'd for C₂₅H₂₂N₄O₂.0.25 H₂O | C, 72.35; | H, 5.47; | N, 13.50. |
| Found: | C, 72.30; | H, 5.68; | N, 13.49. |

1-(3-Benzyl-5-imidazol-1-ylphenyl)-3-(4-methylpyridin-2-yl)-propane-1,3-dione (16D)

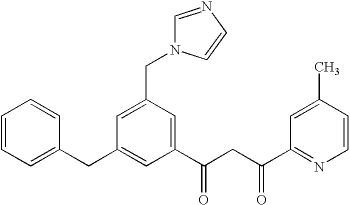

In a manner similar to that for 16C, 1-(3-Benzyl-5-imidazol-1-ylphenyl)-3-(4-methylpyridin-2-yl)-propane-1,3-dione (16D) was prepared.

| | | | |
|---|---|---|---|
| Anal. Calc'd for C₂₆H₂₃N₃O₂.0.35 H₂O | C, 75.10; | H, 5.75; | N, 10.11. |
| Found: | C, 75.07; | H, 5.62; | N, 10.03. |

1-(3-Benzyl-5-[1,2,3]triazol-1-ylmethylphenyl)-3-(4-methylpyridin-2-yl)-propane-1,3-dione (16E)

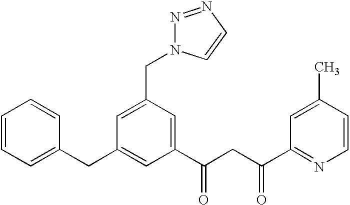

In a manner similar to that for 16C, 1-(3-Benzyl-5-[1,2,3]triazol-1-ylmethylphenyl)-3-(4-methylpyridin-2-yl)-propane-1,3-dione (16E) was prepared.

| | | | |
|---|---|---|---|
| Anal. Calc'd for C₂₅H₂₂N₄O₂.0.20 H₂O | C, 72.51; | H, 5.45; | N, 13.53. |
| Found: | C, 72.46; | H, 5.14; | N, 13.40. |

1-(3-Benzyl-5-[1,2,3]triazol-1-ylmethylphenyl)-3-(6-chloropyridin-2-yl)-propane-1,3-dione (16F)

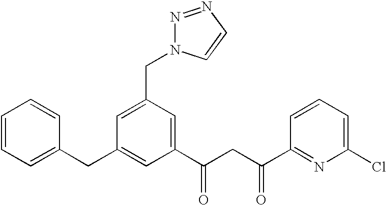

In a manner similar to that for 16C, 1-(3-Benzyl-5-[1,2,3]triazol-1-ylmethylphenyl)-3-(6-chloropyridin-2-yl)-propane-1,3-dione (16F) was prepared.

¹H NMR (400 MHz, CDCl₃) δ8.07(d, J=7.7 Hz, 1H), 7.88−7.82 (m, 3H), 7.73 (br s, 1H), 7.50,−7.16 (m, 9H), 5.61(s, 2H), 4.05 (s, 2H).

1-(3-Benzyl-5-tetrazol-1-ylmethylphenyl)-3-(4-methylpyridin-2-yl)-propane-1,3-dione (16G)

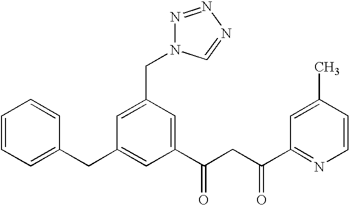

In a manner similar to that for 16C 1-(3-Benzyl-5-tetrazol-1-ylmethylphenyl)-3-(4-methylpyridin-2-yl)-propane-1,3-dione (16G) was prepared.

| | | | |
|---|---|---|---|
| Anal. Calc'd for C₂₅H₂₁N₅O₂.0.25 | C, 69.26; | H, 5.35; | N, 16.16. |
| EtOAc | | | |
| Found: | C, 69.40; | H, 5.11; | N, 16.04. |

1-(3-Benzyl-5-[1,2,3]triazol-2-ylmethylphenyl)-3-(4-methylpyridin-2-yl)-propane-1,3-dione (16H)

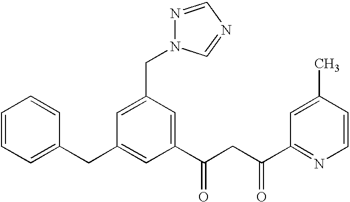

In a manner similar to that for 16C, 1-(3-Benzyl-5-[1,2,3,]triazol-2-ylmethylphenyl)-3-(4-methylpyridin-2-yl)-propane-1,3-dione (16H) was prepared.

| | | | |
|---|---|---|---|
| Anal. Calc'd for C₂₅H₂₂N₄O₂.1.4 | C, 67.54; | H, 4.99; | N, 12.21. |
| TFA & 0.15 H₂O | | | |
| Found: | C, 67.58; | H, 4.93; | N, 12.10. |

1-(3-Benzyl-5-tetrazol-2-ylmethylphenyl)-3-(4-methylpyridin-2-yl)-propane-1,3-dione (16I)

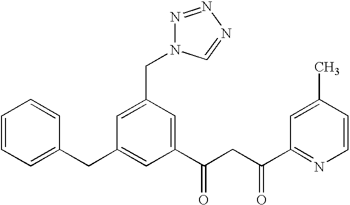

In a manner similar to that for 16C, 1-(3-Benzyl-5-tetrazol-2-ylmethylphenyl)-3-(4-methylpyridin-2-yl)-propane-1,3-dione (16I) was prepared.

| | | | |
|---|---|---|---|
| Anal. Calc'd for C₂₅H₂₁N₅O₂.0.15 | C, 68.27; | H, 5.38; | N, 16.18. |
| EtOAc & 0.45 H₂O | | | |
| Found: | C, 68.22; | H, 5.27; | N, 16.13. |

1-(3-Benzyl-5-pyrrolo[2,3]pyridin-1-ylmethylphenyl)-3-(4-methylpyridin-2-yl)-propane-1,3-dione (16J)

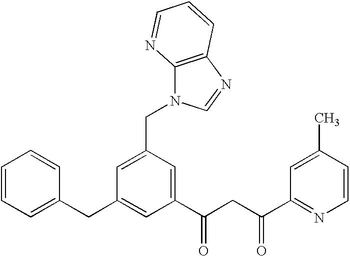

In a manner similar to that for 16C, 1-(3-Benzyl-5-pyrrolo[2,3]pyridin-1-ylmethylphenyl)-3-(4-methylpyridin-2-yl)-propane-1,3-dione (16J) was prepared.

¹H NMR (400 MHz, CDCl₃) δ8.56 (d, J=5 Hz, 1H), 8.34 (dd, J=1.5, 3.8 Hz, 1H), 7.97 (br s, 1H), 7.93 (dd, J=1.3, 7.7 Hz, 1H), 7.81 (d, J=6.4 Hz, 2H), 7.46(s, 1H), 7.24−7.07 (m, 9H), 6.49 (br s, 1H), 5.53 (s, 2H), 3.98 (s, 2H), 2.45 (s, 3H).

1-(3-Benzyl-5-indazol-1-ylmethyl)phenyl-3-(4-methylpyridin-2-yl)propane-1,3-dione (16K)

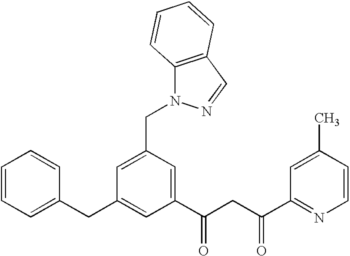

In a manner similar to that for 16C, 1-(3-benzyl-5-indazol-1-ylmethyl)-phenyl-3-(4-methylpyridin-2-yl)propane-1,3-dione (16K) was prepared.

| | | | |
|---|---|---|---|
| Anal. Calc'd for C₃₀H₂₅N₃O₂ | C, 78.41; | H, 5.48; | N, 9.14. |
| Found: | C, 78.98; | H, 5.53; | N, 9.17. |

1-(3-Benzyl-5-pyrazol-1-ylmethyl)phenyl-3-(4-methylpyridin-2-yl)propane-1,3-dione (16L)

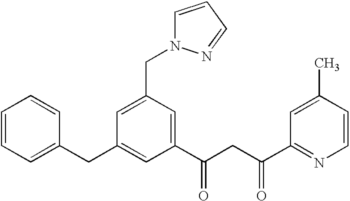

In a manner similar to that for 16C, 1-(3-benzyl-5-pyrazol-1ylmethyl)-phenyl-3-(4-methylpyridin-2-yl)propane-1,3-dione (16L) was prepared.

¹H NMR (400 MHz, CDCl₃) δ8.70 (br d, 1H), 8.04 (s, 1H), 7.88 (s, 1H), 7.80 (s, 1H), 7.60 (s, 1H), 7.50 (s, 1H), 7.42 (s, 1H), 7.41 (s, 1H), 7.36 (br d, 1H), 7.31−7.15 (m, 3H), 6.31 (br s, 1H), 5.37 (s, 2H), 4.03 (s, 2H), 2.51 (s, 3H).

1-(3-Benzyl-5-[1,2,3]triazolo[4,5,b]pyridin-1-ylmethyl)phenyl-3-(4-methylpyridin-2-yl)-propane-1,3-dione (16M)

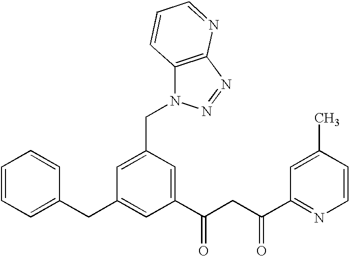

In a manner similar to that for 16C, 1-(3-benzyl-5-[1,2,3]triazolo[4,5b]-pyridin-1-ylmethyl)phenyl-3-(4-methylpyridin-2-yl)propane-1,3-dione (16M) was prepared.

¹H NMR (400 MHz, CDCl₃) δ8.74 (br d, 1H), 8.57 (d,. 1H), 7.99 (s, 1H), 7.90 (m, 2H), 7.61 (d, 1H), 7.49 (s, 1H), 7.34−7.08 (m, 8H), 5.89 (s, 2H), 3.99 (s, 2H), 2.46 (s, 3H).

1-[3-Benzyl-5-(3-methyl-2,6-dioxo-3,6-dihydro-2H-pyrimidin-1-ylmethyl)phenyl]-3-(4-methylpyridin-2-yl)propane-1,3-dione (16N)

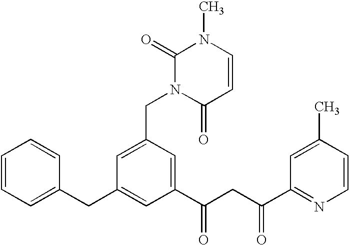

In a manner similar to that for 16C, 1-[3-benzyl-5-(3-methyl-2,6-dioxo-3,6-dihydro-2H-pyrimidin-1-ylmethyl)phenyl]-3-(4-methylpyridin-2-yl)propane-1,3-dione (16N) was prepared.

¹H NMR (400 MHz, CDCl₃) δ8.74 (br d, 1H), 8.04 (s, 1H), 7.99 (s 1H), 7.82 (s, 1H), 7.47 (br d, 1H), 7.38 (br d, 1H), 7.30−7.11 (m, 6), 5.77 (br s, 1H), 5.16 (s, 2H), 4.03 (s, 2H), 3.38 (s, 3H), 2.51 (s, 3H).

1-[3Benzyl-5-(2-oxo-1,2-dihydro-2H-pyrimidin-1-ylmethyl)phenyl]-3-(4-methylpyridin-2-yl)propane-1,3-dione (16O)

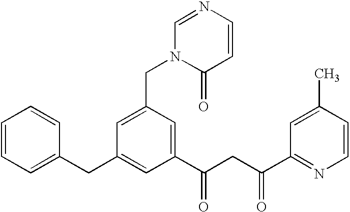

In a manner similar to that for 16C, 1-[3-benzyl-5-(2-oxo-1,2-dihydro-2H-pyrimidin-1-ylmethyl)phenyl]-3-(4-methylpyridin-2-yl)propane-1,3-dione (16O) was prepared.

¹H NMR (400 MHz, CDCl₃) δ8.70 (br d, 1H), 8.27 (s, 1H), 8.05 (s, 1H), 7.90−7.87 (m, 3H), 7.52 (s, 1H), 7.38−7.16 (m, 7H), 6.51 (br d, 1H), 5.15 (s, 2H), 4.05 (s,2H), 2.51 (s, 3H).

1-(3-Benzyl-5-purin-9-ylmethyl)phenyl-3-(4-methylpyridin-2-yl)propane-1,3-dione (16P)

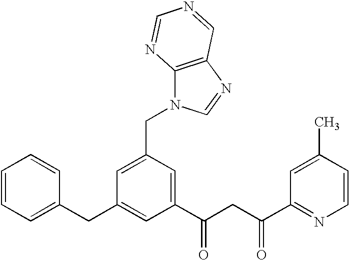

In a manner similar to that for 16C, 1-(3-benzyl-5-purin-9-ylmethyl)-phenyl-3-(4-methylpyridin-2-yl)propane-1,3-dione (16P) was prepared.

| | | | |
|---|---|---|---|
| Anal. Calc'd for C₂₈H₂₃N₅O₂ | C, 64.57; | H, 5.46; | N, 11.77. |
| Found: | C, 64.53; | H, 5.11; | N, 11.40. |

1-[3-Benzyl-5-(1,1-dioxoisothiazolidin-2-ylmethyl)phenyl]-3-(4-methylpyridin-2-yl-propane-1,3-dione (16Q)

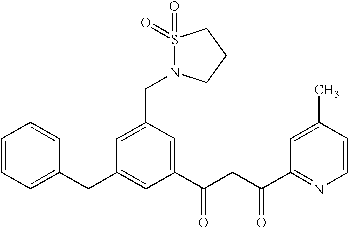

In a manner similar to that for 16C, 1-[3-benzyl-5-(1,1-dioxo-isothiazolidin-2-ylmethyl)phenyl]-3-(4-methylpyridin-2-yl)propane-1,3-dione (16Q) was prepared.

| | |
|---|---|
| HRMS. Calc'd for C₂₆H₂₇N₂O₄S (M + 1) | 463.1649. |
| Found: | 463.1686. |

1-[3-Benzyl-5-(1,1-dioxothiazinan-2-ylmethyl)phenyl]-3-(4-methylpyridin-2-yl)-propane-1,3-dione (16R)

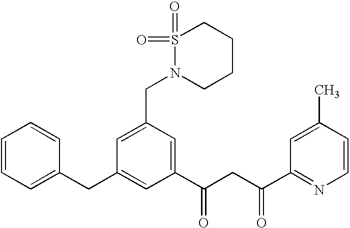

In a manner similar to that for 16C, 1-[3-benzyl-5-(1,1-dioxothiazinan-2-ylmethyl)phenyl]-3-(4-methylpyridin-2-yl)propane-1,3-dione (16R) was prepared.

| | | | |
|---|---|---|---|
| Anal. Calc'd for C₂₇H₂₈N₂O₄S | C, 68.04; | H, 5.92; | N, 5.88. |
| Found: | C, 67.70; | H, 5.64; | N, 5.64. |

1-[3-Benzyl-5-(1,1-dioxo-[1,2,6]-thiadiazinan-2-ylmethyl)phenyl]-3-(4-methylpyridin-2-yl)propane-1,3-dione (16S)

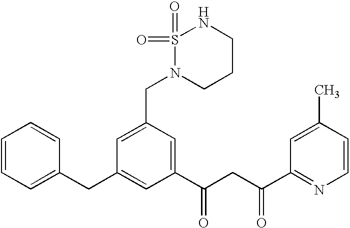

In a manner similar to that for 16C, 1-[3-benzyl-5-(1,1-dioxo-[1,2 6]-thiadiazinan-2-ylmethyl)phenyl]-3-(4-methylpyridin-2-yl)propane-1,3-dione (16S) was prepared.

| | | | |
|---|---|---|---|
| Anal. Calc'd for C₂₆H₂₇N₃O₄S 0.4 TFA | C, 61.53; | H, 5.28; | N, 8.03. |
| Found: | C, 61.64; | H, 4.81; | N, 7.79. |

1-[3-Benzyl-5-(2-oxo-2H-pyrimidin-1-ylmethyl)phenyl]-3-(pyridin-2-yl)propane-1,3-dione (16T)

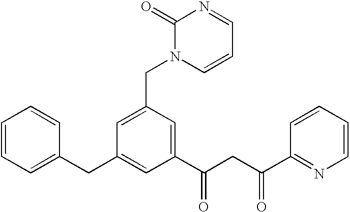

In a manner similar to that for 16C, 1-[3-benzyl-5-(2-oxo-2H-pyrimidin-1-ylmethyl)phenyl]-3-(pyridin-2-yl-propane-1,3dione (16T) was prepared.

| | | | |
|---|---|---|---|
| Anal. Calc'd for C₂₆H₂₁N₃O₃ | C, 58.51; | H, 4.65; | N, 7.69. |
| Found: | C, 58.56; | H, 4.45; | N, 7.30. |

1-[3-Benzyl-5-(1,1-dioxotetrahydrothiophen-2-ylmethyl)phenyl]-3-(4-methylpyridin-2-yl)propane-1,3-dione (16U)

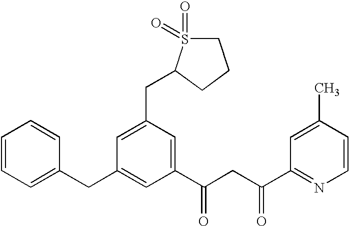

In a manner similar to that for 16C, 1-[3-benzyl-5-(1,1-dioxotetrahydrothiophen-2-ylmethyl)phenyl]-3-(4-methylpyridin-2-yl)propane-1,3-dione (16U) was prepared.

| | |
|---|---|
| HRMS. Calc'd for C₂₇H₂₈NO₄S (M + 1) | 462.1713. |
| Found: | 462.1734. |

1-[3-Benzyl-5-(1,1-dioxotetrahydrothiophen-2-ylmethyl)-2-isopropoxyphenyl]-3-(4-methylpyridin-2-yl)propane-1,3-dione (16V)

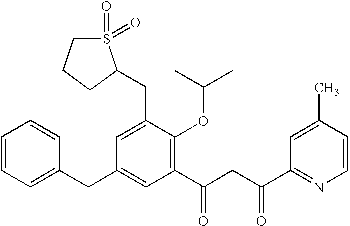

In a manner similar to that for 16C, 1-[3benzyl-5-(1,1-dioxotetrahydro-thiophen-2-ylmethyl)-2-isopropoxyphenyl]-3-(4-methylpyridin-2-yl)propane-1,3dione (16V) was prepared.

| | |
|---|---|
| HRMS. Calc'd for C₃₀H₃₄NO₅ (M + 1) | 520.2160. |
| Found: | 520.2152. |

1-[3-Benzyl-5-(1,3-dimethyl-2,3,6,1-tertrahydro-2,6-dioxopurin-9-ylmethyl)phenyl]-3-(4-methylpyridin-2-yl)propane-1,3-dione (16W)

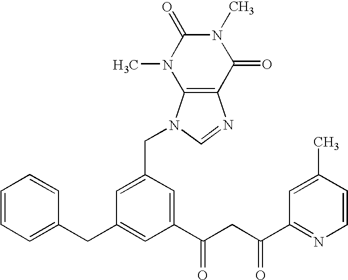

In a manner similar to that for 16C, 1-[3-benzyl-5-(1,3-dimethyl-2,3,6,1-tertrahydro-2,6-dioxopurin-9-ylmethyl)phenyl]-3-(4-methylpyridin-2-yl)propane-1,3-dione (16W) was prepared.

| | | | |
|---|---|---|---|
| Anal. Calc'd for C₃₀H₂₇N₅O₄ | C, 62.14; | H, 5.02; | N, 12.08. |
| Found: | C, 62.40; | H, 4.76; | N, 11.68. |

1-[3-Benzyl-5-(6-dimethylaminopurin-7-ylmethyl)phenyl]-3-(4-methylpyridin-2-yl)propane-1,3-dione (16X)

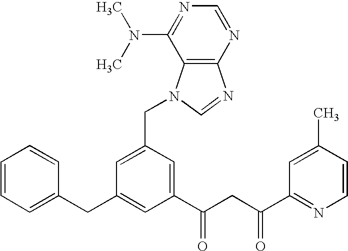

In a manner similar to that for 16C, 1-[3-benzyl-5-(1,3-dimethyl-2,3,6,1-tertrahydro-2,6-dioxopurin-9-ylmethylphenyl]-3-(4-methylpyridin-2-yl)propane-1,3-dione (16X) was prepared.

| | | | |
|---|---|---|---|
| Anal. Calc'd for C₃₀H₂₈N₆O₂ | C, 53.41; | H, 3.92; | N, 10.71. |
| Found: | C, 53.77; | H, 3.69; | N, 10.31. |

1-[3-Benzyl-5-(4-methyl-5-thioxo-3-trifluoromethyl-4,5-dihydro-[1,24]-triazol-1-ylmethyl)phenyl]-3-(4-methylpyridin-2-yl)propane-1,3-dione (16Y)

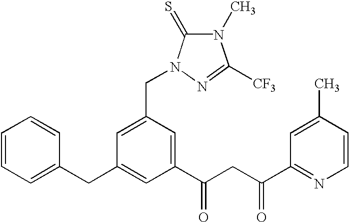

In a manner similar to that for 16C, 1-[3-benzyl-5-(4-methyl-5-thioxo-3-trifluoromethyl-4,5-dihydro-[1,24]-triazol-1-ylmethyl)phenyl]-3-(4-methylpyridin-2-yl)-propane-1,3-dione (16Y) was prepared.

| | | | |
|---|---|---|---|
| Anal. Calc’d for C₂₇H₂₃F₃N₄O₂S | C, 51.67; | H, 3.57; | N, 8.03. |
| Found: | C, 51.74; | H, 3.51; | N, 7.86. |

1-[3-Benzyl-5-(3,7-dimethyl-3,7-dihydro-2,6-dioxopurin-1-ylmethyl)phenyl]-3-(4-methylpyridin-2-yl)propane-1,3-dione (16Z)

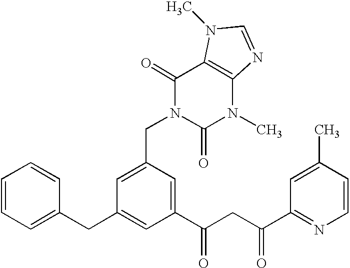

In a manner similar to that for 16C, 1-[3-benzyl-5-(3,7-dimethyl-3,7-dihydro-2,6-dioxopurin-1-ylmethyl)phenyl]-3-(4-methylpyridin-2-yl)propane-1,3-dione (16Z) was prepared.

| | | | |
|---|---|---|---|
| Anal. Calc’d for C₃₀H₂₇N₅O₄ | C, 58.98; | H, 5.47; | N, 10.00. |
| Found: | C, 59.04; | H, 5.20; | N, 9.99. |

1-[3-Benzyl-5-(2-oxo-2H-pyridin-1-ylmethyl)phenyl]-3-(4-methylpyridin-2-yl)propane-1,3-dione (16AA)

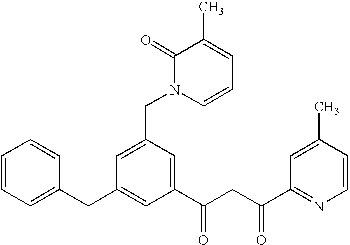

In a manner similar to that for 16C, 1-[3-benzyl-5-(2-oxo-2H pyridin-1-ylmethyl)phenyl]-3-(4-methylpyridin-2-yl)propane-1,3-dione (16AA) was prepared.

| | | | |
|---|---|---|---|
| Anal. Calc’d for C₂₉H₂₆N₂O₃ | C, 58.75; | H, 4.52; | N, 4.23. |
| Found: | C, 58.98; | H, 4.54; | N, 4.14. |

¹H NMR (400 MHz, CDCl₃) δ

1-[3-Benzyl-5-([1,2,3]triazolo[4,5-b]pyridinyl-2-ylmethyl)phenyl]-3-(4-methylpyridin-2-yl)propane-1,3-dione (16AB)

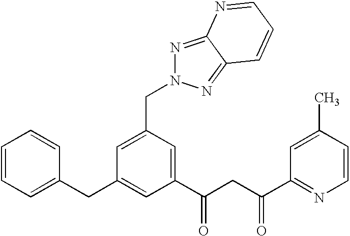

In a manner similar to that for 16C, 1-[3-benzyl-5-(1,2,3]triazolo-4,5-b]pyridinyl-2-ylmethyl)phenyl]-3-(4-methylpyridin-2-yl)propane-1,3-dione (16AB) was prepared.

¹H NMR (400 MHz, CDCl₃) δ8.73 (d, 1H), 8.69 (d, 1H), 8.39 (d, 1H), 8.07 (s, 1H), 8.04 (s, 1H), 7.88 (s, 1H), 7.51 (s, 1H), 7.45 (s 1H), 7.38 (m, 2H), 7.26−7.12 (m, 6H), 5.95 (s, 2H), 4.01 (s, 2H), 2.52 (s, 3H).

### EXAMPLE 17

1-(3-Benzylphenyl)-3-(4H-[1,2,4]triazole-3-yl)propane-1,3-dione (17D)

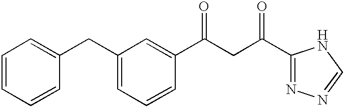

4-trityl-4H-[1,2,4]triazole-3-carboxylic acid ethyl ester (17B)

A suspension of 4H-[1,2,4]triazole-3-carboxylic acid ethyl ester (17A) (0.25 g, 0.002 mole, prepared by the method of P. Vemisletti et. al. J. Heterocyclic Chem 1988, 25, 651) in 10 mL of DMF was treated with diisopropylethylamine6 (0.67 mL, 0.004 mole) followed by trityl bromide (0.63 g, 0.002 mole) and stirred overnight at room temperature. The suspension was poured into H₂O and extracted with EtOAc three times, the combined organic layers were dried over Na₂SO₄, filtered and evaporated to give 4-trityl-4H-[1,2,4]triazole-3carboxylic acid ethyl ester (17B) contaminated with some trityl bromide. The mixture was taken on to the next step.

¹H NMR (400 MHz, CDCl₃) δ7.3 (m, 20H), 7.1 (m, 8H), 4.45 (q, 2H), 1.4 (t, 3H).

1-(3-benzylphenyl)-3-(4-trityl-4H-[1,2,4]triazol-3-yl)propane-1,3-dione (17C)

To an oven dried three necked round bottomed flask with a stirring bar, septum, argon inlet was added THF (1.2 mL) and 4B (1-(3-benzylphenyl)ethanone 0.074 g, 0.00035 mole. The reaction was treated with NaOEt (0.052 g 0.0007 mole). To this well stirred solution was added 17B (0.13 g, 0.00035 mole,). This solution was aged 60 min then quenched with a solution of NH₄Cl. The mixture was diluted with EtOAc and the layers separated. The aqueous layer was extracted further with EtOAc and the organic layers were combined. Drying (Na₂SO₄), filtration and removal of the solvent in vacuo gave crude 1-(3-benzylphenyl)-3-(4-trityl-4H-[1,2,4]triazol-3-yl)propane-1,3-dione (17C) that was taken on to the next step.

¹H NMR (400 MHz, CDCl₃) δ7.8(m, 2H), 7.4−7.0(m, 16H), 4.0(s,2H).

1-(3-benzylphenyl)-3-(4H-[1,2,4]triazol-3-yl)propane-1,3-dione (17D)

To an oven dried three necked round bottomed flask with a stirring bar, septum, argon inlet and thermometer was added 17C (0.13 g, 0.00024 mole) and trifluoroacetic acid (4 mL). The reaction was cooled to 0° C. and to this well stirred solution was added EtSiH (0.045 mL, 0.00024 mole). The reaction was warmed to room temperature. After 45 min the reaction was quenched with a saturated solution of NaHCO₃. The mixture was diluted with EtOAc and the layers separated. The aqueous layer was extracted further with EtOAc and the organic layers were combined. Drying (Na₂SO₄), filtration and removal of the solvent in vacuo to gave crude product that was purified by HPLC and lyophilized from dioxane to give pure 1-(3-benzylphenyl)-3-(4H-[1,2,4]triazol-3-yl)propane-1,3-dione (7D).

¹H NMR (400 MHz, CDCl₃) δ8.8 (bs, 1H), 7.92(s, 1H), 7.85(m,7H), 7.5 (m, 3H), 7.3−7.1(m,4H), 7.2(s,1H), 7.08 (s, 2H).

| | | | |
|---|---|---|---|
| Anal. Calc’d for: C₁₈H₁₅N₃O₂.0.2 | C, 69.92; | H, 5.18; | N, 13.01. |
| dioxane | | | |
| Found: | C, 70.03; | H, 4.65; | N, 12.45. |

Exact Mass calc. 306.1237, fnd. 306.1251.

### EXAMPLE 18

1-(3-Benzylphenyl)-3-(3-isopropoxypyridin-2-yl)propane-1,3-dione (18D)

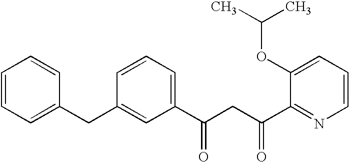

3-Isopropoxypicolinic acid (18B)

Into a 20 mL flame dried round bottom flask equipped with a nitrogen inlet, magnetic stirring bar and reflux condenser was placed 10 mL THF and 2- propanol (2 g, 33.3 mmol). To this was added sodium metal (0.761 g, 33 mmol) and the mixture stirred until all the sodium was dissolved, after which 3-chloropicolinic acid (1 g, 6.3 mmol) was added and the mixture refluxed for 18 hr. The reaction was cooled and the solvent removed in vacuo. Water was added the pH adjusted to 7.0 by the addition of 10% HCl. The water was then removed in vacuo and the residue stirred vigorously with 3×50 mL 10% MeOH/CH₂Cl₂. The organic extracts were combined and the solvent removed to afford 3-isopropoxypicolinic acid (18B).

¹H NMR (400 MHz, d6-DMSO) δ7.91(d, j=4 Hz, 1H), 7.26(d, j=8.4 Hz, 1H), 7.08(m, 1H), 4.55 (m, 1H), 1.21(d, j=6 Hz, 6H).

3-Isopropoxypicolinic acid methyl ester (18C)

3-Isopropoxypicolinic acid (0.35 g, 1.9 mmol) and 140 mL MeOH where mixed in a 250 mL flame dried round bottomed flask equipped with a reflux condenser, magnetic stirring bar and nitrogen inlet. To this solution was added thionyl chloride (1.15 g, 9.7 mmol) dropwise. After the addition, the reaction was heated to reflux for 48 hr., cooled and the solvent removed in vacuo. The resulting residue was partitioned between ethyl acetate/Na₂CO₃ saturated H₂O and extracted. The combined organic extracts were washed with H₂O, brine, dried over anhydrous sodium sulfate filtered and the solvent removed to give 3-isopropoxypicolinic acid methyl ester (18C).

¹H NMR (400 MHz, CDCl₃) δ8.22(d, j=4 Hz, 1H), 7.29(m, 2H), 4.60 (m, 1H), 3.97(s, 3H), 1.29(d, j=6 Hz, 6H).

1-(3-Benzylphenyl)-3-(3-isopropoxypyridin-2-yl)-propane-1,3-dione (18D)

1-(3-benzylphenyl)ethanone (4B) (0.1 g, 0.48 mmol) was dissolved in 5 mL THF in a 20 mL round bottomed flask fitted with a magnetic stirring bar and nitrogen inlet. To this solution was added sodium ethoxide (0.128 g, 1.9 mmol) followed by 3-isopropoxypicolinic acid methyl ester (0.19 g, 0.97 mmol). After stirring 1 hr., the reaction was quenched by the addition of 10 mL water and extracted with ethyl acetate. The combined organic extracts were washed with H₂O, brine, dried over anhydrous sodium sulfate, filtered and the solvent removed. The product was purified by preparative HPLC to yield 1-(3-benzylphenyl)-3-(3-isopropoxypyridin-2- yl)-propane-1,3-dione (18D) as the TFA salt.

¹H NMR (400 MHz, CDCl₃) δ8.75(d, j=3.6 Hz, 1H), 7.89-7.98(m, 2H), 7.81(s, 2H), 7.42-7.50(m, 2H), 7.38(s, 1H), 7.16-7.35(m, 5H), 4.91(m, 1H), 4.07(s, 1H), 1.52(d, j=6 Hz, 6H)

ES MS: m/z 374 (M+1),

1-(3-Benzylphenyl)-3-(3-propoxypyridin-2-yl)-propane-1,3-dione (18E)

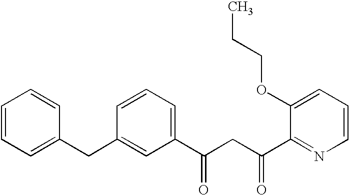

In a manner similar to that for 18D, 1-(3benzylphenyl)-3-(3-propoxypyridin-2-yl)-propane-1,3-dione (18E) was prepared.

ES MS: m/z 374 (M+1).

### EXAMPLE 19

1-(3,5-Bis-pyrazol-1-ylmethyl-phenyl)-3-(4-methyl-pyridin-2-yl)-propane-1,3dione (19E)

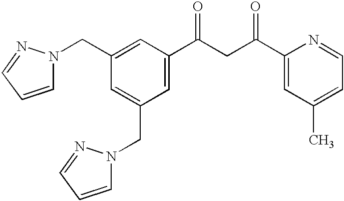

1-Bromo-3,5-bis-bromomethyl-benzene (19B)

To a solution of 1-bromo-3,5-dimethylbenzene (19A) (5.00 g, 0.027 mole) in 50 mL of CCl₄ was added N-bromosuccinimide (9.6 g, 0.054 mole) and benzoyl peroxide (0.18 g, 1.35 mmol). The reaction was refluxed for 4 hours. The reaction as cooled, filtered, and the solvent was removed in vacuo to give a clear yellow oil. This material was chromatographed on silica gel using 100% hexanes as the eluant to give 1-bromo-3,5-bis-bromomethyl-benzene (19B) as a white solid.

Rf=0.21 (100% hexanes)

1-Bromo-3,5-(bis-pyrazol-1-ylmethyl)-benzene (19C)

K₂CO₃ (3.99 g, 28.9 mmol) was added to a flame dried containing a solution of pyrazole (1.79 g, 26.3 mmol) dissolved in 60 mL anhydrous CH₃CN. After 15 minutes 1-bromo-3,5-bis-bromomethyl-benzene (19B) (3.0 g, 8.75 mmol) in CH₃CN was added and the resulting mixture was stirred overnight at room temperature. The reaction was quenched with saturated NH₄Cl solution and extracted with EtOAc. The combined organic extracts were washed with brine, dried over Na₂SO₄, filtered, and concentrated to give a yellow oil. This material was chromatographed on silica gel using 60% EtOAc in hexanes as the eluant to give 1-bromo-3,5-(bis-pyrazol-1-ylmethyl)-benzene (19C) as a white solid.

Rf=0.42 (60% EtOAc/hexanes)

¹H NMR (400 MHz, CDCl₃) δ7.55(m, 2H), 7.39(m, 2H), 7.23(m, 2H), 6.94 (m, 1H), 6.30(m, 2H), 5.26(s, 4H).

1-(3,5-Bis-pyrazol-1-ylmethyl-phenyl)-ethanone (19D)

In a dried sealable pressure tube under argon was dissolved 1-bromo-3,5-(bis-pyrazol-1-ylmethyl)-benzene (19C) (140 mg, 0.47 mmol) in 3 mL DMF. TEA (131 mL, 0.94 mmol), butyl vinyl ether (303 μl, 2.35 mmol), thallium acetate (136 mg, 0.54 mmol), 1,3-bisdiphenylphosphinopropane (48 mg, 0.12 mmol), and palladium acetate (21 mg, 0.09 mmol) were then added. The tube was tightly capped and the mixture was left to stir at 100° C. for 48 hours. After cooling to room temperature the mixture was filtered through a thin celite pad washing with DMF and the solvent was removed in vacuo. The crude was dissolved in 10 mL THF and 3 mL of 1N HCl was added. The reaction was diluted with NaHCO₃after 1 hour and extracted with EtOAc. The combined organic extracts were washed with brine, dried over Na₂SO₄, filtered, and concentrated to give a yellow oil. This material was chromatographed on silica gel using 80% EtOAc in hexanes as the eluant to give 1-(3,5-bis-pyrazol-1-ylmethyl-phenyl)-ethanone (19D) as a yellow oil.

Rf=0.31 (80% EtOAc/hexanes)

¹H NMR (400 MHz, CDCl₃) δ7.69(m, 2H), 7.56(m, 2H), 7.41(m,2H), 7.21(m, 1H), 6.30(m, 2H), 5.34(s, 4H), 2.52(s,3H).

1-(3,5-Bis-pyrazol-1-ylmethyl-phenyl)-3-(4-methyl-pyridin-2-yl)-propane-1,3-dione (19E)

NaOMe (42 mg, 0.78 mmol) was added to a dried flask under argon containing a solution of 1-(3,5-bis-pyrazol-1-ylmethyl-phenyl)-ethanone (19D) dissolved in 2.5 mL distilled THF. 5-methyl 4-methylpyridin-2-carboxylate (3H) (119 mg, 0.78 mmol) in THF was added and the reaction was stirred 1.5 hours. The reaction was quenched with water, the pH of the solution was adjusted to 4 using 1N HCl, and the solution was extracted with CHCl₃. The combined organic extracts were dried over Na₂SO₄, filtered, and concentrated to give a yellow oil. The oil was crystallized from Et₂O to give 1-(3,5-bis-pyrazol-1-ylmethyl-phenyl)-3-(4-methyl-pyridin-2-yl)-propane-1,3-dione (19E) as a yellow solid.

¹H NMR (400 MHz, CDCl₃) δ8.57(m, 1H), 7.99(m, 1H), 7.85(m, 2H), 7.69(m, 1H), 7.56(m, 2H), 7.49(s, 1H), 7.41(m, 2H), 7.16(m, 1H), 6.30(m, 2H), 5.36(s, 4H), 2.46(s, 3H).

| | | | |
|---|---|---|---|
| Anal. Calc'd for: | C, 68.44; | H, 5.41; | N, 16.56. |
| C₂₃H₂₁N₅O₂ 0.55 CH₃CN 0.05 H₂O | | | |
| Found: | C, 68.81; | H, 5.25; | N, 16.29. |

1-(4-Methyl-pyridin-2-yl)-3-(3-pyrazol-1-ylmethyl-phenyl)-propane-1,3-dione (19F)

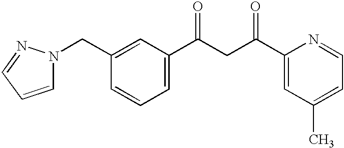

In a manner similar to that for 19E, except that 3-bromobenzyl bromide was used instead of 19B, 1-(4-Methyl-pyridin-2-yl)-3-(3-pyrazol-1-ylmethyl-phenyl)-propane-1,3-dione (19F) TFA salt was prepared.

| | | | |
|---|---|---|---|
| Anal. | C, 57.72; | H, 4.24; | N, 9.62. |
| Calc'd for: C₁₉H₁₇N₃O₂.TFA 0.20 H₂O | | | |
| Found: | C, 57.72; | H, 4.23; | N, 9.37. |

1-(4-Methyl-pyridin-2-yl)-3-(3-pyrrol-1-ylmethyl-phenyl)-propane-1,3-dione (19G)

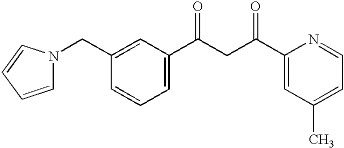

In a manner similar to that for 19E (except that in the second step NaH was used as the base in DMF), 1-(4-methyl-pyridin-2-yl)-3-(3-pyrrol-1-ylmethyl-phenyl)propane-1,3dione (19G) was prepared.

| | | | |
|---|---|---|---|
| Anal. Calc'd for: C₂₀H₁₈N₂O₂ 0.15 | C, 75.76; | H, 6.12; | N, 8.46. |
| hexanes | | | |
| Found: | C, 76.05; | H, 5.99; | N, 8.50. |

1-(4-Methyl-pyridin-2-yl)-3-(3-tetrazol-2-ylmethyl-phenyl)propane-1,3-dione (19H)

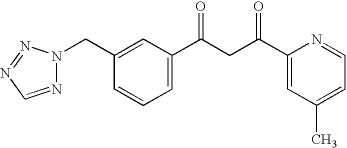

In a manner similar to that for 19E, 1-(4-methyl-pyridin-2yl)-3-(3-tetrazol-2-ylethyl-phenyl)-propane-1,3-dione (19H) was prepared.

| | | | |
|---|---|---|---|
| Anal. Calc'd for: | C, 56.20; | H, 4.25; | N, 18.21. |
| C₁₇H₁₅N₅O₂ 0.35 H₂O 0.50 TFA | | | |
| Found: | C, 56.22; | H, 4.27; | N, 18.05. |

1-(4-Methyl-pyridin-2-yl)-3-(3-[1,2,3]triazol-2-ylmethyl-phenyl)-propane-1,3-dione (19I)

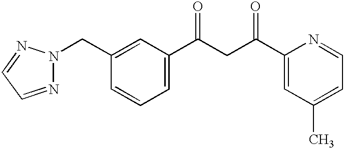

In a manner similar to that for 19E, 1-(4-methyl-pyridin-2yl)-3-(3-[1,2,3 ]triazol-2-ylmethyl-phenyl)-propane-1,3-dione (19I) was prepared.

| | | | |
|---|---|---|---|
| Anal. Calc'd | C, 64.61; | H, 4.87; | N, 16.47. |
| for: C₁₈H₁₆N₄O₂ 0.15 TFA 0.15 H₂O | | | |
| Found: | C, 64.69; | H, 4.82; | N, 16.29. |

1-[3-(3-Methyl-pyrazol-1-ylmethyl)-phenyl]-3-(4-methyl-pyridin-2-yl)-propane-1,3-dione and 1-[3-(5-Methyl-pyrazol-1-ylmethyl)-phenyl]-3-(4-methyl-pyridin-2-yl)-propane-1,3-dione (19J):

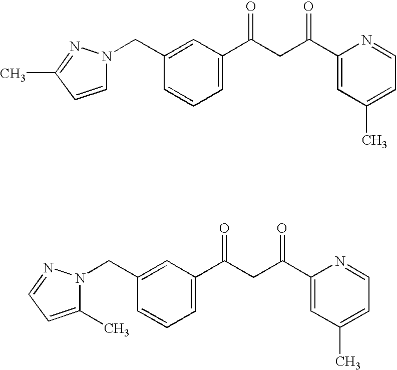

In a manner similar to that for 19E, 1-[3-(3-Methyl-pyrazol-1-ylmethyl)-phenyl]-3-(4-methyl-pyridin-2-yl)-propane-1,3-dione and 1-[3-(5-Methyl-pyrazol-1-ylmethyl)-phenyl]-3-(4-methyl-pyridin-2-yl)-propane-1,3-dione (19J) was prepared.

| | | | |
|---|---|---|---|
| Anal. Calc'd for: C₂₀H₁₉N₃O₂.TFA | C, 58.12; | H, 4.61; | N, 9.24. |
| Found: | C, 58.15; | H, 4.53; | N, 9.19. |

1-(4-Methyl-pyridin-2-yl)-3-(3-[1,2,3]triazol-2-ylmethyl-5-[1,2,3]triazol-1-ylmethyl phenyl)-propane-1,3-dione (19K)

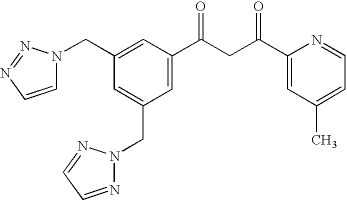

In a manner similar to that for 19E, 1-(4-methyl-pyridin-2-yl)-3-(3-[1,2,3]triazol-2-ylmethyl-5-[1,2,3]triazol-1-ylmethyl phenyl)-propane-1,3 dione (19K) was prepared.

| | | | |
|---|---|---|---|
| Anal. | C, 61.90; | H, 5.09; | N, 23.56. |
| Calc'd for: C₂₁H₁₉N₇O₂ 0.20 Et₂O | | | |
| Found: | C, 62.73; | H, 4.97; | N, 23.59 |

1-(3,5-Bis-pyrrol-1-ylmethyl-phenyl)-3-(4-methyl-pyridin-2-yl)-propane-1,3-dione (19L)

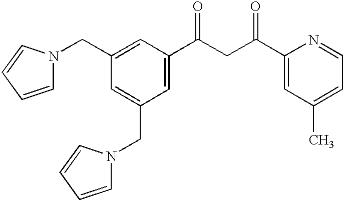

In a manner similar to that for 19E, 1-(3,5-bis-pyrrol-1-ylmethyl-phenyl)-3-(4-methyl-pyridin-2-yl)-propane-1,3-dione (19L) was prepared.

| | | | |
|---|---|---|---|
| Anal. Calc'd | C, 73.79; | H, 5.90; | N, 10.24. |
| for: C₂₅H₂₃N₃O₂ 0.05 Et₂O 0.50 H₂O | | | |
| Found: | C, 73.72; | H, 5.74; | N, 10.48. |

1-(3-Indazol-1-ylmethyl-phenyl)-3-(4-methyl-pyridin-2-yl)-propane-1,3-dione (19M)

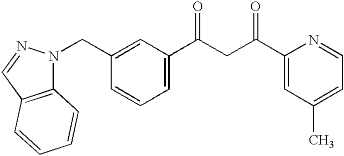

In a manner similar to that for 19E, 1-(3-indazol-1-ylmethyl-phenyl)-3-(4-methyl-pyridin-2-yl)-propane-1,3-dione (19M) was prepared.

| | | | |
|---|---|---|---|
| Anal. Calc'd for: C₂₃H₁₉N₃O₂ | C, 74.78; | H, 5.18; | N, 11.37. |
| Found: | C, 74.48; | H, 5.03; | N, 11.16. |

1-(3,5-Bis-pyrazol-1-ylmethylphenyl)-3-pyridin-2-yl-propane-1,3-dione (19O)

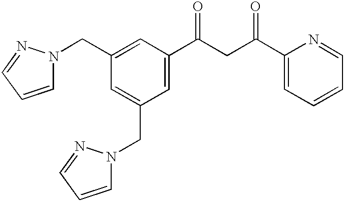

In a manner similar to that described for 19E [except that methyl picolinate (TCI-US) was used instead of 5-methyl 4-methylpydridine-2-carboxylate (3H)], 1-(3,5-bis-pyrazol-1-ylmethylphenyl)-3-pyridin-2-yl-propane-1,3-dione (19) was prepared.

¹H NMR (400 MHz, CDCl₃) δ8.72(m, 1H) 8.15(m, 1H), 7.85(m, 3H), 7.41-7.56(m, 6H), 7.17(m, 1H), 6.30(m, 2H), 5.36(s, 4H).

| Anal. Calc'd | C, 67.27; | H, 5.06; | N, 17.67. |
|---|---|---|---|
| for: C₂₂H₁₉N₅O₂ 0.05 Et₂O 0.40 H₂O | | | |
| Found: | C, 67.27; | H, 4.96; | N, 18.02. |
| Exact Mass: C₂₂H₁₉N₅O₂ | | | |
| Theor. Mass | 386.1611 | | |
| Meas. Mass | 386.1609 | | |

1-(4-Methylpyridin-2-yl)-3-(3-pyrazol-1-ylmethyl-5-[1,2,4]triazol-1-methylphenyl)-propane-1,3-dione (19P)

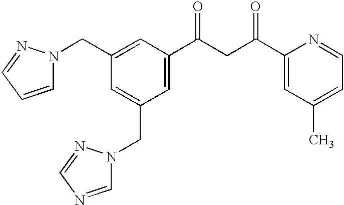

In a manner similar to that for 19E (except that in the second step a 1:1 molar ratio of pyrazole and 1,2,4-triazole replaced the amount of pyrazole used), 1-(4-methyl-pyridin-2-yl)-3-(3-pyrazol-1-ylmethyl-5-[1,2,4]triazol-1-ylmethylphenyl)-propane-1,3-dione (19P) was prepared.

¹H NMR (400 MHz CDCl₃) δ8.57(m, 1H), 8.10(m, 1H), 7.99(m, 2H), 7.90(m, 2H), 7.57(m, 1H), 7.50(m, 1H), 7.43(m, 1H), 7.22(m, 1H), 6.32(m, 1H), 5.38(s, 4H), 2.46(s, 3H).

| | | | |
|---|---|---|---|
| Anal. Calc'd for: | C, 64.12; | H, 5.20; | N, 20.03. |
| C₂₂H₂₀N₆O₂ 0.65 H₂O 0.10 Et₂O | | | |
| | C, 64.04; | H, 5.09; | N, 20.05. |

1-[3,5-Bis(3,5-dimethylpyrazol-1-ylmethyl)phenyl]-3-(4-methylpyridin-2-yl)-propane-1,3-dione (19Q)

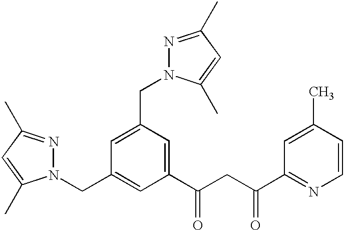

In a manner similar to that for 19E, 1-[3,5-bis(3,5-dimethylpyrazol-1-ylmethyl)phenyl]-3-(4-methylpyridin-2-yl)propane-1,3-dione (19Q) was prepared.

| | | | |
|---|---|---|---|
| Anal. Calc'd for C₂₇H₂₉N₅O₂.1.49 TFA | C, 54.18; | H, 4.61; | N, 10.19. |
| Found: | C, 54.18; | H, 4.48; | N, 10.33. |

1-(3,5-Bis-pyrazol-1-ylmethylphenyl)-3-pyrimidin-2-yl-propane-1,3-dione (19R)

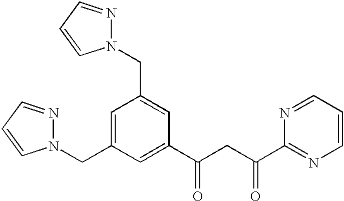

To a solution of 19D (0.35 g, 1.25 mmol) and 5L (0.173 g, 1.25 mmol) in THF (7 mL) was added sodium methoxide (0.135 g, 2.50 mmol). After stirring at room temperature under argon for one hour, the resultant dark yellow mixture was pored into a saturated solution of ammonium chloride. The mixture was extracted three times with ethyl acetate. The organic extract was dried over sodium sulfate, filtered and concentrated to give a dark brown sticky solid. This material was triturated with toluene and white solids precipitated. The solid was collected by filtration to give 19R.

¹H NMR (400 MHz, DMSO) δ9.07−9.05 (d×d, j=1.0 Hz, 4.9 Hz, 2 H), 7.87 (m, 4 H), 7.74-7.71 (t, j=4.9 Hz, 1H). 7.56 (s, 1H) 7.48 (m, 2H), 7.38 (s, 1H), 6.29−6.28 (t, j=2.1 Hz, 2H), 5.44 (s, 4H).

| | | | |
|---|---|---|---|
| Anal. Calc'd for C₂₁H₁₈N₆O₂ + | C, 64.37; | H, 4.79; | N, 21.45. |
| 0.30 water. | | | |
| Found: | C, 64.54; | H, 4.67; | N, 21.09. |
| ES MS M + 1 = 387.1560. | | | |

1-(3,5-Bis-pyrazol-1-ylmethylphenyl)-3-(4-methylpyridin-2-yl)propane-1,3-dione (19S)

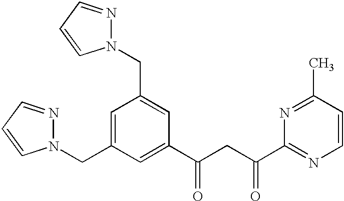

In a manner similar to the preparation of 19R, 19S was synthesized.

¹H NMR (400 MHz, CDCl₃) δ8.77−8.76 (d, J=5.0 Hz, 1 H), 7.83 (s, 2 H), 7.57 (d, J=1.8 Hz, 1H), 7.52 (s, 1H), 7.42 (d, J=2.2 Hz, 2H), 7.28−7.27 (m, 2H), 7.20 (s, 1H), 6.62−6.31 (t, J=2.1 Hz, 2H), 5.35 (s, 4H), 2.68 (s, 3H).

ES MS M+1=401.1718.

1-(3,5-Bis-pyrazol-1-ylmethyl-phenyl)-3-(1H-imidazol-2-yl)propane-1,3-dione (19T)

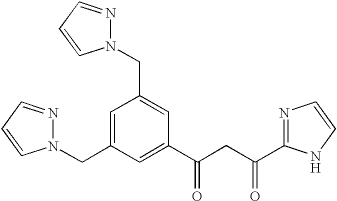

Ethyl 3,5-Bis-pyrazol-1-ylmethylbenzoate

To a solution of 1-[3-bromo-5-(1H-pyrazol-1-ylmethyl)benzyl]-1H-pyrazole (19C)(0.44 g, 1.39 mmol) in EtOH in a high pressure reaction vessel (40 mL) was added triethylamine (1 mL) and triphenylphosphine palladium chloride (0.487 g, 0.69 mmol). Following addition, the vessel was closed and filled with CO gas up to 100 p.s.i. The reaction was stirred for 3 days at 100° C., then it was cooled to room temperature and filtered through a a pad of celite. The celite pad was washed several times with ethyl acetate. The solution was concentrated under vacuum and the resultant residue was partitioned between water and EtOAc. The organic layer was then dried over sodium sulfate, filtered and concentrated. This was purified by flash chromatography on silica gel (hexane/ethyl acetate) to give a clear oil.

MS M+1=311.1.

3,5-Bis-pyrazol-1-ylmethylbenzoic acid

To a solution of ethyl 3,5-bis-pyrazol-1-ylmethylbenzoate (0.31 g, 1.00 mmol) in THF (15 mL) was added 1 N sodium hydroxide (3.01 mL). Methanol was added to make the mixture homogenous. After 3 hours the solution was acidified and the aqueous layer was extracted with ethyl acetate two times, the organic layer was dried over sodium sulfate, filtered and concentrated to give title acid as a pure white solid.

MS M+1=283.1

3,5-Bis-pyrazol-1-ylmethylbenzoyl chloride

A solution of 3,5-bis-pyrazol-1-ylmethylbenzoic acid (1.0 g, 3.54 mmol) in thionylchloride was stirred for three hours at which time the solvent was removed. This residue was concentrated from benzene three times to afford the acid chloride.

1H NMR (400 MHz, DMSO) δ7.85 (s, 2H), 7.66 (s, 2 H), 7.48 (s, 2 H), 7.36 (s, 1 H), 6.28 (s, 2 H), 5.38 (s, 4 H).

1-(2-Trimethylsilanyl-ethoxymethyl)-1H-imidazole

To a solution of imidazole (2.5 g, 36.72 mmol) in THF (150 ml) was added sodium hydride (2.5 g, 60% dispersion in mineral oil, 62.8 mmol) at 0° C. After stirring for 10 minutes [2-(chloromethoxy)ethyl](trimethyl)silane (9.24 g, 9.81 mL, 55.45 mmol) was added to the mixture. The suspension was stirred for four hours. The mixture was poured into water and extracted with diethyl ether. The ethereal extract was dried over sodium sulfate, filtered, and concentrated under vacuum. Flash chromatography on silica gel with 3% methanol/chlorform afforded title compound as yellow oil.

1H NMR (400 MHz, CDCl₃) δ7.61 (s, 1H), 7.12 (s, 1 H), 7.06 (m, 1 H), 5.29 (s, 2 H), 3.51−3.47 (t, j=8.2 Hz, 2 H), 0.94−0.90 (t, 8.2 Hz, 2 H), 0.00 (s, 9H).

1-[1-(2-Trimethylsilanylethoxymethyl)-1H-imidazol-2-yl]ethanone

To a cold (−78° C.) solution of 1-(2-trimethylsilanyl-ethoxymethyl)-1H-imidazole (2.2 g, 11.09 mmol) in THF (75 ml) under argon, n-butyl lithium (5.77 ml, of 2.5 M in hexanes) dropwise. N-Methoxy-N-methylacetamide (1.25 g, 12.2 mmol) was then added dropwise after the reaction had aged for one hour. The reaction was then allowed to warm to room temperature. The solution was poured into a aqueous solution of ammonium chloride and extracted with ethyl acetate two times. The organic extract was combined, dried over sodium sulfate, filtered, and concentrated. This residue was flash chromatographed on silica gel eluting with ethyl acetate/hexanes to give title ketone.

1H NMR (400 MHz, CDCl₃) δ7.32 (d, j=0.9 Hz, 1H), 7.22 (d, j=0.9 Hz 1 H), 5.8 (s, 2 H), 3.61−3.57 (t, j=7.6 Hz, 2 H), 2.70 (s, 3 H) 0.97−0.93 (t, j=7.6 Hz, 2 H), 0.00(s, 9H).

1-[3,5-Bis(1H-pyrazol-1-ylmethyl)phenyl]-3-(1-{[2-(trimethylsilyl)ethoxy]methyl}-1H-imidazol-2-yl)-1,3-propanedione

To a solution of 1-[1-(2-trimethylsilanyl-ethoxymethyl)-1H-imidazol-2-yl]ethanone (0.526 g, 1.75 mmol) in THF (10 mL) under argon was added dropwise a solution of potassium tert-butoxide (2.2 mL, 1M). After 10 minutes of stirring at 0° C., a solution of 3,5-bis-pyrazol-1-ylmethyl-benzoyl chloride in THF was added dropwise and the reaction was allowed to warm to room temperature and concentrated under vacuum. The residue was purified by reverse phase HPLC. Collection and lyophilization of appropriate fractions afforded title compound as a brown oil.

1H NMR (400 MHz, CDCl₃) δ11.97 (b, 1H), 7.93 (s, 2 H), 7.62−7.61 (m, 2 H), 7.54 (s, 2 H), 7.50 (s, 1 H) 7.43 (s, 1 H), 7.25 (s, 2H), 6.34 (d, j=1.8 Hz, 2H), 5.96 (s, 2H), 5.41 (s, 4H), 3.70−3.66 (t, j=8.3 Hz, 2H), 1.02−0.98 (t, j=8.2 Hz, 2 H), 0.01 (s, 9H).

1-(3,5-Bis-pyrazol-1-ylmethylphenyl)-3-(1H-imidazol-2-yl)propane-1,3-dione (19T)

A solution of 1-[3,5-bis(1H-pyrazol-1-ylmethyl)phenyl]-3-(1-{[2-(trimethylsilyl)ethoxy]methyl }-1H-imidazol-2-yl)-1,3-propanedione (10 mg, 0.02 mmol) in 8:1 acetic acid/water (0.4 ml) was heated under reflux for two hours. The solvent was removed under reduced pressure. The residue was dissolved in DMSO and purified by reverse phase HPLC.

1H NMR (400 MHz, CDCl₃) δ7.94 (s, 2H), 7.58−7.54 (m, 3 H), 7.42−7.37 (m, 3 H), 7.23−7.14 (m, 2 H), 6.29 (s, 2 H) 5.32 (s, 4H).

1-(3,5-Bis-pyrazol-1-ylmethyl-phenyl)-3-(1-methyl-1H-imidazol-4-yl)propane-1,3-dione (19U)

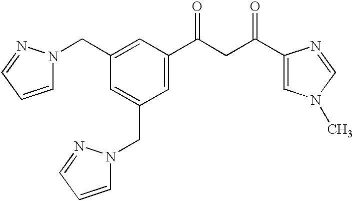

In a manner similar to that described for 19E [except that methyl 1-methyl-1H-imidazole-4-carboxylate was used instead of 5-methyl 4-methylpyridin-2-carboxylate (3H)], 1-(3,5-bis-pyrazol-1-ylmethylphenyl)-3-(1-methyl-1H-imidazol-4-yl)-propane-1,3-dione (19U) was prepared.

1H NMR (400 MHz, CDCl₃) δ8.11 (s, 1H), 7.99 (s, 1H), 7.86 (d, 2H), 7.76 (d, 2H), 7.47 (d, 2H), 7.31 (s, 1H), 7.02 (s, 1H), 6.28 (br s, 2H), 5.41 (s, 4H), 3.76 (s, 3H).

### EXAMPLE 20

1-(4-Methyl-pyridin-2-yl)-3-(3-pyrimidin-2-ylmethyl-phenyl)-propane-1,3-dione (TFA salt) (20C)

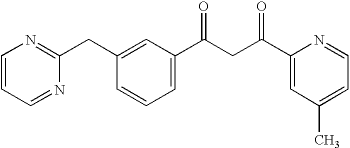

2-(3-Bromo-benzyl)-pyrimidine (20A)

3-bromobenzyl bromide (1 g, 0.00402 mole) was added to a mixture of ZnCu couple (0.39 g, 0.00603 mole) and N,N-dimethylacetamide (0.75 mL) in 10 mL of toluene under argon. This was heated at 75° C. for 2.5 hours followed by cooling to room temperature and the addition of dichloro-bis(triphenylphosphine)palladium (0.14 g 0.000201 mole) and 2-bromopyrimidine (0.43 g 0.0027 mole) in 1.5 mL of toluene. After stirring for two hours the reaction was poured into 10 mL of water and extracted 3 times with EtOAc. The combined organic layers were dried with NaSO₄, and filtration and concentration gave crude material. Flash chromatography starting with 20% EtOAc/hexanes and eluting pure compound with 50% EtOAc/hexanes, afforded (20A).

Rf=0.27 (40% EtOAc/Hexanes)

¹H NMR (400 MHz, CDCl₃) δ8.69 (d, j=4.8 Hz, 2H), 7.51 (s, 1H), 7.35 (d, j=7.9 Hz 1H), 7.28 (t, j=7.0 Hz, 1H), 7.19−7.13 (m, 2H), 4.26 (s, 2H).

1-(3-Pyrimidin-2-ylmethyl-phenyl)-ethanone (20B)

To a pressure tube containing 4 mL of DMF with bubbling argon was added 20A (0.75 g, 0.00301 mole), triethylamine (1.68 mL, 0.0120 mole), butyl vinyl ether (1.95 mL, 0.0151 mole), Tl(OAc)₂ (0.87 g, 0.00331 mole), 1,3-bisdiphenylphosphino)-propane (0.33 g, 0.000812 mole), and Pd(OAc)₂. After stirring at 100° C. for 2 days the reaction was allowed to cool and filtered through a pad of celite which was washed several times with EtOAc. The solvents were removed and the black residue was treated with 40 mL of THF and 15 mL of 1 N HCl. After two hours the solution was neutralized with a solution of NaCO₃ and extracted three times with EtOAc, dried over NaSO₄, filtered and evaporated to give a crude brown oil which was taken on as is (20B). NMR is contaminated with DPPP (extra aromatic protons).

Rf=0.15 (20% EtOAc/Hexanes)

¹H NMR (400 MHz, CDCl₃) δ8.68 (d, j=5.1 Hz, 2H), 7.96 (s, 1H), 7.83 (d, j=7.8 Hz 1H), 7.72−7.64 (m, 1H), 7.49−7.39 (m, 4H), 7.15 (t, j=4.9 Hz, 1H), 4.36 (s, 2H), 2.59 (s, 3H).

1-(4-Methyl-pyridin-2-yl)-3-(3-pyrimidin-2-ylmethyl-phenyl)-propane-1,3-dione (tfa salt) (20C)

In an oven dried flask equipped with a rubber septum and with bubbling argon was dissolved 20B (0.5 g, 0.0024 mole) and 3H (0.36 g, 0.0024 mole) in 1 mL of anhydrous THF. NaOMe (0.25 g, 0.0046 mole) was added and the reaction turned a brown color. After 0.5 hour the reaction was poured in 30 mL of a saturated NH₄Cl solution. The mixture was extracted 3 times with EtOAc, dried over NaSO₄, filtered and evaporated solvent to give a crude product. The oil was purified by HPLC. The solvent was removed from desired fractions to give a yellow oil. The oil was crystallized with Et₂O and hexanes and a couple drops of CH₂Cl₂ to give a yellow powder (20C).

Exact mass calculated=331.1321, found=331.1320.

| | | | |
|---|---|---|---|
| Analysis Calc'd for: | C, 53.16; | H, 3.23; | N, 7.92. |
| C₂₆H₂₁N₅O₂ + add'l 1.75 TFA. | | | |
| Found: | C, 53.11; | H, 3.32; | N, 7.63. |

### EXAMPLE 21

1-(3-Benzylphenyl)-3-(5-dimethylaminopyridin-2-yl)-propane-1,3-dione (21E)

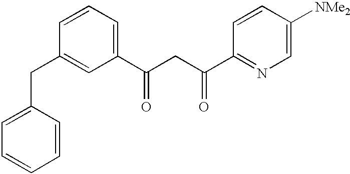

5-Dimethylaminopyridine-2-carboxylic acid (21B)

A solution of 5-bromopyridine-2-carboxylic acid (1 g, 4.9 mmol) in 40% dimethylamine in water (20 mL) was heated in a pressure vessel at 160° C. for 5 days. The solution was concentrated under vacuum. The residue was dissolved in a mixture of methanol (20 mL) and diisopropylethylamine (10 mL), and concentrated under vacuum. The latter procedure was repeated two times, and the resultant residue dried under vacuum overnight. The resultant diisopropylethylamine salt was taken on to the next step without further purification.

N-Methyl-N-methoxy-5-dimethylaminopyridine-2-carboxyamide (21C)

A mixture of the diisopropylethylamine salt of 21B (4.9 mmol), ethyl-3-(3-dimethylamino)-propyl carbodiimide.HCl (1.15 g, 6 mmol), 1-hydroxy-7-azabenzotriazole (0.68 g, 5 mmol) was dissolved in dimethylformamide (10 mL). The mixture was neutralized by addition of diisopropylethylamine, and the resultant mixture was stirred at ambient temperature for two weeks. The mixture was concentrated under vacuum. The residue was subjected chromatography on silica gel using 3% methanol in chloroform as eluant to give 21 C.

¹H NMR (400 MHz, CDCl₃) δ8.09 (br s, 1H), 7.73 (br d, 1H), 6.98 (br d, 1H), 3.80 (s, 3H), 3.47 (s, 3H), 3.06 (s, 6H).

2-Acetyl-5-dimethylaminopyridine (21D)

To a cold (−78 C.) solution of 21C (0.55 g, 2.6 mmol) in THF (10 mL) under an atmosphere of dry argon, a solution of methylmagnesium bromide (1.3 ml, 3 M, 3.9 mmol) in THF was added. The solution was allowed to warm to room temperature, and stirred at room temperature overnight. The resultant mixture was cooled to 0 C., and treated with 1 M dilute HCl. The mixture was dilute with ethyl acetate. The organic extract was washed with brine, dried over magnesium sulfate, filtered and concentrated under vacuum. The residue was subjected to column chromatography on silica gel eluted with 3% methanol in chloroform. Collection and concentration of appropriate fractions provided 21D as a crystalline solid.

¹H NMR (400 MHz, CDCl₃) δ8.10 (d, J=3.1 Hz, 1H), 7.95(d, J=9 Hz, 1H), 6.95 (dd, J=9, 3.1 Hz, 1H), 3.09 (s, 6H), 2.64 (s, 3H).

1-(3-Benzylphenyl)-3-(5-dimethylaminopyridin-2-yl)-propane-1,3-dione (21E)

To a cold (0 C.) solution of 21D (0.12 g, 0.72 mmol) and methyl 3-benzylbenzoate (0.24 g, 1 mmol) in THF (2.5 mL) under an atmosphere of dry argon, a solution of sodium bis(trimethylsilyl)amide (1.9 ml, 1 M, 1.9 mmol) in THF was added. The solution was allowed to warm to room temperature, and stirred at room temperature overnight. The resultant mixture was cooled to 0 C., and neutralized with trifluoroacetic acid. The mixture was concentrated under vacuum. The residue was triturated with methanol. Filtration and drying under vacuum provided 21E as yellow solid.

¹H NMR (400 MHz, CDCl₃) δ8.17 (d, J=3.1 Hz, 1H), 8.05 (d, J=9 Hz, 1H), 7.91 (s, 1H), 7.88 (d, J=7.7 Hz, 1H), 7.5−7.1 (m, 8H), 7.00 (dd, J=9, 3.1 Hz, 1H), 4.05 (s, 2 H), 3.11 (s, 6H).

1 -(3-Benzylphenyl)-3-(5-bromopyridin-2-yl)propane-1,3-dione (21F)

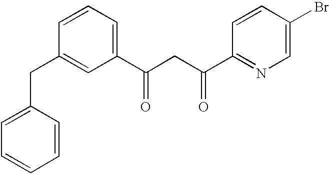

In a manner similar to that for 7B, 1-(3-benzylphenyl)-3-(5-bromopyridin-2-yl)propane-1,3-dione (21F) was prepared.

| | | | |
|---|---|---|---|
| Anal. Calc'd for C₂₁H₁₆BrNO₂ | C, 63.97; | H, 4.09; | N, 3.56. |
| Found: | C, 63.76; | H, 4.16; | N, 3.36. |

1-(3-Benzylphenyl)-3-(5-methoxypyridin-2-yl)propane-1,3-dione (21G)

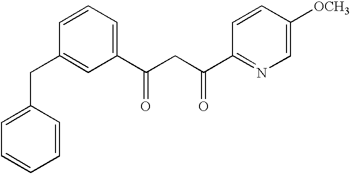

In a manner similar to that for 7B, 1-(3-benzylphenyl)-3-(5-methoxypyridin-2-yl)propane-1,3-dione (21G) was prepared.

| | | | |
|---|---|---|---|
| Anal. Calc'd for C₂₂H₁₉NO₃ | C, 76.50; | H, 5.54; | N, 4.06. |
| Found: | C, 76.35; | H, 5.26; | N, 3.98. |

### EXAMPLE 22

1-(1H-Imidazol-2-yl)-3-(5-phenethylthiophen-2-yl)propane-1,3-dione 22E

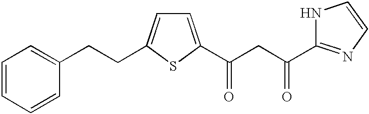

1-(5-Phenethyl-thiophen-2-yl)ethanone (22B)

A mixture of 1-(5-phenylethynylthiophen-2-yl)ethanone (22A) (850 mg, 3.76 mmol) and 5% Pd on C (817 mg ) in 20 mL of ethanol was stiffed under an atmosphere of hydrogen gas (1 atm) at room temperature. After 4 hours, the reaction was filtered through Celite and concentrated in vacuo to afford 1-(5-phenethylthiophen-2-yl)ethanone (22B) as a clear colorless oil.

¹H NMR (400 MHz, CDCl₃) δ7.5 (d, J=3.8, 1H), 7.25 (m, 5H), 6.8 (dd, J=3.8, 0.9 Hz, 1H), 3.2 (t, J=7.8 Hz, 2H), 3.0 (t, J=7.8 Hz, 2H), 2.5 (d, J=0.9 Hz, 3H)

3-Hydroxy-1-(5-phenethylthiophen-2-yl)-3-(1-trityl-1H-imidazol-2-yl)propan-1-one (22C)

To an oven-dried flask under an argon atmosphere was added 1-(5-phenethylthiophen-2-yl)ethanone (714 mg, 3.10 mmol) and 12 mL of anhydrous THF. The solution was cooled to −78 C. and LDA (1.75 mL of a 2.0M solution in heptane/THF/ethylbenzene, 3.50 mmol) was added dropwise. This was stirred at −78 C. for 60 minutes, followed by addition of 1-trityl-1H-imidazole-2-carbaldehyde (1.05 g, 3.11 mmol) in 20 mL THF. The reaction was warmed to room temperature over 2.5 hours and then treated with 1N HCl to obtain a pH of 9, and extracted with ethyl acetate three times. The combined organic layers were washed with brine and concentrated in vacuo. This material was chromatographed on silica gel using 1% MeOH/CHCl₃-5% MeOH/CHCl₃ as eluant to afford 3-hydroxy-1-(5-phenethylthiophen-2-yl)-3-(1-trityl-1H-imidazol-2-yl)propan-1-one (22C).

3-Hydroxy-3-(1H-imidazol-2-yl)-1-(5-phenethylthiophen-2-yl)propan-1-one (22 D)

To 3-hydroxy-1-(5-phenethylthiophen-2-yl)-3-(1trityl-1H-imidazol-2-yl)propan-1-one (0.60 g, 1.05 mmol) was added 8 mL of TFA followed by triethylsilane (0.185 mL, 1.16 mmol). The reaction was stirred at room temperature one hour and then concentrated in vacuo. It was partitioned between CH₂Cl₂ and NaHCO₃ (aq) and extracted three times with CH₂Cl₂. The combined organic layers were washed with brine, dried over Na₂SO₄, filtered and concentrated in vacuo. Recrystallizing in CH₂Cl₂/hexanes afforded 3-hydroxy-3-(1H-imidazol-2-yl)-1-(5-phenethylthiophen-2-yl)propan-1-one (22D).

¹H NMR (400 MHz, CDCl₃) δ7.7−6.8 (m, 9H), 5.4 (m, 1H), 3.7 (dd, J=17.6, 2.75 Hz, 1H), 3.4 (dd, J=17.6, 7.75 Hz, 1H), 3.2 (m, 2H), 3.0 (m, 2H).

1-(1H-Imidazol-2-yl)-3-(5-phenethylthiophen-2-yl)propane-1,3-dione (22E)

To a solution of 3-hydroxy-3-(1H-imidazol-2-yl)-1-(5-phenethyl-thiophen-2-yl)propan-1-one (140 mg, 0.37 mmol) in 8 mL CHCl₃ was added MnO₂ (540 mg, 6.21 mmol) and stirred 5 hours at room temperature. After which, more MnO₂ (250 mg, 2.88 mmol) was added and stirred another 30 minutes till no starting material remained. The reaction was filtered through Celite and concentrated in vacuo. It was purified by preparative HPLC on C18 reverse stationary phase eluted with a water/acetonitrile/TFA mobile phase. Lyophilization afforded 1-(1H-Imidazol-2-yl)-3-(5-phenethyl-thiophen-2-yl)propane-1,3-dione (22E).

| | | | |
|---|---|---|---|
| Anal. Calc'd for: C₁₈H₁₆N₂O₂S | C, 54.80; | H, 3.92; | N, 6.52. |
| 1.00 TFA and 0.05 CH₃CN: | | | |
| Found: | C, 54.80; | H, 4.14; | N, 6.58. |

¹H NMR (400 MHz, DMSO-D₆) δ7.8 (m, 1H), 7.5 (s, 1H), 7.3 (m, 6H), 7.0 (m, 1H), 6.9 (br s, 1H), 4.6 (s, 1H), 3.2 (m, 2H), 3.0 (m, 2H)

### EXAMPLE 23

1-(5-Benzyl-thiophen-2-yl)-3-pyridin-2-ylpropane-1,3-dione (23D)

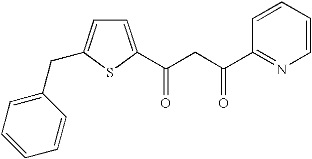

2-Benzyl-5-bromo-thiophene (23B)

To an oven-dried flask under an argon atmosphere was added n-butyl lithium (20.8 mL of a 2.5M hexane solution, 52.0 mmol) and 100 mL of anhydrous diethyl ether. This was cooled to −78 C. and 2,5-dibromothiophene (5.63 mL, 50.0 mmol) was added over 30 minutes. Stirred 90 minutes at −78 C., and then added benzaldehyde (5.30 mL, 52.0 mmol) over 15 minutes. The reaction was warmed to room temperature over 2.5 hours and then treated with 1N HCl to obtain a pH of 4, and extracted with diethyl ether three times. The combined organic layers were washed with NaHCO₃ (aqueous), and brine, dried over MgSO₄, filtered and concentrated in vacuo.

The crude oil (3.09 g, 11.5 mmol) was dissolved in CH₂Cl₂, cooled in an ice bath, and treated with triethylsilane (2.56 mL, 16.1 mmol) and BF₃OEt₂ (2.03 mL, 16.1 mmol) and stirred 2.5 hours at room temperature. After which, NaHCO₃ (aq) was added until the pH was 9, and extracted three times with CHCl₃. The combined organic layers were washed with brine, dried over MgSO₄, filtered and concentrated in vacuo. This material was chromatographed on silica gel using hexanes as eluant to afford 2-benzyl-5-bromothiophene (3B) as a clear oil.

¹H NMR (400 MHz, CDCl₃) δ7.3 (m, 5H), 6.85 (m, 1H), 6.55 (m, 1H), 4.0 (s, 2H)

1-(5-Benzylthiophen-2-yl)ethanone (23C)

To an oven-dried flask under an argon atmosphere was 2-benzyl-5-bromothiophene (2.99 g, 11.8 mmol) and 35 mL of anhydrous diethyl ether. The solution was cooled to −78 C. and n-butyl lithium (7.5 mL of a 1.6M hexane solution, 12.0 mmol) was added dropwise. This was stirred 60 minutes at −78 C., followed by addition of N-methoxy-N-methylacetamide (1.45 mL, 14.3 mmol). The reaction was warmed to room temperature overnight and then treated with 1N HCl to obtain a pH of 7, and extracted with diethyl ether three times. The combined organic layers were washed with brine, dried over MgSO₄, filtered and concentrated in vacuo. This material was chromatographed on silica gel using 10% EtOAc/hexanes as eluant to afford 1-(5-benzyl-thiophen-2-yl)ethanone (23C) as a clear oil.

¹H NMR (400 MHz, CDCl₃) δ7.5 (d, J=3.7 Hz, 1H), 7.3 (m, 5H), 6.8 (dt, J=3.7, 0.9 Hz, 1H), 4.15 (s, 2H), 2.5 (s, 3H)

1-(5-Benzyl-thiophen-2-yl)-3-pyridin-2-yl-propane-1,3-dione (23D)

To an oven-dried flask under an argon atmosphere was added 1-(5-benzylthiophen-2-yl)ethanone (217 mg, 1.00 mmol) and 7 mL of anhydrous THF. The solution was cooled to −78 C. and LDA (0.55 mL of a 2.0M solution in heptane/THF/ethylbenzene, 1.1 mmol) was added dropwise. This was stirred at −78 C. for 35 minutes, followed by addition of pyridine-2-carboxylic acid methoxy-methyl-amide (200 mg, 1.20 mmol). The reaction was warmed to room temperature overnight and then treated with 1N HCl to obtain a pH of 8, concentrated in vacuo, and extracted with CH₂Cl₂ three times. The combined organic layers were washed with brine, dried over Na₂SO₄, filtered and concentrated in vacuo. The crude compound was dissolved in diethyl ether, filtered through glass wool and precipitated out with hexanes. The yellow solid was filtered to provide 1-(5-benzylthiophen-2-yl)-3-pyridin-2-yl-propane-1,3-dione (23D).

| | | | |
|---|---|---|---|
| Anal. Calc'd for: C₁₉H₁₅N₁O₂S 0.05 | C, 70.93; | H, 4.81; | N, 4.31. |
| Et₂O: | | | |
| Found: | C, 70.91; | H, 4.66; | N, 4.22. |
| FAB MS found [M + 1] = 322 m/z | | | |

1-(5-Benzylthiophen-2-yl)3-(4-methyl-pyridin-2-yl)propane-1,3-dione (23E)

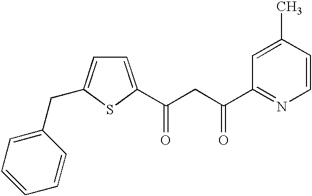

In a manner similar to that for 23D, 1-(5-benzyl-thiophen 2-yl)-3-(4-methyl-pyridin-2-yl)propane-1,3-dione (23E) was prepared.

| | | | |
|---|---|---|---|
| Anal. Calc'd for: | C, 71.35; | H, 5.22; | N, 4.12. |
| C₂₀H₁₇N₁O₂S 0.05 Et₂O and | | | |
| 0.05 water: | | | |
| Found: | C, 71.31; | H, 5.27; | N, 4.26. |

1-[5-(3-Chlorobenzyl)thiophen-2-yl]-3-pyridin-2-yl-propane-1,3-dione (23F)

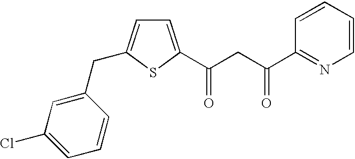

In a manner similar to that for 23D, 1-[5-(3-chlorobenzyl)thiophen-2-yl]3-pyridin-2-yl-propane-1,3-dione (23F) was prepared.

| | | | |
|---|---|---|---|
| Anal. Calc'd for: | C, 63.97; | H, 4.08; | N, 3.89. |
| C₁₉H₁₄CINO₂S 0.05 Et₂O and 0.05 water: | | | |
| Found: | C, 63.98; | H, 4.09; | N, 3.80 |

### EXAMPLE 24

1-5-(4Fluorobenzyloxy)thiophen-2-yl]-3-(4-methylpyridin-2-yl)-propane-1,3-dione (24C)

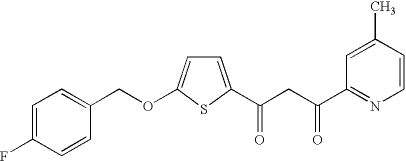

1-[5-(4-Fluorobenzyloxy)thiophen-2-yl]ethanone (24B)

To an oven-dried flask under an argon atmosphere was added sodium hydride (530 mg, 22.1 mmol) and 30 mL of DMSO and heated at 60 C. for one hour. The mixture was cooled back to room temperature and treated with 4-fluorobenzyl alcohol (2.40 mL, 22.0 mmol), followed by addition of 1-(5-chlorothiophen-2-yl)ethanone. (3.22 g, 20.0 mmol). The reaction was heated overnight at 80 C. The product mixture was partitioned between KHSO₄ and CH₂Cl₂. The combined organic extracts were washed with water (twice), brine, dried over MgSO₄, filtered and concentrated in vacuo. This residue was chromatographed on silica gel using 1% MeOH/CHCl₃ as eluant to afford 1-[5-(4-fluorobenzyloxy)thiophen-2-yl]ethanone (24B).

¹H NMR (400 MHz, CDCl₃) δ7.4 (m, 3H), 7.1 (t, J=8.6 Hz, 2H), 6.3 (d, J=4.3 Hz, 1H), 5.1 (s, 2H), 2.45 (s, 3H)

1-[5-(4-Fluorobenzyloxy)thiophen-2-yl]-3-(4-methylpyridin-2-yl)propane-1,3-dione (24C)

To an oven-dried flask under an argon atmosphere was added 1-[5-(4-fluorobenzyloxy)thiophen-2-yl]ethanone (247 mg, 1.00 mmol) and 7 mL of anhydrous THF. The solution was cooled to −78 C. and LDA (0.55 mL of a 2.0M solution in heptane/THF/ethylbenzene, 1.1 mmol) was added dropwise. This was stirred at −78 C. for 35 minutes, followed by addition of 4-methyl-pyridine-2-carboxylic acid methoxy-methyl-amide (216 mg, 1.20 mmol). The reaction was warmed to room temperature overnight, treated with 1N HCl to obtain a pH of 8, concentrated in vacuo, and extracted with CH₂Cl₂ three times. The combined organic layers were washed with brine, dried over Na₂SO₄, filtered and concentrated in vacuo. The crude compound was dissolved in diethyl ether, filtered through glass wool and precipitated out with hexanes. Filtration of the precipitate provided 1-[5-(4-fluorobenzyloxy)thiophen-2-yl]-(3-(4-methyl-pyridin-2-yl)propan-1,3-dione (24C) as yellow solid.

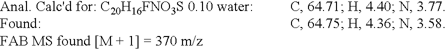

1-[5-(4-Fluorobenzyloxy)thiophen-2yl]-3-pyridin-2-yl-propane-1,3-dione (24D)

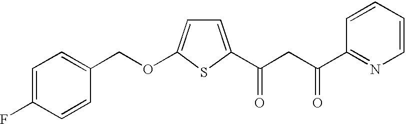

In a manner similar to that for 24C, 1-[5-(4fluorobenzyloxy)thiophen-2-yl]-3-pyridin-2-yl-propane-1,3-dione (24D) was prepared.

| | | | |
|---|---|---|---|
| Anal. Calc'd for: | C, 63.90; | H, 4.20; | N, 3.84. |
| C₁₉H₁₄FNO₃S 0.10 Et₂O and 0.10 water | | | |
| Found: | C, 63.93; | H, 4.01; | N, 3.47 |

### EXAMPLE 25

1-(3-Benzyl-5-pyrazin-2-yl phenyl)-3-(4-methylpyridin-2-yl)-propane-1,3-dione (25B)

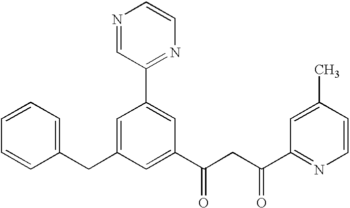

2-(3-Benzyl-5-bromo-phenyl)-pyrazine (25A)

Step 1: (3,5-Dibromo-phenyl)-phenyl-methanol (25a1)

To a solution of 1,3,5-tribromobenzene (10 g, 0.0318 mole) in ether (500 g), under argon was added nBuLi in hexanes (13.4 mL, 0.0318 mole) dropwise at −78° C. During the initial cooling of the tribromobenzene, in ether some solids crashed out of solution. After addition of nBuLi was complete the reaction was allowed to stir for 0.5 hours at which time neat benzaldehyde (3.55 mL, 0.035 mole) was added dropwise to the vigorously stirred reaction mixture. Once addition was complete the reaction was allowed to reach 0° C. and 100 mL of HCl was added to the mixture. This was extracted with ether two times, dried with brine and over sodium sulfate and concentrated to give an oil. The crude product was purified by chromatography with 5% EtOAc/Hexanes to afford 25a1 as a colorless oil that solidified on the bench. Rf=0.44 (5% EtOAc/Hexanes) 1H NMR (400 MHz, CDCl3 ) δ7.56−7.55 (m, 1H), 7.48−7.47 (m, 2H), 7.39−7.29 (m, 5H), 5.75 (d, 1H, j=3.48 Hz), 2.28−2.27 (d, 1H, j=3.48 Hz).

Step 2: 3-Bromo-5-benzyl-bromobenzene (25a2)

A solution of 25a1 (2.0 g, 0.00548 mole) and triethylsilane (1.39 mL, 0.00877 mole) in methylene chloride (20 mL) was chilled to 0° C. under argon with stirring followed by addition of boron trifluoride etherate (1.10 mL, 0.00877 mole). The reaction was stirred at room temperature overnight. The reaction mixture was poured into 75 mL of saturated sodium bicarbonate and extracted with methylene chloride two times. The combined organic layers were dried over sodium sulfate, filtered and the solvent removed. Chromatographic purification using 1% EtOAc/Hexanes afforded 25a2. Rf=0.72 (5% EtOAc/hexanes) 1H NMR (400 MHz, CDCl3 ) δ7.5 (s, 1H), 7.37−7.21 (m, 5H), 7.16−7.14 (m, 2H), 3.91 (s, 2H).

Step 3: 2-(3-Benzyl-5-bromo-phenyl)-pyrazine (25A)

To a solution of 25a2 (1 g, 0.00307 mole) in THF (20 mL) under argon at −78° C. was added 2.4M nBuLi in hexanes (1.4 mL, 0.00337 mole) dropwise. After, 45 minutes of stirring, the solution was treated with 0.5 m ZnCl₂ in THF (6.14 mL 0.00337 mole) and this was warmed to 0° C. To the reaction was added a cold mixture of chloropyrazine (0.35 mL, 0.00307 mole) and tetrakistriphenylphosphine palladium (18 mg, 0.0000154 mole) in THF (5 mL) and the reaction was heated to reflux for two hours. The reaction was then cooled, concentrated and treated with EtOAc and washed with 6% aqueous solution of diaminotetraacetic acid disodium salt dihydrate two times. The EtOAc layer was dried over sodium sulfate, filtered and concentrated. Chromatographic purification with 15% EtOAc/hexanes afforded a clear oil 25A. Rf=0.17 (20% EtOAc/hexanes) 1H NMR (400 MHz, CDCl₃) δ8.95 (s, 1H), 8.62−8.61 (m, 1H), 8.52 (m, 1H), 8.02 (s, 1H), 7.77 (s, 1H), 7.43 (s, 1H), 7.32−7.20 (m, 5H), 4.04 (s, 2H).

1-(3-Benzyl-5-pyrazin-2-yl-phenyl)-3-(4-methyl-pyridin-2-yl)-propane-1,3-dione (25B)

In an oven dried flask equipped with a rubber septum with bubbling argon was dissolved 25A (0.15 g, 0.00052 mole) and 3H (0.078 g, 0.00052 mole) in 1 mL of anhydrous THF. NaOMe (56 mg, 0.00104 mole) was added and the reaction turned a brown color. After 0.5 hour the reaction was poured into 8 mL of a saturated NH₄Cl solution. The mixture was extracted 3 times with EtOAc, dried NaSO₄, filtered and evaporated to give a crude product. The oily solids were crystallized with and hot petroleum ether in a sonicator. Solids were collected by vacuum filtration to give pure light yellow powder, (25B).

¹H NMR (400 MHz, CDCl₃) δ9.05 (s, 1H), 8.65 (s, 1H), 8.58 (d, j=5.0 Hz, 1H), 8.4 (m, 2H), 8.04 (s, 2H), 8.02 (s, 1H), 7.62 (s, 1H), 7.33−7.21 (m, 6H), 4.17 (s, 2H), 2.47 (s, 3H).

| | | | |
|---|---|---|---|
| Anal. Calc'd for: | C, 75.83; | H, 5.50; | N, 9.56. |
| C₂₆H₂₁N₅O₂ + add'l 0.50 water | | | |
| and 0.25 toluene | | | |
| Found: | C, 75.88; | H, 5.73; | N, 9.61. |

1-(3-Benzyl-5-pyrimidin-2-yl-phenyl)-3-(4-methyl-pyridin-2-yl)-propane-1,3-dione (25 C)

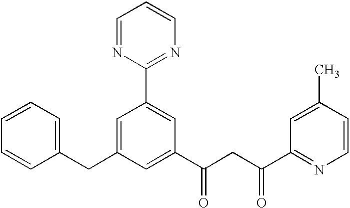

In a manner similar to that for 25B, 1-(3-benzyl-5-pyrazin-2-yl-phenyl)-3-(4-methyl-pyridin-2-yl)-propane-1,3-dione (25C) was prepared.

| | | | |
|---|---|---|---|
| Anal. Calc'd for: C₂₆H₂₁N₅O₂ | C, 76.64; | H, 5.19; | N, 10.31. |
| Found: | C, 76.48; | H, 4.73; | N, 10.03. |

### EXAMPLE 26

1-(3-Benzylphenyl)-3-(4-imidazol-1-ylmethylpyridin-2-yl)-propane-1,3-dione (26H)

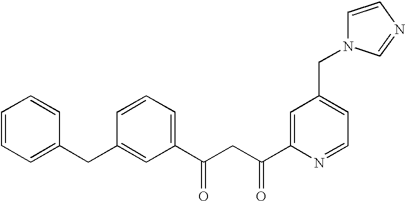

4-tert-Butyldimethylsilanyloxymethyl)pyridine (26B)

To a solution of 4-pyridylcarbinol (26A) (40.0 g, 366.53 mmol) in 400 mL of anhydrous DMF under a nitrogen atmosphere was added imidazole (27.4 g, 403.18 mmol) and tert-butyldimethylchlorosilane (58.0 g, 384.86 mmol). The reaction was stirred overnight at room temperature. The solvent was removed in vacuo. The residue was partitioned between 750 mL EtOAc and 750 mL H₂O. The layers were separated and the organic layer was washed with H₂O, brine, dried over MgSO₄, filtered and concentrated in vacuo to afford the title compound (26B) as a beige oil.

¹H NMR (400 MHz, CDCl₃) δ8.4(m, 2H), 7.25(m, 2H), 4.74(s, 2H), 0.96 (s, 9H), 0.12(s, 6H).

4-(tert-Butyldimethylsilanyloxymethyl)pyridine 1-oxide (26C)

4-(tert-Butyldimethylsilanyloxymethyl)pyridine (26B) (56.6 g, 253.34 mmol) was stirred in 200 mL CH₂Cl₂ under a nitrogen atmosphere. This solution was chilled to 0° C. with an ice bath. In another flask, m-CPBA (57.8 g, 335.30 mmol) was dissolved in 400 mL CH₂Cl₂, dried over MgSO₄, filtered, and added to the reaction. The reaction was stirred overnight at room temperature. The mixture was diluted with CHCl₃, and washed with 1N NaOH, H₂O, brine, dried over MgSO₄, filtered and concentrated in vacuo. The resulting oil was placed under high vacuum which afforded the title compound (26C) as off-white crystals.

¹H NMR (400 MHz, CDCl₃) δ8.19(m, 2H), 7.25(m, 2H), 4.74(s, 2H), 0.96 (s, 9H), (s, 9H), 0.12(s, 6H).

4-(tert-Butyldimethylsilanyloxymethyl)pyridine-2-carbonitrile (26D)

To a cold (0° C.) solution of 4-(tert-butyldimethylsilanyloxymethyl)-pyridine 1-oxide (26C) (30.0 g, 125.30 mmol) and TMSCN (20.0 mL, 150.30 mmol) in anhydrous CH₂Cl₂ (200 mL), dimethycarbonylchloride (19 mL, 150.30 mmol) in CH₂Cl₂ (20 mL) was added dropwise. The ice bath was allowed to expire and the reaction was stirred at room temperature overnight. The reaction was stirred with 200 mL 10% K₂CO₃ for 30 minutes. The layers were separated and the organic layer was washed with brine, dried over MgSO₄, filtered and concentrated in vacuo. This material was chromatographed on silica gel with 15% EtOAc/hexanes to afford the title compound (26D) as a colorless oil.

¹H NMR (400 MHz, CDCl₃) δ8.64(m, 1H), 7.67(m, 1H), 7.46 (m, 1H), 4.77(s, 2H), 0.96 (s, 9H), 0.13(s, 6H).

Methyl 4-hydroxymethylpyridine-2-carboxylate (26E)

To a stirred solution of 4-(tert-butyldimethylsilanyloxymethyl)-pyridine-2-carbonitrile (26D) (21.0 g, 84.54 mmol) in 300 mL MeOH was added H₂O (1.52 m, 84.54 mmol). This solution was cooled to 0° C. with an ice bath and saturated with HCl gas. The resulting solution was refluxed for 5 hours. The reaction was concentrated in vacuo and the residue was dissolved in 300 mL EtOAc. This solution was made basic with saturated NaHCO₃. The layers were separated and the aqueous layer was extracted twice with EtOAc. The combined organic layers were washed with brine, dried over MgSO₄, filtered, and concentrated in vacuo to afford the title compound (26E) as a white solid.

¹H NMR (400 MHz, CDCl₃) δ8.69(m, 1H), 8.12(s, 1H), 7.51 (m, 1H), 4.84(s, 2H), 4.01 (s, 3H).

Methyl 4-bromomethylpyridine-2-carboxylate (26F)

To a solution of methyl 4-hydroxymethylpyridine-2-carboxylate (26E) (4.13 g, 24.70 mmol) in 50 mL CH₂Cl₂ was added carbon tetrabromide (12.3 g, 37.05 mmol). This solution was cooled to 0° C. with an ice bath. Triphenylphosphine (9.66 g, 36.81 mmol) in 50 mL CH₂Cl₂ was added dropwise. The ice bath was allowed to expire and the reaction was stirred at room temperature overnight. The reaction was concentrated in vacuo and the residue was chromatographed on silica gel using 50% EtOAc/hexanes as eluent to afford the title compound (26F) as a white solid.

¹H NMR (400 MHz, CDCl₃) δ8.73(m, 1H), 8.15(s, 1H), 7.50 (m, 1H), 4.57(s, 2H), 4.03 (s, 3H).

Methyl 4-imidazol-1-ylmethylpyridine-2-carboxylate (26G)

To solution of methyl 4-bromomethylpyridine-2-carboxylate (26F) (460 mg 2 mmol) in dry CH₃CN (17 mL) under nitrogen was added imidazole (149 mg, 2.2 mmol) and K₂CO₃. (199 mg, 24 mole). The reaction was stirred at room temperature overnight. The solvent was removed in vacuo and the residue was partitioned between saturated NaHCO₃ and EtoAc. The layers were separated and the aqueous layer was extracted three times with EtoAc. The combined organic layers were washed with brine, dried over Na₂O₄, filtered, and concentrated in vacuo. This material was chromatographed on silica gel using 5% MeOH/CH₂Cl₂ to afford the title compound (26G) as a white solid.

¹H NMR (400 Mz CDCl₃) δ8.73(m, 1H), 7.92(s, 1H), 7.59 (s, 1H), 7.17 (s, 1H), 7.13 (m, 1H), 6.92 (s, 1H), 5.24(s, 2H), 4.01 (s, 3H).

1-(3-Benzylphenyl)-3-(4-imidazol-1-ylmethylpyridin-2-yl)propane-1,3-dione (26H)

To a solution of 1-(3-benzylphenyl)ethanone (4B) (100 mg, 0.48 mmol) and methyl 4-imidazol-1-ylmethyl-pyridine-2-carboxylate (26G) (162 mg, 0.75 mmol) in 2 mL anhydrous THF was added NaOEt (163 mg, 2.4 mmol). This was stirred at room temperature for 1 hour. The reaction was quenched with saturated NH₄Cl and stirred for 15 minutes. The mixture was diluted with EtOAc and the layers were separated. The aqueous layer was extracted twice with EtOAc. The combined organic layers were washed with brine, dried Na₂SO₄, filtered, and concentrated in vacuo. The residue was purified by reverse-phase HPLC to afford the title compound (26H) as a TFA salt.

HR MS: m/z (M+1) Calc'd for: 396.1707 Found: 396.1714

1-(3-Benzylphenyl)-3-(4-(1,2,4-triazol-1-yl)methyl)pyridin-2-yl)propane-1,3-dione (26I)

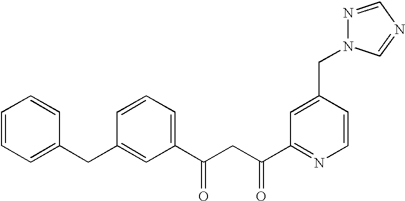

In a manner similar to that for 26H, 1-(3-benzylphenyl)-3-(4-[1,2,4]-triazol-1-ylmethylpyridin-2-yl)propane-1,3-dione (26I) was prepared.

HR MS: m/z (M+1) Calc'd for: 397.1659 Found: 397.1639

1-(3-Benzylphenyl)-3-(4-(pyrazol-1-ylmethylpyridin-2-yl)propane-1,3-dione (26J)

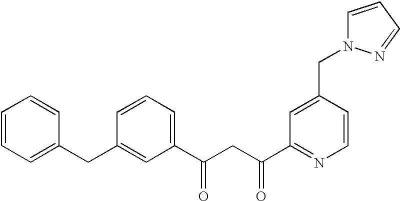

In a manner similar to that for 26H, 1-(3benzylphenyl)-3-(4-pyrazol-1-ylmethylpyridin-2-yl)propane-1,3-dione (26J) was prepared.

HR MS: m/z (M+1) Calc'd for: 396.1707 Found: 396.1671

1-(3-Benzylphenyl)-3-(4-(1,2,3,4-tetrazol-2-yl)methyl)pyridin-2-yl)propane-1,3-dione (26K)

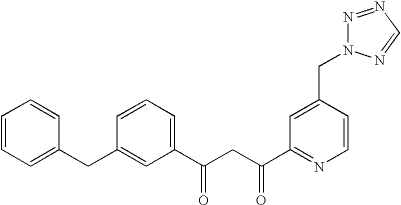

In a manner similar to that for 26H, 1-(3-benzylphenyl)-3-(4-tetrazol-2-ylmethylpyridin-2-yl)propane-1,3-dione (26K) was prepared.

HR MS: m/z (M+1) Calc'd for: 398.1612 Found: 398.1618

1-(3-Benzyl-2-([1,2,3]-triazol-1-ylmethyl)phenyl)-3-(4-imidazol-1-ylmethylpyridin-2-yl)propane-1,3-dione (26L)

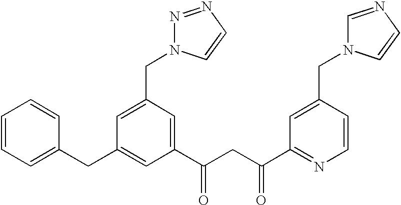

In a manner similar to that for 16C, 1-(3-benzyl-5-[1,2,3]-triazol-1-ylmethylphenyl)-3-(4-imidazol-1-ylmethylpyridin-2-yl)propane-1,3-dione (26L) TFA salt was prepared.

HR MS: m/z (M+1) Calc'd for: 477.2037 Found: 477.2033.

1-(3-Benzylphenyl)-3-(4-methoxymethylpyridin-2-yl)propane-1,3-dione (26M)

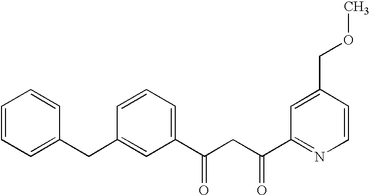

Methyl 4-methoxymethylpyridine-2-carboxylate (26E1)

To a solution of methyl 4-hydroxymethylpyridine-2-carboxylate (26E) (500 mg, 2.99 mmol) in 10 mL anhydrous DNF under nitrogen was added Cs₂CO₃ (1.65 g, 5.08 mmol) and iodomethane (0.223 mL, 3.59 mmol). The reaction was stirred at room temperature overnight. The solvent was removed in vacuo and the residue was diluted with EtOAc. The solids were filtered and the filtrate was concentrated in vacuo. This material was chromatographed on silica gel with 50% EtOAc/hexanes as eluent to afford the title compound (26E1) as a clear oil.

¹H NMR (400 MHz, CDCl₃) δ8.71(m, 1H), 8.10(s, 1H), 7.46 (m, 1H), 4.55(s, 2H), 4.02 (s, 3H), 3.46 (s, 3H).

In a manner similar to the last step for 26H, 1-(3-benzylphenyl)-3-(4-methoxymethylpyridin-2-yl)propane-1,3-dione (26M) TFA salt was prepared.

HR MS: m/z (M+1) Calc'd for: 360.1594 Found: 360.1587

1-(3-Benzylphenyl)-3-(4-hydroxymethylpyridin-2-yl)propane-1,3-dione (26N)

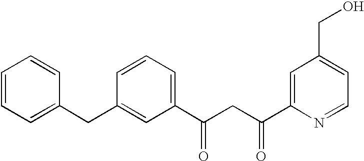

To a 25 mL, oven dried round bottomed flask with a stirring stirring bar and a nitrogen inlet was added THF, methyl 4-hydroxymethylpyridine-2-carboxylate (26E) (0.238 g, 1.43 mmol), 1-(3-benzylphenyl)ethanone (4B) (0.30 g, 1.43 mmol) and sodium ethoxide (0.204 g, 3.00 mmol). This mixture was stirred at ambient temperature for 20 min. The reaction was quenched by addition of saturated aqueous ammonium chloride solution. The product was extracted into ethyl acetate. The ethyl acetate solution was washed with brine, dried (MgSO₄), filtered and concentrated in vacuo. This material was chromatographed on silica gel using ethyl acetate as eluant. The purified product was converted into the HCl salt with 1N HCl and lyophilized.

¹H NMR (300 MHz, DMSO-d₆) δ8.68(d, j=5 Hz, 1H), 8.19(s, 1H), 8.05(s, 1H), 7.91(m, 2H), 7.57(m, 2H), 7.45(br d, j=5 Hz, 2H), 7.24(m, 3H), 5.20(br s, 1H), 4.71(s, 1H), 4.08(s, 2H). (HCl salt).

1-(3-Benzylphenyl)-3-[4-(tetrahydrofuran-2-yl)-pyridine-2-yl]propane-1,3-dione (26V)

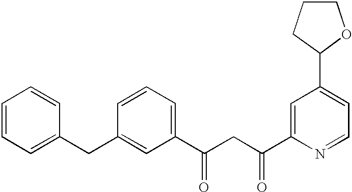

Cyclopropyl-4-pyridylcarbinol (26O)

To a 11 round bottomed flask with a stirring bar, reflux condenser, addition funnel and nitrogen inlet was added Mg turnings (7.09 g, 291.7 mmol); freshly distilled THF (300 mL) Cyclopropyl bromide (20.6 mL, 257 mmol) was added slowly with stirring, at such a rate that the Grignard reaction proceeded at a gentle reflux. When the reaction was complete, the solution was cooled in an ice bath and pyridine-4-carboxaldehyde (25 g, 233.4 mmol) was added as a solution in 50 mL of THF. The mixture was aged 2 h at 0° C. The reaction was quenched with 200 mL of saturated aqueous NH₄Cl followed by 200 mL of water. The resulting mixture was stirred overnight at ambient temperature. This solution was extracted with several portions of ethyl acetate. The combined ethyl acetate were washed with brine, dried (MgSO₄), filtered and concentrated in vacuo. The crude product was chromatographed on of silica gel using ethyl acetate as eluant to give cyclopropyl-4-pyridylcarbinol as a colorless oil which crystallized on standing.

Cyclopropyl-4-pyridylmethanone (26P)

To a 2L round bottomed flask with a stirring bar and a nitrogen inlet was added cyclopropyl-4-pyridylcarbinol (12.5 g, 83.8 mmol), chloroform (600 mL and activated MnO₂ (28 g, 837.9 mmol). This mixture was stirred vigorously at ambient temperature for 7 days. The MnO₂ was removed by filtration and the solvent was removed in vacuo. The crude product was chromatographed on silica gel using 80/20 ethyl acetate/hexanes as eluant to give cyclopropyl-4-pyridylmethanone as a colorless oil.

Cyclopropyl-4-pyridylmethanone N-oxide (26Q)

To a 1L round bottomed flask with an addition funnel, thermometer and a stirring bar was added cyclopropyl-4-pyridylmethanone (10.90 g, 74.06 mmol) and 100 mL of chloroform. This solution was cooled in an ice bath to 0° C. and an anhydrous solution of m-CPBA (16.61 g, 96.28 mmol) in chloroform (200 mL) was wadded dropwise over 45 min. The cooling bath was allowed to expire and the mixture was stirred at ambient temperature for 5 days. The mixture was washed with 15% aqueous NaHSO₃ solution, 10% aqueous K₂CO₃ solution and brine. Drying (MgSO₄), filtration and removal of the solvent in vacuo gave an oil. This material was chromatographed on silica gel using 3/97 methanol/chloroform as eluant to give cyclopropyl-4-pyridylmethanone N-oxide as a white, crystalline solid.

Cyclopropyl-4-(2-cyanopyridyl)methanone (26R)

To a 500 mL, three necked round bottomed flask with an addition funnel, nitrogen inlet, and a thermometer was added cyclopropyl-4-pyridylmethanone N-oxide (8.25 g, 50.56 mmol), methylene chloride (100 mL) and trimethylsilylcyanide (8.45 mL, 63.32 mmol). This solution was cooled in an ice bath to 0° C. and the addition funnel was charged with N,N-dimethylcarbamyl chloride (8.00 mL, 63.32 mmol) in methylene chloride (20 mL). The N,N-dimethylcarbamyl chloride solution was added dropwise over 30 min. The cooling bath was removed and the mixture was stirred at ambient temperature overnight. The reaction was quenched 200 mL of 10% aqueous K₂CO₃. This mixture was stirred 30 minutes then diluted with chloroform the layers were separated and the organic phase was washed with brine, dried (MgSO₄), filtered and concentrated in vacuo. The crude product was chromatographed on 400 g of silica gel using 1-2% methanol in chloroform as eluant to provide cyclopropyl-4-(2-cyanopyridyl)methanone as white crystals.

¹H NMR (300 MHz, CDCl₃) δ8.93(dd, j=1, 10 Hz, 1H), 8.19(m, 1H), 8.01 (dd, j=1.10, 1H), 2.60(m, 1H), 1.35 (m, 2H), 1.21 (m, 2H).

1-(3-Chloropropyl)-4-(2-carbomethoxypyridyl)methanone (26S)

To a 200 mL round bottomed flask with a stirring bar, gas dispersion tube and a reflux condenser was added cyclopropyl-4-(2-cyanopyridyl)methanone (2.77 g, 16.09 mmol) methanol (200 mL) and water (0.29 mL). This wall stirred mixture was saturated with a vigorous stream of HCl gas for 30 min. The resulting mixture was heated at reflux for 20 h. The mixture was cooled to ambient temperature and the methanol was removed in vacuo. The residue was partitioned between aqueous NaHCO₃ solution and ethyl acetate. The layers were separated and the organic phase was washed with brine, dried (MgSO₄), filtered and concentrated in vacuo. This material was chromatographed on silica gel using 1:1 ethyl acetate/hexane as eluant to give 1-(3-chloropropyl)-4-(2-carbomethoxypyridyl)methanone as an oil.

¹H NMR (300 MHz, CDCl₃) δ8.96(dd, j=1, 10 Hz, 1H), 8.57(m, 1H), 7.94 (dd, j=1.10, 1H), 4.05(3, 3H), 3.70 (t, j=6 Hz, 2H), 3.25 (t, j=6 Hz, 2H), 2.27 (m, 2H).

1-(3-Chloropropyl)-4-(2-carbomethoxypyridyl)carbinol (26T)

To a 100 mL round bottomed flask with a stirring bar and a nitrogen inlet was added 1-(3-chloropropyl)-4-(2-carbomethoxypyridyl)methanone (2.00 g, 8.28, mmol) methanol (25 mL) and sodium borohydride (0.31 g, 8.28 mmol). This solution was stirred at ambient temperature for 2 h. The reaction was quenched with aqueous NH₄Cl solution and the methanol was removed in vacuo. The aqueous residue was extracted with ethyl acetate. The ethyl acetate solution was washed with brine, dried (MgSO₄), filtered and concentrated in vacuo. The crude product was chromatographed on silica gel using ethyl acetate as eluant to provide 1-(3-chloropropyl)-4-(2-carbomethoxypyridyl)carbinol as a colorless oil.

¹H NMR (300 MHz, CDCl₃) δ8.70(dd, j=1, 10 Hz, 1H), 8.12(m, 1H), 7.51 (dd, j=1.10, 1H), 4.88(m, 1H), 4.01 (s, 3H), 3.58 (m, 2H), 2.50 (br s, 1H), 1.92 (m, 4H).

2-Carbomethoxy-4-(2-tetrahydrofuryl)pyridine (26U)

To a 100 mL round bottomed flask with a stirring bar, reflux condenser and a nitrogen inlet was added 1-(3-chloropropyl)-4-(2-carbomethoxypyridyl)carbinol (1.25 g, 5.13 mmol), dry THF (20 mL), sodium hydride (0.18 g, 7.50 mmol) and a catalytic amount of sodium iodide. This mixture was heated at reflux for 3 h. The cooled reaction mixture was treated with aqueous NH₄Cl solution and extracted with ethyl acetate. The ethyl acetate solution was washed with brine, dried (MgSO₄), filtered and concentrated in vacuo. The crude product was chromatographed on silica gel, using 80/20 ethyl acetate/hexanes as eluant to give 2-carbomethoxy-4-(2-tetrahydrofuryl)pyridine.

¹H NMR (300 MHz, CDCl₃) δ8.69(dd, j=1, 10 Hz, 1H), 8.09(m, 1H), 7.46 (dd, j=1.10, 1H), 4.97(t, j=7 Hz, 1H), 4.21(m, 1H), 4.00 (s, 3H), 3.98 (m, 1H), 2.44 (m, 1H), 2.04 (m, 2H), 1.79 (m, 1H).

1-(3-Benzylphenyl)-3-[4-(tetrahydrofuran-2-yl)-pyridin-2-yl]propane-1,3-dione (26V)

A mixture of 2-carbomethoxy-4-(2-tetrahydrofuryl)pyridine, (26U) (0.25 g, 1.21 mmol), 1-(3-benzylphenyl)ethanone (4B) (0.25 g, 1.21 mmol), and sodium ethoxide (0.170 g, 2.50 mmol) in THF was stirred at ambient temperature for 20 min. The reaction was quenched by addition of saturated aqueous ammonium chloride solution. The product was extracted into ethyl acetate. The ethyl acetate solution was washed with brine, dried (MgSO₄), filtered and concentrated in vacuo. The residue was chromatographed by reverse phase HPLC using 0.1% aqueous TFA/acetonitrile as eluant. Collection and lyophilization of appropriate fractions provided 1-(3-benzylphenyl)-3-[4-(tetrahydrofuran-2-yl)pyridin-2-yl]propane-1,3-dione as a crystalline solid.

¹H NMR (300 MHz, CDCl₃) δ8.69(d, j=10 Hz, 1H), 8.09(br s, 1H), 7.46 (m, 2H), 7.62(br d, j=10 Hz, 1 H), 7.58 (s, 1H), 7.40(m, 2H), 7.25(m, 5H), 5.08(t, j=7 Hz, 1H), 4.19(m, 1H), 4.07 (s, 3H), 4.02 (m, 1H), 2.51 (m, 1H), 2.06 (m, 2H), 1.83 (m, 1H).

1-(3,5-Bispyrazol-1-ylmethyl-phenyl)-3-(4-[1,2,4]triazol-1-ylmethyl-pyridin-2-yl)-propane-1,3-dione (26W)

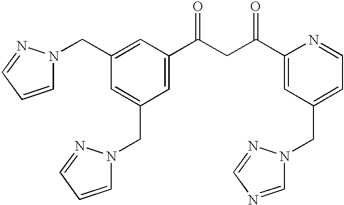

Methyl 4-(1H-1,2,4-triazol-1-ylmethyl)-2-pyridinecarboxylate

A solution of methyl 4-(bromomethyl)-2-pyridinecarboxylate (26F) (1.5 g, 6.6 mmole) in 50 mL CH₃CN was treated with 1,2,4-triazole (2.27 g, 33 mmole) and allowed to stir at room temperature overnight. The solution was evaporated and the residue dissolved in saturated Na₂SO₄ and extracted repeatedly with chloroform. The organic layers were combined and evaporated and the residue was chromatographed in 5% MeOH/CHCl₃ to give methyl 4-(1H-1,2,4-triazol-1-ylmethyl)-2-pyridinecarboxylate as a yellow oil.

Rf=0.27 (5% MeOH/CHCL₃)

¹H NMR (400 MHz, CDCl₃) δ8.75 (s, 1H), 8.21 (s, 1H), 8.04 (s, 1H), 7.98 (s, 1H), 7.28 (s, 1H), 5.46(s, 2H), 4.02(s, 3H).

In a manner similar to that described for 19E [except that in the final step, methyl 4-(1H-1,2,4-triazol-1-ylmethyl)-2-pyridinecarboxylate replaced the 5-methyl 4-methylpydridine-2-carboxylate (3H)], 1-(3,5-bis-pyrazol-1-ylmethyl-phenyl)-3-(4-[1,2,4]triazol-1-ylmethyl-pyridin-2-yl)propane-1,3-dione 26W was prepared.

¹H NMR (400 MHz, CDCl₃) δ8.70(d, 1H, J=4.84), 8.21(s, 1H), 8.05(s, 1H), 8.00(s, 1H), 7.83(s, 2H), 7.56(d, 2H, J=1.65), 7.49(s, 1H), 7.41(d, 2H, J=2.28), 7.23(m, 1H), 7.17(s, 1H), 6.30(t, 2H, J=2H), 5.47(s, 2H), 5.36(s, 4H).

1-(3,5-Bis-pyridin-2-ylmethylphenyl)-3-(4-imidazol-1-ylmethyl-pyridin-2-yl)propane-1,3-dione (26X)

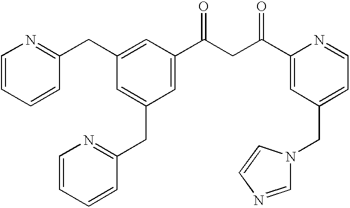

In a manner similar to that described for 3M using methyl 4-imidazol-1-ylmethylpyridine-2-carboxylate (26G), 1-(3,5-bis-pyridin-2-ylmethylphenyl-3-(4-imidazol-1-ylmethyl-pyridin-2-yl)propane-1,3-dione (26X) was prepared.

¹H NMR (400 MHz, DMSO) δ9.29 (s, 1H), 8.82 (d, 1H), 8.57 (d, 1H), 8.07 (s, 1H), 7.95−7.35 (m), 5.64 (s, 2H), 4.25 (s, 4H).

### EXAMPLE 27

1-{3-Benzyl-5[(1-dioxido-1,2-thiazinan-2-yl)methyl]phenyl}-3-(1,3-thiazol-2-yl)-1,3-propanedione (27A)

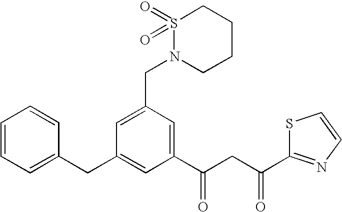

Step 1: Ethyl 1,3-thiazole-2-carboxylate

A mixture of 2-(trimethylsilyl)thiazole (1 g, 6.36 mmole) and ethyl chloroformate (1.38 g, 12.71 mmol) in benzene (10 mL) was stirred at rt for 3 days under a nitrogen atmosphere. The reaction was then poured into 50 mL saturated aqueous Na₂HCO₃ solution and extracted with 3×20 mL ethyl acetate. The combined organic extracts were washed with water, brine, dried (Na₂SO₄), filtered, and concentrated to provide ethyl 1,3-thiazole-2-carboxylate.

¹H NMR (400 MHz, CDCl₃) δ8.03(d, J=3 Hz, 1H), 7.64 (d, J=3 Hz, 1H), 7.27 (s, 1H), 4.49 (q, J=7 Hz, 2H), 1.45 (t, J=7 Hz, 3H).

Step 2: 1-{3-Benzyl-5-[(1,1-dioxido-1,2-thiazinan-2-yl)methyl]phenyl}-3-(1,3-thiazol-2-yl)-1,3-propanedione (27A)

Similar to 16R, 1-{3-benzyl-5-[(1,1-dioxido-1,2-thiazinan-2-yl)methyl]phenyl}ethanone (0.057 g, 0.16 mmol) was reacted with ethyl 1,3-thiazole-2-carboxylate (0.03 g, 0.19 mmol) to afford 1-{3-benzyl-5-[(1,1-dioxido-1,2-thiazinan-2-yl)methyl]phenyl}-3-(1,3-thiazol-2-yl)-1,3-propanedione (27A).

¹H NMR (400 MHz, CDCl₃) δ8.04 (d, J=3 Hz, 1H) 7.81 (s, 1H), 7.78 (s, 1H), 7.69 (d, J=3 Hz, 1H), 7.41 (s, 1H), 7.14-7.34 (m, 7H), 4.33 (s, 2H), 4.06(s, 2H), 3.21 (t, J=6 Hz, 2H), 3.08 (t, J=6 Hz, 2H), 2.21 (m, 2H), 1.59 (m, 2H).

1-{3-Benzyl-5-[(1,1-dioxido-2-isothiazolidinyl)methyl]phenyl}-3-(1,3thiazol-2yl)-1,3-propanedione (27B)

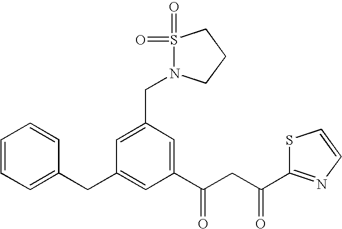

Similar to 16Q, 1-{3-benzyl-5-[(1,1-dioxido-2-isothiazolidinyl)methyl]phenyl}ethanone (0.05 g, 0.15 mmol) was reacted with ethyl 1,3-triazole-2-carboxylate (0.027 g, 0.18 mmol) to provide 1-{3-benzyl-5-[(1,1-dioxido-2-isothiazolidinyl)methyl]phenyl}-3-(1,3-thiazol-2-yl)-1,3-propanedione (27B).

¹H NMR (400 MHz, CDCl₃) δ8.04 (d, J=3 Hz, 1H), 7.81 (s, 2H), 7.69 (d, J=3 Hz, 1H), 7.40 (s, 1H), 7.14-7.34 (m, 7H), 4.20 (s, 2H), 4.05 (s, 2H), 3.20 (t, J=8 Hz, 2H), 3.10 (t, J=7 Hz, 2H), 2.30 (m, 2H).

1-{3-Benzyl-5-[(6-oxo-1(6H)-pyrimidinyl)methyl]phenyl}-3-(1,3-thiazol-2-yl)-1,3-propanedione (27C)

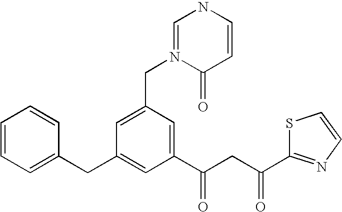

Similar to 16O, 3-(3-acetyl-5-benzylbenzyl)-4(3H)pyrimidinone (0.05 g, 0.16 mmol) was reacted with ethyl 1,3-triazole-2-carboxylate (0.029 g, 0.19 mmol) to provide 1-{3-benzyl-5-[(6-oxo-1(6H)-pyrimidinyl)methyl]phenyl}-3-(1,3-thiazol-2-yl)-1,3-propanedione (27C).

¹H NMR (400 MHz, CDCl₃) δ8.25 (d, J=3 Hz, 1H), 8.04 (d, J=3 Hz, 1H), 7.79 (s, 1H), 7.77 (s, 1H), 7.67 (d, J=3 Hz, 1H), 7.41 (s, 1H), 7.14-7.34 (m, 7H), 6.18 (s, 1H), 5.10 (d, J=6 Hz, 1H), 4.46( m, 2H), 4.02 (s, 2H).

### EXAMPLE 28

Oral Composition

As a specific embodiment of an oral composition of a compound of this invention, 50 mg of compound 16C is formulated with sufficient finely divided lactose to provide a total amount of 580 to 590 mg to fill a size 0 hard gelatin capsule.

### EXAMPLE 29

Determination of Log P

The procedure for determining partition coefficient P at ambient temperature is as follows: 10 ml of pH 7.4 KH₂PO₄ buffer and 10 ml of 1-octanol which have been mutually saturated with each other are added to an accurately weighed sample of the order of 1-2 mg of a standard solution of the compound of interest in a suitable solvent (e.g., methanol, ethanol, acetonitrile, or water). The mixture is agitated in an ultrasonic bath for 5 minutes and on a flatbed shaker for 2-4 hours, and then centrifuged. 1 ml of each layer is removed from the resulting biphasic system and the concentrations of the compound in the standard, octanol, and buffer solutions is determined by ultraviolet absorption spectrophotometry or by determining peak areas in HPLC.

Using the foregoing procedure, representative compounds of the present invention were determined to have log P's greater than zero.

### EXAMPLE 30

HIV Integrase Assay: Strand Transfer Catalyzed by Recombinant Integrase

Assays for the strand transfer activity of integrase were conducted according to Wolfe, A. L. et al., J. Virol. 70, 1424 (1996) for recombinant integrase. Representative compounds tested in the integrase assay demonstrated IC₅₀'s in the range of from 0.01 to 5 micromolar.

### EXAMPLE 31

Assay for Inhibition of HIV Replication

Assays for the inhibition of acute HIV infection of T-lymphoid cells were conducted according to Vacca, J. P. et al., (1994), Proc. Natl. Acad. Sci. USA 91, 4906. Representative compounds tested in the present assay demonstrated IC₉₅'s in the range from 0.01 to 50 micromolar.

While the foregoing specification teaches the principles of the present invention, with examples provided for the purpose of illustration, the practice of the invention encompasses all of the usual variations, adaptations and/or modifications that come within the scope of the following claims.

What is claimed is:

1. A compound of formula: 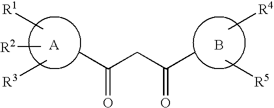
or a tautomer thereof; wherein
A is (i) a benzene ring; (ii) an 8- to 10-membered fused bicyclic carbocycle, wherein the ring, of the carbocycle attached to the central dione moiety is a benzene ring, and the other ring of the carbocycle is saturated or unsaturated; (iii) an 8 to 10-membered fused bicyclic heterocycle containing carbon atoms and from 1 to 3 heteroatoms selected from nitrogen, oxygen and sulfur, wherein the ring of the heterocycle attached to the central dione moiety is a benzene ring, and the other ring of the heterocycle is a saturated or unsaturated heteroatom-containing ring; or (iv) a 5- or 6-membered heteroaromatic ring containing from 1 to 3 heteroatoms selected from nitrogen, oxygen and sulfur; and wherein A is attached to the central dione moiety via a carbon atom;
R¹, R² and R³ are substituents attached to nitrogen or carbon in A;
R¹ is hydrogen, halo, nitro, C₁-C₆ alkyl, C₁-C₆ alkoxy, fluorinated C₁-C₆ alkyl, fluorinated C₁-C₆ alkoxy, C₂-C₈ alkoxyalkyl, fluorinated C₂-C₈ alkoxyalkyl, N(R^{a})(R^{b}), (CH₂)₁₋₃N(R^{a})(R^{b}), (CH₂)₀₋₃R^{c}, or O(CH₂)₀₋₃R^{c};
R² is hydrogen, halo, nitro, C₁-C₆ alkyl, C₁-C₆ alkoxy, fluorinated C₁-C₆ alkyl, fluorinated C₁-C₆ alkoxy, C₂-C₈ alkoxyalkyl, fluorinated C₂-C₈ alkoxyalkyl, N(R^{a})(R^{b}), (CH₂)₁₋₃N(R^{a})(R^{b}), (CH₂)₀₋₃R^{c}, O(CH₂)₀₋₃R^{c}, (CH₂)₀₋₃R^{d}, O(H₂)₀₋₃R^{d}, C(═O)CH₂C(═O)R^{e}, or R^{f};
R³ is hydrogen, halo, nitro, oxo, C₁-C₆ alkyl, C₃-C₇ cycloalkyl, C₃-C₇ cycloalkyloxy, C₁-C₆ alkoxy, fluorinated C₁-C₆ alkyl, fluorinated C₁-C₆ alkoxy, C₂-C₈ alkoxyalkyl, fluorinated C₂-C₈ alkoxyalkyl, N(R^{a})(R^{b}), (CH₂)₁₋₄N(R^{a})(R^{b}), C(═O)N(R^{a})(R^{b}), (CH₂)₁₋₄C(═O)N(R^{a})(R^{b}), N(R^{a})C(═O)R^{b}, (CH₂)₁₋₄N(R^{a})C(═O)R^{b}, SO₂R^{a}, (CH₂)₁₋₄SO₂R^{a}, SO₂N(R^{a})(R^{b}), (CH₂)₁₋₄SO₂N(R^{a})(R^{b}), (CH₂)₁₋₄N(R^{a})SO₂R^{b}, (CH₂)₀₋₃R^{c}, or (CH₂)₀₋₃R^{g};
B is (i) a 5- or 6-membered heteroaromatic ring containing from 1 to 4 nitrogen atoms, 0 to 2 sulfur atoms, and at least 1 carbon atom, or (ii) an 8- to 10-membered fused bicyclic heterocycle containing from 1 to 4 nitrogen atoms, 0 to 2 sulfur atoms, and carbon atoms, wherein the ring of the heterocycle attached to the central dione moiety is a 5- or 6-membered heteroaromatic ring containing at least one nitrogen or sulfur atom and the other ring of the heterocycle is a saturated or unsaturated ring; wherein B is attached to the central dione moiety via a carbon atom and at least one nitrogen or sulfur atom in B is adjacent to the point of attachment;
R⁴ and R⁵ are substituents attached to nitrogen or carbon in B, and are each independently selected from hydrogen, halo, hydroxy, (CH₂)₁₋₄OH, C₁-C₆ alkyl, C₁-C₆ alkoxy, fluorinated C₁-C₆ alkyl, fluorinated C₁-C₆ alkoxy, C₂-C₈ alkoxyalkyl, fluorinated C₂-C₈ alkoxyalkyl, N(R^{a})(R^{b}), (CH₂)₁₋₄N(R^{a})(R^{b}), C(═O)N(R^{a})(R^{b}), (CH₂)₁₋₄C(═O)N(R^{a})(R^{b}), N(R^{a})C(═O)R^{b}, (CH₂)₁₋₄N(R^{a})C(═O)R^{b}, SO₂R^{a}, (CH₂)₁₋₄SO₂R^{a}, SO₂N(R^{a})(R^{b}), (CH₂)₁₋₄SO₂N(R^{a})(R^{b}), (CH₂)₁₋₄N(R^{a})SO₂R^{b}, and(CH₂)₀₋₃R^{h};
R^{a} and R^{b} are each independently hydrogen, C₁-C₆ alkyl, or fluorinated C₁-C₆ alkyl;
R^{c} is (i) phenyl or substituted phenyl, wherein each substituent on the substituted phenyl is independently halo, cyano, hydroxy, (CH₂)₁₋₄OH, N(R^{a})(R^{b}), C₁-C₆ alkyl, fluorinated C₁-C₆ alkyl, C₁-C₆-alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, and fluorinated C₂-C₈ alkoxyalkyl; or (ii) an 8- to 10-membered fused bicyclic carbocycle in which one ring is a benzene ring and the other ring is a saturated or unsaturated ring, wherein the fused carbocycle is unsubstituted or substituted with one or more substituents selected from halo, cyano, hydroxy, (CH₂)₁₋₄OH, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, and fluorinated C₂-C₈ alkoxyalkyl;
R^{d} is (i) a 5- or 6-membered monocyclic heterocycle which is saturated or unsaturated and which contains at least one carbon atom and from 1 to 4 heteroatoms selected from nitrogen, oxygen, and sulfur; (ii) an 8- to 10-membered fused bicyclic heterocycle in which either ring is saturated or unsaturated and which contains carbon atoms and from 1 to 4 nitrogen atoms; wherein the heterocycle of (i) or (ii) is unsubstituted or substituted with one or more substituents selected from halo, cyano, hydroxy, (CH₂)₁₋₄OH, oxo, thio, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, fluorinated C₂-C₈ alkoxyalkyl, phenyl and benzyl; or (iii) a 5- or 6-membered monocyclic heterocycle which is saturated or unsaturated and which contains carbon atoms and from 1 to 3 nitrogen atoms, the heterocycle being substituted with spiro-C₁-C₂ alkylenedioxy or with one of pyrrolidinyl, piperidinyl, piperazinyl, and morpholinyl, unsubstituted or substituted with one or more substituents selected from halo, cyano, hydroxy, (CH₂)₁₋₄OH, oxo, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, fluorinated C₂-C₈ alkoxyalkyl, phenyl and benzyl;
R^{e} is a 5- or 6-membered heteroaromatic ring containing from 1 to 4 nitrogen atoms, 0 to 2 sulfur atoms, and at least 1 carbon atom; wherein the point of attachment of the ring is a carbon atom and at least one nitrogen or sulfur atom in the ring is adjacent to the point of attachment; wherein the ring is unsubstituted or substituted with one or more substituents selected from halo, cyano, hydroxy, (CH₂)₁₋₄OH, oxo, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, fluorinated C₂-C₈ alkoxyalkyl, phenyl and benzyl;
R^{f} is X—NH(CH₂)₁₋₃Y, wherein X is a 5- or 6-membered monocyclic heterocycle which is saturated or unsaturated and which contains carbon atoms and from 1 to 3 nitrogen atoms and which is unsubstituted or substituted with one or more substituents selected from halo, cyano, hydroxy, (CH₂)₁₋₄OH, oxo, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, and fluorinated C₂-C₈ alkoxyalkyl; Y is pyrrolidinyl, piperidinyl, piperazinyl, or morpholinyl, which is unsubstituted or substituted with one or more substituents selected from halo, cyano, hydroxy, (CH₂)₁₋₄OH, oxo, N(R^{a})(R^{b}), C₁-C₆alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, and fluorinated C₂-C₈ alkoxyalkyl;
R^{g} is a 5- or 6-membered monocyclic heterocycle which is saturated or unsaturated and which contains one or more carbon atoms and from 1 to 4 nitrogen atoms, the heterocycle being unsubstituted or substituted with one or more substituents selected from halo, cyano, hydroxy, (CH₂)₁₋₄OH, oxo, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)¹⁻⁴N(R^{a})SO₂R^{b}, C₂-C₈ alkoxyalkyl, fluorinated C₂-C₈ alkoxyalkyl, phenyl and benzyl;
R^{h} is (i) C₃-C₆ cycloalkyl; (ii) phenyl; (iii) substituted phenyl, wherein each substituent on the substituted phenyl is independently halo, cyano, hydroxy, (CH₂)₁₋₄OH, C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, N(R^{a})(R^{b}), (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, or fluorinated C₂-C₈ alkoxyalkyl, or (iv) a 5- or 6-membered monocyclic heterocycle which is saturated or unsaturated and which contains at least one carbon atom and from 1 to 4 heteroatoms selected from nitrogen, oxygen, and sulfur; wherein the heterocycle is unsubstituted or substituted with one or more substituents selected from halo, cyano, hydroxy, (CH₂)₁₋₄OH, oxo, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, (CH₂)₁₋₄N(R^{a})(R^{b}), (CH₂)₀₋₄N(R^{a})C(═O)R^{b}, (CH₂)₀₋₄SO₂N(R^{a})(R^{b}), (CH₂)₁₋₄N(R^{a})SO₂R^{b}, C₂-C₈ alkoxyalkyl, fluorinated C₂-C₈ alkoxyalkyl, phenyl and benzyl;
or a pharmaceutically acceptable salt thereof.

2. The compound according to claim 1, or a tautomer thereof, wherein
R⁴ and R⁵ are substituents attached to nitrogen or carbon in B, and are each independently selected from hydrogen, halo, hydroxy, C₁-C₆ alkyl, C₁-C₆ alkoxy, fluorinated C₁-C₆ alkyl, fluorinated C₁-C₆ alkoxy, C₂-C₈ alkoxyalkyl, fluorinated C₂-C₈ alkoxyalkyl, N(R^{a})(R^{b}), (CH₂)₁₋₄N(R^{a})(R^{b}), C(═O)N(R^{a})(R^{b}), (CH₂)₁₋₄C(═O)N(R^{a})(R^{b}), N(R^{a})C(═O)R^{b}, (CH₂)₁₋₄N(R^{a})C(═O)R^{b}, SO₂R^{a}, (CH₂)₁₋₄SO₂R^{a}, SO₂N(R^{a})(R^{b}), (CH₂)₁₋₄SO₂N(R^{a})(R^{b}), (CH₂)₁₋₄N(R^{a})SO₂R^{b}, and (CH₂)₀₋₃R^{h};
R^{a} and R^{b} are each independently hydrogen, C₁-C₄ alkyl, or fluorinated C₁-C₄ alkyl;
R^{c} is (i) phenyl or substituted phenyl, wherein each substituent on the substituted phenyl is independently halo, cyano, hydroxy, C₁-C₆ alkyl, fluorinated C₁-C₆ alkyl, C₁-C₆ alkoxy, fluorinated C₁-C₆ alkoxy, N(R^{a})(R^{b}), (CH₂)₁₋₄N(R^{a})(R^{b}), (CH₂)₀₋₄CO₂R^{a}, (CH₂)₀₋₄C(═O)N(R^{a})(R^{b}), (CH₂)₀₋₄SO₂R^{a}; C₂-C₈ alkoxyalkyl, or fluorinated C₂-C₈ alkoxyalkyl; or (ii) an 8- to 10-membered fused bicyclic carbocycle in which one ring is a benzene ring and the other ring is a saturated or unsaturated ring, wherein the fused carbocycle is unsubstituted or substituted with one or more substituents selected from halo, cyano, hydroxy, C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, C₂-C₈ alkoxyalkyl, and fluorinated C₂-C₈ alkoxyalkyl;
R^{d} is (i) a 5- or 6-membered monocyclic heterocycle which is saturated or unsaturated and which contains at least one carbon atom and from 1 to 4 heteroatoms selected from nitrogen, oxygen, and sulfur; (ii) an 8- to 10-membered fused bicyclic heterocycle in which either ring is saturated or unsaturated and which contains carbon atoms and from 1 to 4 nitrogen atoms; wherein the heterocycle of (i) or (ii) is unsubstituted or substituted with one or more substituents selected from halo, cyano, hydroxy, oxo, N(R^{a})(R^{b}), C₁-C₆ alkyl, fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, C₂-C₈ alkoxyalkyl, and fluorinated C₂-C₈ alkoxyalkyl; or (iii) a 5- or 6-membered monocyclic heterocycle which is saturated or unsaturated and which contains carbon atoms and from 1 to 3 nitrogen atoms, the heterocycle being substituted with spiro-C₁-C₂ alkylenedioxy, or with one of pyrrolidinyl, piperidinyl, piperazinyl, and morpholinyl, unsubstituted or substituted with one or more substituents selected from halo, cyano, hydroxy, N(R^{a})(R^{b}), C₁-C₆ alkyl, fluorinated C₁-C₆ alkyl, C₁-C₆ alkoxy, and fluorinated C₁-C₆ alkoxy;
R^{e} is a 5- or 6-membered heteroaromatic ring containing from 1 to 4 nitrogen atoms, 0 to 2 sulfur atoms, and at least 1 carbon atom; wherein the point of attachment of the ring is a carbon atom and at least one nitrogen or sulfur atom in the ring is adjacent to the point of attachment; wherein the ring is unsubstituted or substituted with one or more substituents selected from halo, cyano, hydroxy, oxo, C₁-C₆ alkyl, fluorinated C₁-C₆ alkyl, C₁-C₆ alkoxy, fluorinated C₁-C₆ alkoxy, (CH₂)₀₋₄CO₂R^{a}, N(R^{a})(R^{b}), (CH₂)₀₋₄C(═O)N(R^{a})(R^{b}), (CH₂)₀₋₄SO₂R^{a}, C₂-C₈ alkoxyalkyl, and fluorinated C₂-C₈ alkoxyalkyl;
R^{f} is X—NH(CH₂)₁₋₃Y, wherein X is a 5- or 6-membered monocyclic heterocycle which is saturated or unsaturated and which contains carbon atoms and from 1 to 3 nitrogen atoms and which is unsubstituted or substituted with one or more substituents selected from halo, cyano, hydroxy, N(R^{a})(R^{b}), C₁-C₆ alkyl, fluorinated C₁-C₆ alkyl, C₁-C₆ alkoxy, and fluorinated C₁-C₆ alkoxy; Y is pyrrolidinyl, piperidinyl, piperazinyl, or morpholinyl, which is unsubstituted or substituted with one or more substituents selected from halo, cyano, hydroxy, N(R^{a})(R^{b}), C₁-C₆ alkyl, fluorinated C₁-C₆ alkyl, C₁-C₆ alkoxy, and fluorinated C₁-C₆ alkoxy;
R^{g} is a 5- or 6-membered monocyclic heterocycle which is saturated or unsaturated and which contains one or more carbon atoms and from 1 to 4 nitrogen atoms, the heterocycle being unsubstituted or substituted with one or more substituents selected from halo, cyano, hydroxy, N(R^{a})(R^{b}), C₁-C₆ alkyl, fluorinated C₁-C₆ alkyl, C₁-C₆ alkoxy and fluorinated C₁-C₆ alkoxy; and
R^{h} is C₃-C₆ cycloalkyl, phenyl or substituted phenyl, wherein each substituent on the substituted phenyl is independently halo, cyano, hydroxy, C₁-C₆ alkyl fluorinated C₁-C₆ alkyl, C₁-C₆ alkoxy, fluorinated C₁-C₆ alkoxy, (CH₂)₀₋₄CO₂R^{a}, (CH₂)₀₋₄C(═O)N(R^{a})(R^{b}), (CH₂)₀₋₄SO₂R^{a}, C₂-C₈ alkoxyalkyl, or fluorinated C₂-C₈ alkoxyalkyl;
or a pharmaceutically acceptable salt thereof.

3. The compound according to claim 1, or a tautomer thereof, wherein
A is a benzene ring;
or a pharmaceutically acceptable salt thereof.

4. The compound according to claim 3, or a tautomer thereof, wherein
B is a 5- or 6-membered heteroaromatic ring containing from 1 to 4 nitrogen atoms, 0 to 2 sulfur atoms, and at least 1 carbon atom;
or a pharmaceutically acceptable salt thereof.

5. The compound according to claim 4, or a tautomer thereof, having the formula: 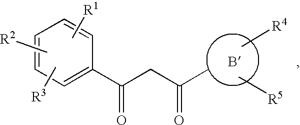
wherein
R¹ is hydrogen, fluoro, chloro, bromo, methyl, ethyl, propyl, isopropyl, OCH₃, OCH2CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, N(R^{a})(R^{b}), CH₂N(R^{a})(R^{b}), (CH₂)₀₋₂R^{c}, or O(CH₂)₀₋₂R^{c};
R² is hydrogen, fluoro, chloro, bromo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, N(R^{a})(R^{b}), CH₂N(R^{a})(R^{b}), (CH₂)₀₋₂R^{c}, O(CH₂)₀₋₂R^{c}, (CH₂)₀₋₂R^{d}, O(CH₂)₀₋₂R^{d}, C(═O)CH₂C(═O)R^{e}, or R^{f};
R³is hydrogen, fluoro, chloro, bromo, oxo, methyl, ethyl, propyl, isopropyl, C₃-C₆ cycloalkyl, C₃-C₆ cycloalkyloxy, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, (CH₂)₀₋₂R^{c}, or (CH₂)₀₋₂R^{g};
B′ is a 5- or 6-membered heteroaromatic ring containing from 1 to 4 nitrogen atoms, 0 or 1 sulfur atoms, and one or more carbon atoms, wherein B′ is attached to the central dione moiety via a carbon atom and at least one nitrogen atom in B′ is adjacent to the point of attachment;
R⁴ and R⁵ are substituents attached to any nitrogen or carbon in B′ except for the ring carbon attached to the central dione moiety, and are each independently selected from hydrogen, fluoro, chloro, bromo, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₂OH, (CH₂)₁₋₂O—C₁-C₄ alkyl, (CH₂)₁₋₃O(CH₂)₀₋₁CF₃, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, and (CH₂)₀₋₂R^{h};
R^{a} and R^{b} are each independently hydrogen, C₁-C₄ alkyl, or fluorinated C₁-C₄ alkyl;
R^{c} is (i) phenyl or substituted phenyl, wherein each substituent on the substituted phenyl is independently fluoro, chloro, bromo, hydroxy, (CH₂)₁₋₂OH, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R^{b}; or (ii) an 8- to 10-membered fused bicyclic carbocycle in which one ring is a benzene ring and the other ring is a saturated or unsaturated ring, wherein the fused carbocycle is unsubstituted or substituted with one or more substituents selected from fluoro, chloro, bromo, hydroxy, (CH₂)₁₋₂OH, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R^{b};
R^{d} is (i) a 5- or 6-membered monocyclic heterocycle which is saturated or unsaturated and which contains at least one carbon atom and from 1 to 4 heteroatoms selected from nitrogen, oxygen, and sulfur, wherein each ring sulfur is in a form selected from S, SO and SO₂; (ii) an 8- to 10-membered fused bicyclic heterocycle in which either ring is saturated or unsaturated and which contains carbon atoms and from 1 to 4 nitrogen atoms; wherein the heterocycle of (i) or (ii) is unsubstituted or substituted with one or more substituents selected from fluoro, chloro, bromo, hydroxy, (CH₂)₁₋₂OH, oxo, thio, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl; or (iii) a 5- or 6-membered monocyclic heterocycle which is saturated or unsaturated and which contains carbon atoms and from 1 to 3 nitrogen atoms, the heterocycle being substituted with spiro-C₁-C₂ alkylenedioxy, or with one of pyrrolidinyl, piperidinyl, piperazinyl, or morpholinyl, which is unsubstituted or substituted with one or more substituents selected from fluoro, chloro, bromo, hydroxy, (CH₂)₁₋₂OH, oxo, methyl ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl;
R^{e} is a 5- or 6-membered heteroaromatic ring containing from 1 to 4 nitrogen atoms, 0 to 2 sulfur atoms, and at least 1 carbon atom; wherein the point of attachment of the ring is a carbon atom and at least one nitrogen or sulfur atom in the ring is adjacent to the point of attachment; wherein the ring is unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)0-1CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl;
R^{f} is X—NH(CH₂)₁₋₂Y, wherein X is a 5- or 6-membered monocyclic heterocycle which is saturated or unsaturated and which contains carbon atoms and from 1 to 3 nitrogen atoms and which is unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R^{b}; Y is pyrrolidinyl, piperidinyl, piperazinyl, or morpholinyl, which is unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), and (CH₂)₁₋₂N(R^{a})SO₂R^{b};
R^{g} is a 5- or 6-membered monocyclic heterocycle which is saturated or unsaturated and which contains one or more carbon atoms and from 1 to 4 nitrogen atoms, the heterocycle being unsubstituted or substituted with one or more substituents selected from fluoro, chloro, bromo, hydroxy, (CH₂)₁₋₂OH, oxo, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl; and
R^{h} is (i) C₃-C₆ cycloalkyl; (ii) phenyl; (iii) substituted phenyl, wherein each substituent on the substituted phenyl is independently fluoro, chloro, bromo, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, (CH₂)₁₋₂OH, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂N(R^{a})C(═O)R^{b}, SO₂R^{a}, (CH₂)₁₋₄SO₂R^{a}, SO₂N(R^{a})(R^{b}), (CH₂)₁₋₂SO₂N(R^{a})(R^{b}), (CH₂)₁₋₂N(R^{a})SO₂R^{b}, or (iv) a 5- or 6-membered monocyclic heterocycle which is saturated or unsaturated and which contains at least one carbon atom and from 1 to 4 heteroatoms selected from nitrogen, oxygen, and sulfur; where in the heterocycle is unsubstituted or substituted with one or more substituents selected from fluoro, chloro, bromo, hydroxy, methyl, ethyl, propyl, isopropyl, OCH₃, OCH₂CH₃, OCH₂CH₂CH₃, OCH(CH₃)₂, CF₃, CH₂CF₃, OCF₃, OCH₂CF₃, (CH₂)₁₋₃O(CH₂)₀₋₁CH₃, (CH₂)₁₋₃O(CH₂)₀₋₁CF₃, CO₂R^{a}, (CH₂)₁₋₂CO₂R^{a}, (CH₂)₁₋₂OH, N(R^{a})(R^{b}), (CH₂)₁₋₂N(R^{a})(R^{b}), C(═O)N(R^{a})(R^{b}), (CH₂)₁₋₂C(═O)N(R^{a})(R^{b}), N(R^{a})C(═O)R^{b}, (CH₂)₁₋₂(R^{a})(C═O)R^{b}, SO₂R^{a}, (CH₂)₁₋₂SO₂R^{a}, SO₂N(R^{a})(R^{b}), (CH₂)₁₋₂SO₂N(R^{a})(R^{b}), (CH₂)₁₋₂N(R^{a})SO₂R^{b}, phenyl, and benzyl;
or a pharmaceutically acceptable salt thereof.

6. The compound according to claim 5, or a tautomer thereof, wherein
B′ is pyridyl, pyrazinyl, pyrimidinyl, imidazolyl, triazolyl, tetrazolyl or thiazolyl
R^{c} is (i) phenyl or substituted phenyl or (ii) an unsubstituted or substituted fused bicyclic carbocycle selected from 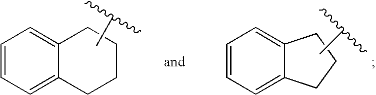
R^{d} is (i) an unsubstituted or substituted 5- or 6-membered monocyclic heterocycle selected from pyrazolyl, imidazolyl, pyrrolyl, pyrrolidinyl, pyridyl, piperidinyl, pyrazinyl, piperazinyl, pyridazinyl, pyrimidinyl, 1,2,4-triazolyl, 1,2,3-triazolyl, tetrazolyl, morpholinyl, tetrahydrothienyl, tetrahydrodioxothienyl, thiadiazinanyl dioxothiazinanyl, thiazinanyl, dioxothiazinanyl, thiazolidinyl, dioxothiazolidinyl, isothiazolidinyl, isodioxothiazolidinyl, thiazolyl, and isothiazolyl; (ii) an unsubstituted or substituted fused bicyclic heterocycle selected from 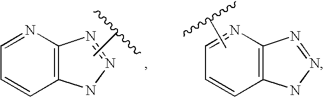 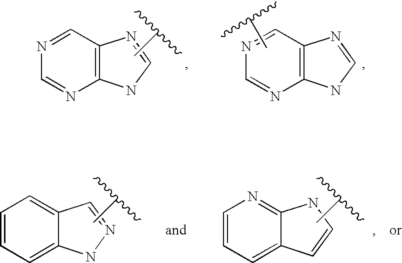
(iii) a monocyclic heterocycle selected from pyridyl, piperidinyl, pyrazinyl, piperazinyl, and pyrimidinyl, the heterocycle being substituted with spiro-C₁-C₂ alkylenedioxy or with one of unsubstituted or substituted piperidinyl, unsubstituted or substituted piperazinyl, or unsubstituted or substituted morpholinyl;
R^{e} is an unsubstituted or substituted heteroaromatic ring selected from pyridyl, pyrazinyl, and pyrimidinyl;
R^{f} is X—NH(CH₂)₁₋₂Y, wherein X is selected from unsubstituted or substituted pyridyl, unsubstituted or substituted pyrazinyl, and unsubstituted or substituted pyrimidinyl; and Y is unsubstituted or substituted pyrrolidinyl, unsubstituted or substituted piperidinyl, unsubstituted or substituted piperazinyl, or unsubstituted or substituted morpholinyl;
R^{g} is an unsubstituted or substituted monocyclic heterocycle selected from pyridyl, pyrrolyl, 1,2,3-triazolyl, 1,2,4-triazolyl, pyrazolyl, imidazolyl, tetrazolyl, piperidinyl, and piperazinyl; and
R^{h} is C₃-C₆ cycloalkyl, phenyl, substituted phenyl, or an unsubstituted or substituted monocyclic heterocycle selected from pyridyl, pyrrolyl, 1,2,3-triazolyl, 1,2,4-triazolyl, pyrazolyl, imidazolyl, tetrazolyl, piperidinyl, piperazinyl, and tetrahydrofuranyl;
or a pharmaceutically acceptable salt thereof.

7. The compound according to claim 6, or a tautomer thereof wherein B′ is pyridyl;
or a pharmaceutically acceptable salt thereof.

8. The compound according to claim 7, selected from the group consisting of
1-(3-Benzyl-5-pyrazin-2-ylmethylphenyl)-3-(4-methylpyridin-2-yl)-propane-1,3-dione;
1-(3-Benzyl-5-pyrazin-2-ylmethylphenyl)-3-(4-methylpyridin-2-yl)-propane-1,3-dione;
3-[3-(2-Chlorobenzyl)-5-pyridin-2-ylmethylphenyl]-1-(4-methylpyridin-2-yl)propane-1,3-dione;
3-[3-(3-Chlorobenzyl)-5-pyridin-2-ylmethylphenyl]-1-(4-methylpyridin-2-yl)propane-1,3-dione;
1-[3-Benzyl-5-(2-oxo-2H-pyridin-1-ylmethyl)phenyl]-3-(4-methylpyridin-2-yl)-propane-1,3dione;
1-[3-Benzyl-5-(2-oxo-pyridin-1-ylmethyl)phenyl]-3-(4-methylpyridin-2-yl)-propane-1,3dione;
1-[3-Benzyl-5-(4-methylpiperazin-1-ylmethyl)phenyl]-3-(4-methylpyridin-2-yl)-propane-1,3dione;
1-(3-Benzylphenyl)-3-(4-methylpyridin-2-yl)-propane-1,3-dione;
1-[(2,6-difluoro-benzyl)-phenyl]-3-(4methoxy-pyridin-2-yl)-propane-1,3-dione;
1-(3-Benzyl-phenyl)-3-(4-methoxy-pyridin-2-yl)-propane-1,3-dione;
1-[3-(2,6-Difluoro-benzyl)-phenyl]-3-(4-methyl-pyridin-2-yl)-propane-1,3-dione;
1-{3-Benzyl-5-[6-(2-morpholin-4-yl-ethylamino)-pyrazin-2-yl]-phenyl}-3-(4-methyl-pyridin-2-yl)-propane-1,3-dione;
1-[3-Benzyl-5-(6-methoxypyridin-2-yl)-phenyl]-3-(4-methyl-pyridin-2-yl)-propane-1,3-dione;)
1-[3-Benzyl-5-(6-morpholin-4-yl-pyrazin-2-yl)-phenyl]-3-(4-methyl-pyridin-2-yl)-propane-1,3-dione;
1-[3-Benzyl-5-(4-methyl-3,4,5,6-tetrahydro-2H-[1,2′]bipyrazinyl-6′-yl)-phenyl]-3-(4-methyl-pyridin-2yl)-propane-1,3-dione;
1-[5-(4-Fluoro-benzyl)-2,3-dimethoxy-phenyl]-3-(4-methyl-pyridin-2-yl)-propane-1,3-dione;
1-[2,3-Dimethoxy-5-(2-methyl-benzyl)-phenyl]-3-(4-methyl-pyridin-2-yl)-propane-1,3-dione;
1-(5-Benzyl-2-methoxy-3-morpholin-4-ylmethylphenyl)-3-(4-methyl-pyridin-2-yl)propane-1,3-dione;
1-(5-Benzyl-2-isopropoxy-3-pyrrolidin-1-ylmethyl-phenyl)-3-(4-methyl-pyridin-2-yl)propane-1,3-dione;
1-(5-Benzyl-2-isopropoxy-3-[1,2,4]triazol-1-ylmethylphenyl)-3-(4-methyl-pyridin-2-yl)propane-1,3-dione;
1-[5-Benzyl-2-(pyridin-2-yloxy)phenyl]-3-(4-methylpyridin-2-yl)propane-1,3-dione;
1-[3-Benzyl-5-(4-methylpiperazin-1-yl)phenyl]-3-(4-methylpyridin-2-yl)-propane-1,3-dione;
1-(3-Benzyl-5-[1,2,4]triazol-1-ylmethylphenyl)-3-(4-methylpyridin-2-yl)-propane-1,3-dione;
1-(3-Benzyl-5-imidazol-1-ylphenyl)-3-(4-methylpyridin-2yl)-propane-1,3-dione;
1-(3-Benzyl-5-[1,2,3]triazol-1-ylmethylphenyl)-3-(4-methylpyridin-yl)propane-1,3-dione;
1-(3-Benzyl-5-[1,2,3]triazol-1-ylmethylphenyl)-3-(6-chloropyridin-2-yl)-propane-1,3-dione;
1-(3-Benzyl-5-tetrazol-1-ylmethylphenyl)-3-(4-methylpyridin-2-yl)-propane-1,3-dione;
1-(3-Benzyl-5-[1,2,3]triazol-2-ylmethylphenyl)-3-(4-methylpyridin-2-yl)-propane-1,3-dione;
1-(3-Benzyl-5-tetrazol-2-ylmethylphenyl)-3-(4-methylpyridin-2-yl)-propane-1,3-dione;
1-(3-Benzyl-5-pyrrolo[2,3]pyridin-1-ylmethylphenyl)-3-(4-methylpyridin-2-yl)-propane-1,3-dione;
1-(3-Benzylphenyl)-3-(3-isopropoxypyridin-2-yl)-propane-1,3-dione;
1-(3-Benzylphenyl)-3-(3-propoxypyridin-2-yl)-propane-1,3-dione;
1-(3,5-Bis-pyrazol-1-ylmethyl-phenyl)-3-(4-methyl-pyridin-2-yl)-propane-1,3-dione;
1-(4-Methyl-pyridin-2-yl)-3-(3pyrazol-1-ylmethyl-phenyl)-propane-1,3-dione;
1-(4-Methyl-pyridin-2-yl)-3-(3-pyrrol-1-ylmethyl-phenyl)-propane-1,3-dione;
1-(4-Methyl-pyridin-2-yl)-3-(3-tetrazol-2-ylmethyl-phenyl)-propane1,3-dione;
1-(4-Methyl-pyridin-2-yl)-3-(3-[1,2,3]triazol-2-ylmethyl-phenyl)-propane-1,3-dione;
1-[3-(3-Methyl-pyrazol-1-ylmethyl)-phenyl]-3-(4-methyl-pyridin-2-yl)-propane-1,3-dione;
1-[3-(5-Methyl-pyrazol-1-ylmethyl)-phenyl]-3-(4-methyl-pyridin-2-yl)-propane-1,3-dione;
1-(4-Methyl-pyridin-2-yl)-3-(3-[1,2,3]triazol-2-ylmethyl-5-[1 2,3]triazol-1-ylmethylphenyl)-propane-1,3-dione;
1-(3,5-Bis-pyrrol-1-ylmethyl-phenyl)-3-(4-methyl-pyridin-2-yl)-propane-1,3-dione;
1-(3-Indazol-1-ylmethyl-phenyl)-3-(4-methyl-pyridin-2-yl)-propane-1,3-dione;
1-(4-methyl-pyridin-2-yl)-3-(3-pyrimidin-2-ylmethyl-phenyl)-propane-1,3-dione;
1-(3-Benzylphenyl)-3-(5-dimethylaminopyridin-2-yl)-propane1,3-dione;
1-(3-benzyl-5-pyrazin-2-yl-phenyl)-3-(4-methyl-pyridin-2-yl)-propane-1,3-dione;
1-(3-benzyl-5-pyrimidin-2-yl-phenyl)-3-(4-methyl-pyridin-2-yl)-propane-1,3-dione;
tautomers thereof;
and pharmaceutically acceptable salts thereof.

9. The compound according to claim 7, selected from the group consisting of
1-(3,5-bis-pyridin-2-ylmethylphenyl)-3-(4-methylpyridin-2-yl)propane-1,3-dione;
1-[3,5-bis-(2-methyl-2H-pyrazol-3-ylmethyl)phenyl]-3-(4-methylpyridin-2-yl)propane-1,3-dione;
1-(3-Pyridin-2-ylmethyl)phenyl-3-(4-methylpyridin-2-yl)propane-1,3-dione;
1-[3-(1,1-Dioxoisothiazolin-2-ylmethyl)-5-(pyridin-2-ylmethyl)phenyl]-3-(4-methylpyridin-2-yl)propane-1,3-dione;
1-[5-(2,6-Difluorobenzyl)-2,3-dimethoxyphenyl]-3-(4-methylpyridin-2-yl)propane-1,3-dione;
1-(5-benzyl-2-fluorophenyl)-3-(4-methylpyridin-2-yl)propane-1,3-dione;
1-(2-Methoxy-5-[1,2,3]triazol-2-ylmethylphenyl)-3-(4-methylpyridin-2-yl)propane-1,3-dione;
1-(3-benzyl-5-indazol-1-ylmethyl)phenyl-3-(4-methylpyridin-2-yl)propane-1,3-dione;
1-(3-benzyl-5-pyrazol-1-ylmethyl)phenyl-3-(4-methylpyridin-2-yl)propane-1,3-dione;
1-(3-benzyl-5-[1,2,3]triazolo[4,5,b]pyridin-1-ylmethyl)phenyl-3-(4-methylpyridin-2-yl)-propane-1,3-dione;
1-[3-benzyl-5-(3-methyl-2,6-dioxo-3,6-dihydro-2H-pyrimidin-1-ylmethyl)phenyl]-3-(4-methylpyridin-2-yl)propane-1,3-dione;
1-[3-benzyl-5-(2-oxo-1,2-dihydro-2H-pyrimidin-1-ylmethyl)phenyl]-3-(4-methylpyridin-2-yl)propane-1,3-dione;
1-(3-benzyl-5-purin-9-ylmethyl)phenyl-3-(4-methylpyridin-2-yl )propane-1,3-dione;
1-[3-benzyl-5-(1,1-dioxoisothiazolidin-2-ylmethyl)phenyl]-3-(4-methylpyridin-2-yl)-propane-1,3-dione;
1-[3-benzyl-5-(1,1-dioxothiazinan-2-ylmethyl)phenyl]-3-(4-methylpyridin-2-yl)-propane-1,3-dione;
1-[3-benzyl-5-(1,1-dioxo-[1,2,6]-thiadiazinan-2-ylmethyl)phenyl]-3-(4-methylpyridin-2-yl)propane-1,3-dione;
1-[3-benzyl-5-(2-oxo-2H-pyrimidin-1-ylmethyl)phenyl]-3-(pyridin-2-yl)propane-1,3-dione;
1-[3-benzyl-5-(1,1-dioxotetrahydrothiophen-2-ylmethyl)phenyl]-3-(4-methylpyridin-2-yl)propane-1,3-dione;
1-[3-benzyl-5-(1,1-dioxotetrahydrothiophen-2-ylmethyl)-2-isopropoxyphenyl]-3-(4-methylpyridin-2-yl)propane-1,3-dione;
1-[3-benzyl-5-(1,3-dimethyl-2,3,6,1-tertrahydro-2,6-dioxopurin-9-ylmethyl)phenyl]-3-(4-methylpyridin-2-yl)propane-1,3-dione;
1-[3-benzyl-5-(6-dimethylaminoylpurin-7-ylmethyl)phenyl]-3-(4-methylpyridin-2-yl)-propane-1,3-dione;
1-[3-benzyl-5-(4-methyl-5-thioxo-3-trifluoromethyl-4,5-dihydro-[1,24]-triazol-1-ylmethyl)phenyl]-3-(4-methylpyidin-2-yl)propane1,3-dione;
1-[3-benzyl-5-(3,7-dimethyl-3,7-dihydro-2,6-dioxopurin-1-ylmethyl)phenyl]-3-(4-methylpyridin-2-yl)propane-1,3-dione;
1-[3-benzyl-5-(2-oxo-2H-pyridin-1-ylmethyl)phenyl]3-(4-methylpyridin-2-methylpyridin-1,3-dione;
1-[3-benzyl-5-([1,2,3]triazolo[4,5-b]pyridinyl-2-ylmethyl)phenyl]-3-(4-methylpyridin-2-yl)propane-1,3-dione;
1-(3,5-bis-pyrazol-1-ylmethylphenyl)-3-pyridin-2-yl-propane-1,3-dione;
1-(4-Methylpyridin-2-yl)-3-(3-pyrazol-1-ylmethyl-5-[1,2,4]triazol-1-ylmethylphenyl)-propane-1,3-dione;
1-[3,5bis(3,5-dimethylpyrazol-1-ylmethyl)phenyl]-3-(4-methylpyridin-2-yl)-propane-1,3-dione;
1-(3-benzylphenyl)-3-(5-bromopyridin-2-yl)propane-1,3-dione;
1-(3-benzylphenyl)-3-(5-methoxypyridin-2-yl)propane-1,3-dione;
1-(3-benzylphenyl)-3-(4-imidazol-1-ylmethylpyridin-2-yl)propane-1,3-dione;
1-(3-benzylphenyl)-3-(4-(1,2,4-triazol-1-yl)methyl)pyridin-2-yl)propane-1,3-dione;
1-(3-benzylphenyl)-3-(4-(pyrazol-1-ylmethylpyridin-2-yl)propane-1,3-dione;
1-(3-benzylphenyl)-3-(4-(1,2,3,4-tetrazol-2-yl)methyl)pyridin-2-yl)propane-1,3-dione;
1-(3-benzyl-2-[1,2,3]-triazol-1-ylmethyl)phenyl)-3-(4-imidazol-1-ylmethylpyridin-2-yl)propane-1,3-dione;
1-(3-benzylphenyl)-3-(4-methoxymethylpyridin-2-yl)propane-1,3-dione;
1-(3-benzylphenyl)-3-(4-hydroxymethylpyridin-2-yl)propane-1,3-dione;
1-(3-benzylphenyl)-3-[4-(tetrahydrofuran-2-yl)-pyridin-2-yl]propane-1,3-dione;
1-(3,5-bis-pyrazol-1-ylmethyl-phenyl)-3-(4-[1,2,4]triazol-1-ylmethyl-pyridin-2-yl)-propane-1,3-dione;
1-(3,5-bis-pyridin-2-ylmethylphenyl)-3-(4-imidazol-1-ylmethyl-pyridin-2-yl)propane-1,3-dione;
tautomers thereof;
and pharmaceutically acceptable salts thereof.

10. The compound according to claim 7, selected from the group consisting of
1-(3,5-bis-pyrazol-1-ylmethylphenyl)-3-(4-methylpyridin-2-yl)propane-1,3-dione;
1-(3-benzyl-5-tetrazol-2-ylmethylphenyl)-3-(4-methylpyridin-2-yl)propane-1,3-dione;
1-(3-benzyl-5-[1,2,4]triazol-1-ylmethylphenyl)-3-(4-methylpyridin-2-yl)-propane-1,3-dione;
1-(3-benzyl-5-tetrazol-1-ylmethylphenyl)-3-(4-methylpyridin-2-yl)propane-1,3-dione;
1-(3-benzyl-5-[1,2,3]triazol-2-ylmethylphenyl)-3-(4-methylpyridin-2-yl)-propane-1,3-dione;
1-[3-benzyl-5-(4-methylpiperazin-1-ylmethyl)phenyl]-3-(4-methylpyridin-2-yl)propane-1,3-dione
1-(3-benzyl-5-[1,2,3]triazol-1-ylmethylphenyl)-3-(4-methylpyridin-2-yl)propane-1,3-dione;
1-(3-benzyl-5-pyrazin-2-ylmethylphenyl)-3-(4-methylpyridin-2-yl)propane-1,3-dione;
1-[3-benzyl-5-(2-oxopiperidin-1-ylmethyl)phenyl]-3-(4-methylpyridin-2-yl)propane-1,3-dione;
1-(3-Benzyl-5-[1,2,4]triazol-1-ylmethylphenyl)-3-(4-methylpyridin-2-yl)-propane-1,3-dione;
1-(3,5-bis-pyridin-2-ylmethylphenyl)-3-(4-methylpyridin-2-yl)propane-1,3-dione;
1-[3benzyl]-5-(3-methyl-2,6-dioxo-3,6-dihydro-2H-pyrimidin-1-ylmethyl)phenyl]-3-(4-methylpyridin-2-yl)propane-1,3-dione;
1-[3-benzyl-5-(2-oxo-1,2-dihydro-2H-pyrimidin-1-ylmethyl)phenyl]-3-(4-methylpyridin-2-yl)propane-1,3-dione;
1-(3-benzyl-5-([1,1-dioxoisothiazolidin-2-ylmethyl)phenyl]-3-(4-methylpyridin-2-yl)-propane-1,3-dione;
1-[3-benzyl-5-(1,1-dioxothiazinan-2-ylmethyl)phenyl]-3-(4-methylpyridin-2-yl)-propane-1,3-dione;
1-[3-benzyl-5-(1,1-dioxo-[1,2,6]-thiadiazinan-2-ylmethyl)phenyl]-3-(4-methylpyridin-2-yl)propane-1,3-dione;
tautomers thereof;
and pharmaceutically acceptable salts thereof.

11. The compound according to claim 6 or a tautomer thereof, wherein B is pyrazinyl, pyrimidinyl, imidazolyl, triazolyl, tetrazolyl, or thiazolyl; or a pharmaceutically acceptable salt thereof.

12. The compound according to claim 11, selected from the group consisting of
1-(3-benzylphenyl)-3-(1H-imidazol-2-yl)-propane-1,3-dione;
1-(3-benzylphenyl)-3-(1-benzyl-1H-imidazol-2-yl)propane-1,3-dione TFA salt;
1-(3-benzylphenyl)-3-(1H-imidazol-4-yl)propane-1,3-dione;
1-(3-benzylphenyl)-3-pyrazin-2-ylpropane-1,3-dione;
1-(3-benzylphenyl)-3-(2-methylthiazol-4-yl)-propane-1,3-dione;
1-[3-benzyl-5-(5-methylpyrazin-2-ylmethyl)phenyl]-3-(5-methylpyrazin-2-yl)-propane-1,3-dione;
1-(3-benzylphenyl)-3-(4H-[1,2,4]triazol-3-yl)propane-1,3-dione;
tautomers thereof;
and pharmaceutically acceptable salts thereof.

13. The compound according to claim 11, selected from the group consisting of
1-[3-(1,1-Dioxoisothiazolin-2-ylmethyl)-5-(pyridin-2-ylmethyl)phenyl]-3-(1-methyl-1H-imidazol-4-yl)propane-1,3-dione;
1-(3-benzylphenyl)-3-(1-N-methyl-imidazole-4-yl)propane-1,3-dione;
1-(3-benzylphenyl)-3-[1-N-(pyridin-4-yl)methylimidazole-4-yl]propane-1,3-dione;
1-(3-benzylphenyl)-3-[1-N-(pyridin-2-yl)methylimidazole-4-yl]propane-1,3-dione;
1-(3-benzylphenyl)-3-[1-N-(pyridin-3-yl)methylimidazole-4-yl]propane-1,3-dione;
1-(3-benzylphenyl)-3-{1-N-[(1-N-tert-butylcarbamyl)-piperidine-4-yl]methylimidazole-4-yl}propane-1,3-dione;
1-(3-benzylphenyl)-3[1-N-(piperidine-4-yl)methylimidazole-4-y]propane-1,3-dione;
1-(3-benzylphenyl)-3-{1-N-[(1-N-methanesulfonyl)piperidine-4-yl]methylimidazole-4-yl}propane-1,3-dione;
1-(3-benzylphenyl)-3-{1-N-[2-(1-N-tert-butylcarbamylpipeniazin-4-yl)ethyl]imidazole-4-yl}propane-1,3-dione;
1-(3-benzylphenyl)-3-{1-N-[2-(piperiazin-1-yl)ethyl]imidazole-4-yl}propane-1,3-dione;
1-(3-benzylphenyl)-3-{1-N-[2-(1-N-methansufonyl-piperazin-4-yl)ethyl]-imidazole-4-yl}propane-1,3-dione;
1-(3-benzylphenyl)-3-{1-N-[2-(1-N-benzylpiperiazin-4-yl)ethyl]imidazole-4-yl}propane-1,3-dione;
1-[3-benzyl-5-(6-oxo-6H-pyrimidin-1-ymethyl)phenyl]-3-(1-methylimidazole-4-yl)propane-1,3-dione;
1-[3-benzyl-5-(1,1-dioxoisothiazolidin-2-ylmethyl)phenyl]-3-(1-methylimidazole-4-yl)propane-1,3-dione;
1-(3-benzylphenyl-pyrimidin-2yl-propane-1,3-dione;
1-(3-benzylphenyl)-3-(4-methylpyrimidin-2-yl)propane-1,3-dione;
1-(3,5-bis-pyrazol-1-ylmethylphenyl)-3-pyrimidin-2yl-propane-1,3-dione;
1-(3,5-bis-pyrazol-1-ylmethylphenyl)-3-(4-methylpyrimidin-2-yl)propane-1,3-dione;
1-(3,5-bis-pyrazol-1-ylmethyl-phenyl)-3-(1H-imidazol-2-yl)propane-1,3-dione;
1-(3,5-bis-pyrazol-1-ylmethyl-phenyl)-3-(1-methyl-1H-imidazol-4-yl)propane-1,3-dione;
1{3-benzyl-5-[(1,1-dioxido-1,2-thiazinan-2-yl)methyl]phenyl}-3-(1,3-thiazol-2-yl)-1,3-propanedione;
1-{3-benzyl-5-[(1,1-dioxido-2-isothiazolidinyl)methyl]phenyl)}-3-(1,3-thiazol-2-yl)-1,3-propanedione;
1-{3-benzyl-5-[(6-oxo-1(6H)-pyrimidinyl)methyl]phenyl}-3-(1,3-thiazol-2-yl)-1,3-propanedione;
tautomers thereof,
and pharmaceutically acceptable salts thereof.

14. The compound according to claim 11, selected from the group consisting of
1-(3-benzylphenyl)-3-(1H-imidazol-2-yl)propane-1,3-dione;
1-(3-benzylphenyl)-3-(1H-imidazol-4-yl)propane-1,3-dione;
1-[3-benzyl-5-(1,1-dioxoisothiazolidin-2-ylmethyl)phenyl]-3-(1-methylimidazole-4-yl)propane-1,3-dione;
1-{3-benzyl-5-[(6-oxo-1(6H)-pyrimidinyl)methyl]phenyl}-3-(1,3-thiazol-2-yl)-1,3-propanedione;
tautomers thereof;
and pharmaceutically acceptable salts thereof.

15. The compound according to claim 3, or a tautomer thereof, wherein
B is an 8- to 10-membered fused bicyclic heterocycle containing from 1 to 4 nitrogen atoms, 0 to 2 sulfur atoms, and carbon atoms, wherein the ring of the heterocycle attached to the central dione moiety is a 5- or 6-membered heteroaromatic ring containing at least one nitrogen or sulfur atom and the other ring of the heterocycle is a saturated or unsaturated ring;
or a pharmaceutically acceptable salt thereof.

16. The compound according to claim 15, or a tautomer thereof, wherein the compound is
1-(3-benzylphenyl)-3-(5,6,7,8-tetrahydro-[1,8]naphthyridin-2-yl)-propane-1,3-dione;
or a pharmaceutically acceptable salt thereof.

17. The compound according to claim 1, or a tautomer thereof, wherein
A is an 8- to 10-membered fused bicyclic heterocycle containing carbon atoms and from 1 to 3 heteroatoms selected from nitrogen, oxygen and sulfur; wherein the ring of the heterocycle attached to the central dione moiety is a benzene ring, and the other ring of the heterocycle is a saturated or unsaturated heteroatom-containing ring;
or a pharmaceutically acceptable salt thereof.

18. The compound according to claim 17, or a tautomer thereof, wherein the compound is
1-(6-benzyl-3-oxo-3,4-dihydro-2H-benzo[1,4]oxazin-8-yl)-3-(4-methylpyridin-2-yl)propane-1,3-dione;
or a pharmaceutically acceptable salt thereof.

19. The compound according to claim 1, or a tautomer thereof; wherein
A is a 5- or 6-membered heteroaromatic ring containing 0, 1 or 2 nitrogen atoms and 0 or 1 sulfur atoms;
or a pharmaceutically acceptable salt thereof.

20. The compound according to claim 19, or a tautomer thereof, wherein
B is (i) a 5- or 6-membered heteroaromatic ring containing from 1 to 4 nitrogen atoms, 0 or 1 sulfur atoms, and at least 1 carbon atom, or (ii) an 8- to 10-membered fused bicyclic heterocycle containing from 1 to 3 nitrogen atoms and carbon atoms, wherein the ring of the heterocycle attached to the central dione moiety is a 5- or 6-membered heteroaromatic ring containing at least one nitrogen atom and the other ring of the heterocycle is a saturated or unsaturated ring; wherein B is attached to the central dione moiety via a carbon atom and at least once nitrogen or sulfur atom in B is adjacent to the point of attachment;
or a pharmaceutically acceptable salt thereof.

21. The compound according to claim 20, or a tautomer thereof wherein
A is pyrrolyl, thienyl, or pyridyl; and
B is (i) a heteroaromatic ring selected from pyridyl, pyrazinyl, pyrimidinyl, imidazolyl, triazolyl, tetrazolyl, and thiazolyl, or (ii), a fused bicyclic heterocycle selected from 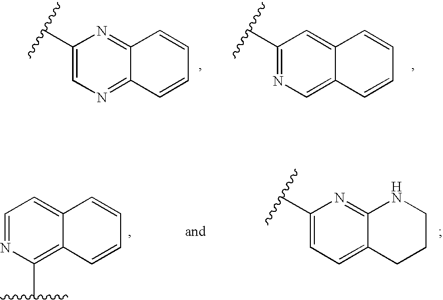
or a pharmaceutically acceptable salt thereof.

22. The compound according to claim 21, selected from the group consisting of
1-[1-(4-fluorobenzyl)-1H-pyrrol-2-yl]-3-pyrimidin-4-yl-propan-1,3-dione;
1-[1-(4-fluorobenzyl)-1H-pyrrol-2-yl]-3-thiazol-2-yl-propan-1,3-dione;
1-[1-(4-Fluorobenzyl)-1H-pyrrol-2-yl]-3-(4-methylpyridin-2-yl)propane-1,3-dione;
1-(1-Benzyl-1H-imidazol-2-yl)-3-[1-(4-fluorobenzyl)-1H-pyrrol-2-yl)propan-1,3-dione;
1-[1-(4-Fluorobenzyl)-1H-pyrrol-3-yl]-3-pyridin-2ylpropan-1,3-dione;
1-(1-(4-Fluorobenzyl)-1H-imidazol-2-yl)-3-[1-(4-fluorobenzyl)-1H-pyrrol-2-yl)propan-1,3-dione;
1-[1-(4-Fluorobenzyl)-1H-pyrrol-2-yl]-3-(4H-[1,2,4]triazol-3-yl-propan-1,3-dione;
1-[1-(4-Fluorobenzyl)-4-(2-oxo-2H-pyridin-1-yl)-1H-pyrrol-2-yl]-3-pyridin-2-yl-propan-1,3-dione;
1-(1H-Imidazol-2-yl)-3-(5-phenethylthiophen-2-yl)propane-1,3-dione;
1-(5-Benzyl-thiophen-2-yl)-3-pyridin-2-yl-propane-1,3-dione;
1-(5-Benzylthiophen-2yl)-3-(4-methyl-pyridin-2-yl)propane-1,3-dione;
1-[5-(3-Chlorobenzyl)thiophen-2-yl]-3-pyridin-2-yl-propane-1,3-dione;
1-[5-(4-Fluorobenzyloxy)thiophen-2-yl]-3-(4-methylpyridin-2-yl)propane-1,3-dione;
1-[5-(4-Fluorobenzyloxy)thiophen-2-yl]-3-pyridin-2-yl-propane-1,3-dione;
tautomers thereof;
and pharmaceutically acceptable salts thereof.

23. A pharmaceutical composition comprising the compound according to claim 1 and a pharmaceutically acceptable carrier.

24. The pharmaceutical composition according to claim 23, wherein the composition further comprises at least one anti viral selected from the group consisting of HIV pro tease inhibitors, non-nucleoside HIV reverse transcriptase inhibitors, and nucleoside HIV reverse transcriptase inhibitors.

25. A method of inhibiting HIV integrase in a subject in need thereof which comprises administering to the subject a therapeutically effective amount of the compound according to claim 1.

26. A method for preventing or treating infection by HIV or treating AIDS in a subject in need thereof which comprises administering to the subject a therapeutically effective amount of the compound according to claim 1.

27. The method according to claim 26, wherein the compound is administered in combination with a therapeutically effective amount of at least one antiviral selected from the group consisting of HIV protease inhibitors, non-nucleoside HIV reverse transcriptase inhibitors, and nucleoside HIV reverse transcriptase inhibitors

28. A method of inhibiting HIV integrase in a subject in need thereof which comprises administering to the subject a therapeutically effective amount of the composition according to claim 23.

29. A method for preventing or treating infection by HIV or for treating AIDS in a subject in need thereof which comprises administering to the subject a therapeutically effective amount of the composition according to claim 23.

30. A method for preventing or treating HIV infection or for treating AIDS in a subject in need thereof which comprises administering to the subject a therapeutically effective amount of the composition according to claim 24.
